(19) United States
(12) Patent Application Publication
(10) Pub. No.: US 2026/0338801 A1
(43) Pub. Date: Oct. 29, 2026

(54) METHODS FOR EARLY DIAGNOSIS AND TREATMENT OF OPEN NEURAL TUBE DEFECTS

(71) Applicant: Temple University Of The Commonwealth System of Higher Education, Philadelphia, PA (US)

(72) Inventor: BARBARA KRYNSKA

(21) Appl. No.: 19/103,706

(22) PCT Filed: Aug. 16, 2023
(86) PCT No.: PCT/US2023/072288

§ 371 Date: Feb. 13, 2025

Related U.S. Application Data
(60) Provisional application No. 63/371,580, filed on Aug. 16, 2022.

(51) Int. Cl. G01N 33/68

(52) U.S. Cl. CPC G01N 33/6896; G01N 2800/28; G01N 2800/52

(57) ABSTRACT

The present invention relates to the discovery that various proteins are enriched in the amniotic fluid of fetuses with open neural tube defects (NTDs). The invention includes methods for the diagnosis of open NTD based upon the levels and/or differential patterns of neurocan and phosphacan in a sample of the amniotic fluid or other bodily fluid of a subject.

### CROSS REFERENCE TO RELATED APPLICATIONS

This application is entitled to priority under 35 U.S.C. § 119 (e) to U.S. Provisional Application Ser. No. 63/371,580, filed Aug. 16, 2022 the contents of which are incorporated by reference herein in their entirety.

### STATEMENT REGARDING FEDERALLY SPONSORED RESEARCH OR DEVELOPMENT

This invention was made with government support under R01NS109064 awarded by the National Institutes of Health (NIH). The government has certain rights in the invention.

### REFERENCE TO A SEQUENCE LISTING

The present application hereby incorporates by reference the entire contents of the XML file named “206017-0221-00WO_SequenceListing.xml” in XML format, which was created on Aug. 16, 2023, and is 295,220 bytes in size.

### BACKGROUND OF THE INVENTION

Myelomeningocele (MMC) is a devastating congenital open neural tube defect (NTD) that affects approximately three per 10,000 live births per year in the United States and is clinically very important (Copp, A. J., et al., 2003, Nature Reviews Genetics, 4:784-793; Parker, S. E., et al., 2010, Birth Defects Research A: Clinical and Molecular Teratology, 88:10008-1016; Canfield, M. A., et al., 2014, American Journal of Public Health, 104: e14-23). The presentation is commonly in the lumbosacral region, and the defect is characterized by protrusion of the malformed spinal cord and meninges through a pathological opening in the overlying vertebrae and skin, leaving the spinal cord exposed to the intrauterine environment (Kaufman, B. A., 2004, Pediatiric Clinics of North America, 51:389-419). The underlying defect leads to prenatal injury to the exposed spinal cord and a spectrum of associated abnormalities resulting in life-long disability including leg paralysis, sensory loss, bowel and bladder dysfunctions, skeletal deformations, Arnold-Chiari type II malformation, hindbrain herniation and development of hydrocephalus (Hunt, G. M., 1990, Developmental Medicine & Child Neurology, 32:108-118; Dias, M. S. and McLone, D. G., 1993, Neurosurgery Clinics of North America, 4:715-726; Hunt, G. M. and Poulton, A., 1995, BMJ, 37:19-29; Tomlinson, P. and Sugarman, I. D., 1995, BMJ, 311:286-287; Hunt, G. M. and Oakeshott, P., 2003, BMJ, 326:1365-6). Affected individuals have profoundly diminished quality of life, often requiring life-long support and institutional care (Bowman, M. F., et al., 2001, Pediatric Neurosurgery, 34:114-120; Sandler, A. D., 2010, BMJ, 311:286-287).

The progressive nature of MMC over the course of gestation with the life-long clinical impact points to the importance of early prenatal diagnosis, which provides an opportunity for management decisions. Currently, a measurement of alpha fetoprotein (AFP) levels and/or fetal imaging is typically used for prenatal diagnosis of open NTDs. While elevated levels of AFP in maternal serum and amniotic fluid (AF) are used for screening and diagnosis of open NTDs, respectively, the specificity and sensitivity of open NTD detection by AFP is limited (Palomaki, G. E, et al., 2020, Genetics in Medicine, 22:462-475; Norem, C. T., et al., 2005, Obstetrics & Gynecology, 106:747-752; Rose, N. & Mennuti, M. T., 2009, Global library of women's medicines; Dashe, J. S., et al., 2006, American Journal of Obstetrics & Gynecology, 195:1623-1628). Therefore, there is a need for the development of biomarkers enabling early and accurate prenatal diagnosis of open NTDs. The present invention addresses this long needed but unmet requirement.

### SUMMARY OF THE INVENTION

The present invention relates to a method of diagnosing an open Neural Tube Defect (NTD) in a human fetus.

In various embodiments, the invention relates to a method of diagnosing open NTD comprising the steps of obtaining a sample of amniotic fluid and/or other bodily fluids from a fetus or an individual carrying the fetus; the detecting of the levels or differential patterns of neurocan and/or RPTPβ/phosphacan and comparing the levels and/or differential patterns of neurocan and/or RPTPβ/phosphacan with that of a normal sample.

In one embodiment, the detecting of the levels and/or differential pattern of neurocan and/or RPTPβ/phosphacan further comprises the step of treating the biological sample with a chondroitinase or any enzyme able to remove and/or modify glycosaminoglycan (GAG) component.

In one embodiment, the detecting of the levels and/or differential pattern of neurocan and/or RPTPβ/phosphacan does not include the step of removal and/or modification of GAG component.

In one embodiment, the detecting of the levels and/or differential pattern of neurocan and/or RPTPβ/phosphacan further comprises detecting the levels and/or differential pattern of at least one member chosen from the group consisting of the glycosaminoglycan (GAG)-containing and/or GAG-lacking neurocan proteins (encoding gene ID number: 1463, neurocan, SEQ ID NO:1), a full length neurocan protein, the proteolytically cleaved N-terminal neurocan fragment, the proteolytically cleaved C-terminal neurocan fragment, and/or other products of their fragmentation, the GAG-containing and/or GAG lacking RPTPβ/phosphacan protein isoforms (encoding gene ID number: 5803, protein tyrosine phosphatase receptor type Z1, also known as RPTPβ, phosphacan, SEQ ID NO:2), full-length RPTPβ/phosphacan protein isoforms and/or products of their fragmentation.

In one embodiment, the detecting of the levels and/or differential pattern of neurocan and/or RPTPβ/phosphacan involves the use of an antibody to at least one molecular form of neurocan and/or RPTPβ/phosphacan.

In one embodiment, the detecting of the levels and/or differential pattern of neurocan and/or RPTPβ/phosphacan is performed without the use of an antibody.

In one embodiment, the detecting of the levels and/or differential pattern of neurocan and/or RPTPβ/phosphacan is accomplished via Western blotting and immunoblotting.

In one embodiment, the detecting of the levels of neurocan and/or RPTPβ/phosphacan is accomplished by employing an ELISA assay.

In one embodiment, the detecting of the levels and/or differential pattern of neurocan and/or RPTPβ/phosphacan is accomplished by employing any biological or chemical assay that specifically detects at least one molecular form of neurocan and/or RPTPβ/phosphacan or any component of these molecules.

### BRIEF DESCRIPTION OF THE DRAWINGS

The following detailed description of various embodiments of the invention will be better understood when read in conjunction with the appended drawings. For the purpose of illustrating the invention, there are shown in the drawings illustrative embodiments. It should be understood, however, that the invention is not limited to the precise arrangements and instrumentalities of the embodiments shown in the drawings.

FIG. 1, comprising FIG. 1A through FIG. 1C, depicts retinoic acid-induced myelomeningocele (MMC) in fetal rats and the experimental strategy employed. FIG. 1A depicts a representative external view of RA-induced MMC defect in the lumbar region of a fetal rat at embryonic day 21 (E21). The arrow indicates the beginning of the exposed spinal cord. FIG. 1B depicts representative imaging of a hematoxylin and eosin (H&E) stained cross section from the MMC defect in a fetal rat at E21, demonstrating the malformed spinal cord and failed development of overlying structures. Scale bar indicates 100 μm. FIG. 1C depicts a schematic representation of an experimental strategy employed.

FIG. 2, comprising FIG. 2A through FIG. 2F, depicts schematic illustration of neurocan and RPTPβ/phosphacan and the representative detection of neurocan and phosphacan in the amniotic fluid of MMC fetuses. FIG. 2A depicts schematic representation of the domain organization of neurocan and its major proteolytic fragments, N-terminal fragment, and C-terminal fragment, and a long splice form of RPTPβ and its extracellular variant, phosphacan. Domains recognized by IF6 and 650.24 or 3F8 antibodies are indicated. FIG. 2B depicts a representative Western blot performed with equal volumes of amniotic fluid samples pooled from three randomly selected MMC fetuses or age-matched normal fetuses collected at E14, E16, E18, and E21 after being digested with ChABC. The western blot analysis depicted was performed with 1F6 antibody, illustrating detection of full-length neurocan core protein and the proteolytically cleaved N-terminal fragment (245 kDa and 130 kDa), respectively and 650.24 antibody, illustrating detection of full-length neurocan core protein and the proteolytically cleaved C-terminal fragment (245 kDa and 150 kDa), respectively. Analogous Western blot analysis with 3F8 antibodies demonstrates detection of phosphacan core protein (~400 kDa) in the AF of MMC fetuses. In lane SC, MMC spinal cord lysates were loaded on the gel as positive controls. FIG. 2C depicts quantitative analysis of total levels of neurocan proteins in the AF of MMC fetuses at all embryonic ages examined using 1F6 antibody as depicted in FIG. 2B. FIG. 2D depicts quantitative analysis of total levels of neurocan proteins in the AF of MMC fetuses and age-matched normal controls examined using 650.24 antibody as depicted in FIG. 2B. FIG. 2E depicts representative quantitative analysis of total levels of phosphacan protein in the AF of MMC fetuses and age-matched normal controls examined using 3F8 antibody as depicted in FIG. 2B. All graphs represent the change in arbitrary fluorescence units (AFU) at each time point. Data are presented as mean±SD, of 15 randomly selected AF samples per group. *, p<0.05; **, p<0.01; ***, p<0.001, ****, p<0.0001. FIG. 2F depicts a representative western blot of neurocan and phosphacan illustrating their detection in all AF samples from individual E21 fetuses with MMC defect, but very weak or no detection in the AF of fetuses with no MMC defect after RA exposure or in normal controls. Data represents 6 AF samples per group.

FIG. 3, comprising FIG. 3A through FIG. 3B, depicts extractions of phosphacan and neurocan from MMC spinal cord tissues. FIG. 3A depicts a representative Western blot analysis of the first (extracted without detergent) and second (extracted with detergent) sequential extracts of spinal cords isolated from MMC fetuses at E14, E16, E18, and E21 that were ChABC-digested and subjected to Western blot analysis using 1F6, 650.24, or 3F8 antibodies, demonstrating the vast majority of neurocan and phosphacan detected in the first extract at all gestational ages. FIG. 3B depicts a representative Western blot analysis of AF samples from E21 MMC fetuses, AF samples from E21 normal controls, and AF samples from normal E21 controls that were incubated with spinal cords isolated from E21 MMC fetuses and then subjected to Western blot analysis using IF6, 650.24, or 3F8 antibodies, demonstrating their release from MMC spinal cord tissue into the AF. Data represent spinal cord extracts from at least two independent sets of spinal cord tissue samples per group.

FIG. 4, comprising FIG. 4A through FIG. 4C, depicts representative images of neurocan and phosphacan expression in MMC spinal cord tissues and its immunohistochemical characterization. FIG. 4A depicts representative micrographs illustrating localization of RPTPβ/phosphacan (white) and Neurocan (yellow) using RNAscope in MMC spinal cords examined at E14 (left panel) and E21 (right panel). Cell nuclei were fluorescently stained with DAPI (blue). FIG. 4B depicts the type of cells that synthetize RPTPβ/phosphacan and Neurocan identified by coimmunostaining for glutamine synthetase (GS; green), a marker of astrocytic cells; MAP2 (magenta), a marker of neurons; and RNAscope for RPTPβ/phosphacan (white); or Neurocan (yellow) in spinal cord sections from MMC defects examined at E21. FIG. 4C depicts representative micrographs illustrating extracellular distribution pattern of phosphacan and neurocan in the immunostained spinal cord sections obtained through the center of MMC defect examined at E21. Cell nuclei were fluorescently stained with DAPI (blue). Scale bar represents 100 μm. Data represent at least three sections from three fetuses per group.

### DETAILED DESCRIPTION

The present invention is based in part on the discovery that elevated levels of neurocan and/or RPTPβ/phosphacan is an effective predictor of an open NTD. Thus, the present invention relates to methods relating to biomarkers (e.g., one or more chondroitin sulfate proteoglycans (CSPGs), neurocan and/or RPTPβ/phosphacan) that can be used for identifying and diagnosing open NTD in human fetuses. CSPGs, such as neurocan and RPTPβ/phosphacan are molecules consisting of a protein core with the attached glycosaminoglycan (GAG) component. For identification of neurocan and/or RPTPβ/phosphacan, biological sample obtained from the subject can be analyzed before and after GAG removal. In some embodiments, the diagnosis is accomplished by comparison of levels and/or differential patterns of GAG-containing and/or GAG-lacking CSPGs. In some embodiments the CSPGs are at least one selected from the group consisting of neurocan, RPTPβ/phosphacan, brevican, aggrecan, versican. In various embodiments, the diagnosis is accomplished by comparison of levels and/or differential patterns of GAG-containing and/or GAG-lacking neurocan and/or RPTPβ/phosphacan proteins in a sample of amniotic fluid or other bodily fluids obtained from a fetus or an individual carrying the fetus. In one embodiment, the detecting of the levels of neurocan and/or RPTPβ/phosphacan comprises detection of total GAG-containing and/or GAG lacking neurocan and/or RPTPβ/phosphacan proteins. In one embodiment, detecting of the levels and/or differential pattern of neurocan and/or RPTPβ/phosphacan comprises detection of individual GAG-containing and/or GAG lacking full-length neurocan and/or full length RPTPβ/phosphacan protein isoforms, the proteolytically cleaved N-terminal neurocan fragment, the proteolytically cleaved C-terminal neurocan fragment, and/or any other neurocan, and/or RPTPβ/phosphacan fragmentation products, or any combination of thereof. In some embodiment, detecting localization of neurocan and/or RPTPβ/phosphacan comprises detection of colocalization of neurocan and/or RPTPβ/phosphacan with markers of astrocytic cells and/or neurons and/or detection of the extracellular distribution patterns of neurocan and/or RPTPβ/phosphacan.

In one embodiment, the step of analyzing the biological sample further comprises a step of treating the biological sample with chondroitinase ABC or any enzyme able to remove and/or modify GAG component. In one embodiment, the analysis of the biological sample does not include the step of removing and/or modifying GAG component. In one embodiment, the detecting of the levels and/or differential pattern of neurocan and/or RPTPβ/phosphacan involves the use of an antibody to at least one molecular form of neurocan and/or RPTPβ/phosphacan. In one embodiment, the detecting of the levels and/or differential pattern of neurocan and/or RPTPβ/phosphacan is performed without the use of an antibody. In one embodiment, the diagnosis involves the use of Western blotting and immunoblotting. In one embodiment, the diagnosis involves employing an ELISA assay, or any other chemical or biological assay e.g., HPLC that detects at least one molecular form of neurocan and/or RPTPβ/phosphacan, their proteolytically cleaved fragments, and/or other fragmentation products. In one embodiment, the detecting of the colocalization or extracellular distribution pattern of neurocan and/or RPTPβ/phosphacan comprises RNAscope and/or coimmunostaining.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of +20%, +10%, +5%, +1%, or +0.1% from the specified value, as such variations are appropriate to perform the disclosed methods.

The term “antibody,” as used herein, refers to an immunoglobulin molecule which is able to specifically bind to a specific epitope of an antigen. Antibodies can be intact immunoglobulins derived from natural sources, or from recombinant sources and can be immunoreactive portions of intact immunoglobulins. The antibodies in the present invention may exist in a variety of forms including, for example, polyclonal antibodies, monoclonal antibodies, intracellular antibodies (“intrabodies”), Fv, Fab, Fab′, F (ab) 2 and F(ab′) 2, as well as single chain antibodies (scFv), heavy chain antibodies, such as camelid antibodies, and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, New York; Houston et al., 1988, Proc. Natl. Acad. Sci. USA 85:5879-5883; Bird et al., 1988, Science 242:423-426).

By the term “synthetic antibody” as used herein, is meant an antibody which is generated using recombinant DNA technology, such as, for example, an antibody expressed by a bacteriophage. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

As used herein, the term “heavy chain antibody” or “heavy chain antibodies” comprises immunoglobulin molecules derived from camelid species, either by immunization with a peptide and subsequent isolation of sera, or by the cloning and expression of nucleic acid sequences encoding such antibodies. The term “heavy chain antibody” or “heavy chain antibodies” further encompasses immunoglobulin molecules isolated from a subject with heavy chain disease, or prepared by the cloning and expression of VH (variable heavy chain immunoglobulin) genes from a subject.

A “chimeric antibody” refers to a type of engineered antibody which contains a naturally-occurring variable region (light chain and heavy chains) derived from a donor antibody in association with light and heavy chain constant regions derived from an acceptor antibody.

A “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity (see, e.g., 1989, Queen et al., Proc. Natl. Acad Sci USA, 86:10029-10032; 1991, Hodgson et al., Bio/Technology, 9:421) A suitable human acceptor antibody may be one selected from a conventional database, e.g., the KABAT database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. The prior art describes several ways of producing such humanized antibodies (see for example EP-A-0239400 and 20 EP-A-054951).

The term “donor antibody” refers to an antibody (monoclonal, and/or recombinant) which contributes the amino acid sequences of its variable regions, CDRs, or other functional fragments or analogs thereof to a first immunoglobulin partner, so as to provide the altered immunoglobulin coding region and resulting expressed altered antibody with the antigenic specificity and neutralizing activity characteristic of the donor antibody.

The term “acceptor antibody” refers to an antibody (monoclonal and/or recombinant) heterologous to the donor antibody, which contributes all (or any portion, but in some embodiments all) of the amino acid sequences encoding its heavy and/or light chain framework regions and/or its heavy and/or light chain constant regions to the first immunoglobulin partner. In certain embodiments a human antibody is the acceptor antibody.

The term “assessing” includes any form of measurement, and includes determining if an element is present or not. The terms “determining,” “measuring,” “evaluating,” “assessing” and “assaying” are used interchangeably and may include quantitative and/or qualitative determinations. Assessing may be relative or absolute. “Assessing binding” includes determining the amount of binding, and/or determining whether binding has occurred (i.e., whether binding is present or absent). “Assessing activity” includes determining the amount of activity, and/or determining whether an activity has occurred (i.e., whether an activity is present or absent).

The term “binding” refers to a direct association between at least two molecules, due to, for example, covalent, electrostatic, hydrophobic, ionic and/or hydrogen-bond interactions.

The phrase “biological sample”, “sample”, or “specimen” as used herein, is intended to include any sample comprising a cell, a tissue, or a bodily fluid in which expression of a nucleic acid or polypeptide can be detected. The biological sample may contain any biological material suitable for detecting the desired biomarkers, and may comprise cellular and/or non-cellular material obtained from the individual. Examples of such biological samples include but are not limited to blood, lymph, bone marrow, biopsies and smears. Samples that are liquid in nature are 5 referred to herein as “bodily fluids.” Biological samples may be obtained from a patient by a variety of techniques including, for example, by scraping or swabbing an area or by using a needle to obtain bodily fluids. Methods for collecting various body samples are well known in the art.

As used herein, the terms “diagnosis” and “diagnosing” refer to the determination of the presence of a disease or disorder. In various embodiments of the present invention, methods for making a diagnosis are provided which permit determination of the presence of a myelomeningocele.

The term “epitope” as used herein refers to the specific group of atoms on an antigen molecule to which a specific antibody binds, causing an immune response.

As used herein, an “immunoassay” refers to any binding assay that uses an antibody capable of binding specifically to a target molecule to detect and quantify the target molecule.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “label” when used herein refers to a detectable compound or composition that is conjugated directly or indirectly to a probe to generate a “labeled” probe. The label may be detectable by itself (e.g., radioisotope labels or fluorescent labels) or, in the case of an enzymatic label, may catalyze chemical alteration of a substrate compound or composition that is detectable (e.g., avidin-biotin). In some instances, primers can be labeled to detect a PCR product.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

In some instances, the terms “specific binding” or “specifically binding”, can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

“Measuring” or “measurement,” or alternatively “detecting” or “detection,” means assessing the presence, absence, quantity or amount (which can be an effective amount) of either a given substance within a sample, including the derivation of qualitative or quantitative concentration levels of such substances, or otherwise evaluating the values or categorization of the substance or the sample.

A “nucleic acid” refers to a polynucleotide and includes poly-ribonucleotides and poly-deoxyribonucleotides. Nucleic acids according to the present invention may include any polymer or oligomer of pyrimidine and purine bases, preferably cytosine, thymine, and uracil, and adenine and guanine, respectively. (See Albert L. Lehninger, Principles of Biochemistry, at 793-800 (Worth Pub. 1982) which is herein incorporated in its entirety for all purposes). Indeed, the present invention contemplates any deoxyribonucleotide, ribonucleotide or peptide nucleic acid component, and any chemical variants thereof, such as methylated, hydroxymethylated or glycosylated forms of these bases, and the like. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in singlestranded or double-stranded form, including homoduplex, heteroduplex, and hybrid states.

The terms “patient,” “subject,” “individual,” and the like are used interchangeably herein, and refer to any animal, or cells thereof whether in vitro or in situ, amenable to the methods described herein. In certain non-limiting embodiments, the patient, subject, or individual is a human.

To “treat” a disease as the term is used herein, means to reduce the frequency or severity of at least one sign or symptom of a disease or disorder experienced by a subject.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention is based, in part, on the discovery that various proteins are elevated in the amniotic fluid of fetuses with an open NTD. Accordingly, in various embodiments the invention is directed towards diagnosing open NTD by detecting the levels of neurocan and/or RPTPβ/phosphacan in amniotic fluid or other bodily fluids.

In some embodiments, the diagnosis is accomplished by comparison of levels and/or differential patterns of GAG-containing and/or GAG-lacking CSPGs. In some embodiments the CSPGs are at least one selected from the group consisting of neurocan, RPTPβ/phosphacan, brevican, aggrecan, versican.

In certain embodiments, the invention is directed towards the detection of the levels of total GAG-containing and/or GAG-lacking neurocan proteins.

In certain embodiments, the invention is directed towards the detection of the levels and/or differential patterns of individual GAG-containing and/or GAG-lacking full length neurocan protein, the proteolytically cleaved N-terminal neurocan fragment, the proteolytically cleaved C-terminal neurocan fragment, and/or other products of neurocan fragmentation, or any combination of thereof.

In certain embodiments, the invention is directed towards the detection of the levels of total GAG-containing and/or GAG-lacking RPTPβ/phosphacan proteins.

In certain embodiments, the invention is directed towards the detection of the levels and/or differential patterns of individual GAG-containing and/or GAG-lacking full length RPTPβ/phosphacan proteins, and/or products of RPTPβ/phosphacan fragmentation, or any combination of thereof.

### Methods of Diagnosis

The present invention provides methods for diagnosing open NTD in a subject in need thereof. In one aspect, the present invention also provides methods for distinguishing a subject with open NTD from a subject without open NTD.

In various embodiments, the method comprises obtaining a biological sample from a test subject. In one embodiment, the method comprises analyzing the biological sample with an assay that specifically detects at least one molecular form of one neurocan and/or RPTPβ/phosphacan. In some embodiments, the method comprises detecting the level of at least one molecular form of neurocan and/or RPTPβ/phosphacan in the biological sample of the subject. In various embodiments, the method comprises comparing the level of at least one molecular form of neurocan and/or RPTPβ/phosphacan to a comparator. In some embodiments, the method comprises determining that the subject has open NTD.

In one embodiment, the method comprises at least one molecular form of neurocan and/or RPTPβ/phosphacan. In one embodiment, the method comprises two or more molecular forms of neurocan and/or RPTPβ/phosphacan. In one embodiment, the method comprises three or more molecular forms of neurocan and/or RPTPβ/phosphacan. In one embodiment, the method comprises four or more molecular forms of neurocan and/or RPTPβ/phosphacan. In one embodiment, the method comprises the detecting of (GAG)-containing and/or GAG-lacking neurocan proteins (encoding gene ID number: 1463, neurocan, SEQ ID NO:1), a full length neurocan protein, the proteolytically cleaved N-terminal neurocan fragment, the proteolytically cleaved C-terminal neurocan fragment, and/or other products of neurocan fragmentation. In one embodiment, the method comprises the detecting of the GAG-containing and/or GAG lacking RPTPβ/phosphacan protein isoforms (encoding gene ID number: 5803, protein tyrosine phosphatase receptor type Z1 gene, also known as RPTPβ, phosphacan, SEQ ID NO:2), full-length RPTPβ/phosphacan protein isoforms and/or products of their fragmentation.

In various embodiments, the method for diagnosing open NTD comprises detecting the levels of total GAG-containing and/or GAG lacking neurocan and/or RPTPβ/phosphacan proteins. In one embodiment, detecting of the levels and/or differential pattern of neurocan and/or RPTPβ/phosphacan comprises detection of individual GAG-containing and/or GAG lacking full-length neurocan and/or RPTPβ/phosphacan proteins, the proteolytically cleaved N-terminal neurocan fragment, the proteolytically cleaved C-terminal neurocan fragment, and/or any other neurocan and/or RPTPβ/phosphacan fragmentation products, or any combination thereof.

In one embodiment, the method of detecting the levels of neurocan and/or RPTPβ/phosphacan comprises the additional step of treating the sample with a chondroitinase, or chondroitin lyase. The skilled artisan will understand that treating the sample with a chondroitinase, while catalytically cleaving the polysaccharide post-translational modifications of neurocan and/or RPTPβ/phosphacan, will not diminish one's ability to accurately detect protein levels. The skilled artisan will recognize that any number of chondroitinases may be employed, including, but not limited to, chondroitinase AC, chondroitinase ABC, and chondroitinase B.

In one embodiment, the detecting of the levels and/or differential pattern of neurocan and/or RPTPβ/phosphacan does not include the step of removal and/or modification of GAG component.

In various embodiments, the method comprises determining if the level of the relevant neurocan and/or RPTPβ/phosphacan is elevated as compared to a comparator. In certain embodiments, the comparator may be the level (e.g., amount, concentration, concentration of one or more molecular forms, concentration of the neutral form, expression, level, etc.) of neurocan and/or RPTPβ/phosphacan in a subject without open NTD. In various embodiments, the comparator is the level (e.g., amount, concentration, concentration of one or more forms, concentration of the neutral form, expression, level, etc.) of neurocan and/or RPTPβ/phosphacan obtained from a human subject, an average of multiple human subjects, an average of multiple human subjects living in the same region, an average of human subjects with the same race, an average of multiple human subjects with the same ethnicity, an average of multiple human subjects living in different regions, an average of multiple human subjects with different races, an average of multiple human subjects with different ethnicities, or any combination thereof.

In one embodiment, the method comprises detecting at least one selected from the group consisting of the levels, ratios, and differential patterns of at least one neurocan and/or RPTPβ/phosphacan in a biological sample obtained from the subject, wherein at least one member is chosen from the group consisting at least one selected from the group consisting of: GAG-containing full length neurocan protein (encoding gene ID number: 1463, neurocan, SEQ ID NO: 1), GAG-lacking full length neurocan protein, GAG-containing full-length RPTPβ/phosphacan protein isoform (encoding gene ID number: 5803, protein tyrosine phosphatase receptor type Z1 gene, also known as RPTPβ, phosphacan, SEQ ID NO:2), GAG-lacking full-length RPTPβ/phosphacan protein isoform, GAG-containing proteolytically cleaved N-terminal neurocan fragment, GAG-lacking proteolytically cleaved N-terminal neurocan fragment, GAG-containing proteolytically cleaved C-terminal neurocan fragment, GAG-lacking proteolytically cleaved C-terminal neurocan fragment, any other product of neurocan fragmentation, any product of RPTPβ/phosphacan, and any combination thereof, comparing the level of the at least one molecular form of neurocan and/or RPTPβ phosphacan to a comparator, and determining that the subject has an open NTD when at least one molecular form of neurocan and/or RPTPβ phosphacan is elevated in the biological sample as compared to the comparator.

In various embodiments, a subject is identified as having open NTD when the level of total GAG-containing and/or GAG-lacking neurocan proteins (encoding gene ID number: 1463, neurocan, SEQ ID NO:1), is increased in the biological sample as compared to the comparator.

In various embodiments, a subject is identified as having open NTD when a ratio of total GAG-containing and/or GAG-lacking neurocan proteins is increased in the biological sample as compared to the comparator.

In various embodiments, a subject is identified as having open NTD when the level of total GAG-containing and/or GAG-lacking RPTPβ/phosphacan proteins (encoding gene ID number: 5803, protein tyrosine phosphatase receptor type Z1 gene, also known as RPTPβ, phosphacan, SEQ ID NO:2) is increased in the biological sample as compared to the comparator.

In various embodiments, a subject is identified as having open NTD when the ratio of total GAG-containing and/or GAG-lacking RPTPβ/phosphacan proteins is increased in the biological sample as compared to the comparator.

In various embodiments, a subject is identified as having open NTD when the level of proteolytically cleaved N-terminal neurocan fragment, is increased in the biological sample as compared to the comparator.

In various embodiments, a subject is identified as having open NTD when the level of proteolytically cleaved C-terminal neurocan fragment, is increased in the biological sample as compared to the comparator.

In various embodiments, a subject is identified as having open NTD when the level and/or pattern of neurocan fragmentation products is increased and/or changed in the biological sample as compared to the comparator.

In various embodiments, a subject is identified as having open NTD when the level of any protein isoform of RPTPβ/phosphacan (encoding gene ID number: 5803 protein tyrosine phosphatase receptor type Z1 gene, also known as RPTPβ, phosphacan, SEQ ID NO:2) is increased in the biological sample as compared to the comparator.

In various embodiments, a subject is identified as having open NTD when the level and/or pattern of at least one RPTPβ/phosphacan fragmentation product is increased and/or changed in the biological sample as compared to the comparator.

In various embodiments of the methods of the invention, the level (e.g., activity, amount, concentration, concentration of one or more ionized forms, concentration of the neutral form, expression, level, etc.) of at least one molecular form of neurocan and/or RPTPβ/phosphacan, or any combination thereof, is determined to be increased when the level of at least one molecular form of neurocan and/or RPTPβ/phosphacan, or any combination thereof, in the biological sample is increased by at least 0.01 fold, at least 0.05 fold, at least 0.07 fold, at least 0.076 fold, at least 0.1 fold, at least 0.18 fold, at least 0.19 fold, at least 0.3 fold, at least 0.36 fold, at least 0.37 fold, at least 0.38 fold, at least 0.4 fold, at least 0.43 fold, at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 16 fold, at least 16.3 fold, at least 16.31 fold, at least 20 fold, at least 25 fold, at least 26 fold, at least 26.7 fold, at least 26.72 fold, at least 30 fold, at least 40 fold, at least 50 fold, at least 75 fold, at least 100 fold, at least 192 fold, at least 192.4 fold, at least 192.44 fold, at least 200 fold, at least 250 fold, at least 500 fold, or at least 1000 fold, or at least 10000 fold, when compared to a comparator.

In various embodiments of the methods of the invention, the level (e.g., activity, amount, concentration, concentration of one or more ionized forms, concentration of the neutral form, expression, level, etc.) of at least one molecular form of neurocan and/or RPTPβ/phosphacan, or any combination thereof, is determined to be increased when the level of at least one molecular form of neurocan and/or RPTPβ/phosphacan, or any combination thereof, in the biological sample is increased by at least 1%, by at least 5%, by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared to a comparator.

In various embodiments of the methods of the invention, the ratio of neurocan to a known biomarker of spinal cord injury in the amniotic fluid is determined to be increased when the ratio is increased by at least 1%, by at least 5%, by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared to a comparator.

In various embodiments of the methods of the invention, the ratio of phosphacan to a known biomarker of spinal cord injury in the amniotic fluid is determined to be increased when the ratio is increased by at least 1%, by at least 5%, by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared to a comparator.

It will be obvious to one of skill in the art that there are a wide variety of methods for detecting the levels of neurocan and/or RPTPβ/phosphacan in a sample, including, but not limited to, Western blotting, quantitative dot blotting, ELISA assaying, mass spectrometry (MS), liquid chromatography (LC), liquid chromatography-mass spectrometry (LC-MS), targeted liquid chromatography-tandem mass spectrometry (LC-MS/MS), high-performance liquid chromatography (HPLC), ultra-performance liquid chromatography (UPLC), ultra-high-performance liquid chromatography (UHPLC), gas chromatography (GC), gas chromatography-mass spectrometry (GC-MS), globally optimized targeted mass spectrometry, targeted assay of about 200 metabolites, aqueous global profiling, liquid global profiling, GC-MS profiling, GC-MS flux analysis, carnitine analysis, lipid targeted analysis, quantitative lipid targeted analysis, tryptophan analysis, absolute quantification, multivariate statistical analysis, dynamic light scattering (DLS), nuclear magnetic resonance (NMR) spectroscopy, ultravioletvisible (UV/Vis) spectroscopy, infrared (IR) spectroscopy, Raman spectroscopy, or any combination thereof. In some embodiments of the invention, methods of measuring neurocan and/or RPTPβ/phosphacan levels in a biological sample obtained from a subject include, but are not limited to, an immunochromatography assay, an immunodot assay, a Luminex assay, an ELISPOT assay, a protein microarray assay, a ligand-receptor binding assay, displacement of a ligand from a receptor assay, displacement of a ligand from a shared receptor assay, an immunostaining assay, an RNAscope assay, a radioimmunoassay (RIA), a radioimmunodiffusion assay, an Ouchterlony immunodiffusion assay, reverse phase protein microarray, a rocket immunoelectrophoresis assay, an immunohistostaining assay, an immunoprecipitation assay, a complement fixation assay, an enzyme-substrate binding assay, an enzymatic assay, an enzymatic assay employing a detectable molecule, such as a chromophore, fluorophore, or radioactive substrate, a substrate binding assay employing such a substrate, a substrate displacement assay employing such a substrate, and a protein chip assay.

The concentration of the neurocan and/or RPTPβ/phosphacan or their fragmentation products in a sample may be determined by any suitable assay. A suitable assay may include one or more of the following methods, an enzyme assay, an immunoassay, mass spectrometry, chromatography, electrophoresis or an antibody microarray, or any combination thereof. Thus, as would be understood by one skilled in the art, the systems and methods of the invention may include any method known in the art to detect a protein in a sample.

In some embodiments, the method of detecting the levels and/or differential patterns of neurocan and/or RPTPβ/phosphacan is accomplished by Western blotting followed by immunoblotting with an antibody to at least one molecular form of neurocan and/or RPTPβ/phosphacan.

In some embodiments, the method of detecting colocalization of neurocan and/or RPTPβ/phosphacan with markers of astrocytic cells and/or neurons and/or detection of the extracellular distribution patterns of neurocan and/or RPTPβ/phosphacan is accomplished by RNAscope and/or coimmunostaining.

In some embodiments, the detecting of neurocan and/or RPTPβ/phosphacan is accomplished by employing an ELISA assay. The colorimetric nature of the ELISA assay provides for a more rapid quantification of the levels of proteins in the sample than is achievable with a Western blot. An ELISA assay, or enzyme-linked immunosorbent assay, is a commonly applied biochemical technique known to one of ordinary skill in the art. In various embodiments, the ELISA assay employed may be a direct ELISA with a single active antibody, an indirect ELISA assay with an unmodified antibody and a secondary reporter antibody, a “sandwich” ELISA with a solid phase-supported antibody and a direct reporter antibody, or any of the other ELISA assays known in the art.

In one embodiment, the method comprises using a multi-dimensional nonlinear algorithm to determine if the level (e.g., activity, amount, concentration, concentration of one or more ionized forms, concentration of the neutral form, expression, level, etc.) of at least one molecular form of neurocan and/or RPTPβ/phosphacan in the biological sample is statistically different than a comparator. In some embodiments, the algorithm is drawn from the group consisting essentially of: linear or nonlinear regression algorithms; linear or nonlinear classification algorithms; ANOVA; neural network algorithms; genetic algorithms; support vector machines algorithms; hierarchical analysis or clustering algorithms; hierarchical algorithms using decision trees; kernel based machine algorithms such as kernel partial least squares algorithms, kernel matching pursuit algorithms, kernel fisher discriminate analysis algorithms, or kernel principal components analysis algorithms; Bayesian probability function algorithms; Markov Blanket algorithms; a plurality of algorithms arranged in a committee network; and forward floating search or backward floating search algorithms.

In some embodiments, the level of at least one molecular form of neurocan and/or RPTPβ/phosphacan of the invention in the biological test sample of the subject is compared to a comparator. Non-limiting examples of comparators include, but are not limited to, a negative control, a positive control, standard control, standard value, an expected normal background value of the subject, a historical normal background value of the subject, a reference standard, a reference level, an expected normal background value of a population that the subject is a member of, or a historical normal background value of a population that the subject is a member of. In one embodiment, the comparator is a level (e.g., activity, amount, concentration, concentration of the ionized form, concentration of the neutral form, expression, level, etc.) of at least one molecular form of neurocan and/or RPTPβ/phosphacan in a sample obtained from a subject not having an open NTD. In one embodiment, the comparator is a level of at least one molecular form of neurocan and/or RPTPβ/phosphacan in a sample obtained from a subject known not to have an open NTD.

In one embodiment, the profile of at least one molecular form of neurocan and/or RPTPβ/phosphacan in a subject is compared to a predetermined or comparator profile of at least one molecular form of RPTPβ/neurocan and/or phosphacan or reference profile of at least one molecular form of neurocan and/or RPTPβ/phosphacan to identify open NTD.

Control group samples may either be from a normal subject, samples from subjects with a known open NTD, or samples from subjects with no known open NTD.

### Methods of Treatment

In one embodiment, the present invention relates to a method for treating open NTD in a subject in need thereof comprising the step of administrating to the subject a treatment for open NTD.

In some embodiments, upon diagnosis of open NTD, the fetus undergoes in utero treatment for correction of the open NTD. In some embodiments, the in utero treatment includes fetal surgery to repair the open NTD. In one embodiment, the fetal surgery is an open surgery. In one embodiment, the fetal surgery is fetoscopic surgery. In one embodiment, the fetal surgery is a hybrid of open and fetoscopic surgery. In some embodiments, the in utero treatment includes non-surgical repair. In one embodiment, the non-surgical repair is transamniotic stem cell therapy. In some embodiments, treatment includes surgical and stem cell-based therapies. In some embodiments, the stem cells are derived from placental stem cells. In some embodiments, the stem cells are placental-derived mesenchymal stem cells. In some embodiments, the stem cells are derived from amniotic fluid. In some embodiments, the stem cells are amniotic fluid-derived mesenchymal stem cells. In some embodiments, the stem cells are umbilical cord-derived. In some embodiments, the stem cells are umbilical cord-derived mesenchymal stem cells. In some embodiments, treatment includes non-stem cells. In some embodiments, the non-stem cells are mesenchymal stromal cells. In some embodiments, the mesenchymal stromal cells are placental-derived. In some embodiments, the mesenchymal stromal cells are umbilical cord-derived. In some embodiments, the mesenchymal stromal cells are placental-derived.

In some embodiments, upon diagnosis of open NTD, the open NTD is not repaired until after birth. In one embodiment, surgery is used to repair the open NTD after birth. In some embodiments, the surgical repair includes stem cell therapy. In some embodiments, the stem cells are derived from amniotic fluid. In some embodiments, the stem cells are amniotic fluid-derived mesenchymal stem cells. In some embodiments, the stem cells are umbilical cord-derived. In some embodiments, the stem cells are umbilical cord-derived mesenchymal stem cells. In some embodiments, treatment includes non-stem cells. In some embodiments, the non-stem cells are mesenchymal stromal cells. In some embodiments, the mesenchymal stromal cells are placental-derived. In some embodiments, the mesenchymal stromal cells are umbilical cord-derived. In some embodiments, the mesenchymal stromal cells are placental-derived.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

In MMC, the failed development of the overlying vertebrae, musculature and skin leaves the fetal spinal cord exposed to the AF allowing a communication between the neural and AF compartments. The chondroitin sulfate proteoglycans (CSPGs) neurocan and phosphacan are abundant extracellular matrix (ECM) components of the developing central nervous system (CNS) (Margolis, R. K., et al., 1996, Perspectives on Developmental Biology, 3:273-290; Meyer-Puttlitz, B., et al., 1996, Journal of Comparative Neurology, 366:44-54). Since these are highly soluble ECM proteins (Deepa, S. S., et al., 2006, Journal of Biological Chemistry, 281:17789-17800; Grumet, M., et al., 1996, Perspectives on Developmental Neurobiology, 3:319-330), it was hypothesized that open NTDs, such as MMC could allow their release from the lesion site into the environment surrounding the fetus, in this case the AF. Therefore a wellestablished retinoic acid (RA)-induced fetal rat model of MMC was used (Danzer, E., et al., 2005, Experimental Neurology, 194:467-475; Barbe, M. F., et al., 2014, Birth Defects Research A: Clinical & Molecular Teratology, 100:453-462), to determine whether AF levels of neurocan and phosphacan are elevated in affected fetuses compared to normal age-matched controls. In addition, solubility, and in vitro release of neurocan and phosphacan from the MMC spinal cord tissue into the AF or detergent-free buffer were examined. Immunohistochemistry was performed in parallel to confirm the extracellular pattern of neurocan and phosphacan expression in MMC spinal cords.

### Example 1: Detection of Neurocan in Amniotic Fluid

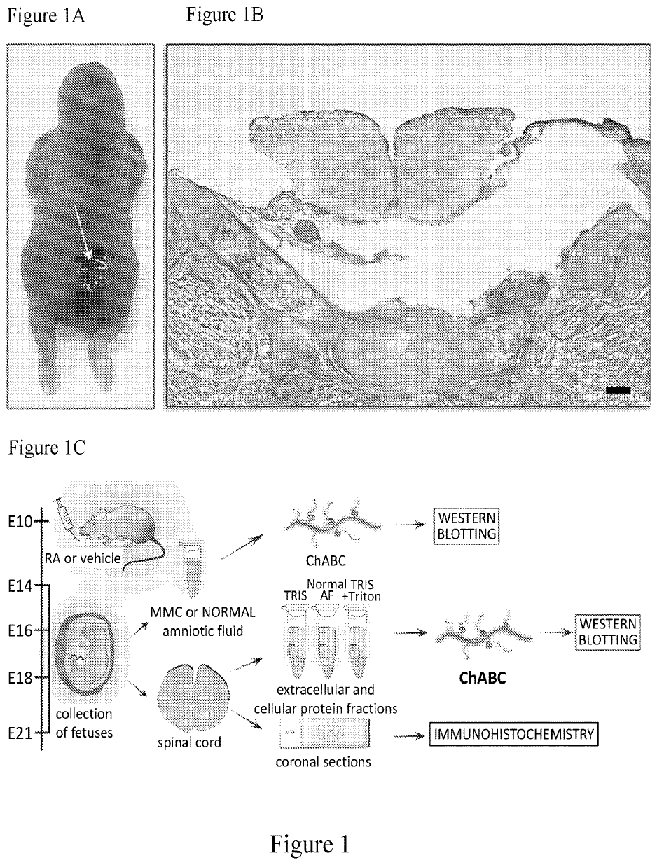

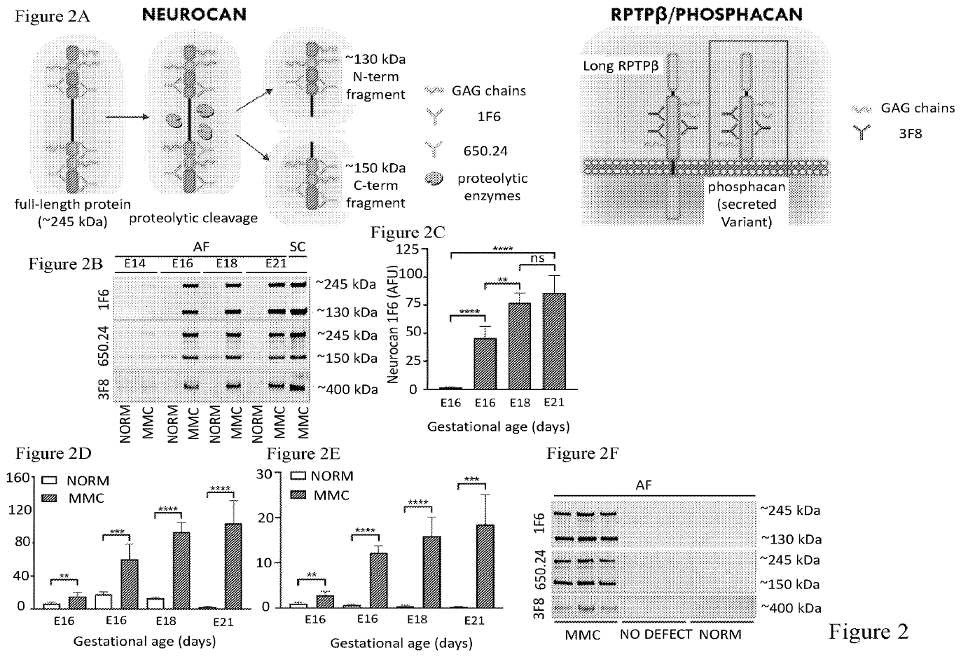

MMC was induced during neurulation by exposure to all-trans RA (Barbe, M. F., et al., 2014, Birth Defects Research A: Clinical & Molecular Teratology, 100:453-462). As previously described, MMC fetuses displayed defects characterized by a pathological opening in the vertebral arch and the overlying skin with the spinal cord exposed at the center of the lesion confined to the lumbosacral area of the fetus (Danzer, E., et al., 2005, Experimental Neurology, 194:467-475; Barbe, M. F., et al., 2014, Birth Defects Research A: Clinical & Molecular Teratology, 100:453-462, Zieba, J., et al., 2019, Journal of Neurotrauma, 36:1965-1973) (FIGS. 1A and 1B). AF samples collected from fetal rats with MMC defects and age-matched normal controls at four time points in gestation (E14, E16, E18 and E21) were treated with Chondroitinase ABC (ChABC) to degrade polysaccharides and examined using western blotting (FIG. 1C). Following immunoblotting with the 1F6 antibody, which recognizes the N-terminal epitope of neurocan (FIG. 2A), a faint band representing a full-length neurocan (245 kDa core protein) was detected in the AF of MMC fetuses at E14 (FIG. 2B). Both molecular forms of neurocan, representing the full-length neurocan and the proteolytically cleaved-N-terminal neurocan fragment (130 kDa core protein), were present in the AF of MMC fetuses starting at E16 (FIG. 2B). Compared with E14, the total level of neurocan in the AF of MMC fetuses was significantly elevated at E16, and followed by smaller but significant increase at E18, reaching the highest level at E21 (FIG. 2C). In sharp contrast to these results, the signal for neurocan was not detectable in the AF of normal age-matched fetuses (FIG. 2B). Immunoblotting with the 650.24 neurocan antibody, which recognizes the C-terminal epitope (FIG. 2A), revealed detection of full length neurocan (245 kDa core protein) as well as the proteolytically cleaved-C-terminal neurocan fragment (150 kDa core protein) in the AF of MMC fetuses starting at E14, with a rapid increase in the intensity of neurocan signal between E14 and E16 and its peak at E21 (FIG. 2B). Only faintly stained bands of neurocan, predominantly corresponding to C-terminal neurocan fragments, were identified in the AF of normal fetuses (FIG. 2B). Quantitative analysis showed that the total AF level of neurocan was significantly elevated in MMC compared to normal controls at each examined gestational age (FIG. 2D). Moreover, the total level of neurocan in the AF of MMC fetuses robustly increased with gestational time, reaching the highest level at E21 (FIG. 2D). Western blot analyses demonstrated the presence of full-length neurocan, the proteolytically cleaved N-terminal neurocan fragment and C-terminal neurocan fragment in MMC spinal cord lysates that were used as positive controls (FIG. 2B), indicating that the molecular forms of neurocan identified in the AF of MMC fetuses and MMC spinal cords were qualitatively similar.

### Example 2: Detection of Phosphacan in Amniotic Fluid

Phosphacan is a secreted spliced extracellular variant of the receptor type protein tyrosine phosphatase beta (RPTPβ); also known as protein tyrosine phosphatase receptor type Z1 (PTPRZ1) that is expressed during CNS development and in pathological processes such as tissue injury (Grumet, M., et al., 1996, Perspectives on Developmental Neurobiology, 3:319-330; Maurel, P., et al., 1994, Proceedings of the National Academy of Sciences USA, 91:2512-2516). Western blot analysis with the 3F8 antibody, which recognizes the phosphacan and long receptor type PTPβ (FIG. 2A) revealed an approximately 400 kDa single band of core protein indicating the presence of secreted phosphacan in the AF of MMC fetuses starting at E14 (FIG. 2B). In contrast to the AF of MMC fetuses, only weakly stained bands of phosphacan were visible in the AF of normal fetuses at E14, and their intensity diminished to a barely detectable level after E16 (FIG. 2B). The total AF levels of phosphacan were significantly higher in MMC than in normal controls at all examined time points, and progressively increased in MMC compared to normal age-matched controls as pregnancy advanced (FIG. 2E).

The analyses aimed to evaluate the presence of neurocan and phosphacan were initially conducted using AF samples pooled from three randomly selected MMC or normal fetuses. Typically, RA induces MMC in the majority of fetuses with the other littermates appearing normal (Danzer, E., et al., 2005, Experimental Neurology, 194:467-475). To verify these CSPGs originated from the MMC defect and were not generated by RA, AF samples from individual E21 fetuses with MMC defects, littermates with no MMC defects after RA exposure, or normal controls were loaded into separate lanes and subjected to western blotting. In contrast to the AF from fetuses with MMC defects, the AF from RA-exposed littermates showing no signs of MMC, similarly to normal controls contained little or no neurocan and phosphacan (FIG. 2F), verifying a direct correlation with the presence of MMC defect.

This study identifies the presence of two CNS-associated CSPGs, neurocan and phosphacan in the AF of fetal rats in a clinically relevant model of MMC. These MMC fetuses showed robust and significantly elevated levels of neurocan and phosphacan in the AF when compared to normal age-matched controls at different developmental time points starting at least as early as E14. Normal appearing littermates from RA-exposed cohort showed no increase in the AF levels of neurocan or phosphacan, unlike those with MMC defect. In addition, it was shown that the full-length neurocan along with its proteolytically cleaved molecular forms and phosphacan identified in the AF of MMC fetuses were identical to those found in the MMC spinal cords and could be efficiently released from the spinal cord tissue into the normal AF or detergent-free buffer. These data provide evidence for the assessment of neurocan and phosphacan levels within the AF as promising diagnostic biomarkers of open NTD-affected fetuses, even at early gestational ages.

### Example 3: Aqueous Solubility of Neurocan and Phosphacan

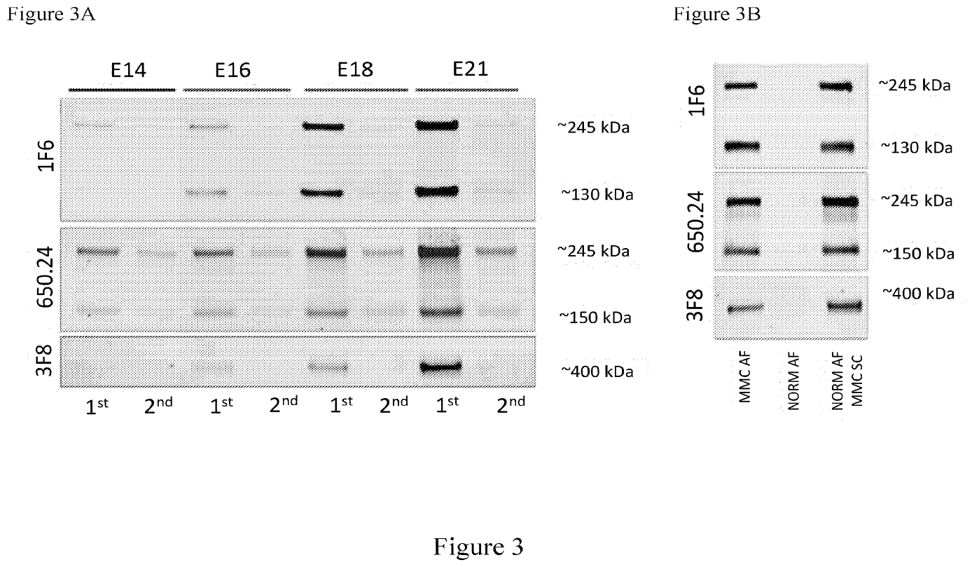

To determine if neurocan or phosphacan from the spinal cord tissue was readily soluble into an aqueous environment, sequential extractions of MMC spinal cord proteins were performed at various points of gestion using detergent-free followed by detergent-containing buffer. Immunoblotting of extracted proteins in all examined samples showed that the vast majority of neurocan and phosphacan were present in the first fraction, extracted without detergent with only a minor component identified within the second fraction extracted with detergent (FIG. 3A). To further evaluate the solubility of neurocan and phosphacan into the AF, MMC spinal cord tissue from E21 fetuses was placed into AF samples of aged-matched normal fetuses that do not contain these CSPGs. Western blotting analysis revealed the presence of full-length neurocan and the proteolytically cleaved N-terminal neurocan fragment and C-terminal neurocan fragment as well as phosphacan detected in all examined samples of AF incubated with MMC spinal cords (FIG. 3B), supporting their release into the AF.

### Example 4: Expression of Neurocan and Phosphacan in MMC Spinal Cord

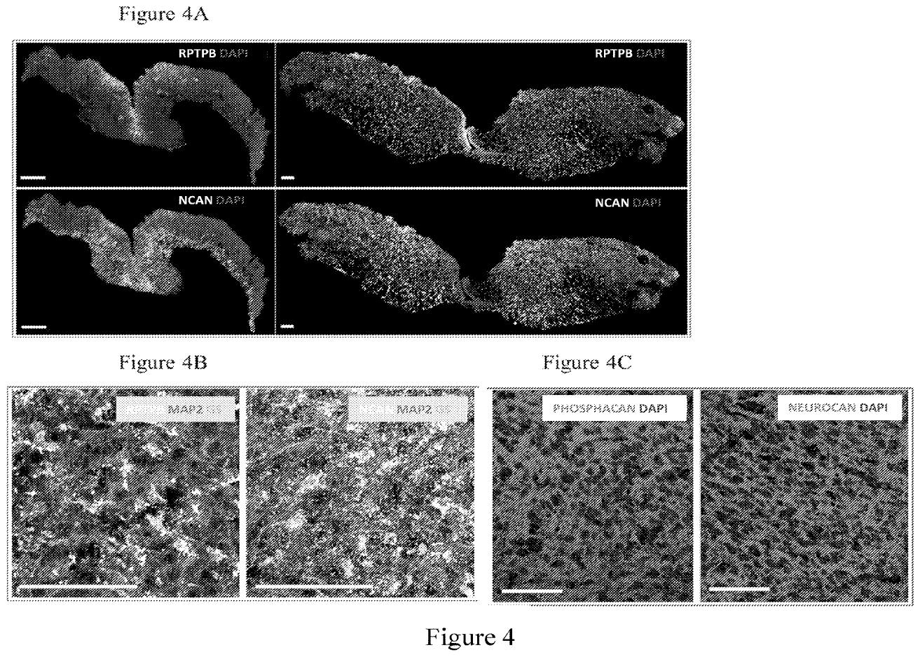

RNAscope analysis in MMC spinal cord sections using specific probes for the Neurocan and the RPTPβ/phosphacan revealed enriched RPTPβ/phosphacan expression in the externally exposed ventricular zone, while the site of neurocan expression was extended to the parenchyma of malformed E14 MMC spinal cords (FIG. 4A, left panel). In E21 MMC spinal cords, Neurocan and RPTPβ/phosphacan were expressed robustly throughout the spinal cord tissue, with RPTPβ/phosphacan expression maintained in the ventricular zone region (FIG. 4A, right panel). To explore the phenotype of spinal cord cells expressing these CSPGs, RNAscope analysis was combined with coimmunostaining for glutamine synthetase (GS), a marker of astrocytic cells (Anlauf, E., et al., 2013, Front. Endocrinol, 4:144) and MAP2, a marker of neuronal cells (Dehmelt, L., et al., 2005, Genome Biol, 6:204). Analysis of spinal cord sections from MMC defects demonstrated that the RPTPβ/phosphacan was expressed by astrocytes, while neurocan was expressed by neurons and astrocytes (FIG. 4B). Consistent with biochemical studies, immunohistochemical analysis of spinal cord sections from MMC defects confirmed extracellular localization of secreted neurocan and phosphacan within the spinal cord tissue (FIG. 4C).

The discovery of significantly elevated levels of neurocan and phosphacan in the AF of MMC fetuses provides novel data demonstrating changes in the composition of AF due to the defect and suggests their potential usefulness in early prenatal diagnosis. Most of the established and potential biomarkers for the diagnosis of pregnancy-related pathologies have been identified by analysis of the AF (Geer, L. A., et al., 2015, Environmental Research, 136:470-481; Tsangaris, G. T., et al., 2011, EPMA, 2:149-155). Currently, testing for alpha fetoprotein (AFP) and acetylcholine esterase (AChE) in the AF and/or ultrasound evaluation of intracranial and spinal anatomy is considered diagnostic of open NTDs, while AFP measurement in the maternal serum is used as a prenatal screening test (Cameron, M. and Moran, P., 2009, Prenatal Diagnosis, 29:402-411; Rose, N. & Mennuti, M. T., 2009, Global library of women's medicines; Palomaki, G. E., et al., Genetics in Medicine, 22:462-474). Although, AFP assessment during the early second trimester is the biochemical marker for open NTDs, non-neurological fetal disorders are also associated with elevated AFP levels and cannot be excluded based on this marker (Weiss, R. R., et al., 1976, Obstetrics & Gynecology, 47:148-151; Rose, N. & Minneti, M. T., 2009, Global library of women's medicines).

Unlike AFP, which is a serum protein in the fetus derived from the yolk sac and fetal liver and is not specific for NTDs (Rose N. & Minneti, M. T., 2009, Global library of women's medicines), neurocan and phosphacan are CNS-associated CSPGs robustly expressed during fetal development (Margolis, R. K., et al., 1996, Perspectives on Developmental Neurobiology, 3:273-290; Meyer-Puttlitz, B., et al., 1996, Journal of Comparative Neurology, 366:44-54). Using diagnostic biomarkers specific for neural tissue would allow distinction from non-neurological fetal disorders, therefore increasing the diagnostic accuracy for NTDs. In addition, AFP tests require specific gestational ages, with the optimal time for AFP testing in the maternal serum between 16-18 weeks of gestation and in the AF between 13-22 weeks of gestation, when serum and AF levels of AFP have been found to be substantially elevated in NTD-affected pregnancies (Palomaki, G. E., et al., 2020, Genetics in Medicine, 22:462-474; Rose N. & Mennuti, M. T., 2009, Global library of women's medicines; Bradley, L. A., et al. 2005 Genetics in Medicine, 7 (5): 344-54). The range of gestational age where AFP levels are markedly elevated is narrow, therefore the interpretation of AFP levels is complicated by adjustment for the fetal age.

In addition, several other factors including race or maternal diabetes influence the results (Palomaki, G. E., et al., 2020, Genetics in Medicine, 22:462-474; Rose N. & Mennuti, M. T., 2009, Global library of women's medicines). In some cases, testing for the presence of AChE in the AF can be indicated as an aid to diagnose open NTDs, however, testing for AChE is a nonquantitative method (Bradley LA 2005, Genetics in Medicine, 7 (5): 344-54). As demonstrated in the present study, AF levels of neurocan and phosphacan were significantly elevated in comparison to controls from as early as E14 and their content in the AF of MMC fetuses increased with advancing gestational age, while decreased in normal AF. These results show that differences in the AF levels of neurocan and phosphacan can be used to discriminate between normal fetuses and those affected by the open NTD at all examined gestational ages. The prominent differences between MMC and normal fetuses are significantly increased AF levels of neurocan and phosphacan detected in MMC fetuses from as early as E14, with their levels in the AF of MMC fetuses raising rapidly at E16 and reaching the highest levels at E21. The comparison of embryonic development between rats and humans indicate that E14 rat embryos are equivalent to approximately gestational week 7 in humans (Butler, H. & Juurlink, B. H. J., 1987, An Atlas for Staging Mammalian and Chick Embryos). In this respect, the identification of significant differences in the AF levels of neurocan and phosphacan between MMC and normal rat fetuses makes these molecules promising early biomarkers for detection of open NTDs. These exploratory biomarkers, however, require validation in human samples, including AF and maternal blood, which is less invasive tissue source from the diagnostic and especially screening and monitoring perspective. Antibodies and ELISA assays for human CSPGs are commercially available and currently being studied for quantification of their levels in human bodily fluids (Minta, K. et al., 2019, Clinical Chemistry and Laboratory Medicine, 57:1565-1573). Use of a well characterized fetal rat model of MMC, which similarly results from incomplete neural tube closure and pathologically resembles human MMC, allows for direct translation from a murid to human system. Additionally, the ability to collect samples at various time points allowed for a systematic analysis of a large number of age-matched samples, which is not possible in human pregnancies.

The differences in AF levels of neurocan and phosphacan have not been previously reported between MMC and normal fetuses, although, changes in AF proteins other than neurocan and phosphacan have been studied in MMC. Observations from previous studies in animal models indicate that the molecular components of AF in MMC fetuses, including neurofilament heavy chain expressed by neurons and glial fibrillary acid protein, an intermediate filament specific to astrocytes, might be considerably different from the AF of normal fetuses (Petzold, A., et al., 2005, Journal of Neurochemistry, 95:594-598; Danzer, E., et al., 2011, American Journal of Obstetrics & Gynecology, 204: e1-11). These molecules are mostly insoluble cytoskeletal proteins with the structural role in axons and astrocytes and are elevated in the AF of MMC fetuses at later stages of gestion, a period corresponding to spinal cord injury in MMC fetuses (Petzold, A., et al., 2005, Journal of Neurochemistry, 95:594-598; Danzer, E., et al., 2011, American Journal of Obstetrics & Gynecology, 204: e1-11). Neurocan and phosphacan are CSPGs expressed in the developing CNS that are secreted locally and assembled in the surrounding extracellular space (Margolis, R. K., et al., 1996, Perspectives on Developmental Neurobiology, 3:273-290; Meyer-Puttlitz, B., et al., 1996, Journal of Comparative Neurology, 366:44-54). Unlike cytoskeletal proteins, neurocan and phosphacan are highly soluble ECM proteins that transfer efficiently from the MMC spinal cord tissue into the AF or detergent-free buffer and are found at exceptionally high levels in the AF of MMC fetuses even at very early gestational ages (E14). This is consistent with previous reports demonstrating low affinity attachment of these CSPGs within the neural ECM and their ease of extraction from the spinal cord tissue using the detergent-free buffer (Deepa, S. S. et al., 2006, Neurosurgery Clinics of North America, 281:17789-17800). Furthermore, immunocytochemistry of spinal cord sections at the site of MMC defect confirmed strong extracellular accumulation of these CSPGs, and western blotting analysis of MMC spinal cord extracts demonstrated identical molecular weight patterns to those identified in the MMC AF. Normal appearing littermates from RA-exposed cohort showed no increase in the AF levels of neurocan or phosphacan, unlike those with MMC defect. These results support the hypothesis that neurocan and phosphacan are elevated in the AF of MMC fetuses as a result of their release from the site of the MMC defect and not because of their induction by RA exposure.

The principal findings indicate that the significant differences in the AF levels of neurocan and phosphacan allow distinction between MMC and normal controls at all examined gestational ages and may constitute potential biomarkers to facilitate early diagnosis of open NTDs. Based on the present results, demonstrating a correlation between the increased AF levels of neurocan and phosphacan and the presence of MMC defect in fetal rats, further studies of human pregnancies will be required to confirm these associations. The identification of an early and specific biomarker can provide information supplementary to diagnostic imaging, beyond what is currently offered by the laboratory tests in the field of early detection and diagnosis of open NTDs. Furthermore, discovering CNS-associated proteins such as neurocan and phosphacan in the AF of MMC fetuses provides new information about changes in the composition of AF in in the presence of the defect and may also contribute to a better understanding of underlying pathophysiology and development of prenatal therapies.

TABLE 1
Neurocan and Phosphacan Sequences

| | | SEQ |
|---|---|---|
| | Gene ID | ID |
| Name | (Accession Number) | NO: |
| Neurocan Encoding Gene | 1463 (NC_000019.10) | 1 |
| Phosphacan Encoding Gene | 5803 (NC_000007.14) | 2 |
| Neurocan mRNA | (NM_004386.3) | 3 |
| Phosphacan Isoform 1 mRNA | (NM_002851.3) | 4 |
| Phosphacan Isoform 2 mRNA | (NM_001206838.2) | 5 |
| Phosphacan Isoform 3 mRNA | (NM_001206839.2) | 6 |
| Phosphacan Isoform 4 mRNA | (NM_001369395.1) | 7 |
| Phosphacan Isoform 5 mRNA | (NM_001369396.1) | 8 |
| Neurocan Core Precursor | (NP_004386.3) | 9 |
| Phosphacan Isoform 1 | (NP_002842.2) | 10 |
| Phosphacan Isoform 2 | (NP_001193767.1) | 11 |
| Phosphacan Isoform 3 | (NP_001193768.1) | 12 |
| Phosphacan Isoform 4 | (NP_001356324.1) | 13 |
| Phosphacan Isoform 5 | (NP_001356325.1) | 14 |

* Accession Numbers correspond to National Center for Biotechnology Information (NCBI) Accession Numbers.

The materials and methods employed in these experiments are now described.

### Retinoic Acid-Induced Animal Model of MMC

All experiments were conducted under the guidelines of Temple University's Institutional Animal Care and Use Committee and the National Institutes of Health Guide for Care and Use of Laboratory Animals. Time-dated Sprague-Dawley pregnant rats (Charles River Laboratories) were placed on a standard dark: light schedule. An MMC defect was induced in fetuses of time-dated pregnant rats by gavage of a single dose of 50 mg/kg of all-trans retinoic acid (RA) (Millipore Sigma; USA) dissolved in olive oil on embryonic day 10 (E10), as described previously (Barbe, M. F., et al., 2014, Birth Defects Research A: Clinical & Molecular Teratology; Zieba, J., et al., 2017., PLOS One, 12: e0174625). Normal control dams were gavage fed with olive oil. Dams were euthanized using chamber inhaled CO₂, and the total number of fetuses were determined following the midline laparotomy and exposure of the uterus. AF samples from fetuses with MMC defect after RA exposure (n=91), no MMC defect after all-trans RA exposure (n=8) and normal fetuses (n=97) were collected at E14, E16, E18 and E21 (term=E22) using aseptic techniques. After collection of AF samples, the uterus and gestational membranes were removed, and each fetus was examined for the presence of lumbosacral MMC defect. The incidence of isolated MMC defects was observed in 92% (91/99) of fetuses. After harvesting, fetuses were collected and euthanized by decapitation according to standard procedures.

### AF Preparation

Immediately after harvesting, AF samples collected from fetuses at E14, E16, E18 and E21 were centrifuged at 4,000 rpm for 10 min to remove cells and debris and then stored at −80 °C until further analysis. Prior to western blotting equal volumes of AF from 3 randomly selected MMC or normal AF samples were combined and digested with 0.03 international units (IU) of Chondroitinase ABC (ChABC; Millipore Sigma; USA) in 0.1 M Tris-HCl buffer, pH=8.0, containing 0.03 M sodium acetate and protease inhibitors cocktail (1:100; Millipore Sigma; USA) for 3 hours at 37 °C with gentle shaking (Deepa, S. S., et al., 2006, Journal of Biological Chemistry, 281:17789-17800). Enzymatic reaction was terminated by adding 4× Laemmli Sample Buffer (Bio-Rad; USA) with 10% β-mercaptoethanol (Millipore Sigma; USA). AF samples were subsequently subjected to gel electrophoresis and analyzed by western blotting as described below.

### Protein Isolation

### Total Spinal Cord Protein Extracts

Lumbar spinal cords were isolated from E21 MMC fetuses collected as described above. Immediately after harvesting, lumbar spinal cord samples from 6-7 randomly selected fetuses were pooled together and collected onto 50 mM Tris-HCl buffer with 0.5% TRITON X-100 (Fisher Scientific; USA) and protease inhibitor cocktail. Samples were subsequently homogenized, rotated at 4 °C for 45 min, and centrifuged for 14.000×g, 30 min at 4 °C. Protein concentration in spinal cord extracts was measured using colorimetric detection and quantification kit Pierce™ BCA Protein Assay Kit (Thermo Scientific, USA), and samples were stored at −80 °C until further analysis. Prior to gel electrophoresis, spinal cord protein samples (10 ug) were digested with ChABC as described above for AF preparation, subjected to gel electrophoresis, and analyzed by western blotting. Total protein extracts of MMC spinal cords served as positive controls for western blotting analyses of AF.

### Sequential Spinal Cord Protein Extracts

Lumbar spinal cords were isolated from MMC fetuses collected at E14, E16, E18 or E21 as described above. Immediately after harvesting, lumbar spinal cords from 6-7 randomly selected fetuses per age group were pooled together and collected onto 50 mM Tris-HCl buffer with protease inhibitors cocktail. Samples were homogenized, rotated at 4 °C for 45 min, and centrifuged for 14.000×g, 30 min at 4 °C (1^{st} extract). The resultant pellets were lysed with buffer composed of 50 mM Tris-HCl with 0.5% TRITON X-100 and protease inhibitors rotated at 4 °C for 45 min, and centrifuged for 14.000×g, 30 min at 4 °C (2^{nd} extract). Protein concentration was measured using colorimetric detection and quantification kit Pierce™ BCA Protein Assay Kit, and samples were stored at −80 °C until further analysis. Prior to gel electrophoresis, 1^{st} and 2^{nd} protein extracts (10 μg each) were digested with ChABC and subjected to western blotting analyses as described below. All analyses were made using spinal cord extracts prepared from at least two independent sets of spinal cord tissue samples per each age group.

### MMC Spinal Cord Protein Extracts in Normal AF

Lumbar spinal cords were isolated from E21 MMC fetuses collected as described above. To determine the solubility of neurocan and phosphacan into the AF, lumbar spinal cords from 6-7 randomly selected fetuses were pooled together and collected into the AF of normal age-matched fetuses. Samples were rotated for 45 min at 4 °C and centrifuged at 14,000×g, 4 °C for 30 min. Prior to gel electrophoresis, equal volumes of spinal cord extracts were digested with ChABC and subjected for Western blot analyses as described below. All analyses were made using spinal cord extracts prepared from at least two independent sets of MMC spinal cord tissue samples and AF collected from normal age-matched fetuses.

### Western Blotting

Amniotic fluid or spinal cord samples were resolved on 8% sodium dodecyl sulfate-polyacrylamide gel (SDS-PAGE) and transferred onto 0.22 μM nitrocellulose membranes (Li-Cor Biosciences; USA). After transfer, blots were blocked for non-specific binding with 5% Blotting-Grade Blocker (Bio-Rad; USA) in TBS and incubated with one of the following antibodies: 1F6 mouse monoclonal antibody (1:500; Developmental Studies Hybridoma BankDSHB, USA), 650.24 mouse monoclonal antibody (1:1000; Santa Cruz Biotechnology; USA), and 3F8 mouse monoclonal antibody (1:250; DSHB, USA). The sizes of the detected proteins were estimated using Precision Plus Protein™ All Blue Prestained Protein Standards (Bio-Rad; USA). Blots were incubated with goat anti-mouse antibody conjugated to IRDye® 680RD dye (LI-COR Biosciences; USA) and signals were detected using Odyssey CLx Imaging System (LI-COR Biosciences). Image Studio Ver. 3.1. (LI-COR Biosciences) was used to quantify the signal intensity presented as arbitrary fluorescence units (AFU).

### Histology and Immunofluorescence

Fetal rats were collected as described above and then fixed in 10% neutral buffer formalin (Fisher Scientific; USA) at 4 °C. After fixation, samples were equilibrated in 15% followed by 30% sucrose in PBS at 4 °C before embedding in OCT (Sakura Finetek USA, USA), and frozen. Serial 15 μm coronal sections were obtained through the center of the MMC defect and mounted on charged glass slides (Superfrost Plus, Fisher Scientific, USA). For histological analyzes sections were stained with hematoxylin and eosin (H&E, Millipore Sigma; USA) according to manufacturer's protocol, mounted with VectaMount® Permanent Mounting medium (Vector Laboratories; USA), cover slipped and visualized using bright-field microscope (Eclipse 80i; Nikon). Sections assigned for immunofluorescence were blocked with 5% goat serum (Vector Laboratories, USA) in 1× bovine serum albumin (BSA; Millipore Sigma, USA) solution with 0.3% TRITON X-100 in PBS for 45 min at room temperature. Sections were then incubated with 1F6 (1:200), 650.24 (1:250) or 3F8 (1:150) antibodies and subsequently with anti-mouse Alexa Fluor® 555 secondary antibody (1:1000; Fisher Scientific) and 4′6′-diamidini-2-phenylindole (DAPI; Millipore Sigma) to visualize cell nuclei. Sections when then mounted with ProLong Gold antifade reagent (Fisher Scientific, USA) and cover slipped. Photographs were taken using Zeiss Axio10 microscope (Zeiss) and Leica SP8 confocal microscope (Leica). All analyses were made using cross sections obtained from the lumbar spinal cord region of three MMC fetuses collected at E21 by examining at least three sections from each fetus.

### RNAscope and Coimmunostaining

For RNAscope, sections were processed using an RNAscope® V2 multiplex fluorescent reagent kit (Advanced Cell Diagnostics Inc. ACD, USA) and probes specifically targeting rat Neurocan (Rn-Ncan-C2; ACD, USA) and rat RPTPβ/phosphacan (Rn-Ptprz1-C3; ACD, USA, which recognizes all alternatively spliced transcript variants) according to the manufacturer's protocol. Cell nuclei were stained with DAPI. Sections were then mounted with ProLong Gold antifade reagent and cover-slipped. Photographs were taken using a Leica SP8 confocal microscope. All analyses were performed using cross sections obtained from the lumbar spinal cord region of three MMC fetuses collected at E14 and E21 by examining at least three sections from each fetus. RNAscope with coimmunostaining were carried out using an RNA protein codetection ancillary kit (ACD, USA), mouse anti-MAP2 (MAB3418; 1:100; Millipore, USA), and rabbit antiglutamine synthetase (ab73593; 1:200; Abcam, USA) antibodies according to the manufacturer's protocol. Cell nuclei were stained with DAPI. Sections when then mounted with ProLong Gold antifade reagent and cover-slipped. Photographs were taken using a Leica SP8 confocal microscope. All analyses were performed using cross sections obtained from the lumbar spinal cord region of three MMC fetuses collected at E21 by examining at least two sections from each fetus.

### Statistical Analysis

Statistical analyses were conducted using GraphPad Prism 8.3.0 Software (GraphPad Software). Data was analyzed using unpaired t-test or ordinary one-way ANOVA with Tukey's comparisons test, when applicable. All numerical data is presented as mean±SD.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of diagnosing open NTD in a human fetus, the method comprising:
(a) obtaining a biological sample from a test subject;
(b) analyzing the biological sample with an assay that specifically detects at least one selected from the group consisting of: GAG-containing full length neurocan protein, GAG-lacking full length neurocan protein, GAG-containing full-length RPTPβ/phosphacan protein isoform, GAG-lacking full-length RPTPβ/phosphacan protein isoform, GAG-containing proteolytically cleaved N-terminal neurocan fragment, GAG-lacking proteolytically cleaved N-terminal neurocan fragment, GAG-containing proteolytically cleaved C-terminal neurocan fragment, GAG-lacking proteolytically cleaved C-terminal neurocan fragment, any other product of neurocan fragmentation, and any product of RPTPβ/phosphacan fragmentation;
(c) detecting at least one selected from the group consisting of:
the levels, the ratios, and the differential pattern of at least one selected from the group consisting of:
GAG-containing full length neurocan protein, GAG-lacking full length neurocan protein, GAG-containing full-length RPTPβ/phosphacan protein isoform, GAG-lacking full-length RPTPβ/phosphacan protein isoform, GAG-containing proteolytically cleaved N-terminal neurocan fragment, GAG-lacking proteolytically cleaved N-terminal neurocan fragment, GAG-containing proteolytically cleaved C-terminal neurocan fragment, GAG-lacking proteolytically cleaved C-terminal neurocan fragment any other product of neurocan fragmentation, and any product of RPTPβ/phosphacan fragmentation;
(d) comparing at least one selected form the group consisting of:
the levels, the ratios, and the differential pattern of at least one selected from the group consisting of:
GAG-containing full length neurocan protein, GAG-lacking full length neurocan protein, GAG-containing full-length RPTPβ/phosphacan protein isoform, GAG-lacking full-length RPTPβ/phosphacan protein isoform, GAG-containing proteolytically cleaved N-terminal neurocan fragment, GAG-lacking proteolytically cleaved N-terminal neurocan fragment, GAG-containing proteolytically cleaved C-terminal neurocan fragment, GAG-lacking proteolytically cleaved C-terminal neurocan fragment, any other product of neurocan fragmentation, and any product of RPTPβ/phosphacan fragmentation in the sample with that of a comparator; and
(e) determining that the subject has open NTD based on these methods.

2. The method of claim 1, wherein the biological sample comprises amniotic fluid, blood, serum, plasma, or urine from a fetus or an individual carrying the fetus.

3. The method of claim 1, wherein step (b) further comprises a step of treating the biological sample with a chondroitinase.

4. The method of claim 3, wherein the chondroitinase is chondroitinase ABC.

5. The method of claim 1, wherein step (b) further comprises a step of treating the biological sample with any enzyme capable of performing at least one function selected from the group consisting of removing glycosaminoglycan (GAG) and modifying a glycosaminoglycan (GAG) component.

6. The method of claim 1, wherein step (c) comprises detecting of at least one selected from the group consisting of: total GAG-containing neurocan, total GAG-lacking neurocan, total GAG-containing RPTPβ/phosphacan proteins, and total GAG-lacking RPTPβ/phosphacan proteins.

7. The method of claim 1, wherein step (c) comprises detecting of at least one selected from the group consisting of: individual GAG-containing full-length neurocan, individual GAG lacking full-length neurocan, individual GAG-containing full-length RPTPβ/phosphacan protein isoforms, individual GAG-lacking full length RPTPβ/phosphacan protein isoforms, the individual proteolytically cleaved GAG-containing N-terminal neurocan fragment, the individual proteolytically cleaved GAG-lacking N-terminal neurocan fragment, the individual proteolytically cleaved GAG-containing C-terminal neurocan fragment, the individual proteolytically cleaved GAG-lacking C-terminal neurocan fragment, any other individual neurocan fragmentation product, and any individual RPTPβ/phosphacan fragmentation product.

8. The method of claim 1, wherein step (d) comprises comparing at least one selected from the group consisting of: total GAG-containing neurocan, total GAG-lacking neurocan, total GAG-containing RPTPβ/phosphacan proteins, and total GAG-lacking neurocan RPTPβ/phosphacan proteins.

9. The method of claim 1, wherein step (d) comprises comparing at least one selected from the group consisting of: individual GAG-containing full-length neurocan, individual GAG lacking full-length neurocan, individual GAG-containing full-length RPTPβ/phosphacan protein isoforms, individual GAG-lacking full length RPTPβ/phosphacan protein isoforms, the individual proteolytically cleaved GAG-containing N-terminal neurocan fragment, the individual proteolytically cleaved GAG-lacking N-terminal neurocan fragment, the individual proteolytically cleaved GAG-containing C-terminal neurocan fragment, the individual proteolytically cleaved GAG-lacking C-terminal neurocan fragment, any other individual neurocan fragmentation product, and any individual RPTPβ/phosphacan fragmentation product.

10. The method of claim 1, wherein step (c) further comprises detecting at least one selected from the group consisting of:
the levels, the ratios, and the differential pattern of at least one selected from the group consisting of:
the levels and/or differential patterns of at least one member chosen from the group consisting of: glycosaminoglycan (GAG)-containing neurocan proteins (encoded by a nucleotide sequence of SEQ ID NO:1, neurocan), GAG-lacking neurocan proteins (encoded by a nucleotide sequence of SEQ ID NO:1), a full length GAG-containing neurocan protein, a full length GAG-lacking neurocan protein, the proteolytically cleaved GAG-containing N-terminal neurocan fragment, the proteolytically cleaved GAG-lacking N-terminal neurocan fragment, the proteolytically cleaved GAG-containing C-terminal neurocan fragment, the proteolytically cleaved GAG-lacking C-terminal neurocan fragment, and any other products of neurocan fragmentation, GAG-containing RPTPβ/phosphacan proteins (encoded by a nucleotide sequence of SEQ ID NO: 2), GAG-lacking RPTPβ/phosphacan proteins (encoded by a nucleotide sequence of SEQ ID NO:2), a full-length GAG-containing RPTPβ/phosphacan protein isoforms, a full-length GAG-lacking RPTPβ/phosphacan protein isoforms and products of RPTPβ/phosphacan fragmentation.

11. The method of claim 1, wherein step (c) is performed with the use of antibodies that specifically recognize neurocan, RPTPβ/phosphacan isoforms or any component of these molecules.

12. The method of claim 11, wherein the antibody is chosen from the groupconsisting of 1F6 antibody, 650.24 antibody and 3F8 antibody.

13. The method of claim 1, wherein step (c) is performed without the use of an antibody.

14. The method of claim 1, wherein step (c) is accomplished via Western blotting and immunoblotting with at least one antibody for neurocan and/or any RPTPβ/phosphacan isoform or any component of these molecules.

15. The method of claim 1, wherein step (c) is accomplished by employing an ELISA assay with at least one antibody for neurocan and/or any RPTPβ/phosphacan isoform or any component of these molecules.

16. The method of claim 1, wherein step (c) is accomplished by employing any biological or chemical assay that detects at least one molecular form of a molecule selected from the group consisting of: neurocan, RPTPβ/phosphacan isoform, and a fragment thereof.

17. A method of determining the progression of open NTD where the increasing levels of neurocan and phosphacan during gestation are used as an indicator of disease progression.

18. A method of determining the efficacy of treatment of open NTD where the decreasing levels of neurocan and phosphacan during gestion are used as an indicator of treatment efficacy.

19. A method of treating an open NTD in a human fetus, where changes in levels of neurocan and/or RPTPβ/phosphacan or any other component of the extracellular matrix are used as a target of open NTD treatment.

20. A method of treating open NTD in a human fetus, the method comprising:
(a) obtaining a biological sample from a test subject;
(b) analyzing the biological sample with an assay that specifically detects at least one selected from the group consisting of: GAG-containing full length neurocan protein, GAG-lacking full length neurocan protein, GAG-containing full-length RPTPβ/phosphacan protein isoform, GAG-lacking full-length RPTPβ/phosphacan protein isoform, the proteolytically cleaved GAG-containing N-terminal neurocan fragment, the proteolytically cleaved GAG-lacking N-terminal neurocan fragment, the proteolytically cleaved GAG-containing C-terminal neurocan fragment, the proteolytically cleaved GAG-lacking C-terminal neurocan fragment, any other product of neurocan fragmentation, and any product of RPTPβ/phosphacan fragmentation;
(c) detecting at least one selected from the group consisting of:
the levels and the differential pattern of at least one selected from the group consisting of:
GAG-containing full length neurocan protein, GAG-lacking full length neurocan protein, GAG-containing full-length RPTPβ/phosphacan protein isoform, GAG-lacking full-length RPTPβ/phosphacan protein isoform, the proteolytically cleaved GAG-containing N-terminal neurocan fragment, the proteolytically cleaved GAG-lacking N-terminal neurocan fragment, the proteolytically cleaved GAG-containing C-terminal neurocan fragment, the proteolytically cleaved GAG-lacking C-terminal neurocan fragment, any other product of neurocan fragmentation, and any product of RPTPβ/phosphacan fragmentation;
(d) comparing at least one selected form the group consisting of:
the levels and the differential pattern of at least one selected from the group consisting of:
GAG-containing full length neurocan protein, GAG-lacking full length neurocan protein, GAG-containing full-length RPTPβ/phosphacan protein isoform, GAG-lacking full-length RPTPβ/phosphacan protein isoform, the proteolytically cleaved GAG-containing N-terminal neurocan fragment, the proteolytically cleaved GAG-lacking N-terminal neurocan fragment, the proteolytically cleaved GAG-containing C-terminal neurocan fragment, the proteolytically cleaved GAG-lacking C-terminal neurocan fragment, any other product of neurocan fragmentation, and any product of RPTPβ/phosphacan fragmentation in the sample with that of a comparator;
(e) determining that the subject has open NTD; and
(f) administering a treatment to the subject to treat an open NTD.

21. A method of claim 20, wherein the biological sample comprises amniotic fluid, blood, serum, plasma, or urine from a fetus or an individual carrying the fetus.


SEQUENCE LISTING (14 sequences)

SEQ ID NO: 1 - DNA, 40276 bp, Homo sapiens
  source 1..40276: mol_type = genomic DNA
ggagcgcagggcgcaggggctggacccggcgcggagctggctgagtcggagcgcagcgtcctttgtgcccggcggccgccccgggatgcgtccgagctaggagccaggtgggggatccgtgagggggccgtgttgcgggagaagacttcgggattagagacagagctcaggattagggagaggacctgagaccagggagaagtccccagaatggggagggggctgaagcgggggaggggagccctaggttgaggagggagcgcgaactgggagggggcttgggaatgctaggtgaacagacatttggggaggaaggttgtccgcggaatccggagactaccccccggtggggagggggccgggctgggggtgggtctcagggttgaggagggagcaatgggaaacagggctaggggaggggccccccgagtttggagggaagaagaggacacccagatgccgaatgggggactctggaacagggcacccccgtatctcagccgagacaccccaggattttaagtagggattcccaatttgcggttgaatggaggattccgggccggactgcagtggggagggggcccccagatcaccctgtgcccaatggaagggtatctgcgctgagagcgaacggccatggggaaggggctccctgccatctccctgtccttttgggtcaggtccggtcaccacgttctgactcagggaggaggtcctgggggtccccatcccaacgtggttccccgttattcccttagaccttgggttgaggtgttgtcccttatcccagggaacacactgggtgtcccaggtgaatgttgggtgggggtcccctagaccccccagctatggatccccaggacctgtgagggataccagctcagagggggggctgtcccccagcaccttcacagcctcccacgtccttacagcatgacacagacccctacccccaccccctttagttccacctgtccatatggacacatgtggaagcgctggcttggtttgggggcacaggggctgggaagtaaggggctgggaagtaagggccctgtctgggcaggagagacccccatccctacgcgtggggctttgttcctctcaggcagcatcaatgagaaacagccaggccagggagagggttggctctgggccccagcgggggacgtgggggcagggcaggcaggaaggggtgggtgcactgtgggaccccagaacccagccgtcgcaatagctgatggacattctgaaatcaagttccccctcaaggatatgggggttttgtgtgtgtgtccaccccagacaggcagggagggggagtatgcccaggggttctagggtcccctgtgagtctgatgtgactgtggttgtgtctttgtcagaaaacttgtgccgagggccacatcaccatgtggcaaaatgatgggtgtccatcggtcaccaaggccacaattatccatctcggtttgtgccaggggctggcatcgccgtgtgagtgaggagctatgtctgatgctgttccgttcatcaaggggtttatgtgggggggggggggcccgagactgtgggtgtgtgtctttgtgcagaagacacgccgcctgtcttgtaagtgcccatattgtctctctttgttaatgtcactgtgaatctgtgtgagtgtgtgtttccccagggtctgtgtctggggtgcgtggctgtgtgtgtcccgcttgtgtatctggaggggccaagggttctttccatgcggttggcagtgtgcacccttggcacgggggtgtgggacgtggtctgtgttcagagttggggcgctaacagggaaagcttgagcaggggttgaaatccccaaggggaagcccgggatgatgactgagcccatctccctttctcctccacggagttccacattcacacccatgcacactaatacaccctgactctcaggtcagaaacacacacatgtgcgcacagccccctgctgagggtcactcagactcacacacatccacagatcacactaggtcccatgtgctccacccggtgcacggagctgctgcaggctcacacacacaacatgcacccaccacatggcacaaccgccttcacacccttgccaggacacaacaccccggacacacacccagtcacaaacactcacaaatgcccattggcaaactcaccttcactcagtgctaacggagacttgaggtgggggtgaagtgaaggaggggacacacacacacttacacacacacactcacacacactctctcacacacacacattcacatggatccgagctgcccctcctccagcaccccgcaggcccctgcttgcatatgcatgaggcttgtacattattcatgagggcacgggccccagacagctgtgtaaatgctcatctctggccgccctggggaggaggtattttaagagaagggtgtgggtgtgtgagggtgcgagtggaagggagtgggagggtgtgtgtggggggtgtgcacctgaaactgtgtctgtcaccatgttgggcaacctcagtatatttttagcgtatacatgaggctttgtgtctaaagagctgtttttaaatttaacatttctaggactttgcaagtcttaggtcatttcatcctcacagtagctgcaggtggtacttgctggataatctccattttacaggtggggaaactgaggcacagagagattgaagcagcttgtcccggattagaagcagagctatctggatgtagtctgacctgttaacggggtgtgtgtgtgtgtgtgtgtgtgtgtgtgtgtgtgtgagagagagagagaaagagaattctctggccttgtcctctcgtaaggccctgctgggggcaggtgcaacccggagtcctgtctctctctaatacctggccaggcctggaacagccatgaggggaccccacacttgctcagggttccaaggggaagaaatgagaacagcctggtgaaggcagaggttgctgaggtttacaagttggtagttgggagcactggtccagggtctggctttgacctctaccctgctgtgcagcctgaggcaaacctctagccctctctggtccctgaggcatcgtgttagcaggcaggaggcaggggtggactaggtgggctttgccgctgcgttgccgcctcagagttcagcaccacggacaggtcccaggccggcctcccagcaggggactctgggagctgggaaggagaactggggaaagggacagctgtcacatacagcctccccttctggactccgttcacaccccttcctcagggataatttcttatgggggcggggagcaaggattgggtttgcagatgatgttctaattttccatctgcgcccacctcctgagccctggtgaccggggttcgcaggagtgggaggttttggggagggaccacctccccaggattcctagtctctggaacaatgtgaagtcaggaaatgcactgctgtgtcccaacgactggcatgggctttgctcacagtaggcgcttaataattgtccactttatccctatgggtcctagcccctatagggaaatggacccacaactgaagtttctctgtgggcctaagaagtatctttgttgagctgtgtgaagtcaggaggaggatgggtgggcaggggtgagccccttcctccagctgggagttggcggctcctccaaaatcccagaaaccaagaatagaatcttggtgacaaccaaatcctggaattgaagctgcgaatcgtgtcagccagcaaacccagattaaaggatggcttagaggccagcctgctgcctggactgaggatgctgggaggactgggacagactgctgccctctaggagccgccagtctgatggaggaggctgacttgaaaaatggtatttcagtcttggctgtgttaaggtcggtaataataatagcagcagctaaaatgtaccccttactgcatgagacagttctaagcattttatatgtattgactcatccattcctttcaactacctgatgttatccccatctgacccacaaagaaagtaaggcacagagaaggtaagcaacttgcccaagatcacacaaaaggaacagtgagccaattccagatttgattcactaccagcacccaggcttctgaccacagacttcattgcttggattgttttttgttgttgtgttgtgttttgttttgttttcagtctcgcgctgtcacccaggctggagtgtggtggtggaatcatagctcactgcagccttgaactcctgggctcaagcaatcctcccacctcagcctcctgcatagctgggaccacaggtgtgcgccaccaagcccagataattttttttttttttgagacagagtctcaccctgtcgcccaggctggagtgcagtggcgtgatctcggctcactgcaacctccacctcccgggttcaagtaattctcctgcctcagcctcctgagtagctgggattacaggcgcctgccaccacgcccagctaatttttgtatttttagtagagacggggtttcaccatgttggtaaggctggtctcgaacccctgacctcgtgatccaccctcctcggcctcccaaagtgctaggattacaggcgtgagccaccacgcctggctgctaattttttaaatagaggtaagatctcactatgttgcccaggctggtctcaagcttctggcctcaagcagtcctcccacctcagcctcccaaagtgctggaactataggcgtgagccaccgcgtccagccactgctattctgaggtcaagtggccctgggttcgaattctggctcagccctttgctggctccttctgtgacctagaataacacactggtctctctgagccctggtttcctgatctgtagagtgggggagtttggtcctgcaatgctgaatatctgggagggttgggctgtggctcacccctccctccctctccattttgttccagatccaggatgggggccccgtttgtctgggccttgggccttttgatgctgcagatgctgctctttgtggctggggaacagggtgagttggtttgtggggagggagagattggggtctaggggatggacaagatggggcagcttttgaattccagagccttctcctgctggtctcctggcatgggctagagaataaaatgagaatagattttaaaaggtctttgaacagtcaaaagcgaacaggatacctaagaggttatttttagtcattgtcagcagaagctggagattcccgcctctcatcgttttgctcagatgaactggagagaaagaaagaaagaaatgagaatagtgcagagattgattagtgatgtctgccaagggtggagcgatggaaataaaggtctgtgtccctctactggtggtcaagagcccaggttttagaaatccctcacagatttggtctgtgaccttaggcaagtataattatctctctgaacttcagtttcctcaactccccccaaagagaatgagacattgctactctgcagatggttatgaaaatcaaatgagctgattgctataaagtgcctgttacaaagatggtactctgcaaacaccatttccttttcattagctgtcttttgctttacctttcggaaaaactggggctcagggaaggaaaatgcctctcccagggccatacagtgaatcagtggcactggagtgaaatccctgtccttataaaaataaagtgaggggctaggcacagtggtttatgcctataattctagcactttgggaggctgaggcaggcagatcacttgaggtcaggggttccaggctagcctgcctaacatggtgaaactccgtctctacagaaaatacaaaaattagccaggtgtggtggcacatgcctgtaatcccatcttctcgggaggctgaggcatgagaatcgcttgaacctgggaggcgaaggttgcagtgagccgagatcatgccactccactccagcctgggtgacagagtcagactcaaaaaaataataataaggctgggtgtagtggctcacccctataatcccagaactgtgggaggccaaggtaggtggatcagttgaggtcaggaattggagaccagcctggccaacatggtgaaacccatctctactaaaaatgcaaaaattagccaggcgtggtggcacacacctgtaatcccagctactggggaggctgaggcaggagaattgcttgaacttaggaggcagaggttgcagtgagctgagatcatgccactgcactccagcctgggagatggagcgagactccatctcaaaagtataaataaataaaagaataataataataaggggctgggcacagtggctcatacctataatcccagcactttcgggaggctgaggtgggaggattgcttgagtctaggagcttgaggctgcagtgaatcatgatcatgtcactgcattctaccctgggcgatagagtgagactcaatctctaaaaatagttttttgtttttttttttgagatggagtttcgctcttgttgcccaggctggagtgcaatggtgcgaccttggctcaccgcaacctctgcctcccgggttcaagcgattctcctgcctcagcctcctgagtagctgggattacaggcatgtgccaccacacccagctaattttgtatttttagtaaagacagggtttctccatgttggtcaggctggtttcaaattcctgacctcaggtgatttgcccgccttggtctctcaaagtgctggggttacaggcatgagctatcgcgtccagccactgcttggattctgaggtcaagtggccctaggttcaaattctggctcagcccttttaaaaaattttttaaaagagggaaatagcagtccctgcttggttcttgcctctgaatcaggccctctctccaccttctccaacccaggcacacaggatatcaccgatgccagcgaaagggggctccacatgcagaagctggggtctgggtcagtgcaggctgcgctggcggagctggtggccctgccctgtctctttaccctgcagccacggccaagcgcagcccgagatgcccctcggataaagtggaccaaggtgcggactgcgtcgggccagcgacaggacttgcccatcctggtggccaaggacaatgtcgtgagggtggccaaaagctggcagggacgagtgtcactgccttcctacccccggcgccgagccaacgccacgctacttctggggccactgagggccagtgactctgggctgtaccgctgccaggtggtgaggggcatcgaggatgagcaggacctggtgcccttggaggtgacaggtcagttgggggcaaaggaggggccgggggccggggagagggagggctgagcctggagcccacccactgaatgtcaccttagaaatcactccctgagacctggcctggtgtctcatgcctgtaatcccagcactttggcaggccaaggcgggtggattgcttgagcacaggagttcgagaccagcctgggtaacatggtgaaacctcgtctccacagaaaatacaaaaattagccaggcatggtggcacatgcctgtagtccagctacttgggaggcataggtgggaggatcacttgagactgggaggttgaggctactccactccagcctggatgacagagggagaccctgtcacaaaaaaaaaaaaaaaaaaaaggaaagaaatcactctctgaaccgggattccctttgaattcttctgataaagtcctaaagtctttatggtgcctataatctgagcactttgggaggccgaggcgtgtggatcagttgaggtcaggagtttgagaccagtgtggccaacatggtgaaaccccgtctttaaaaaaaaaaaaaaaaaattagccaggcatggtgatgcatgcctgtgatcccagctacttgggaggctgaggcaggagaactgcttgaacccagaaggtggaggttgcagtgagctgagattgagccactgcactccagcctgggccacaccgcgagactctgtctcaaaagaaaaaaaacgtctgtatcagtatgggtactatcttgtctttaacacctctcttttttagctatgagagaggagtctccagctcagacttctgggggccacaatatctaggcaggatttttgtttgtttgttttgagatggagtctcactcttgttgcccaggctggagtgcagtggtgctatctaagctcactgcatcctccgtctaggcaggattttacttggagtcaattttatggttatctgattttatgtaagacaaaggatactggctttctatttaaaatagaaataaaaaattccttgtaaaataaacttaagtttttttctaagtgaggcaattttttaaaagtaagttaatttaagatgtatattaaatattaagcaagaacaattctcaaatacctgtttaggcaattcttttttttttttttttttttttttgagatgggagtctcgctctgtctcccaggctggagtgcagtggcatgatctcaactcatcgccagctctgcctcccgggttcacaccattctcctgcctcagcctcctgagtagctgggactacaggcgcccgccacagcgcctggctaattttttgtatttttagtagagacggggtttcaccgtgttagccaggatggtcttgatctcctgacctcgtgatctgcccacctcggcctcccaaagtgctaggattaccaggcatgagccaccgcacctggcaaccaattcctttattaaattcttcttgttgagctgcgtgtggtagtgcacacctgtagtttcagcactttgggaggctgaagaggaagggtcacttcagccaggaattcaagaccagcctgggaaacatagcaagaccctgtctctattagaaaaacaaaaacaaacaaacaaacaaaaaacaaaaaacggggatgggggcccagtggctcatgtctgtaatcccagcactttgggagaccaaggcaggagaatcacttaagctcaggagtttgagaccagcctgggcaacatagtgagaccttgtctatacaaaattccaaattccaaaacattagccaggtatgatggtgtgttcctgtagtcttagctactcaggaggctgaggtgggaggattgcttgagtcccaagagatcgaggctgcagcgagccttgatcatgccactgcactccagcctgggcaacacagcaagaccctgtctcaaaaataataataatatggctgggcgcagtggctcacgcctgtaatcccagcattttgggaggtcgagacgggtggatcatgaggtcaggagttcgagaccagcagatcatgaggtcaggagttcgagaccagcctggccaagatggtgaaaccccttctctactaaaaatacaaaaattagccaggcatggtggcacattcctgtaaccccagctactcgggaggctgaggcaggagaatcgcttgaacccaggaggtggaggttgaggtgagccgagattgtgctattgcactccagcctgggcaacaagagggaaactccatcttaaaaataataataataataataatacaactaaataagccagatgtggcggctcacacctctaatctcagcattttgaaagactaaggcaggaggattgcttgagcccaggagttcaagaccagtctgagcaagatggtgagacctcgtctctacaaaatttttaaaaaaatattagcagggcatgctagtgtgcgcctgtagtctcagcttgtcaggaggctgaggtgggaggatcgcttaagtctggaaggttggaagctgcagtgagctgagatcgcaccactgcacttcagcctgggtgacagagggagaccctgactcaaataaataaataaataaatagtcaaaaactaaaaataaagaacagggtttggcacacagtagatgctcaataaatgactcagtgaatggattattgtacagcaaacatcccgtggactcctgctgggaaacctgtttcccttaacttaaattgaccttgggcaagtccctgattcctgtcttggggcttaacctgcatgcccccaaactgggaactggggacacagtgtcttaccacatagtttatggagtcaaccagtcatcccctttcagaggcctcatttctgaaaattgataaaagctaacaacaacaacaacaacaaactgattggtggtgaggcaagtgaattccacatctaggtaacttcaatttcattcattcagccaaactctttttgttgttgtttgagacagagtctcactctgtcacccaggctggagttcagtggtgtgatcttggctcactgcacctctgcctcgtgggttcaagcgattctcctgcttcagcctctggagtagctgggattacaggtgcacacaaccacacctggctaatttttgtatttttagtagagacagggtttcaccacattggccaggctggtttccaactcctagcctcaagtgatctgcctgcctcggtcttccaaagtgctgggattacaggcatgagctgctatgcccggacattcagccaaacatttattgagcacctacactgtactggtccaatgctgaggacacagagctctcagttggataagccagagcaaagtgtttcatttcattgtcttgttcagccatttctttcatccctaaatcagaatttgaatcacagtgagcctgttaccagggcttcacatcctggcttacatgtcacctcctccaggaagccttccaggaccacatttccaggccaggcaagaaccacatttgggccgggcgcggtggctcatgcctgtaatcccagcactttgggaggctgaggtgggcggatcacgaggtcaggagatcaagaccatcctggctaacatggtgaaaccccatctctactaaaaatagaaaaaaattagctgggcgtgatggcaggcgcctgtagtcccagctacttgggaggctgaggcaggagaatggtgtgaacccgggaggcggagcttgcagtgagctgagatcgcgccactgcactgcagcctggcgacagagtgagattctgtctcaaaaaataaataaaataaaataaataaaataaaaaataaataaataaataaaaaagaaccacatttggtctcccattgctgcctgtgcttccctattagcgcatgcacttaattcctaaaaggcagaaactctctgtcagccttctccacacctcctgccacctccagggcctaaccctgggcatgaagagtgctatctttggagtcagatctctctgaatttgaattctggttctgtttcttatttgctgcgtgggcctggacctgtccctcaacctttctgaccctaagtttcctcctctagaacaatcagatttgttgtaagagttcaataagataatgcttataaatgcttttcttttttttaatgttttagtttttttgagatggagtcttgttctgtctcccaggctggagtgcagtggctcaatcccggctcaccgcaacctctgcctcctgggttcaagcgattctcctgcctcagccgccctagtagctgggattacagacgcatgccaccatgcccagctattttttgtatttttagtagagacagggtttcaccatgttagccaggctggtctcgaactcctgacctaaagtgatctgcccaccttggcctcccaaagtgctaggattacagaagtgagccaacatgcctgaacggccgtaaacgcttttcacattgtaaatatttgctaacttgtaaccctttatggccattgttatcagtttcctcatctgtaaaatgggggcatatggatcttgctccccaccctcgacccacactgtgcctggcgctaggtctattattatcagacaggggtggggagtcattctgcaagaggtaggtctgttcttgaagattccagcataagtcaactgtccccccatcctggatgttcccccacaggtgttgtgttccactaccgatcagcccgggaccgctatgcactgaccttcgctgaggcccaggaggcctgccgtctcagctcagccatcattgcagcccctcggcatctacaggctgcctttgaggatggctttgacaactgtgatgctggctggctctctgaccgcactgttcggtgagggggatacacagggcagggagatgaagactagctcatggggaaggaggttccttgggggctccaggagcacacatctgagagggaccctccccttgtgttgtcaggtatcctatcacccagtcccgtcctggttgctatggcgaccgtagcagccttccaggggttcggagctatgggaggcgcaacccacaggaactctacgatgtgtattgctttgcccgggagctggggggtaagtctgggactggcaggacccggcccctccgcctctctttgagtatctattattctccccaactcccatcctccggggtccttatgcaactcccctgtcttatacctccctaaacctgcaaccaagacatgattccactcattttctgagctttccatgcctccctgagtccccctccctccctttgcccccttcctctctctgatcctctttccctctctaatcccctgtcccctgcctgcctccctgtccctatccccttcctcctctgtgagcccccttcaccgagcccctccctctctctgaagcccgacttgcccatgggcctgagttactccatttgtaaaaagaggcttagctctgcatgagaggtaaggacaaggatgatcccggagctggacgcacagctgcggttgtcaccgcctcttctaaccaccaccctaacacgtccaggtcggaactatttggagttggacccacttcctgggctccaggggagggttccccagcccccctgacctccacccgcccctcccgcaggcgaggtcttctacgtgggcccggcccgccgcctgacactggccggcgcgcgtgcacagtgccgccgccagggtgccgcgctggcctcggtgggacagctgcacctggcctggcatgagggcctggaccagtgcgacccgggctggctggccgacggcagcgtgcgctacccgatccagacgccgcgccggcgctgcgggggcccagccccgggcgtgcgcaccgtctaccgcttcgctaaccggaccggcttcccctcacccgccgagcgcttcgacgcctactgcttccgaggtgcgtgcgtcccctggtggccgcgcccccagggctttcactttggcgaaggccacgtccctgaaagcctcgccaagccaagggagagacacatggaagctcgctttgtgataagccacatccctgggtgagggccacgcccccgggtgaaggccacacccgttacgacaagtctttccctaggagccccgtcctgggtaaagaacagggctccagaggaggccactctggtggggagtcacctttgctgggtgggctgtatctcctgaggtccggaggccccataaccttgaaagacaagcccctaggtggggcatactctacctagagccacgcccatacgcagaaacacccccgtggaagagataatgcctcaattggccacctctgggcaccacaccggatgtgggagcagggatgacactcgcccttgttggggggcagtggggacagctttggaagctgcaagcatcaaccccaaggagccatgtcctctaagtaagctgccccagtcctggcatgggcataccatgcccacggggcaccacctgtaccaggggaggatccgtcaccaaggttgagttgaacccctcaacaggcagacccaagttcatgagagttggacactacctgagctttgcccatgagccaaattgtctttttttcttttttcttttgagacagagtctcactctgtcacccaggctggagtgcagtggtgtgatctcggctcactgcaacctccacctcccttgttcaagcaattctcctgcctcagccttcggagtagctgggactacaggaatgtgccaccatgcccagctaatttttgtatttttagtagagacggggtttcaccatgttggccaggctggtcttgaactcctgacctcaagtgatccacccaccttggcctcccaaagtgctgggattacggacgtgagccaccgcgcctggcccaaattctcttgagtcctcactgcagccagttgactctgaccctgacagtggctcaggtggtctccagcctgaacagtggaggtgaaaattttgcacttccctctcccaggctgggggaatctcagaaactacttaggagagacagaggtacacagaaggcttatgctgcagcatgctggaaagtgggtagacttcaagcagaacttcccaggcaacccctgggcctaacacaacctcttctgctttctcccacagctcatcaccccacgtcacaacatggagacctagagaccccatcctctggggatgagggggagattctgtcagcagaggggcccccagttagagaactggagcccaccctggaggaggaagaggtggtcacccctgacttccaggagcctctggtgtccagtggggaagaagaaaccctgattttggaggagaagcaggagtctcaacagaccctcagccctacccctggggaccccatgctggcctcatggcccactggggaagtgtggctaagcacggtggcccccagccctagcgacatgggggcaggcactgcagcaagttcacacacggaggtggccccaactgaccctatgcctaggagaagggggcgcttcaaagggttgaatgggcgctacttccagcagcaggaaccggagccggggctgcaaggggggatggaggccagcgcccagccccccacctcagaggctgcagtgaaccaaatggagcctccgttggccatggcagtcacagagatgttgggcagtggccagagccggagcccctgggctgatctgaccaatgaggtggatatgcctggagctggtgagttgctctgggggaggcgggacctacctggggatctggaggtgagggtagagaggtagccatggctacactcagaatgagggaggaagctccaaatcccagctgggtcctctggccccagaagccccctctcccttgggcctggagtccaaccaaaggggctgctgagagatcattgtaatgattgccttgatgttgttgcaggttctgctggtggcaagagctccccagagccctggctgtggccccctaccatggtcccacccagcatctcaggccacagcagggcccctgtcctggagctagagaaagccgagggccccagtgccaggccagccaccccagacctgttttggtcccccttggaggccactgtctcagctcccagccctgccccctgggaggcattccctgtggccacctccccagatctccctatgatggccatgctgcgtggtcccaaagagtggatgctaccacaccccacccccatctccaccgaggccaatagagttgaggcacatggtgaggccaccgccacggctccaccctcccctgctgcagagaccaaggtgtattccctgcctctctctttgaccccaacaggacagggtggagaggccatgcccacaacacctgagtcccccagggcagacttcagagaaactggggagaccagccctgctcaggtcaacaaagctgagcactccagctccagcccatggccttctgtaaacaggaatgtggctgtaggttttgtccccactgagactgccactgagccaacgggcctcaggggtatcccggggtctgagtctggggtcttcgacacagcagaaagccccacttctggcttgcaggccactgtagatgaggtgcaggacccctggccctcagtgtacagcaaagggctggatgcaagttccccatctgcccccctggggagccctggagtcttcttggtacccaaagtcaccccaaatttggagccttgggttgctacagatgaaggacccactgtgaatcccatggattccacagtcacgccggcccccagtgatgctagtggaatttgggaacctggatcccaggtgtttgaagaagccgaaagcaccaccttgagccctcaggtggccctggatacaagcattgtgacgcccctcacgaccctggagcagggggacaaggttggagttccagccatgtctacactgggctcctcaagctcccaaccccacccagagccagaggatcaggtggagacccagggaacatcaggagcttcagtgcctccgcatcagagcagtcccctagggaaaccggctgttcctcctgggacaccgactgcagccagtgtgggcgagtctgcctcagtttcctcaggggagcctacggtaccgtgggacccctccagcaccctgctgcctgtcaccctgggcatagaggacttcgaactggaggtcctggcagggagcccgggtgtagagagcttctgggaggaggtggcaagtggagaggagccagccctgccagggacccctatgaatgcaggtgcggaggagggtgagtacaaagtcccggggctctgtccagctctccatggtcagggcttggggagcaggggctgggggatcccagagcagggaggaatggttgtccctggggatctggaagctttctagtggggcatgacgctggaggtaggagcttatcaggtggagttgaatttcagaaaggacattcctggcagaggaacgagcactggcaaaggccaggagatgaagtagcgcttgatcaatttcgggcacagagcatacatcccaatgaacaaagtgtgagagtagtgagtggccgggcatggtgcctcatgcctgtaatcccagcactttgggaggctgaggcgggcagatcacttgaggtcaggagttcaagaccagcctggccaacatggcaaaactccgtctctactagaaatataaaaattagccgggtgtggtggcacacacctgtaatcccagctactggggaagctgaggcataagaatcgcttgaacccgggaggcggaggttgcagtgagctgagattgtaccactgcacttccagcctgggtgacagagtgagactctgtcaaaaacaaaaacaaaaacaaagtgtgggagtggcgaggcattgagtagagtctgatttggggatgagttcaggtacaagccatgtctacatctgctttgagctggacagtggtgtgatggactggctttccaggattccccgagtctgaggaagacagagcaggacacagacgcctcccaggcgagaccagagggaggaggcatggcagggagggtttcctggacaaatgtgaatgggagctggaattcggtgatttgactcgtgggggacctcaagccacagggaaagtgggtgctggggagccatgcaaggggcgagaatcacccccattctacagatagcaaaccggggctcagggaggtaaaacgccgcttggctaatcagtggtcacacctcaactggagcccaagtcagcctgagtcttagctactctgtggccagcagcgtccatagaaatcggctgccctgttctccctggggccaggtcagcctcagctcagcctgcaaattccagcccttcttaggttctgccaaattaagctgagttttttggccaaatgaaatttagaaacaaacaaacaaaaaagaggctgcgtgtgatggctcatgcctgtcatcctagccctttgggaggctgaggtgggagaatctcttgagctcaagagttaagaccaggctggccaacacagcaagaccccatctctactaaaaataaaaagaaattagccaggctgcagtgagctatgatagttccaccgcgagaccctgcatgaaatcaaactaacaaaacaaaacaagcagctaattaataagtctttttttctgagttgcacactttttttttgtttgttttttgagactgagtctcactctgtcatccagcctggagtgcagtggtgtgatctcggcgcactgcaacctccgcctcccaggttcaagtgattctcctgcctcaaccttctgagtagctgggattacaggctcccgccaccacgcctggctaatttttgtatttttagtagagacaaggcttcaccatgttggccaggctggtcccgaactcctgacctcaggtgatccacctgccttggcctcccaaagtgctgggattacaactgtgagccatagcacccagtcgagttgcacacttttattaacaccattgatgtttaactaccgcaccccccacccccaaatttttttttttttttttttagaaagggtctcactctgtcacctaggcttgagtgcagtggcaagatcgtggctcactgcagccttgacctccccaggctcaggtgatccttctgcctcaggctcccaagcagctgggaccaggtgtgcaccaacaggcctggctaatttttgtattttttatagacagggttttgccatgttgcccaagctagtgcccgtttttttgaaggcaaagtgtcatgatgatggaaatagctgatgcaggtttcatcatccagcctgtcacttctgggctgtgttggggtgggatcttttttttttttttccttttttttttttttccagacagggtcttggtcagttgttcaggctggagtgcagtggtgcaatcatagctcactgcaccgaggctcaagtgatcctcccacatcagcttctccagtagcttggactacaggcatgcaaccaccacacccggctgtgtgtgtgtgtgtgtgtgtgtgtgtgtgtgtgtgtgtgtgtgtagagatggggtctccctatgttatccaggctggtctcaaactcctgggctcaagtgatcctcttgccttggcctcccaaagtgttgggattacagacatgagccgtagcgccgggcagttctttttttcttagatgtccaggaggcaggccgaccgccccaccccgtatcgataaaggctgacatttatgggttactgccagtgtgcttggcttattcaaagcactttactggagttatcttggttaatccttttaatgtccttatgggaaaagctccagtgtgatccttactttgcagatagggagacagacacagagaggtgaagtgccttgtgcagccatgcaactaagggagtgtcaaggctggggtttgaatccaggtcatctgaacatggggtttcttttttttttttttttgagagagagagtctttctccgtcgcccaggctggagtacagtggtgatctcaggtcagtgcaacctccgcctcccaggttcaagtgattttcatgcctcagcctcctgagtagctgggattgtaggcgtgcaccaccatgtctggctaatttttatacttttagtagagatggtgtttcaccatgttgaccaggttggtctcaagctcctgactcaagtgatctgcccgcctcagcctcccgaagtgctgggattacaggcgtgagccaccgcacctggccggaaaacagagcttctgtctcagccactaggctgcgaaatgcttagctcgttggcataaaaaagtacatgaggccgggcatgatggctcacacctgtaattccagcactttgggaggctgagacggaggatcacttgagcccaggaattggagaccagccttggcaacatagtgagaccccgtctctacaaaacaaattttaaaattagccgggcatggtgggtacctgtggtcccagctactcaggggtgctgaggtgggaggaccgcttgaccctgggaggttgcagctgcagtgagctatgatcatgcaactgcactccagcctgggggacagagcaagactccatctcaaaaaaaaaaattaatcttggggtggtggtgaggagagaagagacgggctaggtggggatcaaccctggaaagatgatcagaggggaagaagctcaatgcccaaagaaatagagaaggggcagtcagagaggtgggagggcgaccacataggaacactaaggaaacgggggttggagctgcacctgggctgaggctgagtctgtcttggacttttctggatcctgggctccacaatgaagccaaccacagcaggatggagattggcgggcagtgctggggagacccaggcagctacccacccgccgcttgcagaagaaatgctaatgaagccaaaaataaatgcagtaccagcgagtgactgagtcaccgccagtccgtgggcaggtgtcagcagcgcggggcacctggcggggctcatgtgtcccgccgcctggcagccttgtgcgtgtgcacacacgtgcagggccgtcgtccagccacaggccctgccatccacaagcccgtttgccatccatggggccaccccagggacaggcctcagctaccgcaggagggccagctgctaccttcattccgttagcaagcaattactgagtgcctattctgtcccagaccatatgttgaaaccaaatcccctctcctctgaaaactgacgtcctagagcaacattctagaactttcccagggatggaaatgttctctatccacgctaatttgaaaaatggctaggacaactgagggacgacgtttttacttttatttcactttaattgatttaagcgtgaatatctatatgtggctgccatatggaacaccagtgctctagggaaagatacagacaagaaacgattgttctaataaaaaaagtaaagtagtgctcgcttcagcagcacatatgctataattagaacaatatagagaagattagcacagcccctgtgcaaaaaatgaagcatttcattcttttttttttttatgagatggagtctcactctgtcacccaggctggaggcgcgatctcagctcactgcaacctccgccttcggcattcaagggattctcctgcctcagcctcccgagtagctgggattacaggttcctgccaccatacccagctaattttttgtatttttagtagagatggggtttcaccatgttggccaggctggtctcgaactcctgacctcaggtgatccacctgccttggcctcccaaagtgctaggattacaggcatgagccatggcacctggcctctttttttttaatttaatttttcaaaaaatttaattccactgggcagtggaatggtcagatgctgccctgacaggatctgggttggctggagcaggatgaggattttttttcagtctgtcacccaggctagaacctatgtccactcctgtgccccaattcatgagaagcacagtgatgagcagggtgtgcccaaaggaggggaaggacactgaagtcagggaggacactgaagcccatgttctgggagaagacattctacgaggtccttcaggcagccctggtctctgggagccccattccagttcagtctgaggaaggactttgctacaggcaggaaagtccccagatgctttggggcagtttctggaagcagtaaactcctcgtaacaggggtcatatgggagggagttggtgaccgctggttggggaagttataaagagggctccatagagagggtttgattagagtggtttgggggcctggggtcttgattccaggaaggcaaaagatctgtggcctgactcttccccacactacccatccatcctgcagtgcactcagatccctgtgagaacaacccttgtcttcatggagggacatgtaatgccaatggcaccatgtatggctgtagctgtgatcagggcttcgccggggagaactgtgagattggtgagtaccccagactcaggatctgaatctgaatttgtttgactccagagccttgggttgtactccagcagccatgccctcagaattcagagatcacaaaaagctggtttcagatgctccattcccaaaccccactccttgcattcagctaaatcagcccagttagtggaaccagaatcaaacagctgagctttcatctctggtgtgctgtctcccattactaccagtcagaggtgggaaggaaaagacaaaggaattatatgtctgagactcctccctgcaaaggccctttggagatcctgccctttcctgggtcggtgggaggtataagcactgccaccagaactgttgggtgctagcttcaaagcccaacacaagaaatactaccaattatgaaattacaacaacaaatataaatgacataattttttgagctctatgtgtcatcttgacttcacaaccttcacaaccacatgactgattacagaattataacaataaatataaatgatataatttcttgagctcttctattgcttccctactcacaatcatccttgagtaggagcccattattattaccattttcaaaaaaagtaagctgagtttcaaggaggggaagtgacttgcccagtgccatacgtgcctcaagcactgtctacacatggaccagtagaagaggagctcaatgaatatctagctttacccacaaacagctttgtgactctgtgcaagtctttgcaaatgcctaaaagagactccagcttcagcattctatgagcctgtttttgagagtataaccttcacttctctagagtcaggtagaacagtctgcacatcctggctctcagctgccccagtggcatctgctctcattaatgctaatttatgttaattgtatgaggttccacccccttgcctgcagagtcattgggccatctgtgcaaactgtgactgctgcacttaggtccaggatggatggtaggaccatgaccccatcttctgcttagtttcctgtgggggctcagagccaaggggaagctgacctctaactcagtctcttcccctctctgccagacattgatgactgcctctgcagcccctgtgagaatggaggcacctgtattgatgaggtcaatggctttgtctgcctttgcctccccagctatgggggcagcttttgtgagaaaggtgagtttctattgcaacaccagaaacagtaccaagagtggggttgggtgcccagccacaggcttccccacatactccaggtccctgcctccagttccatggctgtgagcctgacacaagataaattattcttttagacaaactgtgcgtaacatagaattaaccattttactttattttattatttttttgagatggaatcttgctctgtcacccaggctggagtgcagtggcgccatctcagctcactgcaacccccacctcccagtttcaagcaattttccctgcctcagcctcccaagtagctgggattacaggcgcccgccaccacgcctggctaatttttgtattttttagtagagatggggttttgctatgttggccgggctggtcttgaactcctgacctcaggtgatccacccaccttggcatcccaaagtgctggaattacaggcatgtgccactgcgcctgccctattttattttattttttttttttgagacagagtctcgctctgtcaccaggctggaatgtagtggcacaatctcagctcactgcaacctccaactccctggtccaagtgattctcctacctcagcctcccgagtagctgggattacaggcacacgccaccacgcctagctaaatttattttattttttgagacagggtctcaccctggcacccaggctggatggagtgcagtggtgcgatcttggctcattgcagctttgacctcccaagctcaagtgatcctccctccttagcctccccagtaggctgggactacaggcacacaccaccatgcctggctaatttttgtattttttgtagagacaaggtttcaccatgttgcccagactggtcttgaactcctgagctcaagcaatctgtccacatcagcctcccaaactactgggattacagccactgagcccagccaaattaaccattttaaagtgtacatttcagtggcattctgtacaatcacaatgttgtacagccatcacctctgtctagttccagaactttttcaacaccccagaaggaaactctgtgcccactaggtggtcactccctattcccattccccagtccctggcaaccactaatctgctttctgtctctgtggatttgcctgttctagacatttcatatgaatggaatctcaaaatatgtgtcttttgtgtctggcttctttcacttggcatgttttcaaggttcacctgtattgtaatatgaatcagtgtttcattccttttcatggctgagtagtattccattgtatggctatatcacattttgtttatccattcatggtttgatggatatttgggttgtttcctccagaggctaaggtcaatagtgctgctgtgaatatattcttgtatgtgtttttgtttgaataactatttgccattcttttttttccctctactctttttggtgtatactctcaggagtggaattgctgggtcatatggtaactctaggtttaatttactttttttttgtagagatggggttttgccatgttgcccaggctgctctaaaactcctgggttcatgtgatcctcctgcctcagcctcccaaagtgctggaattacagacatgagccactgtgcttggccttttttttttttttttgaaacaggttctcactctgtgcccaggctggaaagtacagtggtacaatcatagttcactgtcaccttgaactcccaggctcaagcgattctcttgcttcagcctacccagtagctggaactacaggtttgtgccaccatgcctggctaatttttatttttttaatttctttttatttttattttttgagacagggtctcactctgtcacctaggctggagtgaagtggtgtgatcttggctcactacaacctccacctcccggttcaagtgattcttgtgcctcaatctccccagtatctgggactataggcacctaccaccacacgtgactcaattttttttttttttgaagtttagtagagatggggccaggctggtcttgaactcctggcctcaagtgatccaccagcctcgacctcccaaaatactgggattacaggcatgaaccaccatcctcagcctacttttccttttaaaaaaaaattatatgtaatcccagcactttgggaggccaaggcaggctgatcatgtcaggagatcaagaccatcctggccaacatggagaaaccccatctctactaaaaatacaaaaattagcaggcatggcgacacgtgcctgtaatcccaggtacttgggaggctgaggcaggagaattgcttgaacctgggaggtggtgtctacagtgagccaagatcgcaccattacactccagcctgggcaacagagccagactccatctcaaaaaaaaaaaaattttatatatatatatctccatcctagtgggtgtgaagtgatatctcatggtggttttgacattgacaatgagcatcttttcatgtgcttgttggccatttgtatgtcttctgtggagaaatgtctgagctcccatgtccatacgaagggtgtggccccgacatcagagaaggagccttcagcattggaatgagagggggtgtcagggaatgatctcccagttaaatctcttccagaaacaaagaaaagacattaagatttggccagtgtggtggctcacgtctgtaattccagcattttgggaggctgaggtgggcagatcacttgaggccaggagtttgagaccaacctgaccaacatggtgaaaccctatctctactaaaaatacaaaattaggtgggcgtggtggcgcacgcctgtaatcccagctactcaggaggctgaggtgggaggattgcttgagtatgggaggcggaggtagcagtgagccgagattgtgccattgcacgccagcctgggcgacggagtgagaccctgtccaaaaaaaaaagaaaaaaaagaagtcaatatgggaggtgaaagggttgggagagcagagcctcaggaggaggggccaggcatggggagaggggtgatttcgccccgcagtgaccttggattgggccctctctctggtgggctggctgtgctcatggctggtaggccttgggccaaggccagccccccctcacgctcctatcccatctcccagacaccgagggctgtgaccgcggctggcataagttccagggccactgttaccgctattttgcccaccggagggcatgggaagatgccgagaaggactgccgccgccgctccggccacctgaccagcgtccactcaccggaggaacacagcttcattaatagtaggggctctggggagggggccacctgcctggagggtgggcaggggtggcacttgtagcccttttgccagggcatcgcatccctcccctggttttcaagacgtcatctttccaaagctgtgttcattaacgcaagccctgactgcttcttaatgcatgaaatctgtcaattcacttgttaattcatacattcacttcattcattcattcaccaaatatttactgagcacctgctatatgccaggcaccattccaggaatattgacacagcacagcagcaatcaaggctgactttgctttgtccccagggagtgaccatttcttggggaggatatagacaaacaagtaaatatctaagtaaataagtctatactgtaattcaacagatcctatgtgtgctgataacaaaacagggccaggagatggagggtgatgggagaagcacctgtaatgtgttcaaagacctaggagagagacaagtgttccattcatgtaatccgggacttattgatttattcattatccctctgccttctgctcatccatccatttattctgtttattccgtttgccaccatcccatccctcgtgactccttcctttcttccttccctgatttgtttctttttccttgatgtatcccactgggcctctccctcctgagcgactggctcgcgccttctcacctatctgcccgtccagcgcccactggcgtctcctgtcccctacttctcatttctgcaaagatgcaacagtgcccgggtttactcctgcttctcctggccacaaaccctgggatagcctgggcctacttccggttcttcccatgcctcacccccctaaaagagggtagccagagtccccacttgtggagggtggggtgaggatgcacctccagcctacactgaggctgggagaggggttcatgcctgccccctaccctcctcctccttcctttccctcctacctccccatcttccctcccactcctccccctcctccttctatacctcttcctccctcttccccatcctccctccccctcctctccctcctccttccattctttctcctcctccctcctactcctccccttctccctcctctcctccccttcctccttccactcatcttccacctcctttctcctctccatcctccctcccctcctccctctcctcctcccattcctcctcttcctctctcctctcctcccccttccctccccctcctccttccactcatcttcttcctcctttctgctcctcatcctgcctctccctcctcccattcatcccctcctccctcctgctcctccccaggcctcctcccttctactcctcctccttctccttcctcttccccttgctcttcctcctcccttccactcctcctcttttctcctccccattcatctcccactcttccccctcctccctccaactcctcccctcctccttccattcatctttctcctcctttctccttcccttcctcctccctcttctctccttcctccctcccattcctctgcctcctccttccactcatcttcctcctcctttctcttccccatcctccctcccactcctccccctccttcttccactgatctttctcctcctttctcctccccctcctcctcccattcccccctcctccctcctgctcctcatcctcccttccattcctcctccctcctccccatccttcttcccactcctgcccctcctccctttcactcctcctcctttttcctccccctcatctctctccttctcccttccttcccccttcccttctccagctcttacagaggttagggagggcatctgtgtactccctcacctctgtaaacttcacttctatctccttccctctccacctcaacctccagcccacctccaggctggggaaggggctgagtcttcccctcccatacatacctcacccggcccccagcccacagagaggctgagggaggggctctgggtcctcctccatccctgtacctgcttcttccctcttcatttccaccttctagatctttccccccacccagcccacctccaggctggggaaggtgaggaattctttcctcccacaccctaccccacctcacctgcagcctgtgccctgggccaggagaggcatgggtgaacacccagacccacaacccccgaccctgcaggctttgggcatgaaaacacgtggatcggcctgaacgacaggatcgtggagagagatttccagtggacggacaacaccgggctggtgagtggcaggggctgcgggcctaggctgctgagccacatcagccctgccatctggcctcagtttacccttttgtgcaaagttatcgcaattgggtgtcagaggtctctagtggctccaagatgctggagttggcaaaggagcttcaagtgaaggctaaaccttctcccctgacttccccagcccaagtaaacagaccgcgcccacgaagctgcagaggttacaaacctgagactagggtctgatgcctcccctactcactctgcctggtcccccagccatggtctctcaaatattctcatttctagcctgggcatgatggctcatgcctgtaatcccagcactttgggaggccaaggtgggcacttgagactagcctggccaacatggtgaaaccccgtctctactaaaaatataaaaattagctgggtgtggtggcccacgcctctaatcccagctactcaggaggctgaggcgtgagaatcgcttgaacctgtgaggcggaggttgcagtgagccgagatcactccactgcacttcagcctgggtgatgatagaacgagatcctgtctcaaaaaaaaaaaaaagtatatatatgccaggctggtggtccatgcttgtaatcccagcactttgggagactgagtgaggaggatcacttgagcccaggagttcaaggctgcagtgacctgtgatcacatcactgcactccagcctgggtgacagagcgagaccctgtctctaaaaacatattagctcagcatcctccctgcttcaagccctcccagactctccattgttctcaagataaaaattccccttctcccgctgtgtaaaaatctggcggttcctcacacagttaaacatagaagctgggcacagtggtacgagcctgtagtctcagctatcgggaagctgaggcgggaggatcacttaagcctaggaattggaatccagcctgggcaacacagtgagacccccgtctctaaaaatataaaaataaaaataaaaagttaaacatagagttaccacatgacccagcgattctactactagatacatacccagaagaattgaaaacagagattcaaagtggtacaaaaatgtacattattcgggtcatggatacactaaaagccaagactttgccactagatgatatatattttattatatgcatatgcatataataaaattgcacttgtaccctttaaatttatacacagaggccgggcacagtggctcacgcctatgatcccagcactttggaaggccgaggcaggcagatcacttgaggtcaggagttccagaagagcctggccaatgtggtgaaaccccatctctactaaaaatacaaaaattagctgggtgtggtggcatgtacctggaatcccagctacttgggaggctgaggcaggagaatcacttgaacccgggaggcagaggttgcagtgagccaagatcacaccatttcactccagcctgggcaacagagcaggactccatctccaaaacaacacaaacaaacaaaaaattgtactaagaaagaaaaagaggctgggtgtggtagctcacacctgtaatcccagcactttggggaggccaaagtaggaggatcgctggagcccaggaggttgaggctccagtgagctatgattgcaccactgcactccagcctgggtgacagagcaagaccctgtctcaaacaaaacaaaacaggccaggcgtggtgggtgactcacgcctgtaatcccagcactttgggaggtcgaggcgagtggatcacctgaggtcgggagttcaagaccagcctggccaacatggtgaaaccctgtctctactaaaaataaaaaaatagctgggtgtgatggcacatgcctgtagtcccagctacttgcgaggctgaggcaggagaatcgcttgaacctggaaggcgtaggttgcagtgagccgagatcgcgtcactgcactccagcctgggtgacagagggagactctgtctcaaaaaacaaacaaacaaacaaaaacttgtacatgaatgttcacagccccactattcacaatagccaaggtgggaacatccaaaatacccatcaacagatgaaggaagagacaaaatgtggtccatccatgcaatggaatattgttcggccataaaggagaagttggccaggtgcagtggttcatgcctgcaatcccagcacttttggaggccaaggtgggtggattgcttgagttgtggagtttgagaccagcctgggcaacatggcgaaaccccatctctacaaaaaattcaaaaattgagacagagcgagactccggctcaaaaataaaataaaataaaataataaacaaaatgtggaatgagccaggcatagaaggccagctgttgtatgatttttttttttcatgaagcattcagagtaggcaaatttagagacaggaagtagattagtgtttgccaggggctggggtcgggagaagggggagtcactagtgctcaatgagtacagaattttggtgtgttgtgatgaaaaagttctggaaataggctgtgcggggtggctcaggcctgtaatcccaacactttgggaggctgaggtggatggatcacaaggtcaggagttcaagaccagcctgaccaatatggtaaaaccctgtgtctactaaaaatacaaaaattagccaggcgtggtggcgtgcacgtgtaatcccagctactcaggaggttggggcaggagaatcgcttgaacctgggaggcggaggttgcagtgagccaagatcatgccactgcactccagcctgggcgacagagagagactccatctcaaaaaaaaaaaaaaaaaaagaaaaagaaaaagttctggaaataacaatgatggttatgcaacattgtgaatgtaattagtgcattaaactgcacaccgcctcttgaacccgggaggcggaggttgcagcgagccaaggtagcaccactgcaatccagcctggataatagagactctgtctcaacaaaataaaaataacaataaactgcacacgtaaaaatggttaaaattggccaggtgcggtggctcacgcctgtaatcccagcactttgggaggccaaggtgggtggatcgcgaggtcaggagttcaagaccagcctggccaacatggtgaaaccccatctctactgaaaatacaaaaattagctgggcatggtggcgcatgcctgtaatcccagctactcaggaggctggggcaggagaattgcttgaaccgggacctgggaggcggaggtagcagtgagcgagatcgcaccactacactccagcctgggctctgtctcaaacaaaacaaaacaaaacaaaacaaaaatggttaaaattaggaattatgtgctatatatattttgcgctatatatattttgccacaatttttaaaaattcacttccttttctctgatgcaagatctggtccctctctactctctgagcacaccccagccccatcacgcagctccagccccactgggctttgtctgttcctcaagcagaacaagttcagtcctgcctctgagccttcacactggtggttccctttgctaggaatgctgttccctgtctgaacccttactatctttttttttttttttttgagatagaatctcactccatcacccgggctggagttcagtgatgcaacccagctcactgcaacctctgcctcccaggctcaagggattctcctgcttcagcatcccgagtatcttgggactacaggcgcgcaccatcatgcccggctaatttttgtatttttatgagagacagggtttcaccttgttgtccaaggtggtcccaaactcctgacctcaagtgatctgacccccttggcctctcaaagtgctgggattacaggcatgagccactgagcccggccttgaatccttactatctttgattctttaactctcacattgcctcctcagggaagtcctcctggacttcgggctgagatcaccatttgtcccaccatgtatcttttttctttttcttttcctttttttttttttttttgaggcagagtctcggtcttgtcacccaggctggagtacaatagcgcaatctcggctcactgcaacctctgcctcccgggttcaaacaattctcctgctgggattacaggctcccactaccatgcctggctaattttttgtatttttagtggtgatggggtttcaccatattgaccaggctggtcttgaactcctgacctcaggtgatccgcccacctcggcctcccagagtgctgggattacaggcatgagccaccaggcctggccttgtcccaccacttatctaacactgattcatgttgaaaatgaactgggtgcggccgggcacccagggaggcaggaggaccagggaggagaatgctgcagcctccaaaccagaggaatggtgtattggattacagtggtggcccccgtcaggatgggcaggacagtggccattgtagagatgggaacactgaatccctgtgagtttgaggggtctggccaggggagtcccacagcaggaattgccagaacctgggctggaacagaggtcccctgaacaactccctgccccctattcctgcagcaatttgagaactggcgagagaaccagccggacaatttcttcgcgggtggcgaggactgtgtggtgatggtggcgcatgaaagcgggcgctggaacgatgtcccctgcaactacaacctaccctatgtctgcaagaagggcacaggtatgctgtgccccctgcttcttttcctctctgtcacttttgctcactggttctctgttgcccaggctggagtgcagtggcataatcatggctcatcacagcctccaccgcctgggttcaagccatcctcctgcctcagcttcccgagtaggtgggactataggcatgcaccaacatgcccagctaatttttgtattttttgtagagatggggtcttgccatgttgcccaggctggtcccgaactcctgggctcaagcgatctgcctgtctcagcctctcgaagtgctgggattatatgcataagccattgtgcccagccttgattcatttttttttttcatgaagcattcagagtacacaaatttagagacaagaagtagattagtatttgccaggggctagggtttgggagaagggggagtcactagtgcttaatgggtatagaattttggtgtgttgtgatgaaaaagttctggaaataggccgctcggggtggctcatgcctgtaatcccaacactttgggaggccaaggtgggtgggattatattttatttttttaatttattcttattttgtttttacttatttatgtttttggagacagtttcgctcttgttgtccaggctggagtgcaaaggcatgctcttggctcacttcaacctctgcctcccatgttcaagtgattctcctgcctcagcctcctgagtagctgggattacaggcgtatgccaccacgcccagctggtttttatattattagtagagacggggtttcaccatgttggccaggctggtctcgaactcctgacctcaggtgatccacccacctcggcctcccaaagggcagggattacaagcatgagccactgcacctcgccttcatctttttaagttggtaaatgtcagtgcagccagttctcactttccaccagcgtatgagagtgcttgtttcctcccacccttgctaactctgcacattcttaaacttttgttctttgttcatctaagtggggaaaaatagtatctcattgttatcttagtttgcatgcctttaaggatgagaataaccagttgttttttgtttgtttgtttgagatggagtctcactctgtcatccaagctggagtgcagtattgcagtctcggctcactgcaacctccgcctctgaggttcaagcgattctcctgcctcagcctcccgagtagctgggattacaggcacacaccacacctggctaattttttttttgtttttgtatttttagtggagacggggtttcaccatgttgaccaggctggtctcaaacttctgaccttaagtagtctgcctgcctcagcctcccaaagttctggggttacaggtgtgagccaccgtgtctggcctaaccaactgtttatatgttgacagcctgttggcatatatttctttccctgtgaactgtcaattcatctcttttgcccagttttctattgacttgttgatcttactgatttttaagagatctttatatatgaaagaaatcatttgtatacatttgtacatccacctttattttatactttggtttatttttgtttccatttctttgtcaattttcacatcctcccccccatttattcatgtatttatttatttatttttgaaacagggtcttgcaatgttgcccaggctggagtacagtggcattatcacggctcactgcagatcacttgaactccagggctcaagtgatactcctacctcagtcttaccagtagctggactacgggtgcacaccaccatgcctggcttatttatttatttatttattttgagacggcgtcttgctctgtcgcccaggctggagtgcagtggcgcaatctcggctcactacaagctccgcctcccgggttcacgccattctcctgccttggcctcctcagcagctgggactacaggcgcacgctgccacacccagctaattttttgtatttttagtagagacgatgtttcaccgtgttagccaggatggtctcgatctcctgaccttgtgatccgcctgcctcggcctcccaaagtgctggaattacaggtgtgagccaccgcacccggcccttattttaaaatttttttgtagagacagggtcttgctgtgttgcccaggctggtctcaaactcccggactgaagcaatccttccacctcagcctcccaaagtgctggcattacaggcctgagctgccgcacccggcctttatttgctcagataattattattgccgtctgtctccttcattcccattgtgccctatgtttggtgtaagaattagaaagtgaggtgtgtccgggaggttgctgtccctaaggagctgctgatttgggtagaagagacagacagacacagtcacttccagccctgtgagataagaactgggcagaggcagggacctgttgacgagctgaggggggtgtcattcccaataaaactcagcttgagcccatgagttcaaaactagcccgggcaacatagtgggaccctgtctttacaaaaaatcaaaaaattagctaggctcggtggtgcgcacctgtagtctcagctacttgagaggctgaggtgggaagatcccttgagcctgggaggttgaggctgcagtgagttgtgattgtgccactgcactccagcctgagcgacaaaggaagacgctcttttaaaaacaacaataagagccaggcacggtggctcatgcctttaatcccagcactttgggaggacaaggtgggcagctcatctgaggtcaggagttcaagacccgcctggccaatatgacgaaacccggtctctactataaatacaaaaattagccagccatggtggtgcatgtctgtaatcccagctcctcgggaggttgaggcaggagaatcacttgagtctgggaggcaggggttgcagtgagccgagatcacgccactgcactccagtctgggagacagagtgagaccctgtctcaaaaaaggaacaaaaaaatacaaaaattagccaggtgtagattacaggcaggtgcctgtaatcccagctacttgggaggctgaggcaggagaatcgcttgaaccagagaggcggaggttgcagtgaggcaagagcgcgccactgcactccagtctgggtgacagagcgaaactctgtctcaaacaacaacaacaacaacaactagtttagccccagatgtgagcacctctctcactagagattcctctgtcccagtgctctgtggtccccctccggcagtggagaatgcctcactcatcggtgcccgcaaggccaagtacaatgtccatgccactgtaaggtaccagtgcaatgaaggatttgcccagcaccatgtggccaccattcgatgccggagcaatggcaagtgggacaggccccaaattgtctgcaccaaacgtaagtagcttctcccagagatctcaacataggtttttggtatttctagtttccaagtaagacatcctatttctccacacattgatctggcttaatgtctgcctgataatacagccttgcagataagtgagaacttaccagcctgtctgatgtttccttcatttgtgtgtgtgtgtgtgtgtgtgtgtgtgtgtgtgtatgtgtatgagagagagatggagttgtgctctgttgcccaggctggagtgcagtggcacaatctcggctcactgcaacctcccgggttcaagcgatccttgtgtctcagcctcctgagtagctgggatcacaggtgtccactgccatgcccggctaatttttgtatttttagtagagacagggtttcactatgttggccaggctgatctcgaactcctgagttcatgtgatcagctcacctcagcctcccaaagtgctgggattacaggcgtgagccaccacatccggcctggttttgttttttttgaaacagagcctcactctgttgcccaggctggagtgcgaggctcaatcacagctcactatagcctcatcctcccagtctcaagggatcctccaaactcagcctcccaaatagctgggactacaagcatgtgccgccacgcctggctaatttttttatttttgtagagataggttctcgctatgttgcccaagctggtctcaaactcctggcttcaagagatcctcccactttggcctcccaaagtgctgggatgacagggatgagccctcgcgtctggcctctttcctctactttctctcccaaggacacccgctgcatcaggccacctgcctctcaccaccttttgtcccttccctgtcctccctcacttccctgcagccagacgttcacatcggatgcggcgacaccaccaccaccaccaacaccaccaccagcatcaccaccacaaatcccgcaaggagcgcagaaaacacaagaaacacccaacggaggactgggagaaggacgaagggaatttctgctgaagaaccagaaaaaagaaagcacaacacctttcccatgcctcctctggagccttcgcctggggagacagaacccagagagaaacaagagagtccagaagtccctgaaccccaaaccacatcccccacacacataatcctttgtacaaagctcctcttttccctttttttacatacacaagatcctcttggcaggtggagccaggtgtctgaaaagttcattctcgtctggctgaactctgggagtgtgtcccagctgagggaagcacaagtagcaaagctcattggtctggtctcttgtttgccaggctgattgaagcaggccttgatgagggtgcatgagtgtatgtttgcattcacatgaaggaattgcttttcacaccagaaattcagacttagtcaatgttggctgaattcctaaatccaggaagaagcctggacgtagggtcattagctttgggaatagaaggctacacagaagcacactgtttttgaacttgacaacagctctccctttaccctggacttcagcccaagttccgtctttggtcttggtggataaacacacagtgtggagatcccacgtactgcattttagggatgtttttaggacaacctccctccatgccttcagagttaggagtgagaatgatcaaagcaatatgtaggtgatggagggagagtgtattgctaacccttccaggtctagtccagcgctgagatttggtggttctgcatgtgtgatgaatctctttcacacaaatagacgagaggatatttagggctagatgagcccagatttcttccccctccatctctcagggagacaaagaacctccttcctggaccaaggaggtgctgccaagttttctagcccagtgcacatacccagtccttaagcagacattggtagtgcccctgccctgggtcccactcctgccccaccccacccttgtccctggccattgcctggtggtctagaaacacttaaaacttgaagtagtgacacctacctgcggtcatattgtagagagatgctcagtgttaaaactgaaacacacaaacacacacacacacacatttttctcttgtagattttaattttttaagtgggaaagaactcaccttgccttcctcccccaaatgtgcaacctgtaaaaggtctctccacaccaggggccaggatccagttccctcatctctggcaggaaagatccacagcttttcctccatgtctgttactcactttcagcagtccgggtaaaatctgtggatcagggttaaaaaagcaccgtggagaatggccctcttcaggaaagaaaaataagcaaatgaatggtccacctaggggttcagtaaagaaagaaatgtgttaactgagcctgaatcccttctgggaagtaataatgaccattgacaactaagaagtagacaccatgctaaagacttacatacaatctccttgaatcttctcaatagcccattgacttagaaactgttactttcccattttacacacagtgaaactgaggctcagatataaaggaaaggtactggcttgaagtcacaaccacgacaggagtaaggatttggaataaggatttggtcctgttttctggaccaaatccttactctggctctgcttacactttctctccatcaccaaatccttactccaaatccagaagtcagagccaactcccatcttggttctgacccaaatcctgctctggactctggagaggagattgaaatataattgcaccctcatacacatttaggaaatggttaagaagtgtaaactgaacccttatccttgtcttcaatcttcctccctgtagacatctatcttattatggttattattcagaaaacccagggatacaggtttgtcttcttactttgataactcttcttagtttaaaataataataataacacatctttggtcatctatgtcacacaaaaattttcctttgtttgcggggggctggggatgcagtgttttttggggggtcttggtttatgctccctgcccttgagcccctcagccgtttgccctgcccccacctcggctccatggtgggagggggctctggtcttttctaaagtgggcggtttgtcttttgatctttcccttttggatgtgcgtgtgtgtctgcgtgtgccatgtgcgtggcacgcatatgagtgtgtgtgcgtgtgaacggctttgggtcctgctggttttgctgtgagctgcagtgttctgtgggtctgtggtatctgacactgtggacattaatgtacttcttggacattttaataaattttttaacagttcaa

SEQ ID NO: 2 - DNA, 188876 bp, Homo sapiens
  source 1..188876: mol_type = genomic DNA
agaccgcggccgccgcagccggcgaaagaggcaaagtcccgcacgccggaggacatgcgcctcggctagcggccccgggccccaccaccgtgcggctttctccagattattcctctctcgctgtctctgactgtctctctctgtctctgtctctgtctctctctctctcacacacacacacacacacacaaacacacatacgcacgcacgatctcacttcgatctatacactggaggattaaaacaaacaaacaaaaaaaacatttccttcgctccccctccctctccactctgagaagcagaggagccgcacggcgaggggccgcagaccgtctggaaatgcgaatcctaaagcgtttcctcgcttgcattcagctcctctgtgtttgccgcctgggtgagtgagaagagctcggtggggtttcagctccgggatcggtgggatgagggtgggagcatttcagcgtgtccgcgacttgccgccgccgccgccatctagccaggaggaaaaagggacagccaactgggctcccacggagcgggcggccgcgcggtgcaggcagctgcgtgggatttgcgcgccccctccccgccccgcgccctcgccccttccatcggcccgcgggcattcgtgccccgggccccaggagacggagctactggagacggagggctactcggatccctgccgccaccgttcttctctcctcttttgcaaagtgtagaaatcgccttttagatatacataggaagataaatgaccagaaatacagctgtggtgcggggctgtgtactgtactgtcttttttgctgttcatttttatgctataggtatgatgggcttaaatggaaagtacacgtgtctggtgtgaattgcagacacacacacacacacacacacacacctgaaaggtagtaacaggcacaagcaccaacctgacttttcagggttttttttattactttcaatttagtcatattcaagtctgcgcaattacttacaaaaaaaaaagttccatctaaatctctaggtttggtcagtgctgccaaagtcagaggagagctgcaggttgatatacttattcacagatttcatttttcatctgcaccctccaggttacatttaaaactgaaattgcattcagcaatagccagtttaagcattccctttacctgcgatgcagactcaagttttccagtcattttcgagaagtgcaagattggacttttgcgttttattaaagaaatgctttttttccttcatgattttccatcgcgatagaatataatgggtttacgcattaacctgaaagttacaaagtgttgtacttcgctttgtgagtacacatatttaattcttttcagaggctcccaatttttcaattttagatggttcatagtagagtagatattaagcgtacattttcatccaatagatggtgctgctcttggcacagtaacctttacaaacagaaactatttttaagttctaataaatgaaaattacattgcaaatacggtgataattgcccatagacccccacacacggacataatttaaatttcaaaggctttatgaaagtgcttggggtggtgtgatgcatagttgacacataagacatgcaagggaatctctggtctccattgattctaaaaccttatagtcagttctggagaggggaagacagctttaaaaagtacgtggtcatcaaacatctgttgaattcacatttctattgtagatctattttaacaaagaaggcaggtcctcttagtaattaaattatgtaagtaaatatcacagtagcaaggaaataaaccagagagtattgctttgtatgtcttcaaaaccacatgcccagagcatgttgcggatctctttgaaaaaaaaaaaaagataaaattcacacattatacaattcatctttttaaaatggtccagtggtttttaatatattcgtgaagttgtgcagccatcaccactaattccagaatgttttaatgaccccaaaaagaaaccccatgttccttagcagccattccccattctccatgaccccagctcctagcaaccactaatctactttctgtctctataaatttgcctatcctggacatttcatataaatagaatcatgcaacttgtggccttttctgtctgtctttgttcacttggcataatgttttcagtttcatccatgttatagtatgtataagtacttcattcctttttgtggctgagtaatattccattatgccaatatactaaatatatcacatttgcaaaggtttttctctgtaaagcattatttgtagcctatttttggttgtatagttttcagtttgttggacattttctttaagtctttggtgatgtaaaatttaagtgtaaagaatttaattaacttttaaagactatcacaagcttatgtaatgctactgaatgtaagcttgcattggaactccaagtgagttttgttgagactggaaggtgactacttttgtattcctcaccattttaacaaagttggccttttacacagttgccaacaggctgacctggaggcattggcaacatcaagtaacataaatatcttgatcttggggcagcatgttttgaaaaagtgaggcacattatcttcaaatgaaaaagttaggctccatttcaattatttttcactcagcacctacttcagtgtttgtggatgcgtttacccctcatcttcagtgttcttagagccaatgaggtagcctgacatatgtattaagtgctcagtaagggtctgtcaactgactaaaacttgctgtacaggagaagagctcaatacaatttgaacattaggctgtcattcttaccattatccaggaacaagttgtagagttcactaagatattgattattggcaattaagaatgtcttagtgatcagatcagatcattttgtatctaaaatgtcctgatgttttgaactggacataatagaccaactgcttctttcaaataactttccagacaaaaaatttatcagaatacatgaaccaaaacaaatttttcttgacccactgaacaggtgtatcttatgggaaaaactacagtggctttcctgtaaggaggcttaactatgagtcatagaggagaggtagatattttaagagcttttaaggaataatttcttccaattaatgaaggaattcaggatctttgaattaagtgaattatagtgctgcaaggtacactaagttcaaatgtttaagtattattattaaatatgtaatgaatcagattctgtacttctcagtcacatcttactgctttaagagccacattaacaataaattatacccaactcctctggaagaattaacttcttaatttatgctggttatagagtctgaaattcaatatgttgtgactctcagtttgatctatttatcctctgaaaacaaattgtaaccacatataaaaaatattcatgcctttttcccaaataataactagcctctattcagaaaattaattccttttcattgcagagttgcaattttctttatttcatttctctctcctttttacgtatactgtggttgcttccagtcagggaagtttgccaggagaaatcatttcattgatgaattcatttctactctcatgtctgcactagagattcaagatcttgcttaaaattttatgttgtgggaattttgcttctggcaaaaattcaggtttttctggaagcataagctgcgttttctcattccctttaactccgaaaagatttgagtactgtctaaagtgagagatttgcttgaccacccatttgctgaccgttcagactgttgcatggaaatggaaccccctcagtcaggaccagaagatctcaaaggaaggcataattaagaagatgagcctgtgtttcattttcagttaccatctgctcaagtgtctactaggtgcagtttcagcactgagcattttatgtacattttctcatttcattctcctcaaaaccctttgatattggtgttttcatcttacaccttttacaaatgagggaacccggcacagagaactcagataagttgcctaacatcacaaatcctgattcagcctgtgtattccttcccaatccagagcctgaggcttttcctcttttcattcccaggatcaatttgctgtttggcattcactagcttgctttcatgtgatgtgatggtacatttcacagtcccatttgcttttctgaggatccctttcatgattttgcccatgtactcttgtattacttttttatccttttttgaaaattaaggcataatttacatttttaccatcttgaaagttttcaccgatgcatatggtcatgtagcaatcactgtgatccagatacagaatcgttccagccatgctactttaactactcattcactagatgttcactaagtgcccgacatctgcaaggcaccatgctaggcgctgataattcagctatgcatcaacatacagagacttaaactcatgatgctcacaacccagttaatctgattcaatcccacaatgaaaactttctttaaatcttgttttaaagatactttttctgttcacaactagtcgaaaataagtcttttccaaatatctcttaatgtgttgaatatgttaactctcaaatactttaaaatgcttcctgctccattaagaattccaatggtaaaagtcagagccaatttaaacaagagagagatatgtgtgtatatacatatataaatgtatatatatgcatatacataaaagtgtatatatatatatgtgtttgtgtatatatatataaaatatatacatttaaattgtatttcaacttttagtgtcttattgagattaattcctagaagtaacttcagattacaaaaggaaaccaatttttctctgtgtttaaagaaattaaagaaattaaagaaagaaaaaatatcatatagcacaatcttaaaacacttgtattttagaaatgtcctgtttaataccatagccactaggcagatgtagctatgtaatttaaaatttattaaaataaaacaattcagttccctaatcacaccatccacatttcaagtgctcagtaatctgtgtggctagtggcaatcacatgggacagtgcagacatggaacatctccatctggcagtgttgttttggaacaacaacaaaaaacgctggtaaaataatgattgatagaactcatgaggagtcatcaactcaactagaaaaatttctaaagtcattgaaattgtttgaagtgttcgtgcatgtgagttagttatggtttgtgttgtgttagcctgcaaacatcctagaaacatgatggggcctttggtagagcagtaataggtgaaaagagttcagtggccactttgtagcctttatggactgaggttgaacagaagagattaactgctggttcgggagtgagtgctctgctcccatagtacattttccataatttgttctgttatccactgtattgtttttcaatttatctgtctccttgactcgaccacattatttgagggtaaagaatccattcttacattcatcattcattcattgttaaattcattcattgaagaattcaatgaaaaattcagtcaacattgcattcttgaacttagcacttacagtaggtgccaataaatctttaatgtataaggttatagaaagattgtaccatgaagcttcttagaaaaaaaaatagttgtttgttgttgagactggccaattttcactggtctttccctcttttgattatgatctcagtccgggagtaaggtcagcattcaccttgacctgtccatctttaatgtcacttctggtacgcgaacttcactttcactgattctcccagaaatacacgtaagcatataggcatcctctatgaggttggagctgtcatgaatgtgattaatgcctgtacaagcattacatagatccagacactatctgtgcatggaaaataaagttcctgtgattgatactgagtgtgtacttacagaagcagtcttgagtgagcttttaattctagaggagttggcaccaatgagttttggatacatcccagggggccagaacatcaagtcagctagtgtgccactttgtatgtgtacaagtgcatgcgcctgtgctgctctggtgttggggaggagagggcaatgagggatgcaggatggtcagtaatgagtaaataaagtatcttgagcccatgaacactgtattttcagtcagccaaaagctatcatattggtttaagtcttctctagtcaagaaggccgtaagagatagtgacacaaaacagttttgaaaaccaggcaaatgatgggaagtgcagttttatcatgtggatgtacgtgcttttggagtttgtcaatagttttatcattaatcctagagaggatgtgctacacaagtttaactctgcacatagcaaatatttgaactgagttgaattcaaatggaatgggacatgggatcagttaaaaatcttcataggagtcaaaagcagaaaaaaaggagatgatcttttgttaggatatttgctttattttcttctcaactaccttcctcccttccttttttctttccctccttccttctatttttccttccttcccctttcctcccttcttccctcccttcctccttttatccttcattcctttaatccttccttcttttctttttctttttttgttgctatttcctcttcagcttcactttcttttagcatgagtttaagataattaaataaacttaagtttgctcagcaccttgatgaaaagtttgatagggaagagattaatgctatttgaaaccattagttaaaaatatttttactgtgttaatatcatttgggggtctttgtagttaacaaaatttttaaagtatgagttggcagatgtttttgcctaaatattccagtgtggtaaaatgggctgattgtctttgttttccccttctgatatttgcccacagatattaaagtaaactcagttattttgggtactggttaatgtgaagtttttggccttattcaataagtacaagtcaatttatgtgcatgctaacaggggatagttttttttacaagtttccttggaaatgtagtacttgggtacttggaaataatttattcattagtaagaaagaaggattttctgttagaatatccataactatgatgagaatacagatattttcctgctcttggagaaacattctaccctctctctctgccttcccctttagcacttactggataaattttctactttttcactgttactgactgaactgcctatttcttgaatcctaaagctctaatgtatttacctaaggatgggtctcactatgagctcttttctctgaactgattaactcagtaccttgagtaggaataaaataaaacattgatttttaaaatgactcctttaagcagagatcatggtaataaacagagtcttgattacatgcttagaacatgctggccagatataaggaagtttttagattttctaatggtttttattcatattgtcatcagattgaaaattatttctattattaaatttttacccacttgcattctgacacattttcttgaataggatatgtcaaggtggtattgtctcagctgctacacagagttgtgcaaaatcatgatctgcccaaaggagcataaggatacccttgaggagcatgtgggatattattgtcatgtgtgttttagtgcacaaaaacgctgacaaccatttatctaggttgataattctcaatcctggctgcattttgaacttacttggtgttatggactgaatgtttgtgtcctctccaaaattcatgttgaagccctaacctgccattgtgatcgtatttggggacagggcctttgcagggtaattaggtcatgagggtgaagctgtcattgatgggtttagtgtctgtataagaagagaaaaagaccagaatcctctctctattcaccatgtaaagacacagcaagaaggcagccatctgcaatccagaaagagagccctcgccaggaatcaaatctgctggccttttgatcttaaacttcccagcctccatatctgtgagaaataaatgtctgttatgttagccacccagtaaatggtattttgttatagtagcccaagctgactatgcacctgggagacttttaaatcatactgatgcctgggcatcaaatcaagtacttcataatgtatgggtgctgagcccagacatggacattttcttaaaactctccaggtgattatagagcacagcctgggactgaaaccacagatctaggccaacttcatgacactactaatcttagttcatttttcttaaggtggagatatttgcaggtcttatgctgtcctaaaacaaagctagtgccacaagacaaaggtcctggaaattttcccttctctctctcttcttaaggcagtttcagcttcagagtaaccctagaaggaggagtatgctctatggtgattgacctcatagtcttttaaagccagggaaactttaggaggccaggcagtcttggagtgggtccaaagattcaagtctatcagctggtaggagggtaaatgaaccagcttccgagatggggactccaagtgacatttatccagacacaagcacataatatcgttcatttcatcaatgatacatattcttgttctctgttatttattttctttctccatgcataatttgttagaacttggctttttgtttactgttatgaaataaatattaagatttagctattgctttttcattttagcaatttgtgtgttttatgaaattattaatcaggtataggctatttctttttactgtatttgaaaagcaccaataactccatttaaaaattaacaatgaacttttctaggtgtttcaaataactcctttaatatacttattagtgatgatgcaagtacaaattaatttaatgaatcatttttgagcatcttccttgggataaatattcctccagagagactattcttctcatggccagctgaacttttacaagatagaaagatgcatacgtaagtaggtctcatacatgatggagtttctggcatatgagactgtgagctgtgtggagatatatttagtcttaaattcacctcttagaaggtttctagtaggagatgggattggatatagaccattcaggataaatggatagagcttcaggggtcaccatgggagcagaaggcatcagagggattaaggttggcatgcagagaaacatggactgaaagtgagaacgtggtaattggagggaggggacagtggggagaggagtataacagtaaaaggtctcaaatggtaggtaggatttacaatggccaattgaagagttttgtgtttgaaatctactcattgtacaatgggaaattactaaagggttttgagcagtgaaatgactggaatagatctctttatttcaggaagataactcaggtatcagagcataggatggattgaagccgagggagacagggaatcaggaagcttggttaaaagcctcttacacggtggcagtcacagtggaaatttatagatcatttgggatataataatatcatcctctttatagaattctttaatgtttaacaagcactttcagatacattatctcacttgatatgaatgtaattggtataattggtaggacattgtgccattctctggagtgagtcactggtgatccttaagggagtgtcttctctgggaaggtgaagatgtagcatggaacacagggaatcagagaaatatccaggaaggaaaagagaaggtagcaaatacatacaagtccacagaagttgataacttggagggaagaaagaagagagagaaacagaggatgatcgcttgaaacctgattatttattgaatggtgaaactcttaaatttgttaagacaaggcagtaattgactctagacattttctctgaacagattaactcagtgccttgagtagaaataagaggaaatagtgctgctaaaaatgattattttttttagagattagagtcacaacttaactaaaacaagcaaacttttttaaaggaggattttagtatatttatagtttcaggttaatcttatcaaatgccaatatgaaaactacttacttcattcacctttcaaagccttgtatttgtcatttttacagtgaacacatgtactattgggacatttcagccagcattctgccagtacacaggctttcttaaccacaagttcagattacttgaaggccatcatatttatgtccataaactagcatctttgtaaattgcttacaacttgtattttatataaaaatagtgtaatggaaaataatgcatagattaggcttctcagaatttgtttaaaaattgactttggcagttttcgttgttgctgtttttgagacggagtttcactcttgttgcccaggctggagtgcaatggtgcgatcttggctcactgcaacctccacctcctgggttcaagtgagtctcctgcctcagcctcccaagtatctgggattacaggcatgtgccaccatgcccagctaattttgtatttttagtagagatggggtttctccatattggtcagggtggcctctaactcccaacctcaggtggtccaccctcctcggcctcccaaagtgctgggattacaggtgtgagccactgtgcctggctagctttttctttaattcatgaaaaatacataggattgtttactctatgtctcttaagatacattaagatacatgaattggtaaattaatcaattactaagtttaggaaagagagaaggcagcagctgtcaagactgaaatgtaagcatattttttccttatgtaatgaaaaaatatagacagttgtgctatatatgagttgaatgccaaacttttgtctattttaaaagtatgactgaatcgattttcaatagaagtatttggttactgtaaagtttcctttgatggctttcaggtggggaaatgacatgtaatggtttatatatataaatacatttatataagtatgttatatggaaacatatatgtatatgtgcatatatgcatctatatacacacctaaataaggacacatataaatgcatatatgtaagtgcatatatatacacaaatgcacgtgtatatttcatatatatgcattttttttcctgctgctaaaaactgatactgctttattttgaatgttaaaagattacatcctttggaaacatcattatgtttaattgtagcatatattctgggtattttgagcctgacattttcctgcctccaacataaaggacagaaagaatgccgcttcttcctgtgacctgaatacactgtcagccctctccaaagggtttgggcccagttagagcagtgaaactcagttcagcacatgtttgttgaacatttactgtgtgctccacactctccttggtcttttttaataaattctttttgtctttgaagagttaatctggttacagatatgaattgtaccctggtgggagcataccagaaaagagaataataatgtaaatgctagactatttggatgagattttaagtctgtggaaattcagaacaaggaaacacaaagacatgaattcatttttttaaaaaagtctctgagcatgtgagcagcacttcttccttttgaacatggatggaccatttgcagttgcattcagtattacgagcctcctttgtgtccctccccgtcatttttactacattcaagaaacataacaataaataacaataagcctgtattactcactttaggttatttggttaaggagaagcttgaatatggaagtttgttggtttttgtctccaaaatgcatttagggttccccagcattccactttatcacattcaaaataattgggtaagtaggaaaagatataagcaaaatgtgtatgttaaaacactcttcttatatgttctgttcaacaccaaccactagaaaaatgtcacataatttccacagttaagtctatcccaacttgagccttaatttttggtggtagcagctgctattttgcgacaaaggtaaactcttggtgccaaaatttccaagaatgtttaggatgcatatgatttttggccactctggagaattttgaacgtagatggcaatgctgtctgaaaaatggatccctgtgaaagaagcataaagcgtgaaatcacatttaattagccaagagtggctagtgtatgttgccattaaatcctttaaattacagcacatctgggcaccgtccatcccacaagaatgtatccaatgatgcctctgtcttacctgtgagtccagcttattttctcctaacttctcattactttttaactattcagtattcttggcataagaaatcaagaaaacacaagatgcttccatgtttctcaataaattattcaaaatgttataccaacccttccccacccccaaaatggcaaaatgcccataagaaagtatttcaattccctatttggaaggagcttttcaagacatcctcttttacttcacattttaaaattagaattctatagcatctgtcaatttggagagggaagaaaacacttataatttttacaaacatcacaagagtctaagttgtattagacttgatggcattcaggcaagcaaagatcaaatattcagccaaatagtctctctgctaatgataatgacatataaacccttttatggtgacatgggttgggtcttctactggcagaggaagtgtatctttatgcgatgagttctcactcttaaatacaaggggagtgctggtttttaaaacatgaggctttgagactgtagtacacagtttgctggagatgtgtttgggtgattgcgctactggggaaaatgcactgaaaataggtcatcaagaaaagattgatatagacttttttgtgtggaagtaattatgcttgattattgcagtctgttacaattcaatatgccagactatgaagtttcagtgagaaaatattaatatgtgcacatataatcctacctgggatctgtagactggagtttaaattcctactgattaatcctcacaggttctttatctggtattcagtaataaagaatggaaagttttttatatatcatatatgttcttgttcattcacttaaaaatcccaaacacatacgttagttcatcaaaaacacagctgaaacattccactctattaaatgtaacacacacacacacacacacacacacacacagccgtaaaaataacaacaaacccatctactatcagagtaggaaatcttcaccagcctactttcttgccaagatatatactgttcaataaacactttataattattacttatttgacataaattattgcctaaggccaatgttaagagactttttgcaagtttatagcaatgactgttgaaatgatttaaggaatcggattcttaggactttccttttacataatgtatttctgtaattctcagaaggcaattctacaactgactttaataacaaaacacaatacaggtatgccagggtccccatccatggaaacctttgagaaaataatgaacaaaattttatgttttttctactcatcaggggaaaggacctataggttcttttgagttttgatgtcatgtgatactcttaccagggcatgcaaagtgcttcaaaacctgaactcaagctacctgtccagctttacttgaatctccacacgtgtgctccttccctttggctcaatcttttcctcgtcttggggtggatttgggccccatgcattcctgttgtcttttactttgtctgtgctaacacatcattgtccccttccacaggaagggaaacatcatctcacccatgatggcacagcactcacattgcgtgtctgttgtttccatgactgtatcttcaaggagtctggtctcctcatgggatatagccactgggctctaaagacttcttgctcatatagtaggtgctcagtaaatatttgtggcataaatgagtgagtgaaattacttcaaaagtgatcacttgaaaaatagaaaatacaatcatttctgcctaatcaaatgttaaacgttgctaccacctcaactaattctgtacccccatttggcagagtaagcaactcgagatcacatttgcagcatttggaattagaattcagttcaccctcaaattcactcaatctctcttaatttttttattttaaaaaaaggtaaacagaaagtaggtcaatcaaatcacattctcattttaacaggtccaagagtctatatagattttgaactgaattggcacacaatcatggacaacttcctaagggcttttactcatggcattcagagttttaagaaagaaaattaagtgattggaatagagtacaatagtgaatgcagaaaaagggaagaaagatgactgtagccagaaacacgagagaaagagaactataagtttctttattctaagtagcggtgggagaatctactttttggcctgcacagcggatgcaccatttctatgggtaagttttccctgtgtttcctgtcaagccggggttttaatgaaacaaaaagataccggatccctctcgctaacacctcttcatccccccaacaccccagctagtgccagccaagagcatttctctcagttccttttatccctttctaccaagaacaagatctgagatatgtgagatcctgtttgattcaagcttggagaaagcactgtatattttacagccattggttacctgttgctgtcaatagctgtattgtcagcattttatcatggattgaatgctttaaaatttttgagcagcataaaagatttgtagaggaaaacctattttccttagaaacgagagacagtgttttttgatgcttcaaagtcttaaaaaatgtaggaatattagagttatgccttgccctgcttggtgggttttgagtgacattctgagaccatacattttgtgtcaagaatcgaatcaatttctcttctttgggctgctgacattgtttagagtatagatgaagactccccagtaagacatgtaaaacagtaagggactgtgaaaaaaatagattgtgaaaaataaaagtctatcaagatggggcaaacgtttaggggaatactatcaaaggtagtgttatgttcaataaaagatggctgaaatacttatgagactaatagacaaatggaaattatttacacaaacttctgtaactgcccagttctgtgaaaaacaataggaaaatcgaattcagaaaacacttatccctgtcagaattagaattaggataatggatccaccaaaatatgtgattgcttacactgaaagaatagccattatttttaacttagggtgaatttgactaatattgaataataggtgatttaagaaattattttgaaatgtttgttttaatatgccaggacaaaatgtaattgttataatttaagaaatacataattaagagaaaacaacacatctgaggaaagtaattacagtgctcagatgttaattaagagacactaaaaaacagactacttgttgaaatagtgatgtttgatagaatattagctatgttattaaaagcttaaaatccattattttattacataatagctaaaagcagcaatatttggtctaatgaagaattggcaaatagctataaggaatgattaaggcttctgcaggacttaatttggataaatcaggttttaaaacacaattatatagggaatgaattaaattattgaagttttaaaattatgagcacagaaaggaattatataggtttaaaattaatttttaagaatgaatactttcatcccactttcaaaaggctttattttttaaaaaaagtatttaaaatgaggcctgagtcaaacatttcagtatgagaagccttgcacaaaatttagctattgtagttatggtcctttggagacggtattagtcataaaactatgtatggtctttgtattaggataaatttactttggcttctgaagtttttgataaggatatatcttctgtgcaaaagcagaaagggctgtaaaatactagagaatgtagacaaaggtaccataaattactttggaaggacttcttgccatcctgaagcaaggttttgtgtgaatgtttacagctgactgtcacatctttagttacaaaattcaattttgttgcattatatccattcataattgaaacattggtgttttatttcaaattactagcttagcatatgataagttaatcagcaaatgtggttaaaataataggtgtttcttaacatgctattatttctaccattagagaaaaataaaacaaaaccaaaaactcctctcttgacttcttccacatttctccccttccattgcctgctaaacttttgggaagagttttctatagtcactatctattttctctcctgtcctttgctcctgaactcagtttaaaaagggttctgctcccaccattccaaaacaaaaaaaaataccttatcagtctcaccagtgcctttgacgttggtacatccaatattcagtgttcttggtcctcactttatggacctaccaagagctttcaattgttatcattgtgtcattctggagatgctttgttcacctgacttccgggatgccatacttgtctggctttcctgccatctcactagccacatacattgtactccactgtcttcctcaatctctttttctagttccttctcatccccccaacttctaaacattgtagtcctgagagttcttccttgggctacttctgttttgcatctacactatttgcatgactatctcatccctgtattctctacattgataactcccaaatttgtctttctagaccagaagttgtacctgaaccccagtgtcatatattcaactatccatttagatatgtaacacagttctcaaacttgacatgttcaaacttgatctcctgatcttcctcccatcccaactcctacttccactgtcttctttatcccaataaataacaactgcactcagttgtttaggacaaaaaccttggagccacttttaattcttccatctctcttacagcctacacataatcttccattgcaaatgtcagtattgctcctgggttacattttttcatgatctccatccactgctaccacctatccacacttgcatcatgtcttcttgggttatttcactggcttcccagctggtctccctacttcaccatacaaatcagccagggtgattttgttcaaatgtaagtcagattatgtcactctccaatttcttgtttaactcagaataaaagccaaaatctctactcatgaagtgtccatcaagccccaaatgctctggcctcctattatttctctttttatttttttaaaaaattactttattattatttagagacaggtctccctctgtcacccaggctggagtgcagtggtatgatcagagctcactgtagccctgaccttctaagctgaagcaatcctcctacctcggcctcctgagtaactgggactacaggcatgtgctaccatgcctggctattttttttttcttcaaatttattgtagagacggaatttatctatgttgtctgggctagtcttgacttcctggactcaagtgaacctcctgcctcagcctcccaaagttttgggattaaaaacttgagccaccatgcttagctctcctgttacttcttcagtgacctcccactccactctcccctcactcccctcttctggcctcactgtcctcctagcctttctggatcaccaaggcacattcttttcaacgcctttgttgttgctgttctctccatttggaatgcctttcttttataattatttatgatccacttcctcacttcctttggtttttgttcaatgatactttttcatttgaggacttccctggacacactgcagtgatattgcaaccacctctcttctcaaatgtcctctgtcttcccctctttatttttctccttagttcttatcactcatgcatttttctaaattattttattctctatctcttcccattggaatgtaagccccttgaaggtttttgtgtttgttcaccatggagccccagaccctagcataatgcctggtatatgataggtgcttagtaaatatttgttgaataaaacaacctcttttttttttttttttttttttttttttttagtttgattttgcttgaatcctattttggctgatatcactgcttctttttttagcttgtattgccatatataacttgcattgtcattttatttttgacattttatgcaactttttattaatgttaaatgaaccttgcaaacatcatataatcacattttttgaccttgtctgaaatcttagtttactttttcaacattctttcttcatcttgtttgatttttcatatttatccatactaattgtgttacatactctccagtttttgctttctgcactgtctggtctttgcatttatcgtttctaggtcacatttttaattattctagacattaactagtaaaatgccttaagttttttttctgtcattgtcaaaagtgaagctgttttctgagtcctgcactacagcagagaagaaatgcattaccacccaccaccagcagctttcttccagctccctctaacttctctgtcactgtcatttagtaatgtaaatacaaatttgttattttaaataaagaatgttcttaagtttttttgttttttacattttctttggatagtcatacagcagccagcctaacaaaaattatttgtattttttatgtcttagcgcttatagtttgttgctttataccataattttagcatttggggttgggctgttccttgaagcaattgtttgaagtgttgttagataacttgttttctgtgtaagacatatatgttatgtcttttttctcttcacatgtgcaagacaacctggatgcaaataaaattatttggttacaacgttttttcttacaaaacatatggtccctgcttttttatgttatggcatttagtgttactgagaaaagaacggaaagttccacctcaaggtcacttcttttatgccagatttggcatatttgatggctgcaagaggaggtggggtggtgggataggaaatcttcaaaacttagtacctgcctggtaaaaggaagagacacagaactctgactagttgaggtattagaccataagagtattttaattttctctccatcaacaaaataattaaatatattaaaatttatgtatttgaatctttaatatttaacaagattattcacaagtattaaatttggcttttcagtttcaccatttaaagggcaaaatcattctgcctgatatatcttagagttcttcaagcatctcatatatatatatatatatatatatatatatatatatatatatatgtaatgtttatataaatgtcttgcagtgactcttaagaaaataatcagttctccataataaggcagttaggatttacctgaaaataaagataatatatcaagacttagcaatatgctagcttttcaaccaaatatatcattttctgcatggtagtgggtatctttgtttttttgaaaaatatttttgtgattagaaaattatacacaatcattgcaaatattcagaaataacaaaatatataaaataaaatttatccacaattccatcagagatacaaatttttaacattttatttaacttctttcctggcatttttctacatgcacatatatacacacagagaggcaggaaggagggagggggagagagaggaagagagaggtgtacatttttaacaatgattgccctatacaaatggcttggtgatctgccttttgaatctaaaatatactgaaactttgtcatgtcgttaaacatcatttttagtcactgtatcagttagaatatagtttgattgcatgcccaaaaaaaccccaaatggctgtgtggttcaaacaagaatagatttgtttctcttttcttttccttgtcagtccagactggtggacctctcttttacaaagtcagtcaagatggtttgcaccgtggtgtcgacatccaatgttcttttatcttgctccactgtcttggaagtattactctcttccatatttcctggttagttcacaatcctgtcagtattctgtcaaatgaaatggagaaatggagaaatggcaaagcctgtgtattccctttacagatacaatgcagaaattgcactcttcagtttgccagaacttagccgcaagtccacaaagaagactggaaaatttagcatttattctggatggccaggtgctcaggtaaacaatttgtgactgtagaaaaagggggaaatggttattggggagcaattggcaatcactgtcaaactagtatttcattaaaatatgttttgacaggtttttttctgttattcaccatgcattcatgaatattcttacatttggttcccctgtaacaacttcttaatgatagccaagatttcctaaatatttatagggttccagatagtattttaattatcttgtatgtattttttcaactgaatataatgatattattaggtagctacacttctttatcctccttcagggatatgactgaggaagtctagacttataataggttaaatgagaaagtgttgaacaacccgatcgtgtgcacagggctagtaaatggttgattgacatgagactcagatctatgtctgaattccaagtccttaactcctactatctaatcattttttagaaaaaatttttcgaaggataagttctgccacaaagttaggcatatttctaagactgttgattcacattgtcaaactgcactctgaaaagcaggtgccagttggtgctcctgtcagcagtatacggaaggacccctttgccaaaacccttcccagcattaaatgcttaacataccaaaagaacagaaacaccactctaatacaccactaactaatacattagttgacatttatttgatgaataatatggttgaacatttacattggtatttattattgtttccatttcttttgtgaactgctttttgtacaaatcttttgtttaaattgtgcttttttttattatttgagaagggatctgcctctgtcactcaggctggagtgcagtggcataatcttggctcaccacaacctccacctcccgggctcaagtgatcctcccacctcagcctcctgtttagctgggactacaggcacgcacttccatgcccggctaatttttgtattttttgtagagacacgattttgctatgttgcgtaggctcacctcaaactcctgagatcaagcaatctgcccacttgggcctcccaaaatgttggcattgcaggtgtgagccaccacacctggtcctaaattgccttttatcccattcaatcattgctcttttgcttatagattattagaattctttgtcttaaggatatgtagtttggattccccctgacatttccttggattttgatagactttattggatttgggggaaggtgtattatgttcattgtctttgtcatatataattttttagataattttgggatttataactatggaagtggatagattttaaaagtgaaagatacgctaaatgtcatcatagttagttcttacttgagagtatcaccttaattttaaaaatgataatccctctcttagaagagaagacagaaggaaaatgtgtcctctgaaggcttttatttgcataaattgtttatatcttttgatcaacaatctttagctattttgatatccaaatgctttaaaagggtatattatttctttctcttctaaaacagaggatgtgcccctggtttttggagcccataaaaataggcattggcctgaaagtctggctgtgaccctaatgagaaaaatagctatgacctgtctctcctgtctgttctcttctagtactgtttcccctaccctctttaagtcttgcaggtgtaggataatcatacttatttctcttttttgggagctagattattgaattgtaggcttttaagaaataaggggacttccaggaagttgatagtgttgggtagaattgatctcataagctaattagaatttttgcagcccatttgcaacaaaaatagcaatgccctttttcttagatttttattaagagagatgtaaaataatattttaaaggaaatgtgaactacaggagaaagtaaagattttgtttattacaagttgtaaaggcttagtttataaattattttattgtaaacattgctacaattttatgaatcttctagtgttacacactagaaataaagatctctcattcgtaaaggatcatatgagagtcttcaagacgactcagattggccttatgaggaaaggatataaaaggcgttggcagcctacagtctgtacactaaaccggttaggaaaataattgtcagtgaaatgcaaaagaccagatccttcagatattaggcagttctcaaagaaataaaagtttggcttagcatcttccctcttccttgatgtattcttctcctatcctccccagatacatattatttcgttgcacttggaaggttagaatccccctttctgccccttgtctgggtcctccttctcgccaagtgttaccaggctccctagtcattctgcctattttcccaatcaaatatccatttctatgagtattcatttatagatatattcactgattatctgatcaacgtaatttatgctggactttgaatttccaaccttcataaggacatatgtacatttttaaaaaggaacacattaacagcaatgattctgtaattgatatagagtagatgttttaaaattttattgtgataaaaacacataacttgaaatctaccttcttaaatttttaagtgtacagtatagaattgttaattatatgcacgttgtacaggatctaactagaatgtcttaatcttgtataactgaaactctatatccattgaacaacttctcatttttccctctccaccagccttggaagctaccattctattttctgtgtctaagagtttgcctactttagatactacagcattattcacaatagctgagagatggaaacaacctaaatgtccattaatggatgaatggacacagaaaatttggtatatgtatcaataaaatattaatcacccttgaaaaacggaagaaaacccgtcatatgctacaacatagatgaaccttaagaacagtatgcccatcctggctaacatggtgaaaccccgcctctactaaaaatacaaaaaattagccgggtgtggtggtgggtgcctgtagtcccaactactggggaggctgaggcaggagaatggcgtgaacccaggaggcggagcttgcaatgagccaagatcgcgccactgcactctagcctgggtgacagggtgagactctgcctcaaataaaaaaaaaaaaagaaacaagaacattatgctaagtaaaataagccagtcacagaaaaataaatactgtatgattccacttgtatagattagatgtttttgagggcttggggtgagatatttctggggtgagatatttttcattgctccgtgcttatggctttaatctatttttccctcaattggcaatttgccttaatctctaaattgtcagtgggtgtatttgataatgaacatttcttgtacaaagaaagagactagcttggtttgtattgtccttattaataataaccatttctcaagacaatcatagtaccagacactttgtatttgtgttctcatataatctttataatgcaatgatagacactattgttgtttttcttgttattattggtcttttagcttccttaaagtcataagtggaaggtaacagaacttggacttgaacccaggtctactgatttcaaagcctctgcttttaacctcatacccttcagtgataacgggctttactcataataatggtggagatacagtttgaaataggtttgacatagatttagcgtacacattcctaagaactagtctctatggaaaaaaaaatgtaacatgacttcttcaaagccagaatctcaagttttttacattgatactgtgggttcgaggctttctgctgccatggtgctttcagagtatggacatttgttcttgtttttctaccttgttttatacattcctctatccccacagcccagcaatgcacgatgctatctggttggttagtctgcactctctgtcactgcccatcttaatatacatatttcaccaaaatccctccttctgctagctagaagatcttgttcactttctccactaagagggaaacctgacccgtggtcttcctgccaatctcacagcattactctacccaaaactatgcacagcagctttctctccctcctttccaaggttgcatgtagtgaatatatttacggctccatgggcagagtgagtgatcaagaaacactaagtgattgtggtggccagaaatgcagaggacatcatggtatatataaggcaaaattatccattgatgacttattacttatttggccattcaactagagtttttaagcactgattcttttccctaaaaccttattatatcataatggatccgttagataatctagtttaaattaaaagaaggaaaaaagaactttgccctcaggaaggaggaaaataacatcctaccaaatgtagaatgtaccacttcggtggtacatttaaatactacagtagcagaaaatgatacctgataatgtatatagagagctatcagtcctctcggtgcaaaaacaaaacagtaaaccatgtaagtacttagagacattcaggaatatgtgcagctctcataagtttcatcctactcactgaattcacttcagagaagatttcttttaattgtagaataatgtgatgggttttttttttcatgttctatgaatgattctatgctgtggaacagccttcaatggagaaattcagaagaagcaggatgtgatgttttcacaatccaaaaatgctgttgttgcatacactccagaaagcggctctgctgttaaaggaatttcttggaaatcttttatctgctgttttgaatagctgtaatataaatgtaatagatattgtctccattgtattcaaattgtattttctagagaatgagaagatggagtctgatgtcagccttcattttttttaatatactgtttttcttccctgccagaataatgccaacttcatgtatgttgtctacacttccttcacgtagatgagggaattatcttgaatatgtttcttttgaggaaattgagaagtctgctcagggagtactactgaatgctttgcttgctaacctatacatcagaacaaggtggtatatctgctgctctaagaataagtacattttgctacaaagagaagtatttcatatacttacttaaaatctaccaaaaagttcagggaagctcttagtaagtagctgaggttacttagcttcaggggaaaaaagctagaacagagtgtgtgttcaagaagtaaaatagggcatggaatcactaatggtggcagtgcatgaggaattgatatgagatgagactacagaaataactcagcaagtccagtgtaaacttttatttattcatatataaagcctgatattattgctgtaatttaaagtatattgctataatttagataaaatttttccatcactgctttcttttccatctctcagctggtcttattattcaagtcccaggacagtaaacattttgtagtttcaaaagcccttcagtactaatggatttttttttaaaactagttgttttaaatgaaactgtaattatgagaacatatctctcttttcttccttttggcaaattccactaggcattaaagcttattacccagttcaaatcgattatatttattttcattcatgactcacaacgtttctaccttctaatacttgcaagtatattcatatgtgatttcaagatctctcaagtacattttctgtatctttcctgctttgtcatatttgtaatattttatatgtaacataaaatatatggaagataaattctgctaactataaaagttcatatttttttaagttatcagaagttttgttctatatatctttgaagttactgaaagctcagatttcatttgaacagaatggtattgtagggacattatctagtttctttcaggctatctctctcctgagaaagcttgagtcctggagctctttggaattacagctgagcacagagaagatgggaacttgggacaggtgaatacccatgccctagacctggggaaaggaccagatctgattggaagaccctatagctctgtgcaagtcagactggggctatggagcagccttcaactggaggttgacagatcccttgagagagtattacatggtgttcacgtattcagagttccttttatggactattggagaccaagcttgaggccttggacaagagagagtttttaaagtacagggaaactctagcaatagtatttatgcttaggaactagttctctaatagcagtgaccactgtttccagtggctaaacctaggggcctgcaaagctattggaatggacacagatcagttttctgaagaaaattctggcccagtcaatagatactctcatggctgaagcctgagcagatgtgagagggtcaattactgagacagaacagggcacgagaataggtattgaaacctgagagacaggttgagagcctgtatttggatttcagagtgcagaggtcaagtttgggagctggaagaaggaagaaacatgaccagggagaaggtaagcagaaggggtgaactgaaactagatgctgaagacacaaggtcataaacgtgaacaaggatcaaagccctggcaactcaagaaaactgctcaacccaggagaggccgctcaccctttacaacagaatacaaactttgccttgtctctgatacctttctgtctgtcccaggaaggtcactccttttgtgaaatcacgctgttttgatgtgcctcttctatttattggattggatcccattgatggtttagaacccattctatggtaattgtttatacatttctcttcccattctactccatgaagtcctcagcaccagagactaacttaccacatgatatgtaaatgatgtacaattggcaaagagatcttgagagtctctgtatctgtttgtagggggaacccaccaacaaacagctttgatatagtggttcatctctaggatatagataagggtaagtcatgctgcccaatttattcagacgcctcttcctgcctggctctaaatctttgcagtaggagaatgacataggaccataaggacgtacctgaagccagaatatgtttatgtattcttttaataggcactgtaaaaggcactgtgatcagtgtttgggatgcaaaataaaacaatgaaatctctaccttcaaagtctaataataagtatgatgaccactgtttcttgtacatgaacgatgtgccaatcactgctgttttccatttatcatgtagttcaacataacaaccctgtaagacttattatatccattttacaaatgaaataaggcatagggaggtcatataacttgttcaatatcatgtagctgctaagtaggtgagcttggattagtgtgtctgaattcaaagaaaaagaattttagtagtatatgtcctgatctatgtaccccgttttctgtttctgatatttaacaccatgaatgcccttagattctgagtgattctttgttgagaatgaaagtagtaacagagagcaatgttataaaaagccccttccataattcaagcaaaaacattatgcatgccagtaatttcattttcgtcttcacaacctactcaaagtgagaaaacagagctaaaaatagggatgaggcaatgttatgtaaagttcaggaaaaatgtaataaaagccagatttaggtaaatattattgcagaaaagatgtattttgttaaaatggccttaaaatcttttctgtatgtttgtttcaagatataaaatatagatcactttttaaagaaatgattcagtgactattacacaattacagagttaccaaaaatgtcttcactgttagtatattctaaaatttgatgaagtattacacaagtcaaatcagaatttatttataattaatctcttgatattttattacagattagtgtttttagaatgtaacaattacttggcctttgatgctatttactgaaataatttttctaggagttatttcataaacgcctatcagagtgtagaagattattttattatcctgaataaagttttattttgtttttaaatgacacataacaattgtacatatttatgggctacaatatgatgtttcaatacgtatatacgttgtataatgatcatatcagggtacttagcatacccaccacctcaaacacttttcattcctttgtggcaaaaacattaaaaattccctattctagctaatttgaagaatacaatacattattgttggctatagtcaccctactgtgcagtagaacaccaggatttatttgtcctatctgactgtaactttgtacctgttggccaatccctctccattcccgactgtccccgctactctcaccagcaggtagtcactcttctactctctacttctatgagatcaatgtttttaggttccaacatgtaagtgagagcatgtggcatttgtctttctgtgcctggcttatttcacttaacataatatcctctaggtttatccatgttgtcacaatcacagtatttcattcttttatggttgatgttctgtgatgtatatataccacattttaaaattcatttatccattgatggacacttagattgattccatatctaggcaactgtgaatagtgctgcaataataggagtgcagatatatctttgacatattgattacatttcctttggattacatttcctttggatatatacccagcagtgcaatggttgacacatgtggtagttctaattttaattttttgaggaacatcatgctttcttccaaaatggctatactagtttacattcccattgacagggaataagagttctcctttctttatatcctcaccagcatttgttattttttgtctttttgataatagccatcctaactgagatgagatgaaatctcattgtggttttgatttgtatttctctgataagtagtcatgttagcatttttttcacttacctatctgccatttgtatgtcttcttttgagaaatatctattcagatctttgtctgttttttaatcaaatgattattattactattatttttactgctgagttgtttgagttcctgaactttcttttacctattacaatccagtgtcaagaagatagtgtattaatttctttgtttagtttattgatcaatgtttattgataatatctgccttcatgcctacaatgaagcaaaaccttataatactgctttctgtatttcttcaagattccatagccatgcagtgtaatgttgaaaaatcagcagatttactttttgatgtaggagaaagaaaaaaaaaataaacagagataacagctgtctaccacaaatttcctattttgaaagttctatatatttgtttaatatggaacatttacatgtcatgcactgggctgactgctgggaatatagaggagaacaagactgtccagtccgttgcttgcatagaatactgctgttgcacttagtactagagcaataccaattatgatagcatcacttaattaaggttgggcaactgtgttattatagttaggtaccatgtaacattgtgatttttaaaatgaatttatctcatttgataaaaatgcatatttttttctggcaggtatgaatttgctagaaattctctagttagactgtatcaactacagtatcatttattcacttaaaaagctgttgtcatgttcattatttgtacaatgaaaaaatgggagagatgcattttattgcaggcttctactgttggtaaagcactagatttttccactttgaattgttcaaaattattagtttatcaaacaaattgaaagaaaagccagggttctgttgctaacttttgaatgaaattttaagcattaaggcacatcaaatcaatggaatataaatatgaagatttttattttttacccagccattgtatccaaccaggctagggtgaattctacttttctatcctgcaatggcattctttgtgctttcggctcactttggtttttaaaagtccatttctttctaaaagattaatctccaggtcaattcaaacttcaccttttactcatcccttagctcgagtcctaaagcaagaagaagagagttaatcctagtaagcctcttattgttgctcattttttggttgagaaatgaagaaagatatggcacatagtatacattctctaaatatcaaatgaaagcatgaatgaacaaaatagcagaataaattaaattcagtgatttgctaaaagttaatctgaaatgtatgcaggattctgttccagtctttaaatctcacacacacacacacacacacacacacacacacaatagtaggtgtttagacgataataaaatttgaacaaaaagttcaaattacagtggaagactgcctttgaatataagttactttttttctgtgttagtctgtgaaataagtaagtattgaaaccttccctgatagatctccaaataatttcccattctggttggttggacatgtgcttagcaagagtgcatgcacctccagttcctccaagtaggggaaaacagaatggggagacaagggagattgagccgtttttcaagtatctcactcaggaggctggctgccaatcaaggacaccccagaaggaagacagtgaagggcacatcactggtgaggaggactaagatggatcgcagatggcagcctggaaccaggcctcccatatcagggagctgtgcagtgggggtgctcacagcctcactcaaagaaagcagacataggcccccaaagatattcaccttgtggaccagaatcttcctgttctggtttcatgttcttaaatgatcataattactataacaattattttgagtttttaatttttgatcttttttgttttaacacagtaagtaatctcaacattttttgccatattcatgaagcaggaaatgtactttaattccttatttataaaaatgttatggaccttataatcagaagagactcagatggtcttatttagtaagatttgaaaggatagattccggggtttctagaatttaaataataacttctgttgttacttctgtagtttccagagttttattgtttggttccaaaaaataaggagaacttgacttagcatagaaaacataacctttcatgtcagctctactttaataaatgcaagcacaattttgttcagctacctagaaaaataatgatattaggtaatctacaatcttttagcttttacatgaacctaagaattaaagctgggtctttaacttgagtttggtagatttaaagtaattacgtttttgctgtctacgaaagttgtttagttaaagatattgtaaatgaactataattatgtagatttacgtctgcttttaaaacacttgtgctctttcactgatttgaaccttcgtatatgctttcatatttattttgccaaagaaactatagataatttgattccatagcaggatgttgaggaagactttttctcttgccttggaatcactgctattggtccttgggagaacacaatgatgtctaagaattccatgttttatagcaaaccaccaaccatagtaaaaggtagcaccttttccaggtcttactaaaagaacagatcctactactgtcaacttttaccccagcatccctcacacctctccctaggccttatgagaagagtcgtggacaaaggagacgaggagagtcatcaaggacagctgcttcacacaatgctttggtaaagggtcttgtccccagtttcccacttcagcttcaacaaggccatgcagatatgaaattggagcatgagcatgagaatcattcagaaagatctaaaatatctgcaacaagtggaaattgacatctatagggagattcaaatctaagattttgttgaaagttaatctgaagtacatgtaacacttaacagtgatttctgcacccggccgaattctgggttctattctttacacacacacacacacacacacacacaaatggtgggcatctgtgctttaaaggtgaaagaaaaagaagggtctgtgatataaattcaaatttagacctgctgtaaaaatacagtatcttttctgtatggttgggccgcaagtaaatctggaagcatttttttgatagatcacctggtcattttaccacttgctctttcttttacagttttgctcaatcaccctgttggtatccctttcctgggtttttggcataatttacaagaggcactgatgatagtgagtacaagtaggtttatttctatttcaactgcctccactaaacaaagggcaaaaaaaccttgactgcatgagagattgcttctcaaataaaagctgtggttttctgtgtatatggtaatactcttaccttccctgtaatgtttcagtggtactttttttctaggaaactggagaacttaattttatacaggaatctctggtcttagaacttagcctagtggtaacagttttaaaaggtctctacctccatttaaaggaagcaattattccaaaagcacaggatctctggttgactaaagactgtatcaaggatgaaagattatgtctggaatatttcctgccttatgaacttttgtgttcaaaatataaattttagatttaaggaggcatgtgggtgtgtattgctcactctctgatatacaacatggttattttatgtttggtagtagtagttaaaataaaactatattaattgccttcttactaatgtactaaaggctaaatgaaagaaatttttcatcaaatgttataaattttgtttgattgtatcaatttgagatacggttcagtattttagaaccaatgtcttaaggtaggcttccatagttgcaattaagcttcataagaaatataattaagttgaagcttggcattgtttggtaggtgtctggctggcaccacgttttccaaaaataagatttgtttgaaggggcttgggtttttcccttaatttcaaggttatgtcttggatggggcttgggaaagggcagtgggttctgactacacgtcaaagatgttaaagacattcactggcttgacatcatgtggacagtggcttgcttattcttcgtttcctagtatcaattaaaataataaatggtcaggttttgctcacactatttttaaaatgaagtttatgatccataattttgtgaaataaacttgagttgtattcagtacacatatggaatagtaagttattacattttaaagatgacagtgatgtaatggtatacttatatattatgtttgcagatagatttttcacagaacaagattcctcggttttaattctacaatgtgatattggaattaaaaacaagtctcttatgtgtgaatattttatctctatttgttattatcaaaataaacactattcatagagttcaatttcatcaataattaataatgtgactaatgcatgataaaaagaataagttaaattatgttttttgcttacaaatatcacattaattgaataactggagtaatattatgctgcttttattaaatatgtagtgacaagtctagaaaataaactgattggcacatttgttaaaatccatgttagttttcctgaagaactacacaccctaataaataacttacttatgttcaaaatgaattaaatgtggcaaataattccaagacatttattttacctaattatatgtggttgaacaactaggaacaaatattttacaagtgaaaaccataagacacatttttttaggacatatgtatatactaatgctttttaccatttattatttgctgatagtccaggaccatatattaaagagtgatttcagtatccatctaaattttcatatggtttcatttaatcataggattaagagaatattttaaatgattagaaaatatcctcaattttttcaaggtttagagtatttatatttatacgttaaaatctttggaaaggcctaaactcagggtgttttgaaattatgtaaattaatatatttaggatctaatttattttatatctacattagcttttccatgtataagtttcacaaataaataaacttcacaaataaaattaataaaagctaattaaaaatatcaaacacctttctttttcaaatgtgtaatttaatgagattctgaattaccttgattatttctaaaagttactattttttcaaatgaaaataaaacttagccaccatttgaaggctatatgctcttagcttaaactgatacacttgcaaatatagctatccaactgcaaaataataatcaaatttagatattcagactctgagtagagaatggaagaagttgtttggctgatgtgaaaattgaaccaatgattttaattttgaacatactaagctctaaccaaatgtgtcaagtgttgccaaataaatatagaggaagaggataccatgttttatattaagaagggttaatcatttgtctgtagaataatttctcttcaaggtgccagtgaaatgtattagtgatggttagttaactataacctgaccacatagttaccttatatggtcaggttatacaacaggtcacctgttgtcaactccatatctttctagttccaccactcctgtagcagctatatgcattgtaagtagcagattttataaaatgtgtagatcaatccctttggaaaagaatatcaactatacatcatctgtatttcttaattttatcctattttttctctttcttttaagaatactaatgtatccaacttttatattcctttttgtttgtgttatattttagtttctgcataagatttgttttttacctagcctttctaatggggtttcagatgttcatactccagtcttacaggtcctctcctctagaatggtatcttttacgtctgtaatgttctctttatttccattaccctttaattacaagtaaccatacttcaagtaagcagcttttaaaaattaaaaacttgaataaattgctattcaaaatgggttattgaataattttaatctaaaaatgttttcaataagtacatttcaggctagaataacattactaatgacactttattaatagtgctactcatctttctgggataaatgtgagatactcttggataaggaatgggaaagtccaaatatagttttgtatatggaaatatatataaaatatatagtgtggaattttttattgaatattcattggtgcctgaatatctcatgggaattcatttaggatttgcagaatatttagcagacatgttagactttagtattgtcacattttggattatttctgattattcgatgtattagatatatgtgctaatgtaattttggaatcaatttgcttgctctcttctaccttcaaattaaacttaaaattagagtataatggcacaacacaatacatgccttaaaaaactggaggacaaagttatttttagcctagaattgtgaagatagaacaagacaatctaaaaacttttatcttgtgactcttttttcagaagctgctggagtatatgctccagtgctcaccaaaataagggaataaaccaataaaggggattcatgggattcaggaaaccagagttgcagccagtgaaacagcaatcaaaatgacctgagcacacaagctaggcagagcctgctgccctcagtccaactggaacgagttccagtccaactggaatgagaggatttcggggaggaaagtggaaattctatattacttattcattgactgcatcagacaatatattaatccatatgtgttttgaaaatctagaagtgttttacagatttggagaaaattagcaattggtcctgagaaaataatcaaatttaaaagaccttaattaactgcaagaaaaagtcgagtagaaagtataatcataatacactactttgctcaacagtcatgaatatttacataatgataacgttgaatacctctaaaatatgatatttatactctaggtttggagagaatagaactatttaagttagcataaatacttatacatgaaagtaaaaaggtaatatatagtatctaaaatttagaagtcaaaaattacaataagagtatgttatctgaaaatatggagttaaattacagaagagatagctgaaagaattgaaaatgtgacctgtgggacactggactggagaggtggagtgaaatgaagcagggtacagctgctttccattatgagtctggcagtagtccttgaagttttaaactacatgcatgtctaacaaaaaagtaaatatttttaaaagggcaaaaaatactgtgataactcttgacagtttgcaatgtagaaaagaatggaagaggctgtcttcatcttttgaagctcatttcctggtaatctcccctgcctgctctcagcttcagccattctggcctttttgctgtttttccaacacatggggcttggtcttgtcttaggaactctgcgcctgtgccagcactgctcttccctcaggtgtctgcagacttcttcgctgatctctttcaaggctttgcttgaatgtcacttctcagcaaggttccccttgaccactcagtctaaaattgcacctgctcatgcatctgccttattccgctctaatgcttttctccatagtgtttatcaccttcttaaatactgcatgattaagtcatttattatggtttttgttaactttctatctccccttgctacaatgtgaactacacaaggacaacatgtttgtggtttggatggctggggggtccaccagtgcatctcaagtgcctatatatatatatgaaacatatatatggaacactttttattttaatgagctattcagtatttcagtaagctattaaatggtgcttggcatacagtatgtaatcagtatatattagttgaaggtatcaattaactaaatgaagaaagaaaagactaaaaaaggaagggaagaaaaaagagaaggtgggacaaaacaagggacacaagttaggaggcaaatgcattatttcataagatggtggtggtttgagtttgggtagaggcacgataaatggagagaagtgaatgaatttgaaataggcttgaacataaatttggcattataatgaaaaatatgattatttctaaaaccaatgtttcatctgttttataagcaggaaatacctgaaaataaggaatgtcaagattctagtatatgttattagaagtgaatggcattcatagtcatggaaaataaagttaaattattaaatgctatttaaagccacagaagttctagacatcgaataaaaataagaatgatacaataacttagctaatgacaacaataaaagcttttgtagagttttcctacaagttttacagtttagcttgcctgtaagttacaaatacgaggatttttgtgctttgaactataaaagctcatacctaagagtaatgaaaggtttaatattgtacttatttaatagtgctttcttctattaaaaacatagtattctattcttatttataattttatggaaaccaatttcactaacatttataaagtttgatttgaaaataaattatatttcttaataattatatgcacagttatgacctgcatggatttaagctttcgcataatttaaaaatccatatcttcattttgactgtatatttgaaaatactgatgacagagtaatctgattgcactgtaactggccatttttgtgaaataaagtccttttttcttgtctcagtagtcataactagttacgtgtatattgtggttttcttccatataaatgtttctagattgcgtacatttaaaataacttttcatgtctaattctgatgcacatctttagcaaatatgaacagcaaccttgccactgtctctgattcttaacttcttccttctcttatcctttccttatcttatcttctttccttttcttatcctttcagagcctcttccttttctgaaacttagatgggagataggaaggtatggttgtaaaagcattacatctgtcatatgatctatcacatatcatataagaaaatgcattatattttggaaactttaattttatacacatatatatgaaatatgttcatatgaaatacatttcactttattaagctattcaatatctcaataagctattcaatattacaggcacctagataactaaaggtcagtcattacatttatttcagtgtaagaacttaacggggaaaaatttaagttctgttaatgagtttctaataaaagtagacctgttttgaaaacagaagtgtctgtttccttgttatatttgaaataaaaaattatgatgaagcattttccagtgtatgaaactaagattcttttatccataagggactaatgtgtagttgaagatgtccgtaattgaacagcaggaagtattagataaattcaagaaagcccagtaaatcctagagggaattagaaataaagttcatatatttattgttaactgcaaccattttatatgaaaattataacaaggcaaagcttacatttcattatcaatatagtgtccctatccactgttctttgatagcagtcaagtatgggaaacaatataagagatggttatgaagttgataacgtgtttagcactggtttgtactcattctgtatccaggaatctctttttattcattgtgttaaatattcatgagcacttactctgtgtctgatactatgatgtagaggatcgaagagtgtactagtttctgctcttaaagtaatccataggcagaagagagacatacaggtgtatgttcattgaaatatgacagctaccatttgaaaaatgcatggaaatatagatgcttagtgcatttggtaaagaagcgggtgagactaagggaaggggaatttttagggtagcagtgcttaagtatgttacaggggcttaaaagaacagttcagtgaaggagagattgaagtccagaaaatcctggagaaggtaggaggatgggaaaagagtggtggcatctgtctctatgagatacaatgagaaagacgatgatgaaaatagaacttagttaaaaggtgaagaggagggaacttgcaaaatttcccgttttctgttttcttggtgagaacagaatgttcagtctgctcctgatcataaaaggaaggatttagcagtcactgatctgaaaaaggttggggaacagagacgggtaaatgaattatgggtgtcagggaacattcctctaagattataaatcctaaatttgtcatttattcatgtaatgaatatttacagagcacgtttgcctaggttaggtgttgaatatgcagcagggagcaagataggcacattttgaattcttaagcagttcatgccttcatgacacagtaaacaaatattaaaaaattattactcaattataattgtgagaaagaccgtaaaagttaaagaaatggtattaagataccatatgatgcatgagaaggcttgggattgggtgaggaggcagagaaaggagaaagtagagatacccagttggagaaatggctgagcaaaggccctgagaccaggtgcttgagggaggcaaggcttcccactcagtgtgctctggtgccactcgtgaattacacgtgtgtcaaatattcatccgctcagctcttatgtgttattccaaggcacaaagaacattatcactttctaactgagccataatatgaaatagtttgagaagctttgcattaggggaagtgacctcagaaagttttcatctgtagcaccagcaaggtgcatggaataaataaaatatctggatgttttattaatttggcccttcttgtgtgtttatagtcattgaattaattggtttattcagcaaataggtagttgataaaatgacactgcatttaacctggctagttttataaatattacaaattgacacagagacggtccacaatatggaagtttatcttggggttttttatgaaaagatttaaaaaattatcttaccttagcaggtatgggggaaagttttctctcatttcttttccattgagtattgtcagtaaaatagaataagattgagtgaaatttcaaattatttaatgttcaaccaaaagaattctcaacttattcacacatatatattaagtaaagaattcagaaaatcattaataaagttcagacaaaaaccgtctaacctaattcccgggaaaaaaactggaactctgcctagaattatccagctacacagtctgtattcattttttaattgcataccttatcctttttatctctgtgtatagcacctatttaaaagaaacagaaacctggcctggtgtggtggctcatgcctctgatcccagcacttttggaggccgaggcgagcagatcccttgaacccaggagttcaagagcagcctgggcaacatggtgaaaccctgtctctaaataaatatgtaaaagaaagaaacagaaaccagacatttctgtttttagctgtgggttgctacacctctgtgaaatcataggtgtttaccactgtcttaaatcactggtttatgattacttgatattttctggatttttaaaacattacttctttttttaagtggtgtagaagtaacgttttagcatactataaatttctggtaataatttgtattacaaaggtgcatattgtttaaggtcccaagtaagctttatgtatagaaaaggttcctctgctctttctttttgtcctcaaacgtaaacttctggagacctggagagaatgaaaggcgtccgagtgtgtgtccatggcaatccgctagctgtgtggaggaagttacagtggcattaagccatgaaaaggatcaattgtttttgtgaaaaatccttgggacgtgccttgaatttaaagaatcaagttcattaaaatcataaatcatctattaatcttattttgttgaaatatccttggtggtccatggtattttaaagaccaaaattgtaaaaaatgtagttaaagtaacaacataatgggattgttgaggaccgtgaaataatatcctcaatgccttttcttcttgagggtttttactggatacttgctctacctatacctctcctaactacagaactgaacatgatgtgagatgtgtaactttcatttattcactcatgtgccaggcaccatgctgacattacatgtgaaaagtttccaaggttttcttgttacgtctacaattactgctaagtttcccgtaagtaatttgggggactggaaacgagggcaaaattacatgaagggcttagaaaactgcatcaattctttttggatgtgtcagggagatgggggtagtctttctccctgctaatcaagacccactagagcaccgccgtatactgcacacatacttctgaatgacaggagctttgtgtgagatgagaaagctgtgtcaggctgatggtattatcatgattccttggaatggatcaccatcccaaatcaccagacactccctgatcaggagggaacagcacattctccagctactgtattatttgggaggttcaggtaggaagtgagtgcatagcctcaaggggtctggtggaggtcctcttgggaaatgcagaacttctttgcctactaatgccccttgctctgagaccggagaggcttctgggaattttatcctcacaagaaacattcatttcatttaaatagtatgtgaagcaagacatttaagttagttagaggtgcatatcatttaaaatggcaggagatgacagtttctgcaagccatttctgaagttaaaaaacaaggtagtttaaaataaattgtatttcagcagtcactattttcttaatttaccaatgaacttgaatgacatattaaagaactttaggcattatactctggtttgtgatccgcttgaacacaaaatgcatattatgtgatcttatttccaaggttaaatcagtgtatccttggctagatctataatagcttatcagataaacatcttctgtcaaaataatatttgccattaatgaaattaatggcagaagtataaacttcagaaagatatgatcacttagatgctaaaaaatctattaaggaaaacaggttacatttcaatatctttgtctttgtgttctatgatatgtaaattccaaggtgtaaatcctttcataaaaacatttttttctttttcctagacttagagagaaacattgacttgcccacagagctagctcctttgatgtgcaataatgctacctgtggtctgttttaagatgctcttttttggccaggcgcagtggctcatgcctgtaatcccagcactttgggaggccgaggcgggcagatcatgaagtcaagagattgagaccatcctggccaacatggtaaaaccccgtctctactaaaaatacaaaaattaactgggcgtcatggtgtgcgcctataatcccagctacttggaaatccgaggcaggagaatcacttgaacctgggagccggaggttgcagtgagccgagatcgtgccactgcactcctgactggcgacagagtgggactccatctcaaaaaaaaaaaaaaaaaagttcatttttatttaaccgttaccattatcacattagacagtgcaggagtatgttaattatctctaggtgtcccagtggcctagtaagtccagggcaaaatgccagcagagtcctggccaaaaacactacattaacttgttcagtctcttgacatcctcacaaacagaacagtaaacctggagagaaaacatgtttgttccaaatgagttaactattccaaaataaataatatctaacccctggccaagaatttgtaattgtgtataaaaagtaactaatgggatgtattctccaagactattaattatgtactgaacaggaatgctttttaaatgttgttcctctgagtgcaataaacctgtgtaatctaggctacctaatgagcatctttgagcatctgccactgcagagtacattcaaatttcaggcaaaatttgggaaatgctgttgcagcagtggcccagtaagacccagtgctgtcattggtcaagataggtcaccaattacaagacttgaagataagcatgttgtgattggtggttgaagtgaatcccctctccttaaggcctcactttggggattcacagaatgggcattgtggaggccccaagttccatggagaatgcaactattaaattgagaaagaaaagaaagtacaagtgagatgttaggcaaagagtagaggcatgagatggggactaaaagtatccagcagaggaatgtgaagccactaaaatgattctgctagccagctagaccgtacttaagctgtactaattgagacaataggatttgcacagtttccgctcacttcaaataaactgtacaatacaaggaaataaaatgataactagtaattatcacttcatagtctactggcaatgaaagatttttatgtaccgataaagcagtagatgtaatcttttttgtacagttatgagcaaaatcagtgatgaatatagctaggtgctcgttaacaatgaaaagcctactaattagagatgtcatcaaagtgtttaatttttttaaattgctagtggggaaatggttcacccttgaatatcaggatttgtattttcaatgtgcagtgagtgtccatacttacccttaaaactaaagagcacttcagactaagaatcagatagcatcagaaaacaagtgaataatttatgaccaaaaaaacatgcagggcacattagataaatgtcacattgctgttgcatttaaaacgaaattgcttcactattgaaagaaaaaacccaaatgcctgaaaaataaaattcaggaaatgataattgtcgttataagcacattttttacaaagagttcaaaatggttttacacatgccctatctcttgttacggtattattgtaagtaaacaggagtttacttacagtaaaaatatatatccctatttttaaattgtggaaatacaagcaaagagatgtgacttgtccaaggtcatcacttaaaggcagaatgtaagattccgatcttaaaaataaagtgaaaagaggcataaaaaaatatgtgtttatagttaaaattagtcagatctcccttgctcatcctggtttctttttgtaatattgcccaagcttaagtagttttaatgagcaatagcaagtggcttataaaagatactggcaaattgaaaatgtcttaaatcattgaagatccattgaaaaattatcaatttattatagtaaatgtgcttcttgggaataaaggtttcttatttctagaccggctaaaaaaaaaaaaaaaaggaattcatgccaaaaatgaattttcagtcaatatactgggaaaggagtcaagaaactctgccttgtccagacataagtgcacagtgaatcagtcagcatcctgacagcaacttaacatgacccgcacctattttagaacaggttgttaacacataaagctgccagattgtcctgcttaatggatagacagtgacatttgggtttaattttatctaagcaaaatgtgctgttttagaaataagataaaaattaaaataagactatttcaaggtttacccacaaaatgcagacccagtgcatctgatcatcatggccaataagatgatctgtgagtttgttgcgtttcctgttttgctatggttcttgtttttcattatcttggtagctgaagtacttaacttgcctactatgtgtcaacttggctattatttcataagcttaactttaaaatgctgttaatgctgcatctatcatgcagtctcttggaagaagattttaaaatggatttcaaatgtttctttacagtcttaaaacatatttacaatctatctttaatttatctagttgtagaatcagatacattctttcagacattctttaaagtgcatagacactttaaattttttgaatatttaacaaaaactcatcaaatatttggaaaacctgaaaacatatgaaaaaataaaaatcactcccaatggcaccacataaatttacatttctgtatatatgtactatttataaaatagtatcataactgttgatattttatatcatgtatatttcaaaaaaaatcaataaatatatttgaaaatatacttataatggcttcacgtcatttcatattaggaaccttatataatttatttaaacttccccactttttgtatattaacatattttatagttattcaacattttttcaatgttgtaaacatcttggtacatcatgttaatctctgttaatcttttaaaatatgttctttggaatagaattattgtgtccaagatatgaagagttttaaggatcttgataacccttgtcaactcacttcccagaaagtttatagtagttcattcttctacctgtatataaagaaacatatcaagatttctggatcatcattgaatatttaaaaagtaataataaacctttgacaactggttagctggaaaactgctatcttcacattattttagtttctgtttctttgacaccagtaaagtctcacagttctttataaatttattaattgtatttgcattcttaaaacttctgtgctcatattttgcccttattgatttgtgataattttgtatgtgtggaatatattgtaatatttgtttaatgtattttaacatttatcaatagcttaattttacaatttgtatttcattatacatttctctatacaagttttaaatgctatataatcaaagctataaatgtttcttcataatatccttttcttcatacatagaaatgcatcccccatcttatgattaatttaatgttcactttgtaaatagtttttatgcatctttttaaaaacattctttattccacttgtaatttatttttatgtatgtgtcaagtacaatttaatttttttcacaaatagtgttcccaacctttgaaccttcatacttttccttatctaattgtgatgtttcctttaacatatattaaatttgcatctatagtaggttctatgttagtgctgtttgcttttttctattgatctgtctatcaattcatttttttagtaattgattaaccaagcctttattaattcacacttcttctaaatgttctagtcaatttttactctccatttattattctatataaattttcaaaaaaattctccagttagaaacatcccagtgagatttgacctattcattaatatggaaaaaaaaatcacaacttccccaaattcactgtttccattgaagaatgtagtagatctcttcatttattcacattttcttttatattaaaaatagttcatatctttgttcctttatgaacttgcttggtgttttgtgcttttcacggtaaatgtgaatggaatcccattgtatcctctaattggttatttgagtgtgccagttgtccagaacttcctgagtgaccccaatcagaaaggggcacagcagatgttcttgctctgcgtcagctttcagtgaaagtacgtgggcataagatttcaaaacagcaatcttatcattgctaaataatactattatggtaagataagttagtagcatataataacataatacataaatataataacacattatttttgtcatttgatgaattgtgtttgctactcccttttatatactttttcttacacagattgggttttcttctcacttctaatattatttttagctcgttcctttaattgttatttgtatatacgttgatatttttgtctctgttcctattatcactgagcagtcaacattgggtttttctcttatgatggaacaaattcactgttatagacataaaactaggtaactataatgattgtagtacaataacaatgagtttgtgcttagtgttttgttgaaattatagaaggttgacaggaaaaaaatgtggtacttcatccacagattttattttatacctgaagctcaagtaaagctagtttaatagaagctatcctcatgcatatcgtttaagctcaagaaataaatcttctgacattgtcaacttttaattttagcacccaaaaagtagagaatgctttaagtgaactgttatgatgctgtaaaataatatttattaaattgagaatatgtcagtgattcattatcatgtaaagaaataggttaccaaacagtgtcattgaatttaagtaaagtccaaatttattatatataaatatataacatctagagaaacatacatgcatgggcaaaactgttaatagcagttatttgtagggcataggattacaagtgatttttaaagtatttttgcctacctgtattttttctaatttatttgaaataggcatgcattattatgtcataaagatcaaaattattaatatgttttaacaaaaaataactcaactttagaaagatagataaattacattagcttaaatgcatctaagtataggaaaatatactaactacttatcaagattagcctgtgaatattttgagctttcattgaaaagtttgaatagtaaacaaattaccaacatagaaattctctctctgaaagcatgtccagtgtctaagaaacataaaatctaattgctcacctttgagactctccttggtcctgtgagacatatgcatttaattggatgtctcactaccaaaataagcatatgtaagaaatgactccctttgttcttttccattatccttatgttcaaactttccatctccttgaatgattttaccattttgattatatagtgcctcaggcatctactttttatctctttaaacttattcttaaataaagaggttacctggactcttataactcatatttttccagaattacctctttttaatgatatgaattaaaatatcctcttattttgcataaagtttctatttaagttttgagacattcaaaagtacttttattttgaagcctttgaagagaggaggaaaccaaaactctgcttcctaacactggaaggtagttgaaattggaaaacaaatggcttgatatatatactataaaaagtggtattttttaggtcttttggttacacctgaagtggttaacagtaagtgcaatgtttgagctagtattatagcctaaacaaaccatactatctgaattaccataattagctaaattaggtttcataaatgtcgttgcatgtatactgaataaaagtttaaattccagatgggtaaagataggctgtatgataatttaacttcacagcaagccaatccttaacattgaatgacaggaagatccacagagaaaatttgccatgtataattttgtagactctgtactcgtgggcaaatttaattttacttggaaagtttgtcttagcaaatcaaggttttgggttagatcatttgggtcataaactcagctttcttatcaaatggagaaagacaacaaaaatagaacatctgaagatacaaagtatttaataaccaggtgatgtgttcataggtgttctttaatcaaaagttgattctggctgggcacggtggctcatgcctgtaatcccaacattttgggatgccaaggcgggcagatcacttgaggtcagatattcgagaccagcctggccaacatggtgaaaccctgtctctactaaaaataaaaaaattagctgggcatggtagtgcatgcctgtaatcccagctactcgggaggctgaggcagaaggattgcttgaacccagggcgtaggcgtggggtgaggtgcagaggttgcagtaagccaagatcatgccactgcattccagcctgggtgacagagcgggactccgcctcaaaaaaaaaagttgattcttttcttaaaggaattcctattttttattttattctcagagaaaagacttattttgtttccagtgacagatgattgtgactgctgtcactatcaaccgttgtttatcaatgccacttcagtcttaagcatgtcttgcttcaaattctttggtggatgataaccactctgatgacagtatactatctccagagggttctaagtccttcttcaagccttcacaaactacctatatttcttcacaagcacacatccaaccctttcatttcagttaaaatattttttggattatttcatactcttctttgaaatttttcctcaaaatatatgcaacatatgaaatacattcaatgccattcagtcaactcagtagactacctcctcacttttaaaagaaatctaggtaagaagagagagtaggatgaaagttaaaagtgtgaagatggatagtgatctgtcaccaaaaataagttcctagtgatctatgaagatcctacatgagcgctccctggactgtcaaatcttgagttctgtgatttctcacctcttgatcctcatgccttttcttctacctggaacattcagtcccttttctcaggtctcttctttgtcagtcttcttcaaggtctttaatggttcatctctaattctatcatttctttgccacatttctagacttctgctatcaactgatctcctttttatgtcctcggatattctttagtaacttctttaaaggacgtgaaatatgcaagaagtatcatctaccttgaacaaattcactttaagagatatggaatcctagaattcaaacttagatctgtggttattttctatctgaaagttatttttcttaggagcactgtatttctccctattacagtaatattttggccacttatggcaataacgtgtgtatgtgtttgtgtgtgtattttgtcagagcactgctgctagaatttttagtagaatgcagttgataaattgttaatcaatatgaaacagagactttctttagggtaagaccacaatcagaagcatttttagaattcaaaatatttataatgattttttttagtagaaaatcagcaccctgattttaacttggtgggggggtggtaaaaattttgagaaaaattggtttccttgatagggagttatcaaggaaaacaaaactttgatttcctttacataagtgtaaacagaaatgaagaaatatgtctttataatacatatactaaataaatatatgtatttttatatatgcttgtatacatacctgtttgcatatttgtaagtgcttgaggtaagttcaaggtggaatgggttgaaaggagaatgtttaggttatctgaaataatataaaccaaattcctctcaatttagttaggaggttagaaagctacttcatgaagaatatttttgggacaaattttaaggtaaccttaaaaaagatttgctaatatgtattttcttgtatatcatgtgaacaatcacttcctgaaatttcttagttttatcactacaaaatagtcacaccataaccgccttgtcaaagattttaaatggaaagaaacaaaatataaaaactacaaagaaactgaaatccacagaaacagaaaaaaaattctatatttcattattttaatggatcctaaaatgcgtaagcactttgatagattcttttttatataggatttcctggttttcaaagtatataatgcaacaaaaaagtgaacccagcttctttttttcttgttcattggagctatcacatctcatctatcagacctctaagtagaatttttaaagtggggtactctctcacgcccaagcactatgtaattagcatatgaatgtgtaacggattgtttgaagtaacaattaagggaacaatacataaaggaactgaagagagctaccaacacctgggaatcttgtattgaaaaagctttcttaatgtaagtaattaaaaaccaaggataaatgttcattgtgtagggataaaactctacaaggaaagctaaaattctgctgatgttttgctgttatcctttagaaggaatcattaaaacaagagcttacttttttatttatttcaataaaatgcttgttaaatacaggttttgcatggtatctgatctcctgaagtcaccacaatgcataacaaaaatgtcttacatttctaaaatattcatatttaaaatgtatataaaatgtcaaggaatcttcttcctatggccagtgcagtttcagagaaaatttcaattgattgccaggttaaatcacgtatatagtcaattctttataatttatttgtccaagtaattcctgctaatgaatctgtagcccgctctattgttttgcactattttgtgtctcttgaaggcaagcatgctatagaggctcctgctgaggcggagcaccttaggagagtaaccagtagctcaggattcttaggaatcgttgatattgagggaggataaatgaagtggaaggcagggaccccgcattatggtttccattttttatcacttgctgcctgtacaaaccacttatgttttttggcataaatcttcttatctctaaaataagaattacttttttctagactgttaagtctcagtcagtaaccttttatggccctttctaatatccagttgtatatattcatttgtaataaagaaactcttaaccccggaatttaacttaaatcagtttagtcttaaataactagcattttatggaacaaccatatttgaaaaataaattgtgagcctaagatttcatttgtaagtagaaagtgaaacaaaaatgtatgaatattcataacagcattgttcataatagccaaaaattagaaacaacacaagtgtctattacttgatgaatggatgaataaaatatggtatattcaattgtcaaaacaaaaattgcaccaaagttaaacaggaaggaaatactttactcaaggctgttgcagtaggggagagaagctggatccgagtctgaactcaacacacatgaagcaaactgggtcaggtttttaagtgctggagtgagcttgtggaaaagaactgcaggacattcagggccataagctatctgtatttgctgattggctttatctaaactacctcttgtcataaagatatgagtttatctaaataaatttaagtttatctaaataaatctaaataaattcatatctttatgacaagaagtagcttttacaacttggagcaaggtgccccactgaagttaggctcctaccctcccacctaaactggaagataaaggtgctgtctctcttgttgattacttttcaaagaggtagttcccaggtccttgagaggtacatttctggattataaagttaccaagtggctactaaaaagatttacgcctcacaggagtagagaaagaattacaagccgggcacagtggctcacgcctgtaattccagcactttgggaggccaaggcgggcggatcacgaggtctggagttcgagaccagcctgaccaacatagtgaaaccccgtctctactaaaaattcaaaaaaattagccaggcgtggtggcatgcgcctgtaatcgcagttactcaggaggctgaggcagaagaatcgcttgaaccggagaggtggaggttgcagtgagctgagatcatgccattgcactccagcctgggctacagagcaagattctgtcaaaaaaaaaaaaaaggattacaattacaatattcttaaagtaaattctataataaagggaaatcaagggcctatggtcaagaacaaccctttctaaagtttagtcaaaccaaggggaactttcaggctgtcctggtcaccatatgatagaatattcttggacagtgaaggaggatgatgtattgatacatgctatgccatgtataaacctcaaaagcattatgtgacgtgaaagaaggtggtcgcaaaaggccatgtattgtatcattctgtttatatggaaatgtctagaatagtcaaatctatatagtcaaaaatattagtgttttcctagggctgggggatgggcgtgggtgtggggtggcagctaagggtatggaatttctttttagagtgatgaaaatgttctaaaattgtttgcaggaatgattacaaaacttcgtaaatattctaaaagccattgacttgtacactttaatggatgaattgtgtgatacgtagattatattttaataaaactattgtaaaatatagatttatgttgaaatgtcttatatagccaatagaatatttacttaatatatagatttttacaaatttaaacacaaccaaaacctttatttttccaaagtacgttttggctttggataacctgtgaattttaagtgtgcatttctgatagctggcctgccttttagtttttataaaatagtaacttattaccatgttgtaataatcagatagtatataacatgaaagaaagaaaaataaattctgaaatgtccttgagatatttgcatccaatagagaaaacaaatgtataaagagaataagaagtaatttcatactatataaacagagtgccaatgtatgaatgaataatgttctacaagtacatttgtaaactagctatttgtagctttctctacagaaacagttctgccatccgcaatcaggttccaaacctatcctgcaaagatttattttacccacagtgtagctgaaatgaagtattaatgttagtacagtcagggattaagggacaatcatacaggaaggaggagaatgaatctgaacactcccatttattggaagcaggcatggctcctggcaaaattttgaattatgcagagtgagactggttttcacccatcttcttttgtctatcccttgccccatgttctagatctagagactctctctgggtcatggggcattgactatttttatggccctaagtttcccattgcattacctaactcaagcatctctgttcctccaagtgacaaattcactcacgctatctgacgtaacatttctccgaaacaacatatgaacatttaaattctactttctgaccgcagctaattttgttttgcattaggaaggaagcaaattttgaaagcgtgataaactctgggtacactagaagcattttcagataaaacttggattgtaaacataaaggtatctttaattaataagatttcaggcattagacttagggaatggtaacaaaaatttggaggcatgacaagctgtcctttctgactccagtccctctttggggctgaacccctctaagctgctctttcattacctaaatatgtactttgtttctgcctgaatgtcagcatcctttaaacacaccctctaaaatttgcactagctaccagaactagggcattggagtgtataatataatggtggtggttgaggttgagagaacacattgagaagatgacatggaaaggagtaatcaagaactatttatggtatgaataatttgggcatcttttatttttggacatatatttttctacatactgattacttggtttttcttttttatagattgggctaatggatactacagacaacagagaaaacttgttgaagagattggctggtcctatacaggtaaacatattacttatataatgagatttagcagaaacttttattgttttgtatctatttttatatataaaaggaagctttgtagcacaacacatcatgtcaacttagctaaatggatagttagattaattaatagatatataaaatataaaacgatatcttatgggctttccataaacaaatttggtaatgtagggcttattgacatccacccaaactgtatttgaagctcaggcccaagctgtctatataccttcaagaaataacataacctggggctctgtttttttcaacggtgaaatggggggctgttctgagcattaaatgagataaataaagtgtgaagcacttgtcacagagcctgcttctaaacagaaagctctcaacaaatgttagctgctggagttgttatgatcatgctaatgattatttttgttcatttaacaaatattgatgggattcttgctctcagaaggggcattcttctgaattactgttgttaatttattggtgttagagtaaaaaatgattttgtcacattactagactttgcctagaacattatgaacatctgaagttggcttgaaatagttacaaccaaaaatcatcagcagaaactctgggagagaaagttctagccctgagtggaagaatatgagtggaatttgggtgaaccatgacatgcctagtaaccatgtgggatggagttgagcattagagttgggtactcattagcagtaaacttgggttgataagataaagtcagattacaagggctttaccatgagcctggccaattttacttaatagatgaagtgcagaagcatgctgaattttgtagataaggatgtattttctttcttctttttaattgctattttaaaagtagtaaatattcattgtagaaaatttagaaataggttcaagtacaaagaaaaacataaaatcattattttatttccctcaggaatagccacttaaaaaactgctatagtccttttagacagtttttattatccaaaatttatatattttatttatagagttatgcatgcagtttactcttaaaattatatttatttcctcattttaatattcctgctgatcatgatagttaatctgcttactaatccattgaataatttttttattatgtaaccacttccttatttttgtatatttaagcttctataagcaatgacagatgaattgacagatagatgtgtttttatttttattacttctgtgctatagaattattgatatgcaattaccgagtaggaaaaaaaaaaaaaaaactttcaagccaggtgtggtggctcaagcctgtaatcccagcactttgggaggctgaggtgggtagatcacaaggtcaggagttcgagatcagcctggccaacatggtgaaaacccatctctactaaaaatacaaaaaaattagccaggcatgttggcgcatgcttctaatcccagctacttaggaggctgaggcaggagaattgcttgaacccaggaggcggaggttgcagtgagccgagatcacgccactgcactccagcctgggtgacagagcaagactccatctcaaaaaaaaaataaaaataaaaaataaataaataaataaataaataaataaatagatttgtgatgcacactgcctaaatgcttcctagatgggttacactcatctgtcatataaatgactaccccactgtatactacccaagattgatgattgtcatattgaaaatctttatgaattcaacagatttttaaaaatcatgttttgttactttaatttgctttttttttattaccactgaagtcatgattttaattttttattagccatttgcactatacctcttcagtgcaataagttaaattagaacctggaaaacaatcaagatgtttatcagagcaggaagagtgaacaaattaaggcacatctcgactgtggagtactctgcagatgggtatgaggtagacctctgtaatattgtacagaaagacctactattaatttactcatttataataaggacccatacatctataaaatactacttttgatattttaaatcctaaaaaatacataaaagcaaaaattattcataatttgactccattttgaattaaatggaattttcttttttatttttctatgtaaacaaattatacccaatttaataaacatcagcaaactctatttacttgaggaatttcttttttcagaaaacatgaagtttaatcaaaaaggttgttggaccaaagggcctacctatttataatattaacagatatgttcaaattaatttcagaaaattgtgttaaaattatctatttccccacattttcactaacactgactctaaaaatctagtcaatttgattagtcaaaaatggagtctaaattttaatttgtatttccctgattacctacaaatttttctattatgtttttgtatgtactaccaattgggtatatgtttattaaataatgtagtattgtcttttaaaaaactttataattttatatactttataactttacatctgacaattgtaactcattttgtcttcttttcctacgaaagagaaatcctagtattttataaaaatcatgactgttttttaatgcccctaataattatgagaaacatgtcaggagggactaagaatgcttagagtgcatccctaaccatggaagatgacattacaaggtgaaatccttagttttcctggaaaatatctgccagtgtcaccatctgggcaatatttaatgcaggacatctttttaaatgattcatttaaatattgctagttgtggtttcggaatgctacttccaggataagcttttccccaagaggttctttatgagtattaaagttctacatgtttgggccttttatgtttatatttttatttttaattcctaagaatatcacaagccatcttttaaaaattcactattgtgttgtttttaagataatgtttgctccattaagagtaatatataactgctttaaaaacgtgatacatagagatgttatcacagaataaaagtcagcctggtttagttttgtcaacagctaactttctagtttcagaagtgaaactagaaggaggttattttcctctaaccaatcaattttcaaacagagaactcagatatttaccaagtatgtatgagtaaaatgaaaaacaaactttaatgcaccctccactctccctaaacccagagttagaaaacagaggacctactgcctagggaataagaaaattatagttctcagctccaagcaagggtggatgtatgttgactaatggagtgatggtggtgtgtgtttcccagcagcccagggaccctatgcatcttgggtgtgcctcataaggtcaggagaagacaacagttttaccagggtttcagcagatatgatagcaacagcacataccattgtgcaatatcttattatcttaggaggcaaccacagagctgctccttttaatgggctgagtatagacacagcacctatagagtggctaggctttgacagatgggtctttgaagaaacaatgtgtagttctttgctttaaaatcaagttgaatcatcttgctcataacctaaggcctattggctttagcttctttcaggaaagtctagtgatcaaagggatagactcattaaagcgtggtatttgttgtaggggggtggggtggcagatctcaaaaagggatgttccagagtcagggtgaggatggaagcttaccaaagttttttaaagagtgaattgctttatttatattagtgcttcccattggtttggaaaattattcctagaatatttgaggatcctaggaagaaactaaagatgagtacattttacaacccttcgtttagctcaaaactgagatgacataattattttgtctgaattactagactatcaaagagaaaacaaataaatgaaattctttatccaaataaatgtatttcacccaagcaaattacattaaaaccaaacctttaacgtggattattcactataaagtgaattatcactttttatttttaatacaaatatttaaagtttggtagtattttttaatttctatgctatttttccatttagaaaatgaacgactatgctaactgcttgaaataactgtatgaaaataatgaaatgttagtcttatcattcttaaatgtaactaattttgtgaatggcttgaggaaattggacttgaccacattcagttctagtttcaccttaacagtttatcttgttgagctgtatgtttgtattccttcccatattgtctcatcatttaaatttatcaaattagtggcaatcacggaagaaatggcttgcccttgagtgtctgaacatataggattctattaataaaaattactaagtacattataaggcttgtctgtaaaagaactaataagccatttagaaatattacattacacatcaaacattatttaaatgcaatcacttgagctaaagtaattatgaaaaaaattacacaattatgatatatgaagtaacaatgtaaatccccgctggctaccagatgggtggagaaagggaggagaggtaatactcttttagttgtgttttatgcataggttaagaaaaactttctattcaggatgtatgctacaattatgctaagaaatgacagggttggtaaatacagttgacagtcccatttttaaaagttaatataggaggttgagcaaggcttgcttgtcactttgtgatgaaagagtgaattgtaaatctgggaatgtcagcactaagaatctgactctttctgtaacaacttcatttcatttaagggtttcagaaaataaatgtcactcttcctagtaaccttaagttttctatgacaattcctaaacaaagacagttcagtgaaaaaaaaaaaaaaagataaagtgaagtcattttattttttttaatctcatgaacacatagcccaagattatcctaggagatgttaaaaatcttcttttcgagttgttagatctttgattgcaacctcaagcaatgataagatcatcagaaatgggagtactttaactgctttattcttagaagtagtacgacagaaatataatctttattttctggactttaaaaatattatattatttctttttcaccaatctttaaagcagcttgaaattgaaaattttatattgatatgttctcaggtattcttaaaaccttaacacctttaaaattttagttaaagaatgcattcgattcaaataagaaagtatggctgttctggtcacacacaagtctgttatacctcgctggatatgctagaatgagagagtctttaaatatcgaattgattctatttctgtaaatagaatatcctgtctaaatagtctttccattttgataactgtatgtagaaaatgctacttatagaaaacaaaaaggccataatttaaggttgtgtatagtatagtttggtgtttggcttttatttttggtttagtgttttatttcttttggcccaggagaaagtaagccaaattttactcagaaagaaaatttgaatccatgtagaataatgacggatttttggcctttctttaacttctataatctagaatatccttttaacatattatattttcctctttgattcaccacttcttatataaacagactgttcctttctaatttccaattgagcagtagaaaagagagttccagaactctaactatagaattatctcatttctgttaaaaaatatgtatttatatggacatagatgaaaggctggaatgatatatactctaccctgttgaccatggttacccctggagagtgagtctctagatgtggatgaaaccataggaggcaggagcttcacttctcactgtatactattcatactggaggaaaaaataatttttgacaaatatgttgacacaaaatattttcaacttaacgtgaattaattttagcaagaagggcaaaggtagttggacaagaaccgtagcttcgtgtagtcacctgtcaaaatgtagggcctctgcttggctgaatctcagtgagaggatatcacagaccctaacaagagcatggacagtgctcagtgcaggttttctgatcatcattaacctacagactgactaggctgcttaagagctctgcatttcgagcttgcagtgagccgagatcgcgccactgcactctagcctgggcaacaaagcgagactccgtctcaaaaaaaaaaaaaaaaaaaaaaaaagctctgcatttcgattttcccgtttgtaaaatacttcttaataatgcccaacttaattgtgtgcagatcaaatagatgcttcacattgttgcagaaaagctctttgagcaagtacattatgaatccaagtaattattattgaacagtgtcttaacttgatcccaaacagattgaacaagcctgggcccatccgataatctcacaagaggatttagaagtggttgatttgcttgaatggatattatagacagatatttcctatcatctgtagattttttttaaagtttagctaagatttatattctggtcaaactttttctatgctagataatggtgactaaagcaagcctcgttgtggaggctgaacagggttcagttaagtgatctgaaactcagaacctatgtgtattgactctattcatgaaaattgctaagagtagattttaggtgttctcaccacacacacacaaaacagataaggatgtgaggtaacgtatatttgaattagctcaatttagtcattccaccgtgtatccatatttcacaaccacatgtgtatacaataaatatatacaatgtgtgtttgttgatcaaaagataaataaatatgcttaacaattttatttggtcgcaactcttatgtagtatggacttaaaaaaattaagatgtggctacagaatagcatcgcaattagataatcattgtttattttctctgctagtaaaactaaatgctaatgtgtctcccctattaaaaaaaaagttaaaattgcatagattctttcctgagtccagcttcacaagtatcattgctggtagcatctagtagaagcttcaatgggatgggcaagtcctagcagctcagaatgaggaaacttggccccttcttcttctgtctgagctggaactgtctgggctttgtgtaggacgcattgtgcttcttccccttgctgggcctcctttacttcaatctctgtggtttttgtttgttcgtttgttcgtttgtttttgtttttgttttttggggttttttttgtttgtttgtttgtttgtttgtttgtttgttttgagaaggagtgtctcactcagtcgcccaggctggagtgcagtggcgcaatctcggcttgctgctgcaatctccgcctcccaggttcaagtgattctcctgcctcagcctcctgagtagctgggactacaggcgcccgccaccacatcccggctaattttttgtatttttagtagagatggggtttcaccgtgttagccaggatggtctcaatctcctgacctcgtgatctgcccacctcagcctccccaagtgctgggattacaggtatgagccacctcgcagagcctactctctgtgttctttactcagtagggctctagttcgtgaaatagacacatttcataaaagaaaaagggaagagaaaattacatggctaattaaaagaaatctaagtctaggctgcagtttgcattgtcacacaaaattcacttaaatgtgttacacttttaggcaaaatgccattataataattttatgttgacctgtgttgccctccaatttcatgcatcagtaagggcctagtggaaggcttatttagtttcaatttcctttgttttctgtatggtaattctgactctttaaaccaagaattttcaaggagcaaaaagataaaaaatcattatcaaggagcaaaaagatagaaaaatcatttctattattcagacaaatttttattaagtgaagcatattttaatgactgaggcacagaatcacaaaaattattttgaaatatagtgagacacattttgacttagggattttatttcctttatcccttaggaggaatgtgtatatggctgtttatcctgttatctgtgaataccatgtgacaactaaagaataaaaatatcaaactattaaagaattaaagttagttttattccgaagcctgactgaggactatagactgaggcctatagccgggagcactgctgctgcacactgttgcagccacggcttttacacaggttgtggaggttcagtatgtgcaaaatcacatcagattcactcaaaagttacgttaaagtagaaacacatcgaggtttgggtgtgagagtctatctaactacagatgactaaagtacattggttatagattacagaagcataaacactaaccccatcagatgttatcttatctgtaagaaaaggcaaagattggggtcatttgtcttctagggaatacagtgacttaggcaagagacgtgggggctttgtgctgtatcctcttttgtcttcaaagcctccctccagagagttgcacattgtcgcagagtcaggagcttgtgaaattacagtggcaaacagaaacgagcaaacatgccttcctacatttaccactttgtctcatacctttttctgcttaagccacatagtgttttgttcagatgagataaccgcttgactgaaccagagctgggccttctcttggctggatgcagaggatcttggagatcgtctgtggcccttactacaccagcatccaaactgttatagactgttatagagcttctaactaccacagaatattctggtatgcttacaacagctgcacgtattcattcttcaattgcataattgtgcaattgcacaattgcacaagtttctttggaagagagggaaacttgaaccctattgagaggtagggtccagaccagaagagccgtgtttgataacctcaggacagtgggcctgttggaagccagaatcaagggagaggcaaggaggagcccctgagagacaggagacagttgggggactgaggaagaaatatggcagcagagactgagggcagagtggagcctggcaccaggaggagcaggttcccagttcattgtgatgcacctggcggctcccctggggccttccccacccagagtgttcctctttctcaaatgcctacatgctttgtcctggaaacaacagagccccagcaactccatagtggggctcctcttctgaaagaggcagaaatgagaagaggaagatgctgggggttcagcagccgaggtgcatgtggtgcaggcattcagtccaattaaacaggcacccagagtcagggtggagactgatggtctggaggaagaagcagcaaccttcaacaaaatcagattctggtccatgcaagctaaacttcattcccttaggggatgctattcagctccctaatttagtagaataacttctgatgaccaataaaaaggagcctatatacacacaggctgaggagacttagtgtgataagatggtagatgccttctgatcggattccattaatgccgagatccttaaagtgtagagatctgagccattatcttcacaagtattgtaagtgccaatctgatatttttttcccacaaggaaaacacttttttatttatcattataaaagtgtgttcatctaagaaaatcatgtatgtatcatatatttcatatttcatatgtttatatatatttatctacctatctattaatatctatctatttatctatataatttcaccttccagagataactactcttaatgtcttctgttggggaaatataattaaaaacaaaatctcccacctcagaaatcctctccataaatgtaataacaacaacaacaaaaagcagttttattatcaaacaagcattaaaccagaatgtaatacacatcataggcaatcactgaaagattgcgaagataggaagaaatctcacccccccatatagccaaataggtacaacccattgcatgttttcaagataaacaataacttgtctttaagtagaaggacttggcagcaactttgtcacacatggttcagctaaactttaactggtaattggagtgactatttgtgttaccctattggcttcatccagagggaaaataaacttcatatttttgagacaagaggtagctttacaagttggagccagaggctcatggaagttaagctcctgccttcccatagaaattgggagagtggagcaccatcttcctcgatgtttgcatttcataaggatggcccccgggtccttgagaaaaacatcctggatcataaagctgacaaagggcctatctagctttcaaaaggattgatatacattgcaaaacaaggagaaagtatttacaacgatctcttttctaaagcaaatgctctttaaaaaaggacggggtgggggtggagagaaaagtgtcttcctggttttcaatggggagaattaagcctcttacttttactttttatttgtccttacacttcctaattatgcatcctgaattctgatggcctcttactagatgtactgcttttatctcaatagtagtacctgcttttatctcaatagtagtttgtgaatatctttctaagtaaatgtaactaaatcagtgtgagggcaacagaaaaaataagcttctaccagggcaaaactttgactatccatgtgtctgaggcagagactcacacatagtaggagcccaatatatatttgatagttgttactttatttccagtctcatggggaacagaaataattttgcctgagaggaattgtgactagtaataacataatttcaggggatcatcccaaatccttcttgcaacaaatagattatacaaaagataaacactgtggagtgagttcagagcaaggcttagcatataagaagtagctaaaaatcgtaatgaattggaaaacagggaaggctgcattttatagtgctttgccacctggaaattgaaacaaaggcagagccacctagaaccagtgccaaagcaaatccaaaacttggcatatgccaaaatatattacctctaaactctgtagtttccattctttcatacttcattagcatatctaggaagtaagatctatttgcagcactttgaaaaaaataaataaatgacatgtagtttttcttttccaagaccctaaaagtttgttcttgaagatcagttgtatttatgcatataacatactcatatatcattcagatttttatgttcagtcaacttgtgttagaatatgaaggagtaaacttctcatcaactccctagcttgccttctaccttaatgatattttaaacttactgacaaaggaaaatttaaagtgtggatattattagtttaatcaatactcattggattgtacagcgtaagccagacactgtactaggcatcagagatgcagtgatgagtgtaagtaatcacaagtatgcaaacaatagtgattaagcgtgattttttttttaaagcatggtaaatacctcatggagatggtgttaatgtaaggtacggaaccctaattggcctaagagtttcagggaaggcttaccagagaaagcgactttagattcagataaagcataaataggagttaaatcaacatgggtgaaaaattgtgccaggcaaagggagcagctatcctaagatctgtaggtgaaggagaaatcactgtgctccagcctctaccgcattcttcctgcttttggacagaaaattaggaatgtgatgagacaagcttcttggggcccactgaattaattcccatacacttaccctattgaaaattctggataacaagatttatgccatagtttgattggcaatggcttacattttaactgttgacttctttgcatttaagagatgttaggcatgattttacatcagcacactagttaggaaacgaaaggaaaagggactagtaaaagagtccaaaagagaggggtatgagaaaaggagctttacctactccaagaggggtacagcttcaagttgataggattaatcaacattgtcacacatagttctggggagttcatagtgagataaagactatggacttggatgtattttaatgaagcaggttgtagtggggattcttttgttagtttgtctgatgggaatacaagctccagagacagtcctactctttccttctaatctgggctccatcttacatgtcatcttttttgtcccaaagtttttcacctgtaaaaagaactaatgttagtaccaagctcagcaggtgctgcaatgattacatgtgtaagtatatacaaagcagttagaatagtgtctggtgcatataaagtgctcaataaaattattatttaaagtcagataaatcttctgtcatattcaggacttctgacaggtttatgtccatcacgcaattatctttcaatagtacagatataattatatgattctccccatcacctgccaaatgaaaaaaaaaaaagggctctttagcttggcatataatggctttaatgagctggcccacctatcctgtttcattcctcatgattaccttccccctgcctgctgctataccagccttttcaaaagcaggtctctccgattctgtttataatcctgcctgaatagcagactatgcaccttcttcaagtgaagccttctttcctgactcttcttttctgcaggccagagcacatcccttgctttcatacactgtccctgtccctctctctttgttgcagtgcagcagcataacaggttgcatgtctcactcatcttcccttgttcagaatgtagtacctcatgtgtgctcaggaagtgtgtatggcatgaatggacaaagctacgtggattgagcttttgttttacaaatggagaaataaagagacgtagtcatttgcctagagtcacaaagtaagcagaagactggaaacttggacctgggttttgactcctagggccctggccagagaatttctgtggccacatctgattttttgtttgtttgtttttgtgttgtttaagtgatgtgggaaataatataagatataaagctgttaggctttgagttcttgtgtaaatataaaaacttctggttccaaagccttgtgtatggtcttcttagcctttatgcatgtcttcttaggaaatttgtaagagcagtcatttctggaaagtggaattgcaagtgccctttattttcatctgtttgcttgtccagcatcttctagtttttctagactaaacatatatttgttttataatcaggtgtgggaaaagggagcttaaatgtctaaattaatttttattccaggattattaagagattgtttttgcatcatggaatgtcttctgccttttgatatttcctcattttataatggtccttttatgtatctgaggggcctgtgtctgggcctatttctacttcactgtaccaggtgaacttctttaccaagcaataaagtcagaaatggtatattaatcacctatgtccttaaggagtgataataaatagtaattagtgtgaaaatatattctcttaggaaaacggtcatattatacatttgcattttatctggacttcatctagccaggattccactgagggcacttaaaggatcacacaacttgattttcttctctgctgtgacacaatagcctcactttgcattcgtttcacaatctgggatcattatccaagctcagtgagaatcaatttctcatagatgattgcctgaatcactgccagctcagccacattttttacatcatttaccaggaagtatagtttttaaaaagtcagaacaattgtgcaggtaacccagaacctgaatatgccctcaaaatattcttcagaggggtcacccacccctgaaactcaaagtcttctgcaaattgattgacaatgttactgacctgtaaatcaaaggattccccatttcaattcaccattttctaagtgagtctctattgacatgaaagagttcattctcttttgaaagaaaagttcatctttagagagcagcctcaattgtttgctttttagcagaataattacatctttaatttctgtggagataatgaagaaagccagtttaggatatatttatgaggtttcagagtatatttggggagaaaattctataacttttaaatatgattctttagcttcaaaacgtacatgtttaggtaaattgcaataactgttttactttcatttatggtgattgcttgttcatgcttgagcacaagagagaaagcttgatttaaaacgtaagtctcattgatgagtttttatgccaccatcattttaaaaagtaaatttagtagaaagctaattttggcatataatgcaaattcctctactttatctggccttcatttaatgattttggagttatcactgcaaggtgtttatatagattagataccacagagatctaaggaactatatttatttgatcctaattaatatttggccttaaatagatagtcacaccactttccaatttcctaattttaagcaattaaacagctagcagctgttggccagataaatattaaactttgccaagtagatatatgcaagttttttaaccatatttggactatattagaatagattacaaaatgtgttgacatcatatactttagaagattgtatgttacaggttcatactgaacaattatttattatctgtttggtattgttaaaactcatgctaaaatatcatggatagttgtatactatctatgcatgccggctcatagtatgtgaagacacatcaaataaaaactcctatgactatctttacaagcgtgctttgtatttaaataacattaaagataattaaatattgtgttagtgaggaactttatttccttatttggaatattcttaaacttttacagtatgttctttttttgacagtgaaattgtcaaatgagcaatcacaatgataaagaatgaaaccataagtagtgtagaatgtatttatgtatttatttatttttttgagatggagtctctatcgcccaggctggagtgcagtggcgcgatctcggctcactgcaagctccacctcccgggttcacgccattctcctgcctcagcctcccaagtagctgggactacaggtgcccgccaccatgcccggctaatttttttttgtatttttagtagagatggggctttaccgtgttagccaggatggtctcgatctcctgacctcgtgatcctcccgcctcggcctcccaacgtgctgggattacaggcgtgagccactgcgcccggccaataatggagaatttaaagcagtggttcctaaattttagagaccatcagaattacctggagggcttactgaaaaaagattactggtcctgattttcagagtttctgattcagtaaatctgagatagaagccacaaatttaatgtctaacaaattctcaggtgttcctgatactgcagatctgaggagccaaacgttgcagtgtctagactctaggtcaatctcagcttatgttatcttaatttggggaatgaatgaatgatggagtgaagaagagaaagcacatgcatgaatatatttatttgtgtaaaagtgcttcagctctctggatgaggtatattgacatttaaaaagtagcagtattgcctcagtgtaacatgtaacaacattgaaaatgctattttcaaaagctgctatctgcatcaatagagatgagttaagtttttggacaatttttaatgcaacttgcattaatttccccatctggatggggcctctttacctacatagggaaattcctggcctaggtggatccagcaaatccttcattactgaatctggctgtaagttatctttaccttacactactcttgacacatttatccctagcaatttccaaggaaatttccttttttataatctttgtatttacctgactccagaagatcctgggtcctgaaaatattaagattgtgtttttcattcttatatatatatttttaagtttttaaagcctggtagtagagtccttttaaattgtgttgttaataatcctaataataataatttccttctaatatttttggctgttggtaatgtaatttatgggtatatccaaaatttttaaaataggatctgaatatttctggatttctattcagtaaaagttgttattcattctttgcttcagtagctgtttggagttaaacaactcatgtaattgtcacttttcaagtcaataatgtaaaccccacaggacaaattattttagtgccatacttacagattacattttttttttaaagacagtcttgtcttgctctgtctcccaggctggagtgcagtggggtgatctcagctcactgcaacctctgcctttcaggttcaagcaattctcctgcctcagcctcccgagtagctgggactacaggcaggcaccaccatgtccagctaatttttatatttttagtagagacggggtttcaacatgttggccaggctggtttctcactcctgacctcaggtaatccacctgattcggcctcctttagcgctggaattacagcgcaagccaccatacccggcccagattacattttttgtcattgggctccatttttcttgaaatttagtctgtaaccctggggagtatgtttgaaagttttaatagttgtccaaaaaaatgttatctgtcagttcccattactctagcagaggatttatttgaaaatctcataatgaatggtagagcctgcatttgttgtgtaataatttcattcacttattcatatattcaacacatcttaattaagcactcctatctgccagcagaaaataaaaaagactaagtgccccagatggagcttgaattctaaaataacagagaacaaatatctataggtaaaggaaatgactcacagtgacacattaaactatactgtgtaagatgcagtctcattcatgaatattgagtacagtcattttagactattattctctggagatattctagaattatagagattaaaaccattgatacatgtagatcaacagctactaaattttgaagatcaagccattgtctttttttaagtaggtaatttttttggtacattttcaaagttgtaagttttgcaaattcatttttttatttttcatttcctcagactcaatattcccccttggcttaggaggattcacagtatacctccaggttacatgagtagctccaatcaagccatgcctaataatgagctagttcatgtggggaactcctgaaaaatgactgagctcatcccagttctcacagatatttgcaaccagtcatacctacttctcctttaaattctgtacctcttcttatacacaccctagagacagtgtgccaagcataacatcttaatctatgcccaatgcaagaaggaattctctctctttttaaagccatggaaaactcctcgtagtttaaagacagcctcttctccacctccctaggaagaatgttagacttagtgatggggtaattttctcaaaagtttgtaaaaaagcagcttgtcattttggtcatattattaccattaagtacaggaagttaccttcagagtagaattcagtaggcaccattccctaagtgtaaatctccttcactcattaaaagttctgcaccacacccacactgtatatagtattggaagttgctcaggagtcttggctgctagtaagagaagcctgagaagtactcatgctctgatctgagtgggaaatagttgttcagtgctcttgaaattgaaacaagaattttctttcaaccagagaaatgggaaaggatttagtgattgcctgtaaaaacctaaggaactgttcaactatatagagccactgtttggccgcatggaggatcttaacacatccctaggtattcctgtatgtggtaataaccacagacatgggaacagcttttgaatcctaagacctgtaccccagtgaatataatgagagagaatataagtcagagagggaattcaaattgttcctctgggagcctgagaaatgtaaaaaactggtttactccctatactaatcctaaaaacagttgtcatcactcttaattcatttcctggaaaagagacaactgacaacaaagtagcgagaaggtcccatagagctccaataaacagtggtagtaatattttgtatcctgttgaacaaaataggaaaccttgagttcaaattaatataaattgagaattgaaaaaaataaatttaatgaagaatgagatattgctataacttcaaaatacctcccctcaaaatactttctaattacaagacagagaataatattgcagtggaaagcctgacagatactttaatcaagtgatcaaagtaaacgtcagcaaaaattacctttacagagatcctgtaccactgactaagatacagtgagaagaaaagagcatattttttgtgacgtttctaataaaaatgcataacctgaagagacatcagacaaactcaaattgagggacattctataaaataactgtagtcttcaaaaggattgagattacgaaagtcgagaagagatggaggaactgtgtcagattgaacgagagaggacaagtaaaggcaacatgtgttactgaatggaaacttattccctaaaggttattatattggcaatatctgaacgaagcataaggactccatggtactaatgttatcaatgttaatttccttattttgaagagtatattgtgattatataggaaaatattcttgtccttaagaagctcaggctgaagtactcagtgtcagtgggatgttggcactattcactccagtggttcaagaaaaaaaagtctgttctgtacttgaaccttttctctaagtttaaaattataaaaaaaaaaaagatatacaacaattacttaataggacttgcattttaaaagagaagtacaagttgaagaaggttaaaatgaagccttttaatatttaactccatagtcctatgtgttgctctgctatgctgacgatgcatgaaaattttaattgcttaataagaaagaaagggaaagataatatcatagagctcacaaatatgagcctaccaaagcagaaattagattatactagtttcaactattgaattaccctgtagtttaaattgaaaaacaaagataaatcaattagtaattctatatccagtttaatgatttttccataaaaattatttggcttataaaacaatacaaaagtctaagaaatatatcatttaatatgggcttccctttattcatgtctgccagcggttctaactaaaataaatggaagcaggatgttgcactgaggatttgctttgtagctctttaccagtttattagcctctatttgaagattttttttttatgaggccaaatccagactaaattaaatagatctggcatggtaacatatcattttgggttctaaataagtgattccttttaatgtgcatactgaaattttctcacatgacaaactgggacacatgaaatggaatttcacgcactattgaactgaattctcggtagctgctgtgaaatgtttggggagttttagtggatttatccattttagtgaccatatttcccctcacatttgtgaaatgcattttgctgttattttagttattctgaaaatgtgaattcatttacctgcctgttcagagaacaggcatgagagctaagggaaatgatgataagcacaataacaacaacaaatacaggtgatattcactaatttattcattcaatactaactacagggactaatattgaatgaataaattggtgagtatcacctgtctgttaacagaacaggtaagtaagtacctgctctaattaagcttacattctattagaaagaaaaagaccaaaaaaagcaaataaataaataattgcagagtatgatagctattgtgaaaagaataaacctggccaaacatggtggctcatgcctgttatcctagcgctctgggaggctgaggcagcgggatctcttgagcccagtagttcaagactagcctgggcaatgcagtgaaaccccatgtctattttagaaatataaataaataagaataaacatgttgatatgatagacaatagcttagcaaaggtggggaggacctttccaaatgggagacatttcatctgaggtatgaagaaagaaaagtagccatctggtgacaagtagaggatgaatgtttcaggcaagggcagggaaagtgcgaaggcactgagataagaaaagtggctgcttatggaagagaaagcagagtgtgataaaatggagagtccagcagccattattttgttatttaaacctcacagtgatgctgtgaggttggtcctcatacaatccattttttttttttttaggtaatattcctgaggaatagagaagtgaagagacttactcaaggttatttgttgataaatggtagagacagtgttttcacccagaaagtgtgacttcagaacccatgcattctgaacattatatagtgttacttcctgtgaaccctaaaaatgtgagacaggtctcagttaatttaaaaagtttatcttgccagggttgaggatgcaagcccataacacagcctcaggaggtcccgatgtgcccaaggtggtcagagcacagtctggctttatacattctagggagacataggacatcagtcaacacacacaagatgaacattggttctgtctggaaaggtgggacaactcgaagcaaaggtgggaagactggaagcaaggagggggcttctaggtcataggtagaaaagagacaaatggctgcattattgtgagtttcttattagcctccccaaaggagacaatcagatatgcatttatctcagtgagcagaggggtgactttaattagaaggggaggtaggttggccctaagcagttcccagcttgacttttccctttagtttagtgatttgggggccccaagttttttttcctttcacatttcccaaagaaagtaccacgcacatatgaattactgttgttataatgataaagttgaaggcatgttttatagcctagaaaactcatctatattatcatattttgctccattcaaaatactgtgcccaactctaggtgctaaactttaaaaagattatggacaaatcggagagtatttggcagaaggccccttgattggtagaatcttgacatatgaggagcagttttgactgtatcatgaaaaagataaggctcaggaaactacaaggtggtcagagcacagtttggctttatacattctagggagacatgggacatcaatgccagtattgatatatttatggggccttcatatggaaaaataactaagcattttggatggcccccagtaggtacaatttggagagaaatataaaagctacagaaacctactttgatagaggaaatattttttttcacattgaaaaatccaaaattggaataaagtattctgggagagatagagactccaatatcaaaggtatttaaatataagtagaaaaacctgttattgagagtgtcctatagtgaattcaagcaataatgggtaattgaactgtattagcccattttcatgctgctgataaagacatacccgcgactaagcaatttacaaacgaaagaggtttaattggacttacagttccacatggctggggaagcctcacaattatggcggaaggcaaggatgaccaagtcacatcttacatggatggtggcaggcaaaaaatgagagagcttgtgcaggagaatgcctgcttttaaaaccatcaggtctcatgagacttattcactatcactagaacagcataggaaagacttgcccccatgattaaattacctgccaccaggtctcttccacaacacatgggaattcaagatgagacttgggtggggacacagacaaaccatgtcaggaactaatgtggtttttcccaatcctgaaattctttgatggacaagttatcatcagaagacaggataatcagaattcaacatacttgatttgacagagcatggaatagaaacctgatgccagatagtgataaagaagcaaagtaaaatccaagatagtaggcttgcaatgaaatacagaaactgccatgtcttctctagttatcaaggggactggtgatctggctcagactagaaagcgggagtagccctgccctgccttagtaagtatgtatgcaaatttctacactgaatattatctcctcttcaaaacctatactgagaccccagggaagtgatcatgtgcacaatccttattagattacctgtaattctcgttgcattgatttgtttatctgtatgtcttcttcttttagcccttagtctcactgaaagaaaattcatatttgtattttcagacttaggagtgttactagaaaatatgtaataactaatacaacaagtagcagactgttgaatatccagggaataagagaagttcaaacaaagtcaaatttagaaacagaacgtatttgtggccataatttattttctaattacccatcaaagttatagatatgtatgctttgaatatggtatcagaagactaagtcataaaatataattttgatttagaagattataagatccaaatgagtcaatacaaggcatgaggaattatgatcaaattagattttagatctggaagatttcttccattttatgttctagagttctaccaagtgaaatcacaatgaattaattgagaataaatgttttattccaaatcagtttgtcagtttttatagagatatgatctagaatcaacttatttgaaacttccaaaattttagttcaatttcttaaacaaaggatactgctgtcttcttggagctgagagagatacaacctcattgctactagcattacctttaccacagaagaaaaaatactttgtaataatttttgatattactagtatcacttttaaaagttttattttgagtagtgaaaaagagtgatttatttgaagaaatgtcctaatatgactctgatgtgaacacctttcagaagtgtggtgtttattatttggtagtggcaaatgtgaaaacataggcattgcattcactaaactatagcgaacagagaatttgctgacctgtaagtgcattgttgacttttaccttgtccttaactttgacccttcctcagtattaatgagttaaaaatgtctcaaacaggtgtcagctggggttctcaacctttcaaagatgtgagttatgctcaaaagccagcctgcctcttcatctcctcaaattccactacctgtagaagtcctaagccctccttagaatttctgtgacttctcaggggttttcacttcttagtgcctgcttcacttcctttgccccagcttcctgataaaccttctctgtccagttaaacaaaggcaaggaacctcctcatccaaaaagagaaactacttaaatatactgttactgaatagactgttagagaatcttcccctgggctgaaaaagtagacacaagtttagatacctttaaatccatgcgagtgtatttatttatttatttttttttttttgagacggagtctcgctctgtcgcccaggctggagtgcagtgacgcgatctcggctcactgcaagctccgcctcccgggttcacgccattctcctgcctcagcctcccgagtagctgggactacaggcgcccaccaccatgcctggctaatcttttgtatttttagtagagacagggtttcaccgtgttagcgagcatggtctgtatctcctgacctcgtgattggccggcctcggcctcccaaagtgctgggattacaggcgtgagtcaccgcgcccggtgagtgtttttttttttgtttgtttttggttttttttagcataataaagacttgaaggtagctggcacctgtaattccagtgctttgggagaccaagacaggaagattgcttgaggccagaagtagaagaccatcctgggcaacatagagagactctatatctacaaaagaattttaaaaattagctaggcgtggtggcacatgcctgtagtctcagctacttgggagactgaggcagtagaatcacttgagcccaggagttcaaggcttcagtgagctacagtcactccactgcattccagcctgtgtgacagaatgagcactgtctcaaaaaaaagaaaagaaaagaaaagaaaagttttgaagaatcaatttattaaaaatcaaatatagttggctttcataatgagtttaataatttactttgagattaataatcccttggcaatgactcctaacattttagtttaaagcttttctcttagcctttttatgtatacatttaaagagcagaggcacatgctaatataatgaactattgcaatgagtctatttgcaatgtaagctgatgatcagcttgttttagctgatttataaattgccatgtaccagaattacttatactaaaacacataatttaaacatcatccttaagaaatgagatagaaaaaaggaaaagggtcagtattcaggcttctgccctgcttatgagtctcttgattgacactaattaaaaaaaaaataagtggaaattcaggaaagaggttagaggcagcaataggcaagctgtttaatgtcacatttgaaaaaattatctctaattcttgaagtcacttttaagattttaaaaagtcaagccataaatttgctatctttaaataggagtttggtttaattatcacttaagcacttttttactttctcattttaggtctttaatttgggtattaatttttcatctattttatatttcacaggttgtattgctctcattagactgtttgttacaaattgcttgtgaagaaatacattttaggtgtcataattttcctcaaatgatatctgtatgagtgtctatcaaactttcacttggtttgaaaagacaatgatgcaatagtcagactgggaaaaggtagatgaatggaaaaggcaaacaggtctttatgaaaaacaatgcagatgatctctggtgtcacataatgtttattattattcagctatgtacatacttgaaaagatccattgtcattaaattattttttatgtcagcctttattatcactttgcaatacaaagaaagcaaggtgaagactaacttttctcttgtacagaatcatcaggctaaatttttggcattatttcagtccttggagacatctgagagattccgggatgccagtggtgcctctctggccacactgacaacaaataattcacctaaggaatagttcacttcagctattttttgctactcattggttgtcagtgccattgaggagagctcagtgtagatcaaagaaaacggtgtagatcaaagaaaacggtgattcggtgattgttccccttcttcccagccacccaccatctgaacctaatgcatcattgtacaatggccgtaaaggatgacaagggactcagcaatcagttcctggaggaaatgatgctgtggcttttggctggtggcaccatcatcctcagtcatcagtcagagtcatcaaagtgatcattcatccattcactccctcctctgtctccaccccacagctaatcaactaaccagttctgaattttttctcaaatattaaatcttaacttttaaatctttctcaaatctatctttcttcttatctctgccctagtttagatccgtaataatcttcaccttaccaattggtagaaccttctaactgacctatctccttcagttttgtcttccttaactctgtaaccacagttacttccataaaatttaaatatgaccgtgacacttgcatcttaaacaccttcaatagcttactggaaaaattttaaagcccttgttctggcatgcaaagcccccaaatttttccatttattcttacagcccctccactcactgccttgcattccatgtctgagcacatagaacctcctactattcttcccaaacagccatgcactctgtcttcctagatgccttcagccatgaatttcttccttaccttcagttggatacctgtttattgagacttatttcaagtaccttttttatctagaaagccttacttgtctcatatcccatctgggttaggtatctctctttggtgtccctggaataccctatgcatatttctgctattatattcattagattatattataattgacagtttttatatctttggttctcactagaacatgagttctttgatgatggcacttttattcatctttgaatcttttgtaccaggtagatattctgtctcacactagacacatcataaatatttgataaattagtaaacaaatgaacaaataaaagctgcaatcagcagcgttaagttttcatgggatctgtggataatataaaatacagaattcccagctgtggcagcagatttattgctatcataatggatccacagcttctttcttgttccttgaatgtgaccagtaacaatgcacctaggagatctgatgtaagagtattctgtgataagtgtatacagaaaggaggaaccgatgactcaaataggaatcaagccagaagcttctgccatatgggcagcatggacttcaaccaaaacaatgaaacagtgctgtggtttagcagaggtcctaccatgaatagaaggataccaggttgccactgtgtctcagactgcaactctgtgtcattgacccttttaccaaaggtaggaaacagagctgtagtcatgccccatatctataataagtatgtggataataagcatcgagtacagccctgatggtagtgtcactgtcagagacttgtgttgttttccattgcactgctatttctgtgctatgagaacttagctcatcagttattgaagaagggggaaagttaatgaccacaagagggcttatgatgaccctaaggcaactttcagagctattctgcaagaagagagatttttatttcccaaaccagggggtaatttccaccagggctttgtttgtttgtttgtttgtttgtttgtttgttttgttctgcatttgccgtctcattggtaataacaagactgcagcaacaaaggaaagaaatctcactccatttctctcataattccataaatagtttgcctgcctttcattgagggcagttagttaacattgagggcagctagttaacagcaagagctacagaaaagtcctgtaattaatcaacctgcattgccttcagtctgaatctgagtattaaaattcagtaggtgcaattagacagaatcaggcaaatataaaggtcataacattagtcacagtgtcaccttatttctgagccttttctttcttctgaagtggtgtcttgtcagtggggtgatagagcaacaagaactctctgttgttatgggagtttgttaaattttatctgcgatttttccaattttaattaaatacttgataccaggctgcagcaacagaaacactgctgtattgggtttcagcactatcactctatcatcttataatttaaagtgaaagataggaggactttggggttctgacggtttgatagctttttaaaaattacctttctttccccataccagattagagatgggggtggtggggagagagcttcagcatcctctcgccagaacttccggttctctagctttttatacatcctactcacactcgtcctactgatttccacttaggactgcaactacccgtcctctctgcttgtccatcaggaatattgactttcccaaacatttcttaaaaattgaaaagttaaaccaaaaatacacacttttattttcttatttttattttttctaagccaaactgtatccagctttattaaagacactttccataaacaatcctggaatttcagtcaggacatgggcagacaattattaacagtatacaacaactttcaaaaatccttctctaatggactaccaaaaatcagaaagccactataaaactcaatgaagtcttcatctgatgcactgagcagggaaagttttgagtgagggttgacatttcacatttagcatgtttttaacaacttttcacaaccgaccctgactttcagaaatgaaatgaaaatggcagaatttatttgaagattcacaatctagaaacataaccactgctcttttgaggcacgccatctcagtggcaccactggaaagtccagattgcctgatgcattggtgaccaattaatgggattccggtcccaacagatgtctgggtttaagggagttaagtctatgctggaaggtggaaagggagaagaaaacataagaactaatttgttttccataccacaaggcttttgcaccaaggtggccatgtatgtcaaagtcaggaaatctttcctcctgggaaccaagaggaagtctctcaaaactagaagggaaaggtgtttcacacatcaatccagatttggagacatttattagtgacatatgctccttcccccccaaaaaacaataatgaaatgttgtgtcttaacaacatagcttaaaaaaaaagtaaaacaaaattctgcatttttataaaacttgataaaaatatttcaaactgtacggtcaccagtgcacagttatcaaaaatgcgcacacttcgcttggcatttccagcaccttcagctttctgtgcctggtctccatttctgcagggttattcccctccttgccagtatcatcttttccctttttccctctgggtaccttctgtcccttctttgcagggcctttttaggcctgggctctggctttggaggagcaggtttagcagacaatctcgcggatcttctctgtggttcatccttcactttggctgtatctcctttcgcatccccttcagcctttctcttgggcatggtggcggcgatggtggtgggatgtaggtgctgggtgtgggatgcagctgggcacggaatgcagcgttgcacgagctttggtctgtccaggggtcattctcaccactgcttcttccaatacacacttttaaatggtgcttctatggtctcccacatcaggagagccctcagtgctttccctcagcctaaaagtccacgattttattcttcactatttcctcaagcctattctcacacctgctcctcccttgcagtgaatgatactgattccagtttttctgagaaaatagggaaaggcgctgaaaagattgtactgtctgcctgcccatttacatatattatctgatttaatttgcacagtctgagatgtggagattattctttcttttacagataaggtaactgaggctcatgtaagtggtggtgctgagactgagctcatgttagttcaactgccaaacttctcctcccatgtgatgggatgcagctgtaaaacagaaatactagggcatgaacatcagatttctcacttccctattctaaatatagctaaatccatatgaatacatattccatgactctcttgtgcttctttcctccgttaggctaataatagtctattcatgctttgttttttctcttttgggatatttgatcatcaactttccataacttcaaactctttcttttaataattctttcctctcaatatatacacatacttatgtttccctctgtcttaaaaaaatccatttctcatccttgcgtctgtctctgtgtgccaccttatcatccttctttcgttcacagcaaactgtttttaaaaagtgctttaattctactggttccctctaattcactctgtagtctgaattgcaagtctatcatttcacggaaattgatttattctatgaatgtggggtttggagttgggaaataaccaaatccaaactcattcttcactcatgacacttagacggtctctgctgtgtatgatacccactttgggatactcattgttgaaaccatgtttctatgactttcatgataccattttctcctgggtttgcaattccctttcaggccactcattcttaagtccatttggtgccactgctcccctttatgctgcctaaatgttagtgctttccaccatcttatcactgatcctcttcttgcttgtcaactttgattgaagtatttcacccatatctgtggttataaatgtatgctatctgtacactgatgttccccagaccagtgcctcccagacccatattttttgttgtttaatgaacttccattgaaatttccaagtctgcaggttcaaaacgaaattcaaagtctttctattcataatctctaccaacgtgactggtaccatgatacactcaggtatgtccccgtcgggattttagaagtcatcctttgccattctcccttcctcactcccaaataagaatcctctggaacattcctaaccttcaaaatgacatcttttgttgttgttgttgttgtttttgagacagagtctcactctgttgcccaggctggagtgcagtggcacaatctcagctcactgcaacctccgcctcccaggttcaagccattctcctgcctcagcctcctgagtagctgggactacaggtgcccaccaccatggctggctaattttttgtatttttagtagaggcggggtttcaccatgttagccaggatggtctcaatctcctgacctcgtgatccgcccgtctcggcctcccaaagtgctgggattacaggcatgagccaccacgcccggcccctagttaatgactttcttaaatatcataatgctaaaaggaatttgtgctagaattttcctaaataaattgataactaccttctctgacctgtaaccccctgccaatagcatatatcagccagacttgaaaagattttttgctgtcctgatgtgattgtcccattagtacagaaaacaaaggaaaatgtagtttgacgaaacaggttctcattctgaaattgtttgcatttgatctgcctttatttcagttttaatagcagctataatcaacgttgggcaactgctttgctctttctagctgcagaaacctctggccaaaaacacaaaaaggcacactgatttgtgcatggaaacacatcctcattcttattttcaacatgagaagttgtcacagacatccctcttcctttgctttgagtcagtttatgtgatggcatttatcattaagacatataatctgttagtgattgttattcagcagcttttggttttagtttcttatctttgaggaagccccttttttcaaacagctaactcttgcatctaaaaagtgtcttcttaagtatatcatctttccattttgctggaattctaaattttatgctacttatcattgaaaaaatactgaattacttcatttttgtattttattaaactaaaatttgaatccaagtcactcaccactgatagctttatactgtttaaatgactttttaattttggtgtttgggtatgtttgttagcatgtagaaagcaagcccaaggtaatttataaagctatgacttacagtgtatggagcactaagtagagtttattcaaggcaactggtgctagatgctatgaagattactgtagtcttcacttcaatattacacttattttgccttttcagaatatttttatatgtgaatatgtggagacacattctttttgaggcggtgtcttgctctgtcacccaggctggggtacagtggcatgctctcggctcactgcaacctccgcctcccaggttccagggattctcctgcctcagcctcccgattagctgtaattacgggcgtgcgccaacacgcctggctaatttttttgtatttttagtacagatggggtttcactatgttggccaggctcgtctcaaactcctgacctcaggtgatctgcccatcttggcctcccaaagtgctgggattacaggtgtgagccacccacgcctagctgagacacaaacattttcttacctcaaaacctggactacacaagaaattcattaagaatgaaagtaatatatattttttcttaacaatatcttgatctttaaagaatcagttctatgtatactgttattaaaatctgattaaatagcaatttataggaatctttaaaagaaatcaacattgagatttttattatatatctattaattttcagtggatctctgctgaataacttacaaacaattataattatttttccattaatctgatatttgaaggtaagactcaacaagaacaatgcaaaaatgagagaaaataagagggaaacattaatttttttctttgacttttatttcatctgcataatttaatcattttttccttcaaatattcaaaaacctgcattggtatttttttctgacacatttcccctgttagactttagtttttcttagttcatattcttgtgttaatggctggtggagtaacgtttcctcacacacaatgttttttaaggtatgtttctatctggaacactaattggtcaaatatgtatagcacatgaacattaaagggtgctaactacggttatctcactgaagcattttttttttttgtcgttcagcaaacgttgatttaattcttaagtgccaggctctaagaaaggtactgaaacattttcttccattaaagaaacataaataagagacagacatattgtagtttaaataagtacagtgatgctgtaaaaatcaaattaagacaaataggagatgtctatgaaaatggtcattacgcagcctctcccttcctgcctatcctgcattcctgcctgcttagtttctctgatgagacatctatgagctccttcctcttccaaagcctccctgcctctcactggtctccccaggcacatggatcataatctgcctttctgtctccctctgtagaatacccctgcattcactctgctcattggactaagctggcctgctcactgctgtcagcattgcctgaacttttctgccaccatgagtttgctcaggctcatcattcagcaagatatgtgcttggtttctcctctgcctgtcataattctgtccatccttcaaggcctgcattagtgtgtcttgttatccatgtttactttccttcattgcatctactcctttttagcatttgtctatttatttaacagacccccacccttctggaatgtaataataaacacacaggcaagctaacaaacttgaactcatgcgtatcccctgcggagcctagaattgctttccattccgcttggaatagaggatgcatacgtatgcaaatattgaagacattcatttgatgcacaaagtctatttcacaagcatttaatttctaacatatgccagactcagagtttggcaatggcatgtagatgaaataggcaagttaacagacaaaatacagtatgttaagtgcttcagtggaggtaagcatgggaaatttataggagtgttactaattcaggtaagtcaatgagtggatgaaagcatggataaatatgtaaatgaataacttaggttaattctctggagctcctatttagtattctgctgtcctctttatgtgttaatcagcctgataatctgctgcaactacaggcagtgtcctaatttcttccttcacaatagttcctaataacactcaggtggctacaatatagtgaattctgtatacctgctttctcagatgcatcatcctggcctttaaccactatatgaactgatgtctccagttgccacattaaacatattaatttgggaattcaaaaaagtaatgtttaaggccaggcacagtggttcacacctgtaatcccagcatttgggaggctgaggcgggaggatcacaaggtcaggagttcaagaccagcctggccaacatggtgaaaccctgtttctactaaaaatacaaaaagttagctgggcgtggtggcacttgcctgtaatcccagctactcaggaggctgagacaggagaatcacttgaacccaggaggcagaggttagagtgagcagagattgcaccactgcactccagcctgggcaatagagagatgctctgtctcaaaataataataataataataataataataataatgttttagaataagattagtctaatgtttcattattaaagcagataaaatgtatttaatggttcttttgtctttgttttattcctgtgcacaattttttttttgagacagtcttgctctgttgcccaggctggagtgcagtggtgcaatcttggctcactgcaacctccacctctcaggttcaagcaattcttctgtctcagcctcctgagtagctgggattacaggcacgcatcaccatacccagctaatttttgtattttttagtagagatggggtttcaccatattggccaggctggtctcgaactcctgacctcgtgatccgcccacctcggcctcccaaagtgctgggattacaggtgtgagtcaccacacccagctcctgtgcacaatattaacacttcactagtttatttcctttgtttttctactcgtggtgatttgaaacagtctttcttttagagtgctttgcatgaagaatctcaagttcttattttttcaacttctgtttatttttctgtttgttggtttttccactaaaaaaaaaatcattttttaagttttattttggcctcttttattttgctttttaatcatgaaaattaagaaagcatgttgagcccacaaggtactaagtactattgttgacagctgaagtgatacctcagttcttcttggtttaaaatagtttaaacaagagctacacagcaaaggaggtgtagcatagagtaatttattacaatggataaagatattttgaaagttaggtgcagaataaacagtataccctgagagagaaagaagtcaggacaggctgcttgtaagggtgagacagcaaaaactggcactagggagacacccttttagggagttttaggtggggacacagccaaaccatatcagacacctactatgtgtcattcgttttttcttcagtgcttggatgttgaaataatgaataagcataagactccctttttgggagttttaaaagatgattcataaggaggtgggaagaggtgttactagtaagcatgttctgggtggtcttctggtgcacatgtgcagtaactgtacatgcttgttcatatgttgcatgtttcattagcatcttaaaactccacccaggggtgtggtttttactattataatgagcatagggtcagtttggggacaggtaaaatcaaaatatgcatgctctctagaagggaaaggccctattgaagatagctttgcttgaatgagctcaattacaatgcaaatactgaggtttattgtgctgactgtatgatcaccatggttgccccatcccaagaacatggtcacttccttgactgcctatcctgcctcactatgatctagtaggtgatttaaatttggtccacactcagtgtaatcattccatgcactttaaacataatctagttacctggtgtcattgcgctatatgttgtcctaacctcgtgtagttcagacttcaggtatctctgatacctagccataaaagtttagtagggactactcaactaaattatcatgtaacaatcaaatgagacctacagctgagtaaagaaatgcttctcttattgatcaaatttttttggttattttttcattttcatgccatgttatatttgatccttacgagctcaaactcataaaacaaaaggccaccacttcttgttggtttgattcagttattcaacaaatgtttattgaacactgtataagttcattctcatgctgctataaggacatacctgagactgggtaatttataaaggaaagaagtttaattgactcacaattccgcagggctggggaggcctcgggaaacttacaatcatggtataaagggaagcaaatatgtctttcttcacaagacagcaggagagagaagaatcagagtcaagcgaagggggaagccccttataaaaccagaagatctcatgagaactaactcactgtcatgagtacaggatgggggaaaccgcctcaatgattcaattgtctccacgtggtccctcccatgacatgtggggattatggggactacaattcaagatgagatttgggtggggacacagccaaaccatatcaaacaccgactatgtgctagtcttttttttttctgaagtactgggatgtagaaacaatgaatgagcataattaatatgttaaaagaagaaaaatgaagcagggagagggcaaggagagttaccaagggaggaagtatactatttaacagggtggttcagcgaaagtctctcctgtagatgcttttgaggagagatttgattgaagttagaaagcaagtcatttgagccttacagacatctgtggagagtattgcgggaataggtgatagccagtgtcaagcttttgcatgctcaagggacagaatggaagccagtgtggctggagcagagtgatcaaagcagaatggtagagaaccggagcagagaggacatgagcggggacatcatataggatctgttggccataacaaggagctcatgttttattttgagtgagatgaaatagcattgtagccaaggagtaatgtgatctggcatatatttttaaagtgtactttagttactgagttgaattggttttcttttgctgcataacaaattactacaaatttaccaccttataatactgcatatgtgttatcacacagtttctgtgggtcaggagctgaggaggaagggctgcttagctggcttttctgcttagggtctcacaaatctgaaattaaggtgtctcatttgaagcctggggtcctcttccaaattcacttaggttgttggcagaattcacttcctggaagttgtaggactaaagtccctgatttttgttgttgtggtttgtttgtttgtttgtttgtttgtttcactggttgggtgttttttgttttttgtttttgtgattataagcctggttatgctctcagctcccagaggcaggtctcagttcttagccacacgccagctcacaacctggtaggtcacttctccaaaactagcaaggaaatctcctacattttgagtctttctccaatttgctgaaatggagctttctataacataatgtaatcaatcacagcagtaatccaccatcctctttgccaacaaatttaacctaatcaaggaagcgactctctcttcatattcataagtcctgttcatgcccaattggagggaattgtacaggatgattacatcatagggtggaaattttgggagccaagtcataattccacttactacataggtggtactactacagaattgtgaaaggagcaagagaagattctgaaaaccagatagaaggcactagtggagtctaagccagtgtttccccaagtttaatgtgtatatcaatatcctaggcattttgttaaaatgtagattcagacttcataaggctataatagtggcctgaatctcccaacaaactcctaggtgatggcgatattgcttgcagagtagcaaggtacaggcaaatgatgacagtgacttgggttagggtggtaatagtagagataagaagaggtctgattagggaaatattttgaaggcagaccctaaagagcttgctgaaggagaaacaaaggcatcaggataacttcttacttcaggggatgtcaaagactgtgtggaaggagcttatttcagagcagaaaagagttcagtttgtgatatgctaagtataagatgccttttaggttcccagtagagagaatcacaaaatgcgatggaaaatagcaggtttatacattatattcagtgttattgaagatttaaaattttgtttctgtgcaccacaactatgtaactctttctaagtgtgcttagaatttattatattgtattaaaaatgtgagcaatgaagaggacctaactgatttgtgctgcagattctctttcattctcttgcactgacattagtaaaataagcagtgttttcttgtccctatgccagagttccttacagtgaaataaacatcattactcaaatcattaaaactgttcctagaatccaaaatacacatcactgtgcatatttaaaacacaatgtattcaaacactctttgggtacagacctagactttaagatgattttcagtacctattgattctggttgatgcctcactatgagatgtattaccagattttccatgactgtatagacggcttcatggtgtgactaattattatttattttcagggcaaaattatgcaatgttattttcatcacattgtgagaggtggccattagatagagcttgcataaaagttagttctcactgactcacttatcacaaaagaagacagtgtagactaggattttatcaaagataaatttacttataatcccctaaacctcaatcatgtaaaatcttggccttacccgttctcattttcagaaagaaaagcattcagaagaatataaaatgaaaactacactgccactcctttcttccttgtgatggccatggaaatgctagcgtttaaccagagacttggactctcaatatttcaaatgttttaaaagaactttaagtagaatatacctgtgcttagcactgcttgggtgctaagttaacatgcagtgaaataagattacgggaggtgattcatggtttcaggtaggggtgaaataaaggcaacaaagggcagtttgggtttagaggacgcttcatactgtggaacatgtgaaaatttagtttattttaagcaaaaatctttatagaaatccctgttttgcttttcttattgttaaacaataaaatgtgctaggctttacgaccagagaaaaatgggaaggagatattttaaggacagcctgatgattatgtttttgtaaataagatcccaaacagatggaaattaaaccaaatctttcaatgacagagggtgacagtaggtctgaaggcagctacttttatggagagtgttgaggaagtcagaactttactcctatatctggattcctttcaatctggaatcctgggctaggactaactaccttacaggacaattaagctttttgttgatttgaaagaattgtgatgactggacaaagccattgctgaaccctgaacttgtcacattatgagaggtggccattagatcaagcttgcataaaagttagttctcattgactccctgatcacaaaaagaagatgttgtagactaaagttgtaccaaaccttggttgttgttctatagacttgtttttttatagtggcatggttgttctacagaccttttgtagtgtttcgtattgaaatccagaatctaaatacaggcataattatttcatgcattagttcaaagatgtcaaacatttttctactattgcatctatttttatgtaatgtattaactataatgtaaagtttaatttttgcatggtaatttaagaaactaaatttcttactccttcttttccctaggagcactgaatcaaaaaaattggggaaagaaatatccaacatgtaatagcccaaaacaatctcctatcaatattgatgaagatcttacacaagtaaatgtgaatcttaagaaacttaaatttcagggttgggataaaacatcattggaaaacacattcattcataacactgggaaaacaggtaaaatatttgcattctgtttgcctttaatatatttttcagacctggagtatgtttagactagtttcatctcattttcctttgaatagtgtactatgaactagatagaagttacatgtagaagaaaccaaatggagcagagaaaatgggaaatttagacaatattttgacaatttgtacaaaaaaaaaatgaccaatgtcatcatgtcatgttttcctacaaggtaacaataatgatgctgcctctagaagatgtgataatgtgatttacagctaatataatttagtcaattacttagtcaatcaggatttctttttcttgagcattatagtttgatcttttctgcctagcctcatgttatagtcagtatttggatatgaaaaaagcaattggctataattgaaatttaagaaaataaattcctagctcttctatatagaaggtttaaagttgtatcccatccttaggaaaccctctctaagtgtctttttattgagcctatctttttttttttttaaaaaaaagcaaaatataatacattaaaatttaagtacaaattctaataagctatacatagataaataatttccaaaaagtgacaccttttcttttttgatattctagtttttttaaaaattgtattttgggaaattatgtaaaggcaaatatacacgtaagacaaagattagaaaaatattcaaaattataatagtagctctatggggataagctttatcattggtgaattttttttatattttctatatttaggtaatgtaattatttgaacataataatttaatttcaaaactcaagagagtagtagtcaggcactgtggctcacgcctgtgatcccagcacttcgggaggccgaagtggctggatcacttgaggtcaagagtaccagactagcctggccaatatggcaaaaacccatctctactaaaaatacaaaaattagctgggtgtggtggcacactcctgtaattccagatactcaggaggctgagacacaagaatcacttgaactcaagagttggaggctgcagtgagccaagatcatgccactgcactccagcctggacaacagagtgagatcctatccaaaaaatcaaatcaaaacaaacaaacaaacaaaaaacaactcctgctgggcgcagtggctcacgcctgtaatcccagcattttgggaggccaaggtgggtggatcacctgaggtcaggagttcaagaccagcttggtcaacatggcaaaaccccacctctactaaaaataaaaaaattagccaggcatggtggtgcatgcctgtaatcccagctactcgggaggctgaggcaggagaattgattgaacccaggagtcggaggttgagtgagctgagattgtgccattgcactccagcctgggcaacaagagcgaaactctgtctcaaaaaaaaaaaaaaaaaaaatctcatgaaagtagagatttttctacatcagtgcctgatatgtaataaacactaagtatttcttgaatgaataatttttaaaattattatgttacatgtaataattacctaaaagtatattgtcccaaatcttgacattattattttcagttttgaagggtgttagaaagcagtagggcatggaacctctgctttggtttttttatttttattctttttatatatatatttttattatactttaagttctagggtacatgtgcacaacatgcaggtttgttacatatgtatacatgtgccatgttggtgtgctgcacccattaacttgtcatttacattaggcatatctcctaatgctatccctcccccctccccccaccccgcaacaggcccaggtgtgtgatgttccccttcctgtgtccaagtgttctcattgttcaaatcccacctatgagtgagaacatgcggtgtttggttttttgtccttgcgatagtttgctgagaatgatggtttccagcttcatccatgtccctacaaaggacatgaactcatcattttttatggctgcatagtattccatggtgtatgtgtgccacattttcttaatccagtctattgttgttggacatttggattggttccaagtctttgctattgtgagtagtgccacaataaacatgcatgtgcatgtgtttttatagcagcatgatttatattcctttgggtatatacccagtaatgggatggctgggtcaaatggtatttccagttctagatctctgaggaatcgccacactgtcttccacaatggttgaactagtttacagtcccaccaacagtgtaaaagtgtttctatttctccacatcctctccagcacctgttgtttcctgactttttaatgatcaccattctaactggtgtgagatgatatctcattgtggttttgatttgcatttctctgatggccagtgatggtgagcattttttcatgtgtctgttggctgcataaatgtcttcttttgagaagtgtctgtttatatcctttgcccactttttgatggggttgtttgtttttttcttgtaaatttgtttaagttctttctagattctggatattagccctttgtcagatgagtagattgcaaaaattttctcctattctgtaggttgcctgttcactctgatggtagtttcttttgctgtgcagaagctctttagtttaattagatcccatttgtcaattttggcttttgttaccattgcttttggtgttttagacatcaagtccttgcccatgcctatgtcctgaatggtattgcctaggttttcttctagggtttttatggttttaggtctaacattttaatatttaatccatcttgaattaatttttgtataaggtgtaaggaagggctccagtttcagctttctacatatggctagccagttttcccagcaccatttcttaaatagggaatcgtttccccatttcttgtttttttcaggtttgtcaaagatcagatagctgtagatgtgtggtattatttctgagggctctgttctgttccattggtctctacctctgttttggtaccagtaccatactgttttgggaacctctgctttattatatattgtaagcaaaaatctttatagaaatccctgttttccttttcttattgttaaacaataaaatgtgctaggctttataaccagagaaaaatgggaaggaggtatttcaaggatagcctgatgattatgtttttataaataagatcccaaacagatggaaattaaaccaaatcattcaatgacagagggtgacagtaggtctgaaggcggctatttttatggggagtgttgagggttttgtttttcttaggggagagtggttattagattattgagctaatagagccacattacttgggttcaccactggctcttgaaatgacagtggggaacctttgagcaacctacttagcctttctgtgactcaatttcctcatctgtaaaataaggtttatacagtatctacttaccaaggttgtgcgaagatcaaatggataatatatgtaaagtaactagtatagttcatggtacatcgtaagtgatatgtaaatgttggctgtcgttgttattgtgcttgttattattattattagctggagtttgtatccctagctggctggccaatatgttgtattcagtccctgatgccaaagtgatttatccctaatggaacagagactatttaaactttattgagggacatccaaatctttcactgggctctcaagagagctctccagaagactgttaaaatatagtcagataatatctgctgtggaactcatacaggggagtttaagcttctgccactcagggttcaaacatgagatggccaaaactcctccataagaatgagtttcctgcctttcctgccacagccagggacccaggttctggaccttgctaatgaagcctgggtgatacatggagggataagcagcagcctgataaggagtagaaatgggctgaaaataggaaggtgagagaactctgaggagaagacttatttttttttttcttttaacccttcagttccttccccctttgctctgtcttcttattaccatctgacatcttagggaagcagagctttcaagtaacagctatcttaggacaccaaacaaacagagcaggtggactggctgtcttctgcccttgaagtcatacactagtcaatatcttcagcttagtttgcatttgtggcaataagatataagtatattgtgcagacctacaaagtttggcttccaaaatggcgacttcatattttgtaaatttatcaaataaatgatttttcttaatgtaccttaagtagatggaaatttagttgaaatttaaaattttgattttcagaagcttaggtgcaattgtatttctttttttagtggaaattaatctcactaatgactaccgtgtcagcggaggagtttcagaaatggtgtttaaagcaagcaagataacttttcactggggaaaatgcaatatgtcatctgatggatcagagcatagtttagaaggacaaaaatttccacttgaggtaagtcaggagatctgctgtgtactattttattttctaaataattgatgtttttatttagcatataatttgattaattttaaaatccaatattaaaatttataaagtgatattttgattacttgattaatatactagactaagctaaattaatgtaagacttatctttgatttgccacttgctggtaagtagcaacccaaagtgccacaaaatggtaatcaataaatgctgatgaattaatgaaggaaggaaaagggggacaatttcattaacatactggagaatcctttgatttaaatcttttgattaaaatgagaaaaattttcagctttaaaattggcaaagtaaataattataaaaaatgagtgtgtagtgaaatgttttcttacatgtctagagaaagtctaaattggtagactttctgggaagcagtttgcttgtttttattaagaattttaaatgatttacactaaatacccaacatttaaatttaaagccatttagtctaaaaatttaaaaattgcaaaaatttatttaaaatataatattgaaccaaaaggcaatgattatttaaatacattatgatggatattatagatttcacaaaaatcatgtttttgaatgatatatatacatataaaaggctcatgataaggtattaatggttttaaaaagaaacatgttaagtggtataaccagtattatttttgcaattttttaatacattaaaattaattaaatattgactgagagtggagtctgaatcaaatggactaggtttgaatcttgactccaccatttggttattggttacatgacatacaatatataaattatttaacttaccaatgcttccatttttcttccgtgaaattgacatagtagctgcacctgccttataacattactatggaaattaaatgagggattgacttattactcataaaagtaattgaggagccaggcgcggtggctcatgcctgtaatcccagcacttcgggaggccgagatgggcagatcactagaggtcaggagttccagaccagcctggccaacatggtgaaatgccacctctactaaaaacacaaaaattagctggtcatggtagcactcacctgtaatttcacctactcaggaggctgaggcatgggaatcacttgaactcaggaggtggaggttgcagtgagccaagatcacaccactgcactccagactgggtaatggagtgagacgctgtctcaaaaaagaaaaaaaaaaaaaaaaaaaaaaagcaattgatggaattcaagaaaatgctagtaatgattttctctgggtgatggcattataggtgcctcttattttctcctttcaagttcccatgttttctaaaatacctagaatgaaactattcattttataattcaaaagttattaataataaaaataagatagggtgaattacctcttaaaagactaaaaagctttttttaaaagatggatatcgacattcatttacagcttatactatggattttttctttattcaatatttttaaagaaaagcacatttatcaaatttgctttgtcaaagtagtcaaagtcaattcattaagcaacatatcacttctgagagtttagcttactcacaatattacctatttagaacaaagatgaagttcaagaaaaattctatcaacaagagccctgagcaatcaaaatacatggtacagaatccatgcactaaacctaatacaattattttaatgcaaaagcctctcaaattgtctctcagtggtattggggttttgttgttgttgttgttgttgctttgagacagagtctcgccctgtcgcccaggctggagtgcaatggcatgatctcggctcactgcaacctccacctcctgtgttcaagcaattctcctgcctcagcctctcgagttgctgggattacaggtgcatgccaccatgcccagctaattttttctatctttaagtagagacggagtttcactatgttggccaggctggtcttgaactcctgacctcgtgatccacccacctcagtctcccaaagtgctgggattacaggcatcagccaccatgcccagtcttctttgttgttttaaaacactctctatgtaattgtgtaagcaggaaacccaaggatcatccctggcttgctccgtatttcttatctcctacatctcacaatcaagtcttctcaattcaccaggcatggtggctcaaactgtcatcccagcactttaggaggctgaggctggaggatcacttaagcccaggagtttgagactagtctgggcaacatagtgagatcccatttctacaaaaaataagagaaaatagctgaagacggtggcatgcacctgttgtcccaactactccagaggctgaggtaggaggatcacttaagcccatgaggtcaacgctgcaatgagccatgatcatgccactgcattccagcctgagtgacagagtgagaccccctctcaaaacaaacaaacaaaagccttatcagtttttctcctaaataccatatgggaggtcagaaaaaagtttgcttacttggattattaaattaataattattaagttttaaatcatatttctttcgatttcccatgcttgtcatactgtcttgcctaataggcctttgccaggtggtacttctgacttgaacacatgcccttctcttctgcctttcacctggctaaaagctgctcattcttgaagggtcagtttagaaatcctgtctctgaaaaggttttggtgactcgcccaaagcaggagccactgctatcctctgttgcacaccctctgtttctcccatcagagcactgactatgctttatgctctcagaggcttcctactcttaccttaagtctacttctatctagcctgagacacctgatttccaaacccagacaggagaagcaccaaattatatagaggctgttacagagagatacatatatggactgaagcagaaattaacaagggatagcctcacagctggaaaggagaatcaaaatatgaaacttaaaactcttttggtattttttaggtagaagctgagagtccttgagagtttcaatattaatagaccacaatatgggaatatcaaagaatggtaactctggactctcaagaaaggcatgcactcttgagaaagacagaaaattatatttaaaataattccataaagcagttaaagtaatatatgattatttattttatgtaattgttctaataaattttacggcacagtcccctggtaatctcaaaatggtcattttggttttagatatgtctaactttctattcccttccttattgaaggatggtaaatgatagcaaatgtagttgtatacatgtaatttataaaagattttggaaatacttagaatgtagaattctctttgctgatttttatgaagtctttgtgtatcataaaactatcattgttacttttatagatgcaaatctactgctttgatgcggaccgattttcaagttttgaggaagcagtcaaaggaaaagggaagttaagagctttatccattttgtttgaggtaatatatatacactttacactaatgtaattcctttttaagtcacattaaatattgacgtgaaatatcaagacatagtttctaacaaagagaggagctcacaagtaaaattaaaactggtggtcattttcaaaaaaagatgacccttgcctggtatttttaaaaggagactcttgtgtttgttaattgggtgaatcttttctagctggggcaaagtgttctattaagtacacagatttgcttatagtacataaaagcatcatattaataattatgtattattatcagtaactatgaatcataaaattttaaaatttacttttcttagtgaaatatgtaagttacctggtttttgtttgctttatttgttttcacctagctataaaaaaagtgtttttggaacttactattttttaatgcacatatttaaaatggaattcattaatcttcacatttttattgaatagttatccaaatgttttattctttttttagaatgtgattcttttttaacaggttgggacagaagaaaatttggatttcaaagcgattattgatggagtcgaaagtgttagtcgttttggtaagctacttggggaactatctttcttcaggattctgctttggatggataagaatgttgacaatacgacaaaagtcacttgttccaaaaggtggaaagtactacatttttaaagaggagcaatctctcactttccacacttgataatttatacatttctcaccaaataaataacaatttatcagaaaattttgttacatcaaaagcatgaaacattaatgtaagtaggaaattgaagtttttaaaattcttgaaaataatctaattatatcatgcaattaaagcctatttatctattatgtctctgcattatacatccgttaaagactaattcctaatctgaaaccagctaattttataaatatcatattttaaagtagaaaaggaattgttactcaagttaaaatttattccctggtggtttccttgataaatcctttgctttatcaacatgaggagatgagatttttggaagaaggtcaacacttacatagaagaagatgcaatatgctttgacattgttctgcctgctatctcatggatcttgtaatttgtcacaacaaatgcttttaacaacatgggcagaggagtgtaagcctattaattttactattttatttttattaagcactcacacacaggatcttaatgctgccctgagttaccaataaaatgaggattattcctctctggaagctgcaaaatttgaaatcagaaacaaaactcatgtagatccttttcttgaaagcagaagcctggcctaggattgatttatcattgtcaattacatgtgaggatctgaattgacccaacttttcagaagtcaggaatatgtgaactcctgcagggtagcttttattttttttttttttttagagacacagcacaagatgaagctgttacctccctccatcaacccagattgcaaaattctacattgaatgcgataagtcacatttaattttccccacttgagtcacaaagtagattctatccagactgaaataaaacaacttgtttgaattcctatacttaaaaaaatgtgtaagatatgaattctgaatgttccagaccttgcaccagcctgtggggacaatgaaagagatgttaacactgggttgttaaccctgacttgtgcaactatgggacctttgagactttctcttaaggcctctttactttcccaggtactttactttttgggggtgccttggaaggcactcatacgagcttgagaataaacttgctcatgttctttaaaatttcctttctaatatctactgtctgaaagccattcaggaagatactttgtcagatacactgtgactacatattctctccctttctgagaatatggattccctaacagcattattctgcctatattctggctcttaatttgaaagcagaacagttcttggattagctgaggacggattctagtcctttaccccctctctctctgtctctctacctcacttgtattagtccattttcacactgagagtaagtgatgtgtaaaagaaagagattgaattgactcacagttctgcataggtggagaggcctcaggaatcttacaatcatagtggaaagcaaagggtaagcaaggcatgtattacatggcagcatgagagagagaatgagagtgtgagcaaaggagaaactgcggaacacttataaagccatcaaatctcgtgagaactcactcactatcatgagaacagcatgggggaaactgcccccatgatccaatcacttcccacttggtccctcctcaacacatggggattacaatttggattacaattcaagatgagatttgggcagggacacagagctaaaccatatcaccactatttgtgcaccaacttcctcatttaccaaaaaccaaaatccttcatgcctatccactcattcatctggatacttagtatttcaatatactcagcaaggacaactacccaaaccaaataagttaagtctttctaaagtcaaacgtttccattttgagagttcttgaattaaatccagtctatttctgactctagaaaatcccctactgcccaataaactgaaggctcccccatattggcaatcagacattgctgctggcaaccttgaggatcaaaggtgggacttgtctttaggcagtgattctggaaccacagctaaatccccaaatttggctctgtattcaccatataatgtaagttattaaaaaaaaaaacaccttttcaaccattattcagattataagatctatcttactatgctgtgggctggttgctcatttcctgtttcctctctaaaggacaaagcccatttctcattcctcccctcaacccctctttctttctttccctcccatccaccatacctactctatgttaggcattatcctatccatggagtggagagagagagagcatgatgttgcagcaatactgagataatgaattaatattttccaagcagagcccacaaaagacgttagttgcaatctttcgagttgcagggaagagacacatcccaggtaacatcatcagtgattatctgatggaaattctcatcagactgatttatagtgctttcttaatcacaatttcagcccagaaatttctgtactgaaaagtcctcataggcattggtgtgaccccatgctaaatatcattgtcctaagtaataggagctggttttatgtagttaccagcctttctctagtgcttggccctattgtggtttcttcatctttgttcctccccatcttttttcttccaccattagccttctcatggacggatctgattagttggttggttgatcatcattacatgttcaagcaaataatgattgcaaaaatgaatgactgaccccaaatagagcagcactggctctgctgtgcttctctcttgtgttaacaatagtgaggttacttggatgaagcaggtgtttttttctcaagccaacaaaacaaaatgaaaaccacaaacacaaacaaaggaaaacactgtagtgaaattttgttagaagaaaatgtaactgaaaagcacttctgtaatttaaaaatcttgtgatatctagttaccacatccgtaagagagaccaggtgactgctattgaggtccagaggcagaagaatggattccacattggggatagggagaggaaaatgaaaaaaaaaaaatcttcttctgattcttttgcttctttttaaattttttcccctttaatttctttcccaataataggaaaattctctgtctcactctccttactcaggtctcacctctgctttcccttccgggcccctggctctcctggggtcaattcatttccacctaacttagcagcgagtcaatttctggctctttacctaattagtattctcaacacacacatttttcatgaaatatttgtaagataaatttagtcttcagtgggaaaaggaacaattacagtccacttgtcagttaaggaggtaatttaactacaacacttttctagtattgaaaacataaatctaaaaaacattcacagattaaaaaaaaatattcttcttagttcctgagaacttgaatctaatctgaattctgatcatgttatcttggcattggagaagaggaggacatacttttagaagttactaatgaatccgttaaatcaaagaaaacttgaactttctaatggattaaaactacatgtaggacatattgacgtgactatcaattcagttaagaaaaggtttttatttctaagattcagttgttaattcagactgaagcagttgtctgtaactttagttaataacagtactccttttcccttcagaagtttctgttacagaaaagttaacatttactaatttaacttttaaataacttatttttctttgaggctttctgagtatgttcagtcttcatagagtttacagctggaatagacttttaagatcagatagtctagtcatttcgtattctaatgtggaaagtgtggttaagaaagactaggccgggcgtggtgtctcacgcctgtgatcccagcactttgggaggccgaggcgggcggatcacgaggtcaggagatcgagaccatcttggctaacacggtgaaaccccgtctctactaaaaacacaaaaaattagccaggcgtggtggcaggcgcctatagtcccagctactcgggaggctgaggcaggagaatggcgtgaaccctggggggcggagcctgcagtgagccgagatagcgccactgcactccagcctgggcggcagcgagactccgtctcaaaaaaaaaaaaaagaaagaaaagaaagactaaaaccttgaaaaggcttcactttacttgggattttctacactagcctactaccctgcatgtttgcatcacctggggatgctttttaaaaatctagatgcccaggcctgaccactagagatcttaatgcaaatggtctagcgtggggcctgaggcattggtatttttctaaaactccctggattattcacataaggattaacaccactgctctacactccgccacctcacttagactacacggattggcttcagggactatttcttaatggccttaatctcatagagagggcagaggacttgcactgggctgggtagttcaggagataggaaaacaaattagacacagatctcactggtaagtagattgctgcctacatgagtgagggctggttcacaaatagctaaatgaccaaggtattgctattggtaagtgctattggacagatacaaatgagactattcaaggagacagatatatttggattgtttcagggtttgaaggccagatggcccacctaagaaaactctaccagtttttggatagaaattctataatctgtatacaagtttatatataagaataaacccatatttattggtgtgtgtgtgtgtgtgtgtgtgtacacacacaaacacatctgtgtatattgtatcaaaaatatttttttccactctttggctttccttttaactctcttaatgactattgatgaatagatcttgattgtcatatactaaattttatcatttttccttttataagtaatattttttgtgtgccatttcagaaacctcaacctaccattaggttatgatgataattttttaatgttttcatttaaaagcattatgaatatgttcatttaaatctgcaatcagtttagaactgatttgtgcttatgatgtgaagtagggaatcaagattcacttttttccatgaggatattcaattgatatagcactgtttattgaaaagaccatccattccacattccataataattttttctatcatagatcaaatgatcatatttttctcttttattttctcttccattgttctcttcatcgatctttgcacaaatacctcactatcataatttctacagctttatatgtaacagtatgacagctcttcctatctggtagtgaaagttttccagctttattatttttcaagattgtcttaacttttctttggatttttattgagattttagaatcaatatgtcaattacacacacaaccttctgggattttaattaggatttcattgaatctatgaatcaatttggggaaaatcaacaccttcacaatactgactcttccaacccatgaacataatctttctgccattttaatatttatttaattctctccagaatgttttgtaattttcagtgtagatattctacacattatttgttagatttatttgtatttgataatgttttcctattttaaatcatattgctttaaattttattttctgttgtttgatgctggtacacagaaatacaattgatttttgtatattgatctacgtgcagtgactttgctaatttcacttattatttctcattctctgtagatgattttctttctttcttttttttttgagatgcagtctcgctctgtcacccaggctggagtgcggtggcgccatcttggctcactgcaacctccgcctcccaggttcaagcgactcactctcctgcctcagactcttgagtagctggaactacagacacgtgccaccatgcccagctaattttttgtatttttagcagagatggggtttcaccgtgttagccaggatggtctcgaactcctgacttcgtgatccacccaccttggcttctttacaggcgtgagccaccacgcccggcctgtaaataattttcaatgttttacatgcacaatcatgttatttgcacactgtgatggtattattttttcctatacaatcttaagattttcgtttcttttttctttattgcactggctcagtcttccaattcaatgttaaatagaagtggtgatggcaggaaatcctggtgtcattcttgatctctaggggaaacactttcaataagtcattcttaaatatgatgactagtacaggctttgatgatcatcttcaccagaataaggttgtgtttttattttccatgaactggtattgagtcttatgaaatgatataactatatctattgagatgatcatctgatttctgagtatttttatatgtaaaatattctgttcaccatcttgatgtaaagataagtcatctaacactgtatagtgacatgtgtaatacaatttggggcatgccagctttattaccatttaaaaatatatctaaaatgtttcaaaatctcagttttgattaaatacataacaaagcatgtgtctctgtttcatagaagttttgaacagctaagttccagtgttgagattccagctgagatgtatattattcctttttagggaagcaggctgctttagatccattcatactgttgaaccttctgccaaactcaactgacaagtattacatttacaatggctcattgacatctcctccctgcacagacacagttgactggattgtttttaaagatacagttagcatctctgaaagccaggtaatcttagaaattcaaacaaatcaagtaattcaaagccatttaaaaaacattatgttctaagtttgctataaaatatccattttaaagcatttaccaatgcctttgaagcagatatgttaaattatttgatctttttttcagttggctgttttttgtgaagttcttacaatgcaacaatctggttatgtcatgctgatggactacttacaaaacaattttcgagagcaacagtacaagttctctagacaggtgttttcctcatacactggaaaggaagagattcatgaagcaggtatgtatttaaatataatcttctacaactctcatagatgcagtgttataggaggtaattttggtaaactgacaaatatagaggacttagagctttagaaatgttttaattattaattttgtagctttagataagctgtctaattttctgaatatcagataccacatctataaaatggcaactaaaaaaatctattcttcccataagaataaccccattttacagatgagaaaaactgagatttaataaggaaaatcatttttccagaatacacagtaataagtagcaaagcaaagtacacacctccaagttcactttctggtatttgcctgggctgagaggtatgagaaaggaatgaataagaggctgaggtgttcagagcttgggagatcatttctggttgggataataaggaaaactttatgggagaaccgcctttgaactgagtcttgacacagtatttgggttttaaagtgctcatctttgtgttggcagcccccaagaaggagcctagcaccttatcagtactgaaaataaatgcactgagttattaatgaataagaagtgaatcttcttcccagtttccttctttggaggaaaagaaatttgctctttttaggtacagccaagggaaatgacaccagaggggtgtctctagaatgccgtctgttttttgaagctgacacagtagttaaaagcatttttaaatgtccagagaacacaatcagatacatctgctgacttgtccttttcagtttttactgaggttgtcacttgttcattaaagaagtcgttgaagtgacctttgcactggaaaggacttctaacctttccaagtagatggacctctttgatatcagtcctgcaatgaattcaatgatttttttttgccatgtataatattaatttaataatcatctgcttttgaagattaggcattaaaacaacaagcttttattgttttaatgtgatatttccaagaatcgtttatcctttaacaaaaaaatagaattcaagacaacagtgatgaatagctatatttactatgacactaaattttaaactaaattgccccaaagtgaggaaatttaattacatttctatcagtagtactaaatactatagcatactgtccagtaaataatagacaatcgattagaaaaaagaaagtggatttaaccatcccctcttcttaaatataatagaccaaattactaattaaataaatatgatttttttgtcacacagttttatcaattctgttttattaaaacccttcactggttctgcatcacttttggaataaagttcaaactgcttgttattgcatggcaggctctgtgtaggtttactagtcttagtgcttaagcctccccaactagcatccttcccttacattgaactccatgaagtcccctgaagatgcaagaccacattcttccaggttgcatttgtacatttggcttccccattgatggcatattcctctccactgtcttcatttccctgcctcctcattcttcaagactcaagttgagcattgcatccactaggaggccatctctgactctgcttctgtccccctaagtctggccctggcaatggccacaggactgcacactcaaggagacaccatacccatagatggtaaatggtgctcctctggaggttgtacgatgtgctcagcttctccagacttcatagcagttaccacattatggtaattgtctatttacttttgtgtctctgccagcaatctctgagccccataatggaagtaaataagtcttttatctctttatctccatggccaaactaaagtaaagctctctttcactaaatattcactgcatggctgaaagactccatttccacataataaaaacatagcaagttatacttaatgggtgcttcctgcactgtttaatgtgtagatctgtatataattctgatttaatgtagaacattgctttcacagttaagtgttaaaggcccaaagtaataatccataataacaaaaacagtaatagctactgtgtattgtgtgactctaccaggcattggaaccagacaatatttgacgtagacctcacttcactcttatgaagttggcctcattgtcttcattttacagtttgggaagctgatattaaatggcttgcccaagtttacacaactagaaaggaatggagtcagtatttgaacccaagtctccctgaatctaaagctttgcaaacttctgtccactgtacttgacatttatacaggaacataatttctgcttacataaaaaactgatgactttaaagacatgaagcttcaaaaaaatagtaatggattttgagtgttttgtttatcctaaggtcaaacacagaaacaacactttcttaactacttggtgttttgggttctagaaatacatgataggacctataaagtaaggaaaataattttcttgaatggagcttgtaggaatcgattttattcccttagactatgtgtgtatattgtcacagaatttcacatccctgctgcaatactattatgactgccatagtaataataatttatttatcacctatttgtctgtggtttggttccaaaataaatggcagaactaactagaaaaacagcaactcaaaatagactcactcatttaaattgaagggacttaaatatgcaatacttaaggaaaaactagaaagtttaacaataaaaatgcacatttacaggctgtttttgttgtctacttatcctatatgtgtgccttccccagcttcctcaagaaaatccagtcacaccaactactttgttatgtttgtatttttgactaaatatttatggagtgatggggagaggctacaaaactcagcatctcatcaaaatatctgcctacagcttagacaatattgcttatatagtgttatccgcttatattgtatatccttaggataaataaaagtgctcaaaagacaatcctagtttttattagtgttttgtatctttatggccatcagtttttattctaaaattatgttagcagaaacacatctcttctgcagtctcccttgctgggccttagagtctttgccttcagattgtttgaccctcctctttggtcagttcccctcacagagtctcagctgctaccctcatcccacccaagtcttcacgtcttcttaaggcaggaaaattagctttgtcccaggtccttcaaacttgcctgcccgtgctcatcctaatgagctcagcaatcaaaaaaggccctttttggtagaatgatttattttcttttgggtgtatacccagtaatgggattgctgggtcaaacggtagctctgttttaagttcttgagaaatctccagattgctttccacaatggctggactaatttatattcccaccaacagtgtataagcattcccttttccctgcagcctcactagatgcaatgtttgttggctttttaataatagccattctgaatggtatgagatggtatctcattgtggttttggtttgtatttctccgatgattagtgatggtgagtagtttttcatatgtttcttagcagttgtatgccttcttctaagaagtgacatatgcacttatatgttcattgctgtgctagtcacaatagcaaagatatggaatcaacccatgtgctcagtggtagattaaaaaaatgtagtacatatacactatggaacactacacagtcataaaaaatgaaatcatgtttttttgcagcaagatggatggagctgcaggtcataatcctaagtgaactaatgcagggacagaaaaccaagtacctcatgttctcacttataagtgggagctaaacattaattacacatagacataaacatgggaacagtagacactgtggtgtacgagagagtggagaggtgggtgagttaaaaagtctccctatcaaatataatgctcactagctgagtgatgggatttgtactctaaacctcagcatcttttagtattctcatgtaacaaacctgcatatgtgctcttctatctaaaataaaagttgaagtaaaaataaataaatacataaatattttttaaaaggggcccttcgtttgccaaaacatatccccttctaggggggatcagagatgatacagaactttttttttctggttacatagactaatatgcacatttatgattttaaagtttcaatcattgtcaaaatatatcattttgagacagcaatgaaatagaaaagtcaggtcatgttggttggatgagtctcatccatacagttacatattcagtcgattcttttttggatttaatacaaaccactgtcaaataattaatatgcccttatgcgtatagggcctttctgattcctctttcttctgaaccatcataatccctgacctgcagctgtggcactttgcctttcttgccttgcttcaggggtgtttatgtgcatgccttagtttcttagtagatccctgtctaattgatctcagtgttacctgtaacatctgataaagtacctggcaggtagtaagagccctttgattccttgatgaatggtgaataagtgaatgaattaaaacattggccaaagcttactcatgtctaagcctctggggttaaaaagagcatatgtgttattggcaacaaacacagaaggaatgactggctttagatttccagacaggttggagccaagttcttcttgctgaacatcctttaaataatttaaaatttgcaaagccttcagaagaatacagtacagtaggattagcatgtgtgtacagttcatgttcaaaattaaagatcattccaggaaaagaaagattctaattttaaaaagatagtatcagtattatgacagaattgttatgaaatagtttcctctgtgactgaaaaaaatagcaaataatttacattcttcattttgactctgttgatcctctgtaaaattattgtacttaacaataatgaacacctagcatatcaagatttattatgtaataatttttatagatgtccattgtataacagtctgcatttactgaaatcattataaagaagtgagctacttgcttttatttttgcagttaaaaaagaggagaaatgaatctgattagacccacagaaattagttgtttctttcttcacaaactaggacttgctgtttccatcacgagtctttacactcaataagcatttattatcatttgtcctgtgcatagaaatactgtgttcctatgaaagttttttttttttttttgagttggagtttcactcttgttgcccaggctggagtgcaatggcacgatatcggctcaccgcaacctccgcctcctgggttcgagtgattctcctgcctcagcctcccaagtagctgagattactggcatgcgccaccacacccagctaattttgtatttttagtagagacagggtttcgccatgttagtcaggctggtcttgaactcccgaactcaggtgatccgcccacctcggcctcccaaagtgctgggactacaggcatgagccaccacgctccaccgaaagttttttcttaatggatatactatctccttcggaactgaatttgagaataagaattgaggattgtatttatattcagttttgatttttattaaattcaatctgaaaatttttattacatgaaattataaagcgtagaaaatatagataagcacacagaagaaaataaaaataccctgtaattacactacccagagataactattatgaagattttggtaaatatccatccagatctttctctgaaattttttaaaaatgttattatacttcacaaaaaatgagagtataccacatgtctgtttactgattttttttacttcatattatacctttaatacatcattcttaattttatgttgtattccatcatatagttataactgtatattaaaccttaaccagttcctcaattttggaccttcagttcatatattttttgccattataaatatctaacatcctcattttgaccaagaggagaagtaaaagctagcataaaaattgtcaaaatcttgctctcaataaatgcattgtaggaaaatgtatttttagaaatacattataaataatcgcaaaaaccatgtgagatttatatattcatataaactctgtaaggttagagaccaggccttattcattttttgcattcacagcaggcgctcaatagattatttgtgaattaatgcatattttatctgtatttccatttgtatggagagtgccatttattaggaaggcatttctttttggcatcatggaatggacactggctcttctaaggatcacagaatttaatagaaccatctggtcctgaaagagttagtgaaaactgtctttttttttttttttttttttttttttgacagagtctctctttgtcacccaggcttgagcacagtgtatcgatctctgcttactgcagcctctacctcctgggttcaagcgattctcctgcgtcagcctccctagtagctgggactacaggcgcatgctaccacgcccgactaatttttgtatttttagcagagacggggtttcaccatgttggccagactggtctcgaactcctgacctcaggtgatccgcctgccctcacctcccaaagtgctgagattacaggcgtgagccatcgtgcttggccaataaaaactgtttgtttgtttgttttttaaataatgagtactacatgtgaataacagtacaaacatagcaaaagtatgaagtagattaagttcaactgtatgcacgccttccaggcatcagatgctttcatgctgccttcttaccaccttacaaggtttttctgctaagaagcggggacctgctaaagcacaggggggaaaatcagaccttaaagttcagtagacttgattttaaactccagcaccaatacttattagtcatataaaaattgtcaacttacttaaactccccaagcttcagtttcttcacctatagaatggaaaagttaaaaatggtcaccctatagggttgtggtgaggtttaaatgacctaaaatttgcaaagcagtacagtcccttgaatgtaacgtgctactgactcataactagtatcattgtcatgtgtatatatggacagccaagatatcacagtgagtatggtatactattcaagtttttcatgattaaggtgttcttaaataattgttgaatagaattatggatgcacttcaaatctcatttgctagcattgtaatatatttaatacaaacttaaaaaaacaaaaacaataaaagataaaatagttgtcaggacagagtagcataaagggagtgtctttgtagcataatattttgacctatgagaaattttgtacaatttcctgaaaaaacgtcataatgaattatacattagtagacttaccttcttgaaggtcagtgggttgttactagcatttgttaaggtcacatgtatgtaaacactgtactgcacctatgttttaaaaagtgaaatgaatattatttacttcttggaagattatatactacaaattgaactgttgatgaagtcactctgggtccctaaagactgagtgaaagaggtagaattagaaagacttcttttggctattaatctaaagtgaatatgtaatcattcagaagtacaaaacagggatagatcttttccaattacattcagcatttgtgtcaatgtttacattagttgttttaggatctgctactgcctgaggagaagtcacaacctcattggttaccttctgggtctgtaagtcattcagcttttctctcagcagtcattgtcttactacttttaaaaatgaggctcataaacttcaaataaggaagtggtggacttattagcatgtatttacgaagcacttttggcatgagctgtgttatactgtacagaacttcaaattagtgggaaataactgagtgagacctagtgtagtctcaggtaattatgacattttctctgccactccctaagaatcacctccctgccatttcgtatcaatccatattttactcagaatacctgaagctaagactgcgccttttccctcagggcagattgtgcttcttccaaggcaggctagagaggaacagaagtggaaagaatctcctatctccccttggtcatctgcatcaactttcttgattaacaaagatttgtcacagccaaggaatgcatttgcatttctctgtctcttttctttttttcttcatcattcagtgctttgcttattaatattttagcaccggttccacagatcacatccattcctacgtactaggtgttcactcttcatttaagtaatgagaggatgaatgataaagcacaggactttgtgtcagctaaaagtttaagaagcttataccatttttagtaggaaatgtgtataaatttgaaaagaatttggcctaaatggcagaatttttcataagttttttatacattcctctatcaatggagtgcagaatttagaatctaaaaccactcgttacatctgagagcttactaaatctaagtgtttgaaagctatcacaggaatgagcatcctaaaggctgagacactatcttcttcttctgtatgtctagagtgcctagaaccaagcagatgagagtggaatgccaaaaataagtttttaaagcaagaacatgtatagctccctgataaaggagactacattctcattctttatagtgaggattctttgctcactgctcatgtatggtgcagtgccatttacttgtactctacgtgacttgacgtgggccccagactcacagccagaagcctgcactaaccagtaactaggttctccttcttagccagtgtgccctgaagaatcagtaactatgacccagatcacatgtagacatggaaaggaatcagtatgtgagatataggcctgagggagagaatgaagcttcaacagtttcgaactcctcatgatcaggaaaattatatttttgttactattttactttatagtagcttgtatttgatgtctgagtacatattgatctggaaaaatgttattgattttcaatccagaaaatgtactttacagagtaggtatgcttctaataagataattgggctggccgggtgcggtgactcatgcctgtaatcccagcacttggggaggctgaggtgagaggatcacgaggtcaggagatcgagaccatcctggctaacacggtgaccatcccggctaacacagtgaaacctcatctctactaaaaatacaaaaaaaaaacagccgggcatggtggtggacacctgtaatcccagctgctcgggaggctgaggcaggagaatggtgtgaacccaggaggcggagtttgcagtaaggcgagattgtggcactgcactccaacctggatgacagagcgagactgtctcaaaaaaaaaaaaaaaaaaaaaacacaaaacaaaaaacaaagataattgggctgatttcaataggaaatatatgcaaaacctgggaaatttagtgtctattaatcacttcttctatggtcattccatggtttgaacaccaaatagtgtgtatggaattaactaaattgttatttaaaagattcagctctatcttatagaaaggaaaccctttggcaagatcataaactacatatagaaatataggcttttctgttaataacattctaaagtatgtatgtttttttcaccataagagcctgtgtctccacatctaaaataaaagatttacctgaagcttgatagcatacaactatattgttgattaacaataaacacaggtatttaaaaagtaaatttctaattaaaatataaactcaagctattaaacaaataactaacaaactcttacagatttacaaggaggagaacaaagaagtgaaaacttatgggcaagaaaatataactttctttttggagctctactttatattaatgttgattcataaattttgacacgtctactgattgctactactaaggcgagacaaacatagacatccaaccaaagtcgtttctgctgcttaggcagtagtcctttggatgttttgcaatgaatcctttatttttttcttaagagattgttatgctttgtatgtatgcataaaattaatgcatttctttttttttttttttttttttttgagacggagtcttgctctgttgccccggctggagtgcagtggcgcaatctgggctcactgcaagctccgcatcccgggttcacgccattctcctgcctcagcctcccgagtagctgggattacaggcgcccgccaccacgcctggctaattttttgtatttttagtagagacagggtttcaccgtgttagccaaaattaatgcatttccaaacagtttcatcttgaagtgtatagttcactttaaatcattcccattctgtaaaaagtggaattgatttttggaggacgaagtctacaaatgatattgtccagagtcacttccaacttttctaaaagtgaacttgggattcattagaagcaagaacgagcttgttgctccaatgatagaaacgtgaatagctcacttgagacaatttgaaggtcttctgccatctggtggacatcagtagaagtctcactcagtagagtaacatcatgtagtatacacctaaattaaaatatattttcttattctgaattcatgttttttgacaatgaaataatttttcttttatttctaaaggaacaccccaacctaagccctccctaaacaaaaaataacatcaaaggtttgaagtgaaaaataatttctaactgatataaagctatacatgtatttatttgtaaaaggtatatatatatattcatatttcctaaataatttctgtaccactgctagcatactgtacatatgctatgccattgctatcatattgtacatatgctaaaattgtgactactaagtattatatgtaaaatttgtttccatttgtaagaccaattgttagttgtgggtgccctccggtgttcatgaggtgaactgcaaactagcagagacaagtcattttggtcacaatcggcggcagttgttaacatatttttgagtccttgtaattgttgtatcagtgatgactgatttacctaagtctactgcatgctctcattgagtggctttgtaacttcaggtgtcacctgaaaacaagtaagtatattaattaatattcagcctgagagatctttctctccctctgctttgcaaatctggcccatgaagagatatttattactctgcttcgttagtaaaatatcataaaatgttatagtttttcttttccaaattagtccaaactatcacattataagtgcattttggaaagtaccagggcactctctgatgggactaaatttggggcatgattcccagcatgggcctgaattcattttaagaagtctctgaatgatttttcataatactactttttcagatgagacaaacaagtctcttaattccctaaaattattttcttaatttacttatgataacaaaatgggatttttgtcagcacgtctgaaaaatactgaaatggcaattttaggttttctttggttttctctggttgggaattttaaattagttcaaggattgtctaataacaaaatgttccgataatagtatgtctttcataattgaaattatcctcatcctgaactgaatatattttcaggtaccaaacaagagaaattaagtcatcatgagcttgcagatattctttgttcatttatttctaaaaatgaatctgtatgccaggtatattcaaaacaatgttctaggcaatatagaactacagagctatggttcttatgtttataatccactagaggagtcaagattcattcaggaaaaatacaatacgaaagtatgcaaataatgcagtaataatgataaaataggcaattgacaatgacagagatcacttcagtagagattaatgtgagtaatgaaggaggtagcatttgaccaagacctttccagaatggaaagctgggatttgaacagatagagttaagaggggtggaaagagtattttagatataggaaatggaataagcaaaagctccacagtgggaaagtgtaactttttactttacattgaaataaaacacaataaaatttttatagaatatgttttatttattcaagtcctcaaggctaagttctatcaacaactgtacttttttctctctgaagtttgtagttcagaaccagaaaatgttcaggctgacccagagaattataccagccttcttgttacatgggaaagacctcgagtcgtttatgataccatgattgagaagtttgcagttttgtaccagcagttggatggagaggaccaaaccaagcatgaatttttgacagatggctatcaagacttggtaactatatgatcagttgttttacatagggtaacattataatttaatttccaaggtaagaacttacaaatggttgtatattattttcctccattacttttagactttatgtgaaggtggggtaggctgagtatttttaaatttaaaaaaaaattttaaattagaagctatactaaattatgtttaaagttacatttaattaaatggatatcataactttgccaacaataacactatagagtagatacatatgacttatgaactggagatcatttagtgtggcctttcttaagatttcagttgtagaatagtgccagaatctcagtgccctgatacattttatattgtgtcttccattacgctatatcagcacaggaaaagtagagtaggggacatacaagtcctctttgttgcaccaaaaaattttcagataacagctgggaagtcatgattgggtcagaactttggggatgtaagaaaacatttcttacaaaaagatccacccctgcctccctccaccagcgcatgcgaataaagtacagattccctttgtggcctgagcatgtcagtattaaactttgctctggtagggaagtgttggccatagattagggtgtagttgacaaaccttcatctggatgtaggtccagaaagtccccactgcaggttaaaggacactggactctgcactcaggcacctagagtcctgcaagtcctgggaacctgcatttaaataaaaatgcactattaattatgtttcatatcatgtggacaaaatggataaaattttagtaaccttttaattcagttgcctggaatatggagacacaatgacctgggaaaatcgtgaaataaatagtaataaaaatgtttatttcataattacgtgaagaagataattctattactgttcttgcatatatattgtcaagaaaaagagataacttagttgttcactttttcacattgctccttgtttgcaaatgccccccatttatttgtctaaaatattaatttttagtttgtagtactaatttatgaatttgatgagttctggctaaaaatgaaacttcctgaaactaaatctgatttttaaaaagcaaaaaaaaaaaaaaagcctagctttccagttcttcataattcacaaataccacaagtttaactaagcaacattgcataaacttttccttaggttaataaaatagaagtattttccacggaccagggagaaaaagttttctaggaaagatacctagtgtgttggtagtcctatgagaataacatttgtataattactaacatctttcttttagggtgctattctcaataatttgctacccaatatgagttatgttcttcagatagtagccatatgcactaatggcttatatggaaaatacagcgaccaactgattgtcgacatgcctactgataatcctggtaagtgccaccagatacatctatatattaactcaataaatgaggttagtttaattactgtatgcattgatgctttctctctatattcttttggccaaaaggcaaagtgattttctcttaagtctggattgccgggtaattttttggggcatgggacccatttctcattcagcaggtctggtgccagacaataagtaaacttatccttaatattggagtttaccatttgtaaaataagagtgactaaacatatttataacattgtaataatcattaaatgaaaattgctatgtaaatgttgagactgttattttggataattaagagttggtttaatttgtatttatttcctcttttcagcccccaaagcattatgtagtaagtgtatacatgatcctaagaccatgtgaggtactttcaaatatctatacaaagcatctcaagagtttgtcttagatctatttttctactttttataatttggttatggggagtggggtgttttcctcttggttgattgcccttactcttcccaaaaataagacaaacaaaagttgtttctacttgtgttttatttgcatgtaccttctctaaatgtatgggaaaatcttttgatcttagagcatatgcaataactgaatcaaatgctgaccatcataatttgacaatttacaagtttacataattaagttagttttcctcttgttagtgtaagcagttttaaataatagaatagacgtgcatatatgtatatatacatatgtataatcacatacatctgcataaatacatacgtaaatatatatattctttggtcatccatattaggagcctactcattgaaacttttacaaattttaatgtaagtgagtggttacaaattatattaattatagcctattttagttttacctatggcaggaaaactgatgtgcacggtttgcattgttgtctaaatgctgtagcagggactttaatccaacatgagcttaaataaagtacttgctcattgatcaccttaccatagctgttcttactagttgactttttaagttaaagattatatgtgagagcatcagatattatagtatgcatcattccaaactacaggctacaaaacttgccaaggtatctcccaacttgatattaacgactaatgaatattactactaccatgatggcaatatcattatttagtatttcttatgacctgcctcatggcctgggtataatcatctttcttattgcctttatttagcattgtttttcaaaatcaggttattttttaatggaatgtgagctctaagtcttatagaaaggcgagtatcaaagtaactgaccatgattttaactatgccatatcacgctacaccattgcatggcaaccatcaccaaagttcctcacaatgagctacccagaaaatgacacctttagccatacaacattgatcatgttggccaacaccatatgtgaattgattgaaatatggcatataaaaaaatctcttctacctttatttatgcattttgagtatgaagtatattgaacacattcacttaattttgttaccttggtttaattatgctactctaatttcgtacctaaaacaatttctcaggagtttgcaaaaaattattacttttacccatttatttttacactgtttcctattttattcgctatgccaaagctttaaactttctgaatttttttgttcttttttattttggagaaacttattgcatatgaaagaaacagattaaatatgtattttctccataagtgatttctgagtgatcctgttagactgactctatattggcacaaagtataatattcttctatcatttgttgtattttcacatttgtactaaataataaatattattaaaacttaaaatttctatttatttaaaggaccgggacactattgaacgttgtaataatgagatgcttgtttggatacaccattaaatctccatggatgcctcagtctcttaaggcattttgtatagtaatgatgtgttagtaaaagatggtatttgatttgatgttttgatatgctatgcctcttaaaaactaagggttacaaacttaggtagattattccctattatttctaccaaaaattcctgcatcaaaaacctgttcctccaaagtttgggattatcctattttttataaagaaaagattggttctcttaggagttactctttctgatctgaaaggctgcaatggcatttttatccacagatttacaatttagtattgaaattacattgtcttaaatattgtttctttggtaaatatatcccaatgaaagtcttgacaataatactatcttgtggcttggtaaaacagatttggtgcctacaagattagtgaattttattatttgtgtgactaatatagatgtaagtggcttttgagtttgctgcttatttcatggtttttattaataccacttcattctgttaatcatgttattttattagcttggtaactgtaatgtagcctatttaataattctattattatttaggataaactgtttactaaatattgtattgctttttgcttttgttaaaaagaaatgaaaaagataatttataattgctgtcattctttgatagatttcatatatatatatatatatatatatatatatatatatatatatatatatatatatatttgaggtggagtcttgctctgtcacccaggctggaatgcagtggtgcaatcttggcctgctgcaccctccacctcctgggttcaagcgattctcctgcctcagcctcctgagtagctgagattacaggcatgcgccaccacacccagctaattttgtatttttagtagagacggggtttcaccatgtgggccaggctggtctcgaactcctgacctcgtgatccacccacctcagcctcccaaagtgctgggatcacaggcgtgagccaccgcacctggccagatttcaaatatttaaatcagtttccctgaagaaaaactcagtcttccaaatagtgcctttaacttcctaaatgtgataaaactatgcatttaaattaataatttgcttacattattctttgtaccttttaattacacttgtgtacaaaagtatcaccataaactatgctcattaagtattatttcaaattgtcgtttaaaaaaatgtgtaagtttgcgttcagaaattaatttaaatttatcaaggagcctttttaatgttaaaaaggaatgtatattcttttaaaagacttcttagttacatttatgaataaataatttcataaatatatttatcttattgttactaagataattatgtttatttctcatataaagataaagctaattaacatacagctctggagggattatttaagtacatgttttagggccttgataattatatgtgttagatttaaatgcatgatgaattaaatccacagttttcttgttactgtgtgctttcagttgtataacaattgtcttaagtcaccatacatctgtcctcactctacctgtaccatccttcctattagtgttattacattattatatgcagaattatagtttgaagcagttgcggtctacattgcttttatttcgtgcactgtgaaacttggcttatcttcaaacaattaaataagccttgcttgctcacctacatcagtgttgcttttttgtgttatagaaaattaatgtgttcctaatgtgttttttttaatttaacattacatgtgtcccaaaacaaaaccagaatgagtcaacagtttttgttcagttattacaaaaatgtgaattctaagtgttcactcaaaacaaaaaatactgatagttcttcactatctgacttttttctagcctttatggagtctattttgatgaagggagatgattctataagccagtataatttttgaactatttaatatattggtagtgcctgactcaaaccagtggttactaaaatatcagttattacttgtgagaaagcagcaggtttaggggtacccaatatgagagcttgtgcccatcagcagagctaaactttgagttcaggatgttgaattcacagttccagaattatggcttcaaaatctttaggaagatagaaacaatcttccaaattattttatttcaaaattgttatgaagagactcagcaaaagacttctggattctaagaaatagaggtattgaagtattcctgggaatttcagtcagttcaaatgatctcagtccagttggaaaccagaataaagagccttcaaggtggttttatatgcagtaatatgaatgtgctaacacttgggggtgctcacccatgagagtgccaaggagacgtgcctaggcctacacatgttttcccagagccaaggaggcccaaagaaaaagtcttgcttttattacagtaaacagcaatccaattataagtcattaatattctttaggggatgtgattggattaaaaccctgtattgtaatttactagaatagaaaccataatccctatagttagttgatgctctggtaaaatgataaaaataagtctaatgcaatacacactaaaaatattaaagaaaaagggaaataaatcctcaatgtgatagttatacatacccttccccaacccttattacgattaaataagaaaattatatttgcccattaaaagtatgaagttaggtggagttagaccattttttaaaaattttgaacaccttcttaggcatgttgaaacaaatggaaaatacttgatatttttgtatagcctcttggcagaggaaaagcctacagtatgtgtaccactaataagtatttgattatgatttgccattatgagcattaaatatttcaacagtctatgaaaaagagtggctacacttcaaattattttcaataaaactaggtattgtctaatgggcttaatttgaagattttttatatgcatgaacttctttagtcatagcttttagaaaatcataaggaaggtcatttgtctgtcacagtttagttgcatctcatttttaaattcaaatgtattaatttgcatggcgattacaaagacacaagtgtttgagatgattgaaatcactttagtttggaaacgtaaagaataaagagcatgaagaacagacagtgaggaaaggatttcataggaagagaaatagaactgggagaccactaatgatcaaaataaaatttccaaagtccttcatggttagttttaacgagaaaaaaagtgagaattaaaagtaagattgtgattgtttctagtgagtactttcctttttaaattacaggagttgtttagggtaagtaattttgaaacagctaagttgtcaagagtatgaactttatctgaatcttcttggaagctaggcatgcttaatagattttacataaattctcattttatggttttcaatatattaatacccagcagtcttggtaaaaaataaacgcaagaaactttgtattggaaaacagacctgggtatgatcaacatttaatgttggttcagttataccttgaaatttctatagagtatgcatttcctttgaggacataaaacatagaacataagcagcattaggatagtgcagagcataaattttggcgatagcctgagattccataaaggcatgggagtttgaagaacatcattataataatgctcaaacctcacagttactttgtctaccattttcctaatgacccaggcagctcagaccaagactaaagaaaaattctctggccgctcagtaacgtggtagagtggctagattccttcttaggagagaaagaaccagcttagtctttcctgacattccctgacaggtcctgttttagaactgagatatatgttgattctgatttgttgggacaaacagacgtgaatgattgtggtcatcagattctttaagtagacagcattagctgagtttccctggggagaatgcacaagattcactctgttagttcttcttgctgacagcaaggctggaacctgctggaaaacactcttcattttacctggtgcagcacccagtatagaaataggtatgaggccagatttgaggagtaaagataattatttgtccctcagtcaaaatagaaacttaaggaagaggttgctaaataagaaaataaattctaccaattctccagaaaatccctggcatttccccaaccatgcttttatcagccctgctaaggatgaaataactctgtactgatgaaaaattatttgcgtttctttacactcagttttatcagtttcactgacctcctctccgttcttagcttcaatcagacttaggggcaggggaaatggggaagggagtggagtgggaggtagagatcaagatttgggtagagttacatataagctcatgtagtgttgtgttgtagtgttcagtaatgttgaaaactaaaatttggaatttttattgtgagaaagtctgtattttcaaagctgacattaacttttttattctttttaattttttctctttcacccaaaggtaaatccacaaaggccgaaatagttgaataaaaactttaataattttgtttttaattttgtagaacttgatcttttccctgaattaattggaactgaagaaataatcaaggtatcatagccatttttatattcaaatgttttaatattaaatggtctattttgaattgttgcttgggtgttaggtattgccacatgaatagaagttgtgtgtatgattcactgtttgtggggttagttgctaagctttatatatagatagtcacataatttatatacatgattctcttatgaagaagttgaaagatgttttgattggtatagtactaaaaaacaagaaatataaatggtgatgaagaaaaaaacaatgaactttaaaaataattatagctatgcactacagctataaactggaatttttttagtctccaaatttttctgaaaaactaaaattatatttaaccagttcttaacattctacagtttatatataaataaccaacgattgatagattaacgctaaatgtggcccaaaatataaattgtatatatattttttgcccatttaaattgtttattatttaagttgataaatattaatttactcaaagaacattgtgctgacaacaggcacttatactacgagtagtctatactttttaaagataaaatgtataggtatttgagactataaattttaacattattttatttttttaaatagagagaaaatgattgtttaatatcttttatatttcatcatatgccctaatgtgctgttcatcataactgaaattatatattcagttcaggtgctattgttgatgataatacgaatgtgtacatgtgtatagattgtgtaggagctgataaaagtgtgacaactagtgctgttatctagcaataaataacaattaggtctttcataggtataaactatcttgatattttatgtatcttatggctagtctctcttacaagggactattttaccaaaatgaccagtctactttgtaacaggagtttatagatttcagctaagaataactttatttgtatatgtacatacatgctcacatgcatatggagatatatatatatatgtatatatacacacacatacaagcgatcaacagaattaagtcaaaaaagaaattatatttgtgctgagttgttaattcaaataatgggtctgacatactgatgtaagcccttttttcagtaattggtgccatgtattaaatgatattctgtttaagttctgtcttttatataagtattacacatcacagactttttagaaaggaccaaggaacaatcaattatttttcatagtaattcccattaccatgattcacgatccatgcttagttactttagttacaagatccatgtttagttatttttcttctctattgccattaaccctaaggcaacttttaccataagcctacaaaagttatttaaaatcttccaatgttaattactagtaaatttctacataaaactataatattgtatttagtataatatttagctgtgtgaataggctacacatgtgactttatatgcattttaaaatattttccattttttaatggacatataatacttctacatatttatgaggtatatagtgatcaaatcagagtagtttgcatatccattatctcaaacatttaccatttttctgtgttggaaacatttgagatccttttttctacctatctgaaaatatatattattgctaactgtagtcatcctatagtgctatcaaacactagaacttattattcctatctagctgtaattttgtatcatttaaccaatctctgcctgtcgccccctcgccactacatttctcagcctctaataaccactattctacactctacttctattagatcaacttttttagcttccacataagagtgagaacatgcagtatttatctttctgttcctggcttattccacttacataatggaggtatatctttatttttgtcatacactctacaatagagatacctaaagctattcctgtcatggtgcctctggtcttgccatgtacatatatgacaaacacaagcagatcttcctgttagagaagaatgtgaagatatgtgagcaggctggcttgttgtagtcagagaaaagttcctagataggaggaaaaaaaaagcctcagtgcatcagcccaggtctcaacaggaaacagatgccacactcaagtggagtcatggaggttaatttaatcaaaaaggatgattcacaggtgtgggaagtcttaatgcaaacaaacaaggggtggcattgtacatttggtatgaattatatcagggactcgttgttacatctaaactgaaaggcaagagggcaaggagaaggtgcagagagaatggcacagcagatgctgtggcctctgtaatgagacagcaccatcctgtggtgaccagttaggtaaggaacctcccaaggaataaccacattatctggccacccgcctagaatgaatggagacaggtggagagaggctgtggaaagcaaaatgagaatatacaacacactgaggaaggagtcacctgagaaagcagagtagaattaaaaatcaatgtcattacttgttggtagaaggctatgatctagaggaaaatgaggaggagaaagaattcttatgacattaggcttatttggtttgtggggctttacccatggtggtagtacccaaagaaacagcagctctaattctttgagctggaagaatccttggcactgccaagggggagggaaccagctaccaccacacagatggggatttaaaataaaggatacaggagcagtgattggtaagcttggcttggtaaattggtagtagttcttgctattgaaattaactattgtattaataatatattgtcaaatcgaaagagacccatgtcacaatatgtcttcctggagcatgattatgagatgggatgaaaacacttcttaaccaatccactattcacttcctttgcttcatgcaggaatgaatgacaggctggaaaatgcatggaatatattcataatgacttttaatccctgaatatagaaataaagagtttgaaggatatttcttagatcctctccaaacagaaatgtatgactttgcaagacaaagtatctgtagcatgatcatggctttacactaacttttaaaaatgaagttgtgaaggataacatatgacacagatcaaaactacagtggaagagtcacaataactctataaagaaattgtaggaaagagtatttgttaaaatatttgtagtcagatatgaataaagtaatcatagggtatgaatgatatgttcttcttaaaatgttgacatagccaatgtgaccttacagcgatcaacaagatatggtagggtaggattaatgaaggtctaatcaccctcactgggttctgagaggttctgaacctcataaagtttatagaacgcatatgtatctgatgtgtgcatagttttttcccaactatgtaataagaagaggtgtgtagcattatgttacaataaaaattttatgaaaatttacattgcttataggaggatatgataaaggatgtttatgtggaaatataagcaaagagaaagaaatgtaatattattgtggaagtcaatagacattgttagatcatatagagaaaatggatgacatatctttggggaacttttgctgtgaaggagagacgagaagtagaatgataactagaaggtgaattagaggttttcctttgtgtttcctttaaataagagagatcagagttgatttaagctgatgagaattatctgtttgagaatttgagaaaacaaatcagaaacacaaaatagactgaactactcaattatgttcaattcccaggttcatgatagggactagatctaaaacacagaagtgcgaattagctttagaaaggaaggaaaacaactctttagttataacaggacaggaggggcagagataacttggttggtccctgtttttatgttaagaagaagtgtgtctttggtggcttctattttccctgaaaacaggaaggccatctgctgagaatgggaggaaaggataggaaaatgaaggtttgacagagttttggtagagagaaggagtgtggagtgataccagaaaactaggcaaatatttaggtttgcaaaaacaaggaagagtttagaaactccacactgccaagcttaacattgatccccagcaaaactctgcatcacattataaaacaaatatttatgagaacttctaaggaaagaagagataattggcaccaaccaattttccctaaaagcaagtcaaggcaaattattctcatttccttttgcattaagtttttaggtttgtagattataggaatattttatttaggaaattatttgggaaaatctctcaggtggtccttgtgaacaaggtatcacacagaatgtaaaacttcataaagaatactgactacttgtaaggaaatttttcaagggaatctgagagagtagccatttttagcactcaatataattcagatcaagtgcactaacttcctaaagttgtcacttatgcttcagaagatactcacgaccagccaaataattaagctgtaaataatgaggcagccattaagcttggaatcgtgtgtttcatacagtgcagatttccgtgtgtatgacacatgctcatgagcacacacagcttaagattcagattctttctactgatactggtcaagttctcaatggctgtactcagtaatttttttcttttaaggaacatggccttaaaatatctaatagatgatcaaataaatgacaattctaacaagctgttgggtacaaagatactgacaaaatcatgtcccacatgaactaaaaaaaaaaaaaaaacttgataactttggcaattgagttttctaagtatataattagatataatatacccagttttatgtatatatacgtatatgtatatatatacatatattgaatcttccatgcaatgaagattatgcttaaagcataggtcttggttaacttctagctagaagactagacgtctcctcctgggtcattgcatggaagattcagcctcctgaatggtgattctaagggaaacacaaaattaaaaaactaggagtagaatcatatattgacaagttgcctttgaaatatctgcctaagtcttaagtggaaaatattaaaaaacaaaaggaaaagaaaaccaaacacctcttttagttctaaatcttccccttattatgaccttaaaattatgatttgctctaaatagatataattaaatattctgaatgttcaactactttggtctttaaaatagtttcatcgccaattcttattacaataagctaatggctatttaccttgatgcctgatgaacaaagaaatataaaaacaataaaataaatgccatgttaaataacactgtcaataaataactacagtgtgtcttccttttaaatatgtggtatctattcatttaaccacttctttccatagcattaaaataaaggcggccataaattaggtgttttcattacgtttaattactcccctgtagagcggatggtaaaggttgtaggttagaggtatgcattgtacagaatggtgttttatgaggatgagaagttcccaagataccacaagtgatgattggcagtgtaagatatgaagattttctttttctcttatttccttatgactcattaaatcttgtttgcttttcaaaggaggaggaagagggaaaagacattgaagaaggcgctattgtgaatcctggtagagacagtgctacaaaccaaatcaggaaaaaggaaccccagatttctaccacaacacactacaatcgcatagggacgaaatacaatgaagccaagactaaccgatccccaacaagaggaagtgaattctctggaaagggtgatgttcccaatacatctttaaattccacttcccaaccagtcactaaattagccacagaaaaagatatttccttgacttctcagactgtgactgaactgccacctcacactgtggaaggtacttcagcctctttaaatgatggctctaaaactgttcttagatctccacatatgaacttgtcggggactgcagaatccttaaatacagtttctataacagaatatgaggaggagagtttattgaccagtttcaagcttgatactggagctgaagattcttcaggctccagtcccgcaacttctgctatcccattcatctctgagaacatatcccaagggtatatattttcctccgaaaacccagagacaataacatatgatgtccttataccagaatctgctagaaatgcttccgaagattcaacttcatcaggttcagaagaatcactaaaggatccttctatggagggaaatgtgtggtttcctagctctacagacataacagcacagcccgatgttggatcaggcagagagagctttctccagactaattacactgagatacgtgttgatgaatctgagaagacaaccaagtccttttctgcaggcccagtgatgtcacagggtccctcagttacagatctggaaatgccacattattctacctttgcctacttcccaactgaggtaacacctcatgcttttaccccatcctccagacaacaggatttggtctccacggtcaacgtggtatactcgcagacaacccaaccggtatacaatggtgagacacctcttcaaccttcctacagtagtgaagtctttcctctagtcacccctttgttgcttgacaatcagatcctcaacactacccctgctgcttcaagtagtgattcggccttgcatgctacgcctgtatttcccagtgtcgatgtgtcatttgaatccatcctgtcttcctatgatggtgcacctttgcttccattttcctctgcttccttcagtagtgaattgtttcgccatctgcatacagtttctcaaatccttccacaagttacttcagctaccgagagtgataaggtgcccttgcatgcttctctgccagtggctgggggtgatttgctattagagcccagccttgctcagtattctgatgtgctgtccactactcatgctgcttcagagacgctggaatttggtagtgaatctggtgttctttataaaacgcttatgttttctcaagttgaaccacccagcagtgatgccatgatgcatgcacgttcttcagggcctgaaccttcttatgccttgtctgataatgagggctcccaacacatcttcactgtttcttacagttctgcaatacctgtgcatgattctgtgggtgtaacttatcagggttccttatttagcggccctagccatataccaatacctaagtcttcgttaataaccccaactgcatcattactgcagcctactcatgccctctctggtgatggggaatggtctggagcctcttctgatagtgaatttcttttacctgacacagatgggctgacagcccttaacatttcttcacctgtttctgtagctgaatttacatatacaacatctgtgtttggtgatgataataaggcgctttctaaaagtgaaataatatatggaaatgagactgaactgcaaattccttctttcaatgagatggtttacccttctgaaagcacagtcatgcccaacatgtatgataatgtaaataagttgaatgcgtctttacaagaaacctctgtttccatttctagcaccaagggcatgtttccagggtcccttgctcataccaccactaaggtttttgatcatgagattagtcaagttccagaaaataacttttcagttcaacctacacatactgtctctcaagcatctggtgacacttcgcttaaacctgtgcttagtgcaaactcagagccagcatcctctgaccctgcttctagtgaaatgttatctccttcaactcagctcttattttatgagacctcagcttcttttagtactgaagtattgctacaaccttcctttcaggcttctgatgttgacaccttgcttaaaactgttcttccagctgtgcccagtgatccaatattggttgaaacccccaaagttgataaaattagttctacaatgttgcatctcattgtatcaaattctgcttcaagtgaaaacatgctgcactctacatctgtaccagtttttgatgtgtcgcctacttctcatatgcactctgcttcacttcaaggtttgaccatttcctatgcaagtgagaaatatgaaccagttttgttaaaaagtgaaagttcccaccaagtggtaccttctttgtacagtaatgatgagttgttccaaacggccaatttggagattaaccaggcccatcccccaaaaggaaggcatgtatttgctacacctgttttatcaattgatgaaccattaaatacactaataaataagcttatacattccgatgaaattttaacctccaccaaaagttctgttactggtaaggtatttgctggtattccaacagttgcttctgatacatttgtatctactgatcattctgttcctataggaaatgggcatgttgccattacagctgtttctccccacagagatggttctgtaacctcaacaaagttgctgtttccttctaaggcaacttctgagctgagtcatagtgccaaatctgatgccggtttagtgggtggtggtgaagatggtgacactgatgatgatggtgatgatgatgatgatgacagaggtagtgatggcttatccattcataagtgtatgtcatgctcatcctatagagaatcacaggaaaaggtaatgaatgattcagacacccacgaaaacagtcttatggatcagaataatccaatctcatactcactatctgagaattctgaagaagataatagagtcacaagtgtatcctcagacagtcaaactggtatggacagaagtcctggtaaatcaccatcagcaaatgggctatcccaaaagcacaatgatggaaaagaggaaaatgacattcagactggtagtgctctgcttcctctcagccctgaatctaaagcatgggcagttctgacaagtgatgaagaaagtggatcagggcaaggtacctcagatagccttaatgagaatgagacttccacagatttcagttttgcagacactaatgaaaaagatgctgatgggatcctggcagcaggtgactcagaaataactcctggattcccacagtccccaacatcatctgttactagcgagaactcagaagtgttccacgtttcagaggcaggttagttacggatcagaaggacagattgaggtgtggtggtttgcttgctctaaaagtaaaatgaaaattatagaaagatagaaatttggtaaaatggtacatcatcatttttaaaatcaaaacattcaacaaatcagtctccagtttctgaagtataattttatttattatttcaaacaattatttaacacattttggtttttaatttaattctctccaaaatagacaaaacaatttttatttattgtcaatatttcatactctacaggatgtcttttatgggtgggggcaattctgtttgcataatacatttttagtacactgcttttaaagcaagatcagagtatgctattttagatgattttaattttcatacttttgttctaaaataagttgtaaactaattctatttaattataaaatgaattattaaatctgagagtaatgtgacataagcacttctctctgacacacccctgtagaaaggaattagttaatttccacattcatatcagcttggggagctttatttattattattatttaattaattaattaattaattttgagacggagtctcgctctgtcgcccaggctggagtgcagtggtgcaatctcggcttactacaagctccgcctcccaggttcacgccattctcctgcctcagcctcccgagtagctgagactacaggcgcccaccaccacgccaggctaattttgtgtatttttagtaaagacggggtttcaccatgttagccaggatggtcttgatctcctgacctcgtgatccacccgcctcggcctcccaaaatgttgggattacaggcgtgagccactgcaccaggccagggaggtatttatgtatttatttatttatttagttttattactaagttttgagagttaataatgaagttaaaaaaaattttaatttttttgtctttctttatcttaatgttgcctaaagagggcagtgtaaaccatcaaaatacattttactttatgcctgctgttacacacttttttaagttaaataatgggcattaatatgtacagtatgtataggtattgtatcacaagatgtaaaacactaaacatttttctgtagaaatgaatttcatttgtgaatgtattttatgcaaattagcttactgaccagatgtttcttagagcagattatgtgcagtgtttataatacagatgtctgctttaaatgccaatggaaagaagagggaagttcaatcctattaagtatagaaaagtaatactttcattgaattggtcataagaattttataatttgcttttattaaatgtgaacgtatcttaaagtttaataatgaaactatttaaatcttaacatcaagttaaatgataggctaatgtaacaaaatgcactcattgtataaaagtgaatatttgtaatccagaacatagacatcagtctcattagaatatttttcctctttgcgatttcatctttttaattagttgtatattttttcttcagtgcagcagcaattaaaagtttgtacattttgatagaaaactttttctgtattctgaattgatacctatttaacattgcattttaaacacttaaatgctgatatgtgagaatgaataatgaatccataggtcactatagtcattagtgctgtcatatatatgaactgttatctgttctcaattatagtagctgctttttagttttcattagatttatgttaaaagaaaatatcaggaatccatctatctgtcttaccaggtagtgacattgatacgttcttgctagactatgctattatagaaggatatatataatgagatgctaaacctaaaattattgactaaagcttagcaaatttctcttcagtgcactaattagttttatttaattatcaagagtgtgttctcccactaattaggataaaacaatatgaatatgacatccaacattatcaagtaacataattagtgaagtaaataagatcaaacaagattaggggaaaattctggcctatttttaaaactgtatctataaaatcatatgtaatctgtcttatccaataattgaatatatccctttatcaaatgcatgcagactagctctgaaacttaagattttatggtgtatttctagaatgcagaacaatttacaggatgtgttctttaaaatactatagataaggcaaatattagtatccacaatttataaatgagtacactgacacccaaagatactagattatttgatccaggtcacatgagtaaatacaaaactgagcctggaaatgtctatctcttaaagattatgctttcatgttaggggccaattaaataaatgtaatgcttaaattgtaataccaaaattatactatcttattctcaaaacatcttttaaaatgttaatcttagttttttatttcttaattacctattaattaaatattttaatttaggtaactcatatatactactgagccatctcaaggagatattttagtctcaaataaataaaaataaaatcagtaaaattaattcctcattatagtacacaaagcaaccaactgattgtctgtgctttcataagatatcctattttgtgtaaaagagctaggtttcatgccatagtttgatcattctgactccttaattaaaggatttaagatcaaatatcccagagcacttttgagaaaagtttatacacaagactatatgtgtgtgcatgtgtgtatacagtgtctgtatataaatatgtgtatatggtatatttacatatataatgcattatatatcttacatatatgacatatatgataggacatatctcatatatatgtatgctatacatatataaatgaaatatgtaattatatatatatatacccatatacatgttgctttgaaaatttgtggctattgaagacctgcataattatataagcttacttcgtacatatattaatctacatcactggttctcaaactttaacatagatcaaaatcaactggaggactcgtcaaaacacaaattgctgacccttacacacagagtttctggttcagaagattttgtatggtttgagcgaagcctaaaatttgcatttctaagttatgatgatgttgatgctgctattctggggatcaaacgtcaagaaaatctgatatagataataatattaaccaccatttgtactaaatgaggtaccaaggactttgctaagtgaatgttttcatttattactgacacacttactttccatctaggtaacaatagttacaatagaaaaaactgtgaagacaaccatgtataaaataagatgtttttctaaattttataaggaaatgctcattgcattcaccatcaggttatttaggtgactagatgattctaaatgagtctccacaaaataatggccagagcttaggattaacattttaaaaaatggagtcttgggcttttctcacaagaatattagctacaactcagggctgagttggcttagaacattttcactttttatacagttttatgatttttaatcacacctttgaattccatttttcctagtaaatgcatttatagttataagaccaaacatcttgggaagtgtgtgtgccatttatattcctgtgatacattacatttatttatttatttatttatttatttatttatttattttgagatagagtctcactctgttgcccaggctgtagtgcagtggcgtgatctcagctcactgcaacctctacttcccgggttcaggccattctcctgactcagcttcccaagtagctgggattacaagtgtgcaccaccacgcctggctaatttttgtattttttattttttttagtagagatggggtttcaccatgttggccaggctggtcttgaactcctgacctcaagtgatccgcctgcctcagcctctcaaagtgctgggattacaggcatgagccaccacgcccagcctgatacattccctttattaatggattaaattcctgtccctttctgctatcacccttgatcaacagaagaaagctattattttaaatcttaagttctctccaattagatatattttaatgctatatatgtgcacttatattagtctcaattttcttaaatacatatttaagttaagtgaagtttctctgtctcctattacttaagcatcagaagtgccaagtgcataagaaggcatgtagattttgatgggaaactcaaatgtggttctttcagaatgtctgcaggtagatataagggctttttgtagcactgtcagcatgctgtaatgttagaaaccgttctcatgctcagtcatcttggtttcccactgaacacacctcaaagcaaagaggaaggattgttattggccacaaacactcactgttctgaaactctttttttaacaacttggaaggtaatctctagttaaagtgattttcaaagtgctagtacagtaatgtacctgactggcccccaaaataatcatgtgtcatcttttaaaattaactcaaggaccaaattcggatttttaattatttacacttgcatatttcagaagaatttcttagatctacatattcctccaattcacctgaaattgcatttttttttttttgtagaataaaacaatgggtaagaagaacattcaaaataacatttaaaaccaatctatatttaaaattgaaatcgattactggattgaaaggttatcctaatataagaagagttatcccaagatttttttcttcatgaaattattaattgcttctaactatttacctaatgcagggttaaaaataatcctatttaaatttattttagaaaattaatatatatggtgattcagtaagtagcgctgtttttcctgaccattctattttaaatcagtagtgatttttaaattacactactttgatttttcttatttagcaaggcttttcaaggcaatattatgcttacctgttgccaaggcacccctatctgggtgccaatctctgctgggatgttgtagttcttcttgtctaatagcctgccttacattttaaactttgttttaattaaattggcttataattgtagacattctttgcatcaaagagtcaaaattttaaaggtaagtaacagcaatgaaggttgcaattaatcagttgaaaaatgctgtgacttttccttcaccttaaatatcaattctctcaattttctctgactacagaggccagtaatagtagccatgagtctcgtattggtctagctgaggggttggaatccgagaagaaggcagttataccccttgtgatcgtgtcagccctgacttttatctgtctagtggttcttgtgggtattctcatctactggaggtaagttgagtatttgttttggaaaatttaattcataaaactcagattttgcccatatttcatctgaaaggtcttcaaagcatattcaaaatgtattttacctgagagctgccattaatttattcaaggtaaatttgtgaatccaaaggcacaataatgaaggagttaattgttcttcatacatcaattaaagataaaatggagaaacttttacccgttactatttaacacagtattttgacagttgtactttcatgttaatctaattaatagatatcatcatagcatttaacataagtctaagcttctcgttagttgttataattccaaaaattaagaaaacattttttatataataacaagtattgtttttcaatgacagaagagtttgtacaagatgtgtattaactgcacatactgataaaaatgataacctgtgcataaaagtaaaatcaagcaattaaactataaatataattagagactacagctaatgactatcttaagatgcacaggcatttaagatacatataatttgctttggggatgaaatatagcttcgctgtcaataatctattaagtccctaaagacaagaataattgtacccatcataaaatttccatcctgtggaaagcagaacacacagcaaattagtgtatttatgaggaacataggtcagtataactgtgtctgttagaaaacaaaggctttatgatgatataaggaatcatatgtgtaaaccaagaaagggcatagcatagtacttttataaagtatagcatacttttattttaaaaaagaattttgttcagaagatacataatttttttgtttagcaacatcattctgtaactttgtatcagcactctttatgtgttctctcaaaatatattctcgtgtttttttctgaaatatttaccattgttattgattgtttcccacatacctagtactgtgttaaccatcaggaaatatcaaaaatgaacaaatgaatgaaaaaagagtcagtgatttgaagaaatatatgcttattgagggggaagaatttgttataaaaatgtataaaatattacacaaaagtgtatgcgtagcagttgaaatctagtgctaaaagtagtcagagaacaaagaagcttggtaggccagaatgctcagggcaagtgtaaactaaaatgtaggactcaagctctgtgggagtcagaaaggaaaggaaaagggcattccaggcagggacacagcaggtgcaaagatcgggaagtagaaatgagctgtcttttgagtaaaagctattggatgtttcggacacagtgaggtctggagcatgtattaatagttgcatgtagttggacattggttggctagggtagaatgtcttcatgatcaactgtatttcatatctgacttcctttactagaacagagtagactgcacactatggagaacagatcatgcttttgttaccccacatgggtcaggagtctattttgttataataagaaacaaacaaacctggtttctttagtctgtatcccggtgattccaataattgccaaagattcttttttttttttgctaaagggttcatgcttagcagagtgctttgaacctaatccataatccataaatgtgtgttcaacaaaaataaatattgcttttctccccgttccccactgaacttgttgaccaaattttttaccacattgatatgtgtattcagattacaggtttctatctaatctgattttccctaccaaactacctttttaattaatgggagtcagtgttaagaattgaaaataagatggagaatatgaccaattctgcttcatagtaaatagtgctagtacaaagttccaataaagatgtcagatggtttataattataagattgaaattcgtttcttaatctgtgttacttttcaagtatattttctgaaaatttggaaatcagtatcttatttaatacaatttgatatttcatggaaaattaaaatttagagaggtttctatcttctattaataatgtgtcatttcatctgcattatatggggaaaagataaaattaccagtcaaaaatatattaatttgaccttcaataagatatttcttttttcttcacaaataacactttaggtaccttgaatgcagagtgtcatagtgtaaaatatatatgagtattttggccaggcacagtggctcacacctgtaatcccagcaatttgcaaaggtgaagtaagtagatcacttgaggccgtgagtttgagaccagcccaggaaacatagtgagacctccatctctacaaaaacaataaaaataaacaatttagctgggcatggtagggcatgtcatactcccagcttcccaggaggctgaggctggaggatcacttgagcccaggagtcaaggttacagtgagctgtgattatgccactgcactccagcctgggcgacaaagtaagacactgtctcaaaaaaaaaatcactttgatggttttatgtatatgttttatatctattaaatgttctgataaaaattgtcaatctttacttattttgaaatgttaagtagtgttagatatctgacctactaacatctgtttgaagttaattcactaaaaaatataaattttcatatcatcatatgctaggggtattatgtgctgaatcacaagatgatagttttagttaattcttcaaggtttatgtgaattattcaaattccagtgaagctacatttgtaaacaatatgatttcactagccaatatataaacattttagacctttgtaaaagctacagcttgagaaaaggtttaaaaatctaatgtcgtaccaataacagtcctaatggtaatgaagtccttaagaatttttccaagtttatatatttaaacttttttaaacagcttaattaggtaatattgaaatatcataaaactcacctttttaaagtgtatgattcaatggtttttaaagtgttctactatttccataatgactaatgatgttaagtgttttttcatgtgcttgttggacagttgtatatctttttttgataaatgtgtattcaaatcctttgctgcctattttttaattgggttatttgtctttctattattgagttgtggttttgtttgtttgttttgttttaaggagcagagagtttaataggtaagaaagatgggagaaggcagaaggaagaagctccccgtacagagacagagggagggggcctccaaagctgaaagaggagaacctgagttgtgtgtttttatatattctagatacaagtcccttatcagatatctgatttgcaattattttttcccattctgtgagttatcttctcactttcatgatggtatcttttttaaagtagaaaagtttttaattttgatgaggccaaattcatctattttttctcttattgcttgcaccttcagtatcatatctaagaaggcattgcctaacccagcaccacatggatttactcctagatttttgtctaagaatttaacaaaattaaaggactcatgcttcccagttgcaaaactttaattgaaagctacaggaatcaagagagtctggtactgccataagaatagacatatagatcaatggaatcgaactgagaacccagaaatcttatgtttacgatcagtggattttagacaaggatccaaagacttttcaatgggcaaagaatagtccttccaacagatggtgctaggacaattgaatattcacatacaaaaagtcaaattaaacctttttctaacactatacacaaaaatgaactcatagggaccatagacatcagtgtaagaactataaaattcttagaagaaaatatttaagtatgtttctaacctattttaatgagaaattaatctcaaagttttataataaatctgctatttcacctaatttacctaactcaagatattccccttattttttgtataaactttcagaggaagcagattttagccaaaagacagaacaaattcaaagtgagaaaatccagatctctttcctagttctgcccggcatttgctatatggccttgggcatgtcaattacccacctaggacttggtttctcctgtttaccatttgggtaggttcactagatcatctttaagattgtagccagcgaggcatggtggctcatgcctgtaatcccaaaactttgggatttatatatatatataattttatatataattatatatatgtataattttatatataattatatatatataattttatatataattatatatattatatgtataattttatatataattatatatattatatgtataattttatatataattatatatattatatgtataattttatatataattatatatattatatgtataattttatatataattttatatattatatgtataattttatatataattatatatattatatgtataattttatatataattatatatataatgtataattttatatataatatatattatatgtataattttatatataattatatattatatgtataattttatatataattatatatatataaaaaaaaaaaagattgtagccagacccaggaatagaataatacacatttctaggttttcattataaatactgtgatgaaaaggcgtaaaccaaataatcagattaccattgattgataggacctatgtctacaagtttaagacagtcatcacttttttcatcatgcctgatggataattagcaggtgacgtagttatcaacacttagcacatagacgtgaagacattcactgcttatactgaacagatggactacagatactgaatcttgtcatacattaaataatatgccttgaaatcattttccagatgttttatggtataatttacatgggccataccataagtttctaagatgcaaaaatgacttcaacagggaagattttcaaaagtttttttttttttaaggcaaatttttgaaagcttttttaagagttggaacctaaaaagtaccaaaggtatacccaaccaaatggcacactatcaaaagcaatatagaaaaggacttcacataaacacaattgttttggctgggggtagggagatgaaaagcaatatgaattctctcttgatcttaaacttgaatttgtggatcattgaagatgtctactaacccagaatggaggtcttctggtaccagattgccttttaagtgtagtgtcacttgctatataaaactgcactacaaagtatacctaaccaatatgcaaatgaccttcatccataatttagcctatactattttaattttttttttttacttacaagctttttggtattatttataaggtcttttagggaaattaatgccaaatagggttattgggctacttattttctagaaccaacaaagatttgattacagatttgtttatttgtttgtgacaagagtagaattggtgtttacccacactactgtgaacactagcaacatattcaacctcaatgccaacatgttttaatttaccattttatattctatgagatgctgccttcttcccattcagtcatagttattggtacatcctctatcgatccatctgcccatccagccacccattcgtccatccttctctacctatatatcttatctctccttccaaaatgttttgagatttcaagttatcccaattgaggctaaggtaggtaagggaatccaggatgcagctgaacaacatatgattggtgggatttacttgctattagaaatccatctaatcattcaacaaatattgaacacctattctactcctggcacagtattaagagttgggtttacataaaatcaagagatacggccccatcttttaaaaatgtaataaagatataagcacaaaacaaatataatacaaaacaattacactaaccaccaattaagaaaaatacttaaaataaataatgtagaataatgtccaggaaatgaagattactcttagattgattaggtgtatttttcaaaacagtcttttctttttttttcttttttttttttttttgagacagagtttcactgctgttgcccaggctggagtgcaatggtgcgatctcagctcaccgcaacctccacctccctggttcaagcaattcttctgcctcagcctcttgagtagcttggattacaggcatgtgccaccatgcccggctaattttttgtatttttagtagagacagggtttctccatgttggtcagactggtcttgaactcccgacctctggtgatctgcctgcctaggcctcccaaagtgctgggattacaggcatgagccaccacgcccagccaaaacagtctttatgtaggcttttgtgaatagaaatttgggttaggtgcagtggaaaaggagaagctgtggtgctaaaggctgattaggaaatgaaaggccatttcacagagaagatacctctgagctgtcttgaaagaaggcagtgtttgtctggcatattggccagtgagatattttggttagaagtagaggaatgtttaaagcagaatagataaatactgagtcaggagagaaaaggcaggtttatttatctacaatgtgattatattgttagagtataaacttcaataagaaaagtctaacagggtgtggaccacagacaggtagggtttaagtattgaaaagccttggatattataatctaaatatctgtaaaatatataactagaatatatgggcaaaagtaaaagggggatagatatgaacccgttagagctgttaacagtggatcaagaagacctattctttgagtaccatttgtaaatacttagaagggaatgagttttgagttagtagaagaaaaaataaaatctaggaaataaggaggattcaaaaggagtgatctccttctcactgaaggtattcaaactggaccttcttaaccataggttagagatgctgttgaagggattcttgtatcagctgtgatactgcactagctttttaaaggcattttacaacgttaagattcgttgaaatattttttaaaacatttcaccagcttgtataactgtattttggagaaatcaaagggattttaattctgtgaccttaaaagcatgtatcaagtccagtgtttccatatcaatatgtcagaatatgctccattcatagttgcacagataagccaaactttctgtatagtttctgccacaatctggtttcatttctgctcatgtgggccttctgtaggatagtttctgtggccctcatgtctctcattcctcatcagtctaccatactctccattttccaggatattaacctagttaggagggtccctttgacacttgtttctgtatccactcacctttctcttattcgacctgctctctcttgaagatatttctgtgttctctcaatatatcttctctcaggtgtgattatcaaaggcttatactaccacagtagatgactaattttgctatgtacgtcattgctttaagttttttgtttatttgcttgtgacatcaaggttataagtaaactatataattttacaccactcctacttatagaagttcattcatcagagaaatgcttcggggttacctccaacatgacacacatgctcccgagcactaaagactaacactgcaatgattgagacaaagtagtttgccttgtggggcttatattctgataagaagagaccaattataaacatataaaacaaataaaaacataatagtatttcaaatagtgatgtgccacagagcaaacaaaaaggcaaagagtagtgacttagtgagatagcctgacacttagctgtcatgatcaggggaaaactcagagggagcggcatttgggccaatgcaaaagtgatgggaaggaggcaggaagccagggggaatggcaagagtcaaggccctgaaaagctactgacagggcttcttcagaggtgaaggagaggaccagtgtgggctggatcctggtgggtgagggagagagttgcaggaaaggaagctactgaagtaggttgggggccaggtcagggattaaaaaggtcttaaagcccttgctaacataactgaatgtaattctaattgcagcgagaagccattcaagaattttatctaaaggaactaatttactagaaaaaaataaagatttaggtgtgtcactatataagaacaaatgttacattaaaactcattaggataaatgccttgtagcttgaaggtttagatggaaagtgagattgtcgttttcagtcaaacagaattcaagtgtttcaatttaaccactcatgaatattgctccaaataaattaaatgacctacatcaaagagtgtcattagcagttttaattattcaatttttaaaattaaagatggttggctttactttatagcagtagttggttctttgctcttgtcgatagtttgtggtgtgctcttaccagatgctactcttattatccgaatgaaaatagattcaggtgatattgtagatcatagttgtcactgaatctgaatatattcattctcgaaaccctcatcaacaagactagcatctgttgggaagaaaatcgcccctatttttggttgtatgctcattattttctcatttaagttctgtcactcctgcattattacagcatgaagccactttcttttttcatcaaaatgttaccacggtgtagtttcatttctacgtagttcatagttcatttctatgtactatggactgtgtaggtaatttctacatagttcatttctacatagcttgctgctttttgctagcagctgtatatgctaagaagaacatggtcatttgagtgaatgttgagtgaaaatggctttggacattatttcttttcttctctggctctatccaagatggcaggacattttgggtattatagccacagtggaagaatatgtagggcattgaggtttcctatattaacaaagtatcactttgaacccagctacaacttcaaattctgtctgtgcttttagcatgcctgatatctacttgctcttctgcatctgctacaggtaaaactgagataaattttataatttaacatcttgtgacttgttaaaatggatgtagatccagcaatccctcttctgggtatatacctgcttttgttttaattctggagatttctatcaaaattttcaagcagctcagaaattgtcttattttccatagcaactagtagtataaattgtgttcttttatttccaggaaatgcttccagactgcacacttttacttagaggacagtacatcccctagagttatatccacacctccaacacctatctttccaatttcaggtaatggcttaaagtgtgaccatgagtagctggtagatgttgaacaaaaagcacaatgttgaaaggtttgtttttatcgggacactatgcaaattgctataatgtagattagtaaatttttgtgttgccccaaatatttattacattatgttccaggtactcaggtttatattctgtcctcattaattttaaacgttcttaatcatacagatgatattgcaaaaatagtttataattgttcaggtgtattatgttaaaaacataaacatttatagcaataaaatattagttaagaataatggtgacttttacttattcctattatgttaatggaaacccaagcaacaaaagtcagattaagtcaatgcagaaaatgtggatgggaaaaaaatgacaaaaataaggataaagtcttgttcctttctgattacttgagtttatgtgagaaaagggttttttttttttaacctacattagaagaaaagatctataaaattgtggtggtaatagcaggaaaagccatgaacacaaacaatatatggttgcttgaaataatctctcaaaatgttgaacagttgaatatttgggaagactttagctatttttatcataatttatctgaataacagtattcaaaggtagccagggtcattttgatagagacatccattaatcctagagtttacactataagtaaaatcataaccactcatttttaatataaattcaaggggtgcatgtgcaggtttgctacatggacatgttgcatattcaagccacttatttaattaacattttataacatcttggattgaaaagtggcaacaaaaaatagtggtctagaattgtgtgatctgggaaatagaggacaataaaaggaaagagaagttaagaaagtagggatacaaaaggagaaagtcatctatgggatcatttcacagatggcactttttaaattctctgactgatgctccatattccattgctagaggctcaagttctacaggatgttatcttctgaaatctcatgacttaaaaaacagaaaaaaagtcaaacattttatttatattttacatttatatgttgatgtttgttatatgtttatgtcatatgttatatatattatttatatatatttaactatgatattttctttttgtgccttataaacttaggaattgtgtttgctatttttcctcttttgtcaagtctgtgtctttaagcaccactgttttatttgtttttctagtttacattaaaatactattattttagtaccacagtgtctcattgccatattcagaatttttagaccaacagctctcagttatctgttacattttgattcccttcacttccaaacatgtctcttctgcataccccatcagcatgggtatgcagattaactaaaattttatccaaagtccaacatggtttatttttaacaaataaaataatacaaaatcagacctagtccaggagatccaaactcccctgttttgagtgtcaaaagagtggtgattcaagggcatctccttatttcacttgttcagttctcaaagattccctcttgatcacctgaatatgcatgttttcgtgtaaaaatcagaaagcaaaatgaatgttagtctcacttacttgcagaccaattaataaaagaaattaccaaaatacctgactgtcaataaaaggtactagtctacttatgaaaggaggttggctgtgcaggataacatcaaggggtttgaaggacatttactaggactaatgacttacatgtgacaaatcatttctgttttcttatctaattataaaagcttctaagcaagatccttctataagtgtgaaggtagaattacacctttgtctagtgtttcttaagcttgactgactaattcaattttagtattcagatccactcatctaagagagagaacagatggattccagtttttttttatcagcaaattcttggtaactgaagctttaaaatattagatggcctaaatgctgaacatctgttgctataaataactgtttctagtgaaatgtgtttgatgtttccttgtcagagaaatcacttgtaaaattgcatactgtgaactagtaatatattcagtctaagaaagtctttccaggaaaagaaaaaaaaatcagaaccagttaaaataatttggccccttcacaattaattaacagatccaatgactttgtttacatagcatattttcaagttagaattcttgaaattttacagattaaaagtcccaaaggcttttgacattatccttaatgaaattctccatataaatgttttctattatttacagagagaaggaaaaaatgttttcaaatgtaaacatctatttgactgtaagacattaaattgtacagttttacaaaaccgaaattacactatttattatgcttggtccacactaaaattgaaactatattatttctacattttttctcagaatttacatgcaacatccaggcatgttaaagaaagttgtccatcaaaagatgcagtccactgataaggagtggggctatggtggtatttataaattaacattgaaaataagtgattttatctttaggtatagatggaaaagagattgctgaacaaatcagatttctgaaatacaggttttgattgataataaatgtagagaaaattggtaattgttgcttaacaattgaatgctgagatttagttagaattatacaaataattgaagttgtttcagaagctactttataagtgataggtacgtttatttctgttaaattatgctctctaacacaatttttttattcacgtagatgatgtcggagcaattccaataaagcactttccaaagcatgttgcagatttacatgcaagtagtgggtttactgaagaatttgaggtatgattttaatatgtctattttaaataatatagaaaatagtaaaatttaaaagattcagcaataggctatttcttgacctaaagacagtgcaaccatttgcacttctagtaaaaatatgagttccataattacatatgtgtttaggagatgtgttatagttactatataatttagacaattggttttctattttaaaagccaagttgtactggtggaaacataaaaaacattgttcattttcttccataaagtacaaatctttaatattcttttttaaaatctgaattaataacaatgattctttttaattttctaaattctataaattatactaaactactaaatttcatgatttccagccaaattacataaaaattcttaactgtgatttgtacaccagtgttaatgcaaaagcatttagataacacttacattataccaggtaaaaatgtatcttcttttcaagaaaatatgcatttctaaaaaaaatttgaacttcaaaatcagtaagttttttccaatgatattctgttaaaatactcttagacagaagggcagagaaatttactgcaaatttatcatggcccactgaactatcagctaagtagatttttcaaaatagagagctgtaagcacaaaggtaacaataaaaaccacaaaaaataaaagctaccctttgtgaacaccttcttacatgtcagtggactacatgctttcacattcaactttcacccatctctgggaattacttgttattatttctattcatattattgatgaaatcaaacttcatagaatttaattaacaactgataaataacagagttccattcaaacctaagtctgtgatttaaaaactcatgtcctttccactgtagctgtaagcaggaaggctgtcttcagcctccccgctggccattccccactagagagcttcatattgcatttttgcttctctctcccctaaccccagtggctctgcggacctccctctttgaagtactttgagctgtaagcctaatcacaaaatccactctctcctgattttctttccccactgcacccagcagttgcttcccaggactcacacccatcattatttatttagggaggcaaaatgttatagattagaaagctttaagaaatcaaaagaaagagaggtatgatatgtttttactaatttaaaaataatagaagccaatttatttaattctaaaagaaatctaaatcatagctacattgaataagaaagcagtgtgaggcagtactaagtagggtggaataatataaataattacatgtctaaaacatcaaattattttttattattttgacaggctagaggttacgcatgctatagtgttaactcaatgttaattttagtgttaactgatagaaattatcaggctaacatctaacaaaataagaaaaaatagacaatttataatataaattactgacaacatatacaaattcatggttttcataactataaaatcattttgaatattattaagcagtccaaaataattaaattaataaaataacttcatgttttcacttttaaagttttttcttaattctatctcttttaactcactgcctgattcaagaaaattttacattaactgaataattagcttagtaactaatagcgttataccttttaagtggttcttacatcaaattgcttttaattgttcagaatgacttgctgcaaaagaagccttaaaaggttatgtggagggagattgggccatttgctgatgaatcagatttttattttggttatctactttctcagaaattataaggcttttattttattatataatactaaataattataaattctaaacagatagaaaatatttctgaaagcatattaactcatttttaataaaaaaatgggcagaaataaattttctttacttactgaagtttaaacaaaaagaggtctgctaattaaatatttagagtgttatgctatctaaattttgtttcaaaatgatgtaatgtgtatgatttttaaaatttttaggaaatgattaatgttttgatagccttaaatctgttacaaaccacctaataggaagctgtttactttttaaagtaaccccccaaaaaataaaaacaaaaaagcacatcactaattcagagtttcaaatagtgctactatggtcaaataaaataaaatttggatattagccaatctgactaatatgacaaaatgtatcatggaatatgtggttcctctcatctgcatttttaaaatcatcatattatgacatcactatctccttttaaaagtgttatgttttcttttacataattttaaagttccaaattattagacattttgatttgcatgcaactgtttgaaacattctgttgtgttagtatttgacaaatgtatttattgcatgatcatagttttccattgtaaacctaatatgaacgcttttttttctcatttgtcgtgtgctgtggaatttgatttctttacttttttggcattcattccctcattagacactgaaagagttttaccaggtaaggcattatttcactgcattttcttttagccaagaagtagttgtaacttttaaaaaatttcatttgatagattattttgtttgttttgtcttcgttattatattttaaggcacgttgtttgaaagaatgtgtgttaaaatgatttacatataattttttacaggaagtgcagagctgtactgttgacttaggtattacagcagacagctccaaccacccagacaacaagcacaagaatcgatacataaatatcgttgcctgtaagtatattcttaaatcagctctgacttcaatatcattgccattttggtgccttttaaaagtgtcctgaagtcatctgagagctgaccttagtgtggctgtttggggcctttttatcagggaataataagacttgtgttagataatatgtagcattatatcactctgacttaaataccttctgcaattaataacatctgttttatttaaacaaaacaaacttagaattttaatccaattctttcttttgttctttctttcatagactgttcatctcccttggtggtttttgtcattatttatatgacttggtcctattttcacctaggatttgcttactcttatgtgaatttaccaaccgaattacattttctttaaactttctcttttttcttctacctttacctgtttcctccctttcttggcattcttcctggggtcttataaagaattagttggattctttcttttaattctttctttctctttgttttcttagcctttctctctccctttctcctcaatgagcagctatctggcttttttcattagtgcaattttacgtaatatttctctatgaaattattgtctttcttctgtcttcttatagcacttctggttgcatttgttccctcttatttttgtttatattccccttcatctcctaaaattcatcttcgtgctccctccccactccaaaaacaaacaaacaaacaaataaacattctacttataccctttctatatattcagtggtgtggcccttcctggcttcataggaccatggtattaccaacgaggttatttaaattctggtttcattctagttctgaaagccaatggagctgcactcttctccctgttactatcatgatctcttatatagtttgtatagatgttgccattctttccacagagtttacactaatttttattttttagtagagggctttatgtttgtcttcctgaactgcgtctatcctggctacacataaaactcacttgcttcttgagataaaattattagactttcctctacaagctaaatagaacatacgcatcaggtaattaagaaggagacagagattaacatttgatgaacacctacctactatgtggctggcactgtaaataagttatttcatttaatccctgaaacaaccaggcaagataggaaattgtccaattttagagataaagaaaaacaatccagaaattctaaaactattaaaaccaaactacaaaaataggtcaccctgacattgagacccatctgtcagttgccatacttccatttgcacatacattcctgtatcagtgtgacctgttaccatcacagaagtattcatatcccagagtatctctttaacttgttttggtttggtctgtattcacttcttcatccttgaaaagaataatcttctcagtgaaactgccatgtttccttcaagtaatactttatttctcatatgtattttctccaaataaatagtttttctgtagaactgaatttaatctcaaactcctgtaatcatactttgaattaccatagttataccttatatcatttaccagattttgcatccatcctgttttcactggcagtgtgctatctacctcctctgtgcttgagcagctgttctcctgcctccttttgaagcagtttctctttgaggctcctcctgaaagagctgtttgctgtaaccattcacagaaacttcctagggaacctttggctttcttctctatctcttcttccttcatcagcctcctgttcatccttacctagctttgtgagcctccccacccatcacatttttatctcttcgagaagctctccactgttcacagcccttgtaaccaaccttaaaattccatttaaaagaccaacttcagatgtcaacctctatagaaacttttatctcaaactaaaacaatttctgttttcatatccctccactacattgcacctctcacttgacacctgtgtatataccctagtttcacctattaccttagaaatgttcctgagggtggagattctttttatatttgtctttgttttctttgctgtctctgtatccccaaaaagcctatgaaagtaaatgattgcttgtgttgattttaattgaatgaaataaattattatgcaaatatgaattatctttgtgctgaaaagtagtctgtgctacaatgaaatatattctctttatattacagatgatcatagcagggttaagctagcacagcttgctgaaaaggatggcaaactgactgattatatcaatgccaattatgttgatgtaagcatgttttaattgaaaattttatagaaatcatattagactgaattattataccataatgccatacatgcatacatatactagtgcttaatgtcatgttattcgatagagaataaaaatggtaaagtaacagtaaataatttttaagagagcctatgactaaaaattttcatattaaatgacattcaagctattttacttggtttatctcctgagtgaggaaaggaattcatgatgggagtgtaccttcagggtggaataggtagattcagataggtttctaactctttagatgtgaagggcagggctgggcgcggtggctcacgcctgtaatcccagaactttgggaggctgaggcaggcggatcacgaggtcagaagatcgagaccatcctagctaacacggtgaaaccccatctctactaaaaatacaaaaaaaaattagctgggcgttgtggcgggcgcctgtagtcccagctactcgggaggctgaggcaggagaatggcgtgaacccgggaggcagagtttgcagtgagccgagatcgcacttctgcactccagcctgggcaacagagtgagactccgtctcagaaaaaaaaaaaatgtgaagggcagggccaaggcttattttaagtttcccatagctcctggcacaaggatgtgcatttagctgatgaacaaaaaatttccatagtagtactgattaaaaggcaaacatgattattaatccagttatcaacctttataattttgggtattcctcaggatacccagctttgtggaacttactgggactctccagagactgtagaccgctcactaactaaattggtttttcactgtgatcacctgaatgtcagcagaactcaattacttccctaagatgcacctctcagtctagctggcttcaggttaatactgtgtggaatactgaattcaaataaatgagtacataagtataaagtgggtttgttacaggcaactgtttcatgcttcctgaggaggaaatatgcatgtcagaatgaacatgagatctggagttagaactgattttgaatcccatctctgccactggctagctgtgggactttggaaagccacttaactcctctgagctttaggttcctcatctccacatcagggttgatgcttccattttgcatagatgaaagattaaggttcacgttctgtattagtctatgaaggacactgctatgaaggaatacccaagactgggtaatttataaagaaaagaggtttaattttctcatagttctgcatggctagggaggtctcgggaaacttacagtcatggtgaaaggcacctcttcacagaatggcaggagagagaatgagtgcccagtaaggaggaaagccctttttataaccatcagatctcatgagaactcactcactctcacgagaacagcgtgggggaaatcaccctcatgattccattatttccacctggttcctcccacaacacacaggaattatggaaactacaatttaagatgagatttgggtgggaaaacagccaatccatattatgcccataaaacatcttgtacaaaatgggacacagtgacgaattgctaataatgattagcacttctcagcacgttgaagtagagtaaggaggttacactccagatgcttttttaagctgttgtagacatctgagcccagtgcctaaaatacatttcaaagcagtgctattaaagacttgattttaagcatagcaagtagaccgcaaatggattaaatacacttggaatacaaatgcacagaattatagttgtgtcaaagcaaagagagctattctttttttttttttttttttttttttacaaaaatgatagggatccttctgcttcctaaccaagagtattttctaaccaagagtagctatcttagttggtaccatcaaatttctccaactttgcaagtttattttttatggttttcttttacgaaaaattatgctgaccttaaaaacttaggcattgtacagttaaagatgacatataaatcagttagtaggaaacatttgtcatttaattcttcagggctacaacagaccaaaagcttatattgctgcccaaggcccactgaaatccacagctgaagatttctggagaatgatatgggaacataatgtggaagttattgtcatgataacaaacctcgtggagaaaggaagggtatgtagataatctgttatgtcacccccaaaaaagtaattagaggtactttaaattagttttaatagagtcagcattaggaaggagccaaagttttgagagttatttgggattcttttttagtaaatatgacagctaaagggaaactgttaatactgttagtcaaaaagtttcattatgaatcatgctatctgccttttaaacgcattcttcatactgtgggagaagtaaatgggactcattgaaaagcaatgtagagtgaaatcgattttaattcacaggacatggattttagtcttggctctgtaacctgaatcttctgactcacttgccttatctgaaaggggaaattttagggtacctcatctcagtctcgttcagctctcagattctatccctagcctcctccacaactaatttcagtcacttataaagaaaataaaacatttagaagagtgaaggagtccattcctgtcaccatctcagacctttactgatttcagggaatgacttttacatggagcatttgaaatacagaagtaacatctacattttcttttgcagagaaaatgtgatcagtactggcctgccgatgggagtgaggagtacgggaactttctggtcactcagaagagtgtgcaagtgcttgcctattatactgtgaggaattttactctaagaaacacaaaaataaaaaaggtgagtcaacaaaatgatgggcataatcaagtgaacaatgcatttggtcattttttaagtgaataagcgatagaaccaagaaagggtaaaaagcatataactaaaggcattaaaaattcttgtaaccaggccaggtgcatggcttatgcctgtaatcccaggacattgggaggccaaggtgggtgaattacttgaggtcaggaggtcgagactagcctggccaacatggtgaaaccccatatctactaaaaatgcaaaaatttgctgagtgtggtggtgcacacttgggaggctgaagcacaggaatcacatgtacccaggaggcagaggttgcagtgagctaagatcatgccactgcactccagcctgggcaacagagtgagactctgtcaaaaaaaaaagaaaagaaaaaaaaaaagaattcttatcacagctgccgtcagtcctaaaggtttctttcaggtctctagatgcctgttgtggctttggagccagactggttttgttgctgatgtttaaagtgaatgtgatacatgaacagaaattcctaccttgtgtggcaactgcataggatggccctgacacaatcccaatattcaactaaaaagagtggagagtgaaacaagaaaaagaaaggaaacacgaagaataacgtattactcaggttcttcaaagaaacagaaccaacagaatagagagatagagaggtttacatttttaagaattgactcacatgatcatggggattagcaaatcttaaacctgtaggacaggctggcagcctagaaattctggtaggatctttacgttgcactgttgagaagaattctttattcttcaggtaacctatcctatcagtctttgttcttacagccttcaagtggttgaatggagctcacccacattatggactatgatttgctttactcaaagtctgctgacttacatgttaatcacatctaaacaaaatacctttacagcaacattttgtgtgattagtgttgaccaaacaatgggccaccatagtatagccaagtggacacaaaattaaccatcacaaatgacactctactttctttcattcattatggaccctcaacatcaaatcaagctatgattcctgacttgctgttgaagaaccgtgtgtgtgtgtgtgtgtgtgtgtgtgtgtgtgtgtgtgtgtgtgtgtgtgtgtttgactgttattaactgtctgaacttttccagggctcccagaaaggaagacccagtggacgtgtggtcacacagtatcactacacgcagtggcctgacatgggagtaccagagtactccctgccagtgctgacctttgtgagaaaggcagcctatgccaagcgccatgcagtggggcctgttgtcgtccactgcaggtgagtctcagagatgtgcctctaaacccatagaattgcttatacttgcaaaaaggaaatcaatggaacaaagcttttgcccaaatgggaatgatttcttgaaatgagggtgatgaaattatatgttcattttcataagtattgatcactaggaaatgaacatgccagactcagaattggattgctgaatatttctaagcaccacatattttttgatgttgattattttatttgattgttcaactgcatttcagcttattaactatttgaggcaggtggaacaatgtttttgttgttaaacaaatatttacctcaacatataataccatgggataaaaatgcttgagctacaacccctgtccctaggagttaaaattctgttttagaattattatgccaggcttgtatcacagaagccaaagcacacagattgtgagaaatttggcaaaatatatttaagttgtttttaaacattttgggaccagaatccctttttcattttcttgagttttaatacattacattgaatgtattgtcaattcattgttgccaagattttttaatttccaagactaggttgataaatagaaataatgatgtttcaatgaaagaggaaaaatgttgaaaatccttatttaatgttttcagttaaaataataatagcagttaatatttattgagtgctgactgtgtgccaagccttagatcacaaaaatcaaatttgtttctatgtgaagtattgttatcaaagcagtgtgtttttagagaatcaagtgtttgcctttgatatatagcttatctaacacattctaattaccatctcaggagttaaatttgagagacccatcttgaggctttttaatctgttagcctgattataaactgaggaaaacaggaagatgcaagaaaaatagtgctgaaggtcagggattgtttcctagcaaacggagaaggaccaggctattgtgcagagcaggtgctaattttccactctagcacttcctacttgtcggaacatttgaaaagaatccctgtccgtgaattctgtccctctgctagaggctttgaagaagtcgatagataatggccctttagaaataactgaagtcatgctttattcctcacaatctcactgaaatgatattttcagcagaaagctttatcaaattctttttcccattaacgcagaaatctttacaaacatgtctacctggaaacatagacctagggactttttcttttcagaatttggtgaagttaaagctttcacttttgtcagattaaatgtctatttgccacactctgatacataatattttcattgtacaatatacaatgaataataaatatggtttttgaaagttttaaaatctccaacagatatagtgaggttgttttgcaattagacacatgacaaaacattagaaagggtacttctaaatttgttgtgtaaagcattacgcttaattactaaactgtttacaatattgaattgccaaatgtattgtgtggctcactcgcttgaagaaggaagttattttaatcaacatgacaaccagtagtgatctatttttcaaatttattttatgcttaacattgaaatatttatttcttggtcttctcttcagtgctggagttggaagaacaggcacatatattgtgctagacagtatgttgcagcagattcaacacgaaggaactgtcaacatatttggcttcttaaaacacatccgttcacaaagaaattatttggtacaaactgaggtatgatttttaaaaagatgattttattcatctaaggtatggaaatgtcactaaaatgtaggtgtattcatctcctaggactgcaaaacaaagtaatacaaactgggtgggttagaacaacagaagtttattgtctcatagttctgatgtctgtaggcccatgcccttccagaagcctctagaagattcttccttgcctctttcatctcctggtagccccatgcattgcgtggcttgtggcagcacaattccagtctcagtcttcacttggctgtttccctctgtgtctgtgtctgtatccaaattttcctcctcttataagcataccagttatcctcctcctccagttgatctcatcttaactgattacatctgtaacaaacttttatgcaaataaaatcacactctgagttatcaggcggttagagctttaatatatctttgtggggggacaccattcaacccatgacaccagtaactgcagctatgtcatgaatgagccagataaattttatctgttttgtgaccccaaatagggaatatcctttaaaatgacactggtcacagagaagatacagcaaaagtctaagtagagtggtccacacacatccttgctagcaaaaatctcaagttcttcatatacatagtggatgaatcaattcattttttaaaaaaatatgaattatgcttaaattatatccattttaagcccatatagtttacccgtaagtgacagaacagtgtcagcaaatctgcattttatcttcactgtttctaaataaaaatgaacttaaatggaaacctatgaatggcagcttgaaagcaatcagaaacacatggctgagtgggcttcaaaacgaaaccacaccaaacatccttccatccattatcctaagcaaacttacacaggaacagaaaaccaaataccacatgttctcacttataagtgggagctaaatgatgagaacacatggagacatagaggggaacaacacacactggggcctttcagagggtggaggatggcaggagggagagaatcaggaaaaataactaatggactaatgggtatgagccttaatacctgggtgatgaaatagtctatacaacaaacccccatgacacaagttgacctgtgtaacaaacctgcacatgtacccctgaacttaaaataaacattaaaaaataacattctggtcatataagaccaagaaagagtcccgtatgtgaaatatcagagaactattaactatatataaaattgagttaaattgtatatgacaagcgaaaaggagagttggcgttgccagaaggggtaggcccaccagaagtgtagaagtcaagaaagccctctcatgaagtatagaggtgtcaagtttgaaggataggcagagaggacattcctttgccttgccaactcttcctgcctcctaacctattgtgtattgccaacaatacataataaatgaagaggtaaggaatgagaagaggttctttcttctgtgaccacttcctttcctctgtctgacttccttccaggttttgtaagcaatcttgtgtgggtcttcccccattctgtaaaactctccttgtctgtcaatggaaggtactatgtttgtaggtagatacatatgaggaaaacttgaactaatgttgcttattgtcattgttttgccaggagcaatatgtcttcattcatgatacactggttgaggccatacttagtaaagaaactgaggtgctggacagtcatattcatgcctatgttaatgcactcctcattcctggaccagcaggcaaaacaaagctagagaaacaattccaggtgagtcctcttggaagcctcttggatgtcactacagaactgaatgtccctgcatcttctcatttgagttgacagggtcacgttgagtgggcagtatgggtacaatgactttgtatttgagccatcgttgttccttactgtgggctacagtgccacatggtaaatttcaaggagaaacataattctgagcagcaagaggaaaagttctcagaaaccttaaactaacttttataaatgcaaacttggtcgagaaaagaagggcacagagcataacagaatgaacagaaagaacttgtaaaacatactatataagtgctctttttactttgaaaaaaaaaaacagaccacttaaacatgagtgcggtggctcaggctgttcctgatactctttggaaaaaatctgtgtctcacttgaaacccctggaccaaagtagtacacactggaataatagcttcttcaaccccagtttctccccctcaccctgtctcaaggccataagccagagacaagagcatgttttgtccatattgtacttcccacaataccagctctctgactttcagaaatcagtaggcaaagaattcagaaagcaggtgttaccggacctgccagctgagaccaaagcatgtgcagaatcgtagctttccacaggaatgggggctatattatttcactccacattgttcctgtgctatctgcttgcttctcagagttcctggagaaatagggccaaattttgaagaatgaatatctgggaagaatttggaagaccttagaaaaacccaggagagttaacaaatgttgagatttcacttatttctcctgctttgctccccagtctacagcataggatgttagccaccatgaattcctttgggccatttgccttttaaattactgcacggtgtgcctttggaggtgtaagagaaagattgggatcaatactgagtttagaacctgttgtcagaaagtcatacatgttttactcatccaagcttatctcagtcataaaggaatgccttaggggggaaaagagtatgcagttggttttcagataataaagaaatgttaaaatgtaattaagttgtaaagatatcttttccctaaataaattacacacacacacacacacacacacacacacaatattacccatttcaaaaaaactctcatttaaatatgatttgcccctgtgtagttgcataaaaacattgatatggtaagctaaataatccaggatcaaaatgaaaatattaactataaggtcagaattcaattgaaaagagagatattttactctctttcctgttaacatcataaatagggaaatgtatttgtgtgtatgtgtgtgtaccatcccataaaaagatatctattcgagtttgtctgtagaaaatttccttgaaataagttgaatatctgtgtattcagcaagtacttatcagacacctgccagatgccaggcttttgtgttattagggcgcaaacaagaatttgtaaaataaataaataaagcacacccagctcctagtcttatggtgcctgcagtctattagaagatacaggtaagaaaacacaaacaggccaggtgcagtgggtcacacctgtaattcctctgttttgggggaccatggtcaagaatttgagaccagcttgggcaacagagcaagaccctgtctctataaaaaaattagctgggtgggtggcatgagcctgtagtcctagctacttcagaggctgaggtgagagaattgcctgagctcagaagttcaagattacagtgagctatgattgtgccactgcactccagtctgggagatagagtgagaccttgtctctaaaaaaataaaaaagaaagaaagaaacagataaacaaaatagtgtgaaaaatgccattatagaggtaaaacaggatgctgtaggagtacaggtaaggatgcaacttaccagaggagaattgcgtaagatatatttaagagttgaacacaggatttggtgattcattggacattgcagagagagaggagagaaaagacacaagcataactactcagattctgtttgggcactgggaaagtagtgatgtcattcaccatgatcgagaattcaggaagaggaggtggtacatgcttgagaggtgtggtgagggagaagacgtgagatatttccaaatctaccttatttttaggttactgggagatgtcccaataaacatgtatatttggcagttggatactggttctgaagcataagagaggtctggcctgaagaaatggatttgggagtgatcagcgtagagatggttccttgaagcctcggaattggataaaatagccaattaaaaatgaaatgtctaagaaaggttagaaaggattacttaagaaaacattttaattaacctatgaatggccagcccacaaaggaaaggggggaaagtggcatcagaaggtaggagaaaattcgggacagcggaatatactaggatccaaagtaaataatgtttcttgaaagaggaagtgaacagagatggcaagtgctaatgacaaatcaagtatgttgggaatggaggaggccgttggatttagacagaagtcattgatgacctttgtctaagagctatttgaaaagtatagcattgacaccagattacagggagttaaggagtaaatggatttgaggaattaaagacaattatataactcttaaaaattgtttactctggatagaatgattaagataactataagaaaactttgagttagtaagatttgggggttgtttatttggggtttctttgcttatgttttaatatgagacatgagagcttctttaaaaactaatgggaagggagataattggagagtacagagaccctgggtggtacaaagaggcgggattcaggacacaggtaaaggaattagtgttcaaaatcagtaaaacctatgtgcatgtttacagtagtacacatataggctctaaggctggaggctgaggaagtactctacggtttctattatctgtgaagagaaggtgggtgtgtgtgagtgtgtgtgtgtggtgtgttgctacatatattcctttttttttttttttctctgagacagggtctcactctgtcacccaggctggagtgcagaggcacaatctcggctcactgcaaccttcctctccctggcttaactgatcctcctacctcagcctcccgagtggctaggactatagtaagtgtacacaaccacgcctggttaattttttaaatgttttttgtagagatggggtttcgccatgttgcccagatggtttcgaactcctgagctcaatcgatctgcctgctgtggcctcacaaagtgctggaattatagccatgagccactgcacctggcccattgcacacatactctaaaataaaactgttaatagatgcagagggaaaagggagataattttaaggaatctatatcatatcagtcattttctcagagtataatacagtaaaaattggaaattgacagtgaaaggtttccataaaaattttgatatttgggcattaaaaatacccttttaaataatatacatatttaagaggaaattataaaatctttagaattaaaggtcaaaagaagcactacataacatttttaaagtttttaaaattaaatagaaaattaaaatgattaaataaagatttaacaaaacctaatgcaggttctatcaaaagactattaaaatagacaaacctgtgtccagattgatgaagggaggaaagaatgtgagtaaataatattgagattaaaaaaagagatataatttagaaacatttttaaacacaaaagcatactttgaatatatttgtgacaatacatttgaaaacctatatgaaatgaataatttattgtatgttctaaaataactaaaagcatatcattggattgtttgtaacacaaagaaaggttaaatgcttgaggtgatggatgccccatttaccctcctgtgattattatgcattgtatgcctgcatcagatagctcatgttttccataaatattacaccttttatgtacccaaagaaatgaaaactaaaaagaatttttaaaggaaaaatataatgaaaatacactaaaaatatatagaaaatttaactaggccaatagccattaaggaaatcaaatcagctgcctaaaatcccaccttcaaaatataaccaggcccaaaataatttaatgtcacattttaccaatttttaaggcacagaaaattccaaatgttagcataaaaaatccaggtggaaaactaacccagctattttattggacaaatataactttgataccaaaactgaacaaagacagaataagacacttataggccagtatcaataatcttatttaaaatatcagcaactcaaatccagcagtgtatttctttaatatcatgagcaagtagagtttatagggtttattccaaaaatgaaatgatggtttaacactggaaaatatgaatagatttacataaagaaacattatgaccctgtcaattaatacaggaaaagtagtcaatcaaattctatattgttcatttaaaaaaagaactcttagtaaaccagaaatagaagaaagctttcttagccttctagaaagtatctgtcataaaactacaataaacatcatacataaaaattaaaattggaaatgctcatagtagattccagaaaagataaggaatgtcaattattggccctgctattcaacattattctggaaatagaattaaactagatagatgtataaaggtagaaaaggatttgcagatagataatataactacctgcaaagaatatcaagagaagcaaaagagaaaataattttctttagttagagtttaataaacatacataaaaatcaatagcattgttatttgttagctttgagtgattagaaaatgttttagaacaaaaaagaacccaatcattaacaaaaacaataacaaaacagtagcaaaaacttcatcatctcaaaaaagacacttaacaaaatgtgcagtaaatatagagaaatttataaaagtgaattgaagagcataaaattaggcccaaataaaatataatgttgatacatggaaatattaagatattgcttctccttacattgactatagaagtcaatgcagtatcaatcgaatttatggcaggatttttcatggaaaatgtagaacagattttaatttagcatggaaaattaaacagccaaaatatctcacctatactgaaaaggaaataataatagaagaattctgttgtactgtatatggaagacttatgatgtgatataatagtattggaaaacggaggaatctagcagaggaatgatgcatttgaggaaatctgttatgtggtagagttaacattacgtatcagtgaagaaacagcagattaatctataaataacattgggaaaattttcatgtcaaaaaaagtaacattgtatctctaccactcctcaaacactaaaatgtattcaagatggattaactacctaaaggtgaaagcaaacctttataactattgtaagatcattttagaaaagaaaatattgggttgggcacagtagctcacaccagtaatcccaatactttgggaggccaaggcagggggattgcttgagcccaggagttcgagaccagcctgggtaatatggtgaaaccccttctctataaaaaataccaaaattagccaggtgtggtggtgcatgcctgtagtgccagccactcgggaggctgaggcaggaggaactcatgagcacaggagattgaagctgcatgcactccagcctgggtgacagagtggacacctgtctcaaaaaagaattgaaggggaggagaggggagggaaaaggagaggaggggaggggaggaaaatgcacagcagggcatatattggaaagtatttcttaataacttcaggatagagaaggacttcttacgtaaaatgataatttgcagaaaacttaaataagaaaaataaattggctgtaaatctctgtgtgattaaagaaactgaagatgtttccaactttttacacactatagatttacattcaagacagcaagaaccccctacaaattaataagtggaaatgaagtaaaaatatgaaaaggcaatcatcaaagaagatatcttaatgactaatgagcaccaaaattcacaatttcacagaaattaagacgatgcaaatcaaaaccatgatatactatttttcatttacccgactgtcaaaaataaaacactgaataactgaatactcctaatgctctatggaaaccgtcacacatgtgcacattaagagacatacaggagatgctcatatctataaagctatacaattgcaaaacactgcaaatacccaaatattcactagctgaggagtagataaacagaatgtagattattcatgtaatggagtagtaacagaaggaataaataaaattgaattatatacatatatatgtatatacacacacacgatgtaatttacctccaaacgtctgtgtgagatttaaatagaggaaatggaggttgcacatctatagtggaagccgtgaggaagattatctcaatgctaaaagaattactagagatttctttgcttcttgtaactcccctggtgtaaaacacagaagctccaggaagttcactaaaacaaggttaaattcaaggaattgttggaaaacatttcatatatatttttagtcagtatcttatcttgattggtgtgtgtgtgtgtgtctgtatgtatgtgtgtgtgtgtgtatttgggggacttaaatgaaataacctatatatttttttactttcttgcccagctcctgagccagtcaaatatacagcagagtgactattctgcagccctaaagcaatgcaacagggaaaagaatcgaacttcttctatcatccctggtaagttgtgttgatccttgaaaaaacaacttttttaaaataataaaagccttgtaggaaatttggcaatatttgtatacctttggagcaaatggagcacattcaaatatttttaattttgtccaatatgtaactgttccaaaggactcataaatccttgaagtcttatctaaatatgcaaaatgtatagaaaaattagcttacagatcagagacacttcaacttgaaaaatgtagctatattttacatctggcatggggaaaagatggtacagtgctgtgagcatgagttgttttgttttgttttgttttgttttgttttacaatgaatgttctgtgttccagtggaaagatcaagggttggcatttcatccctgagtggagaaggcacagactacatcaatgcctcctatatcatggtaagtcagagaagtcactgaggagactgccagcttgtgttacagggatcaaaaccagaatgggctgccagaagcaaagactacaggcatgggcaaatgagtggtaaatttaagttaaatctgttgactgcaagcccacagtccacagcaacagttgtaataatggcatgcttttatgtgtgctgggactgaagaaacctcaagcaagaccaattagcagatgcaggaacacaaactcaccatttagggctgatgagtagatataaaacttatacctgcttatctaaaaggctttatttccttcactaaggcagaacccctgagcaaacccgggattcactggcttcagcaaagcctaacctcactgacatcactacttcaggtttgcagcatgatttttagcctgcaaactgggctcattttaggacctggatttctaaagatacaatgcttttcttctgcatttacatcttttgtgtgagggatgagtgttttagtagctgttgtgtgaccttcattaacatttaaggtcaatcaatcacggagcttggcttctgatgaaaacctaattcttattccaaaacctctgttctatttatccagcaaagaaaatattaaggaatatctatgcaggaaacacagggatgtgtggtagtggcatctaaatacattcacatttcaaatgagccactgggtgataggaatgggatctgaaagcagaaggggctagaagagccaggactttagtaacagtataaatatcacagttaatacaatagctctagcttctaggtatctgtagcaaaatatgcaagatgtctccctggttcctctcttgtagaggcaaccaaggtgtctggacaccaaagtgggaaatgtaaactctcctgtagatttcccactctctgtggagtaagagacagtatccacaaagcagtttggtaagtctacagctagaggtgtgtttttaatgaaactgttcaagtttctctatcagcttagctgatagctaccattctcagtgaccaccagatgcccagatgaaagagcaccaataagggattcacctgcatgggttccattgagcagtctcacctaaagtcttcaatgaaaaaccacagtgtgaaaattcagagtttttagtatttatggaccctgaagggttcatggcactggaggccacatgcatagagcttaggcagagcaagcagggaaagagagaaaaggacccatggaccaatgcccccactgagtccagggcattatccaagtaggttttccatggggagttttacttgatgagtttaaagcaagcaggcacaagtcccaggaggttatgtagtgactgagaggtatttactatggcatatctgcaaagtccatgtaggtgtgagggtcagcgaggccagccaagcaggctgttctgtagtgaaatggtcactatggagcagttttgtaagttagacaactggatcgatcactttaaggaactggtggatgggacgatgtagaactggaaactgtatcaaggtgactgaaccctgcttctggcatgacaaagtccaacttatatttaaaaggaatgccaaggcaacataaaaatcataaacattcactacaagatgataggtaaccttgaatactacatctgacttcttaaggcaaacttgggcacaatgcttaatttctaaagcctcagtttaatcatctgtaaaatgggggtcacagttcctatctcacagtattgctgtgaggaacaaacgatttaagagaaaaaaaaacacatagtgtcacacctaacatacaaagtgcccagtaagcattggctataataatcatattactacatatagacacaatctgcaattcaaaagactattttgacttaaggtccattgatgtataaatgcttatctgaatgtttaaaggcatacgccagtgctgtttttgtcttctctttggttttgtagggctattaccagagcaatgaattcatcattacccagcaccctctccttcataccatcaaggatttctggaggatgatatgggaccataatgcccaactggtggttatgattcctgatggccaaaacatggtaagtcccttagaccacttttgggacttcctttacagaggaatatcaggttacaaacagcaaaaagtccagcatacaaacaagcaaagtaattttgttttcaactaataaactaatgtcattgaaggcatactcttctgctgttgggcttggtttcattatacaatctcctaggtcaaagtgatgaataaaagttttaaatgaaagtagatataaatgaacttaaatgtatgtgttttgtgacattgcttaagaaaattagaatcacagccatgaaagaaatcagtctatatgcttctatttacatttttaaaataaaatgtgttagaatatttagagattccctgagtttaaggaggtctctaattttttctttaaaaaaaagacctagttagaattaagtgatttcctagtataggaaggacttttttagtataagaaaagcagcgatatcccatccccctctttatatatgtttggagaacccaacaagtaaaataaatatttcatttaggctttacatgtttacttagctttgatattcagttcttatctataaagttattgtgtgaccattacacatgaggctggaaatttctgagctcttaaaatcacttgaatcttctgtactcagttgtcaattacagaagcaataagcttatcattgttgataagctaagagcataaaataataaagcaatagtttgggttcctaagtgtgatattttattcagcacatcaatctagttgaaggcacgagaaagatgttaagagtaaatattcagattcaaagaaaaattggagttgaaatttctgtgccaaccaattaactttatttcacaatctgacttattggtcattttattaaaatctagactcttctttcatttgcaattctgcaggcagaagatgaatttgtttactggccaaataaagatgagcctataaattgtgagagctttaaggtcactcttatggctgaagaacacaaatgtctatctaatgaggaaaaacttataattcaggactttatcttagaagctacacaggtaaggatataagttaaatgacaaactttttatcttaaacacatgttaaacatattctgacctaaggatctgtcctagaaatgaaaagcatgattctgattttttccccattgagacaaatacacaaataatcaaattaccttgtcactttcattttcaaaatgatatagaatcattaaacaattagtcatcactacgtcattcctgaattttacacacacaggaaatcaaagctcatttactccagattagtggttgacagaacttaggtgaaaaataaacacagtaaaagaataaatttattaattttaattctcatgttttaatgtaatcccttaccttgtaagtaaaatgggaaaaatactacctacctaaactctcctaagtaaaatgggaaaaatactacctaccgaaacttactattctctaccaatgtagaaagttgatgagtgatagtgatgtagtaagttgataaatgaagtgctttgaaaacacacaggacaataaactaagaatattttactaaagtcttggttatctattacacaagatactgttgttatctacaagataatacattgttttactaaacttaattatatgcaaatatctattattgaattatttgtcaatgattatttacatagaactatagtaaaatactaaaagatactaacaatgatgtctaaggaatgtgagattcagagtaccttctcaatatgacaaagattaaatcatgttccaaataatttgaatgtttcacttttctctagttctgaaaactagtacatatatggtatacaagaagattctctatcctctagaatatttaaatgaccaatacatataaacttgataatacaaataactaatagtgtaaacagcatagcagtggaatagtatcctatctcattaaaacaaaaaagacttaggtctttatttgtaaaatgaagggcagaaactagaaggctcgtaaggtccctgagagctcttaatgttccatgctaatttctatctctgattactccctgatcactttagtagcatccatttagatctgccatctctcatgccttctcattattcctagttagtaagaaagcctattacaagagtaataatcattatatacctattatcttgtcacacccattatcctgatcactgatcatgtttaatatcttgctcagcactgggaaattagcactgtttttctcctagtattattcagaaaataaactagataatacaggttcctgattgatcctaattcagtatgtctcatctatgtaaatcagctattatggacaacttgataatccaggagcccaacccagggataaacagtatttctataacaaagatcaaaaaagaaggaaagactaaactggacagtttcattaacttcagacatttttaggcactttaaccaaatatcacatttgaggttagtagacaagtgaaggtctatgctactgagctcaaaagctaggggccttttatttatgtatttgttatgaggactatttttaagaatttattaaatattgaatatatttaagatgtttatattatatgtgcaaggtttgacaattatgttaaattacttttacccaccattgactttaagaaatagaacatggccttgaccatcaagtctttcatttgctccctatccccttcatcctctcctcacttcagaagtagcctcttcctgaattagtgtttattgttctcttgacttattttatcagttaacccttgtctttgttttccttcacacgcgtgctgcttacttttaaatatctgtgaactttatataaattatgtgtattatgctactcagatttatctatgtatttgggtatagttcaaattcatttactgctgctgcttagcattccagataatacatatccttttttctattttacatgcaaattattacttgtttgttgtgttttgtcttggtttgatgttgctattacgaacagtgctgatatgaatatttttagtcacatgcaaaatttcctcttggggatatcctgggaattcccctgctgtgttattgtgttggatcatcttcaactttctttaactttactaaatgaattcaattgttttccaaagtcatcctacaaaggtatcctctcatcaggaatgtatgagtgcactacatcctcatcagtgcttgtgctgggaatgaaacggcatctcttgttggttttaatttgcatttctcttgttgaaaatgaagttttcaacataactttacatttttacataattatttacataaataatatttttacatacaatatgttttatacatattatttttacataaacatatttttatataaattaattgaccattcagattttatcttttgtactgtgtctgttcatgatttttgcccatttttctgttaggttatttatcatttactgatttatcagaggtctcgtattgtggatactaatcctttgtcagctgcatatattgcaaatactttctcccagtttgggatttgaattttttactttctttgtgattcttttttttttttttgtctttttttttgtgaaagaaaagttttgaagtttaatctatatgaatgtattgatctttttattcctggcctttaaaaggaaaccatcatttagtatcttaaaaaaataccttttaccctgaaatcatgaagatgctctcttacactgttttccaaacgtaccacctgaaattgactttttgtattatattttgtgagacaagggttcggtttcattattttccatgtgatacccattgtcccaccattatttatgaattagcaatatcagttcagtcattcatatagtgtgtgtgtgtgtgtgtgtgtatatatatacatatatatatacatatatatatgtgtgcttgttctaggacatttattttattccattggtcactttgttcatccctcagccacaatggcagtgttttacttcttatagcttagagtaattcttgatatctgatgagttaatttcctctattgtttttcttatttaccagtatcttggctattttggccctttgcacttttatgtaaaatttaggttctgctacttgtttttaaaaaaggtatcatttttttaaaaaagtacacttttggaagctggaggcgagaagtttaagggatttatcaagtcacagtcttgttggtcacttaagtaagaatcagaattgggatccgagttgaacctagatctctacctccttgtcggcgttttctccactgtgtcatgcaatcttttacttaagcctgaggattactgataagtgaagaaagtgtatgctattttcaaaacagattttaacaccaatgatttccttttataaatttgaatttagccgtgcaaatacatctttgatacatagtttcatggtttgtaagcattggcatatttcacccacagatagagtttatttttagaataaaaaaagaaattatgttttttgattctactagtaaatctatgcatgttaaatgtggcatcacaactgtttcttccttctatgatgaagaatgtgctgcaactactgctgctcactcatgcctgccattgggttttattaccttactgtaattcctgattttcctcaatgattccactcctggtaaactgtcactaactaacattactttgtgttttcttgttataggatgattatgtacttgaagtgaggcactttcagtgtcctaaatggccaaatccagatagccccattagtaaaacttttgaacttataagtgttataaaagaagaagctgccaatagggatgggcctatgattgttcatgatgagtaagttccatgtgttagatggtttcacacctgcacattttctgggcatatgtatatttctgttgtcttcagacaaacctaatgctttggacccactgtgatgattcagccatggtggattatctgcatcttatcaataaagttaacacagaacattttttttttttttacttcacttgaagtccctgttgaagacagagaatggggtcatgatcacaaatatcttctcttgcatttgatgctgagaacactttgtatactaagtcggtgttttagcagtgaaattttcaggagccaaccagagaggcattttcagtcattgatttcctggcatagaagtgaaattgatatgatttttatttttcaggaacaagaaaccatccaataaatcttcctaggattccatcatagacaaataaaaaatgcatatgctgaacttatgttataaatatataaaatacagtttgttatatatgtatttatacattatattatatgtgtattatacatgtctttatatgatactctcaggttttttttattattgccaactcctttcatagtatacaaataataaaccataagcaaatactaaacttctgtaaagtattccactattgatatcatgaacacttggcaatatttcagattaaatcatagttctttgtgcaaaaagcgaagagagacaattcattaaaagtttcagtgttttcactgcaaagttgtcaaggctatatgctttgtgagttctaaatgtgagtggtgcatctcaatggagtctttgttccttttcttttttttttttacaaggcatggaggagtgacggcaggaactttctgtgctctgacaacccttatgcaccaactagaaaaagaaaattccgtggatgtttaccaggtagccaagatgatcaatctgatgaggccaggagtctttgctgacattgtaagtaacaaggcagtgaacgaaatttttcactgatagagtacaactaacttcctagaggctgctgaaatcttatgtatcaagaaagatcttattaactgataacattagtatgtagttttcaagctgaattacaagtccacaatccatagaacaacaacatttaaaatttatacttcagaaaatttatggataaaaacttttaaatctcatgtttgcctttgtgcacaatagtaaacaatgcaaataagcttaggtgaatgtagaagtcataggctcaccttcagctctttggtctgtttaagggccttataaagggaccttcttcaaagctccagcctagaccagttggcaggaggtcaaagttatcttcatgcacatgcactgcagaggagcccctaataagtcttattcggggccgctgagttgtatcaagcttgtgctgcatgaaactcctttctggagatgcacagttcgcgaaagcatttaattatgcactgcgtcacatgtaggcctcatggttttcggctatgatgctgctcactggttcaggttctctgtagctacctctccccaaactgcctgagatggcatctgtaagagaaaaactgtttcatgcttctatgtgccactgctctgagaccacagcagagaaggtacttacctttgctcatagcagggcataggaccagttgggctgatagaatgtgaaattcttcttattggtgaacagtgaaggattgcattctacatgggttcctgggctaacagtacagtcattcctgagatctttccgtaaagaggcagacccaatggattttctaacaagtagagaaagaagaaacttgtttctgagtgagttacagatgtccagtgccaaacggtttcgcccacaagttaacagaaccctgtctcatcaccttctgatttcatgaataagaattaagtacttattggcaaatgtatgtaggttaattttctaggtcagtgaatggtctgccgattgcaacgttaatttaatcagaagagacatcagtataggttgtgtgcatctgctgagtataacagtgcccttaacacattgccctttcatgaacagtgctgaagtgtgtctaaaaatatttcttctttttacaaatgtatgtcttcactgagcttcgcatttattacctcccatcttctgttctctaggagcagtatcagtttctctacaaagtgatcctcagccttgtgagcacaaggcaggaagagaatccatccacctctctggacagtaatggtgcagcattgcctgatggaaatatagctgagagcttagagtctttagtttaacacagaaaggggtgggggaactcacatctgagcattgttttcctcttcctaaaattaggcaggaaaatcagtctagttctgttatctgttgatttcccatcacctgacagtaactttcatgacataggattctgccgccaaatttatatcattaacaatgtgtgcctttttgcaagacttgtaatttacttattatgtttgaactaaaatgattgaattttacagtatttctaagaatggaattgtggtatttttttctgtattgattttaacagaaaatttcaatttatagaggttaggaattccaaactacagaaaatgtttgtttttagtgtcaaatttttagctgtatttgtagcaattatcaggtttgctagaaatataacttttaatacagtagcctgtaaataaaacactcttccatatgatattcaacattttacaactgcagtattcacctaaagtagaaataatctgttacttattgtaaatactgccctagtgtctccatggaccaaatttatatttataattgtagatttttatattttactactgagtcaagttttctagttctgtgtaattgtttagtttaatgacgtagttcattagctggtcttactctaccagttttctgacattgtattgtgttacctaagtcattaactttgtttcagcatgtaattttaacttttgtggaaaatagaaataccttcattttgaaagaagtttttatgagaataacaccttaccaaacattgttcaaatggtttttatccaaggaattgcaaaaataaatataaatattgccattaa

SEQ ID NO: 3 - RNA, 6402 nt, Homo sapiens
  source 1..6402: mol_type = mRNA
ggagcgcagggcgcaggggctggacccggcgcggagctggctgagtcggagcgcagcgtcctttgtgcccggcggccgccccgggatgcgtccgagctaggagccagatccaggatgggggccccgtttgtctgggccttgggccttttgatgctgcagatgctgctctttgtggctggggaacagggcacacaggatatcaccgatgccagcgaaagggggctccacatgcagaagctggggtctgggtcagtgcaggctgcgctggcggagctggtggccctgccctgtctctttaccctgcagccacggccaagcgcagcccgagatgcccctcggataaagtggaccaaggtgcggactgcgtcgggccagcgacaggacttgcccatcctggtggccaaggacaatgtcgtgagggtggccaaaagctggcagggacgagtgtcactgccttcctacccccggcgccgagccaacgccacgctacttctggggccactgagggccagtgactctgggctgtaccgctgccaggtggtgaggggcatcgaggatgagcaggacctggtgcccttggaggtgacaggtgttgtgttccactaccgatcagcccgggaccgctatgcactgaccttcgctgaggcccaggaggcctgccgtctcagctcagccatcattgcagcccctcggcatctacaggctgcctttgaggatggctttgacaactgtgatgctggctggctctctgaccgcactgttcggtatcctatcacccagtcccgtcctggttgctatggcgaccgtagcagccttccaggggttcggagctatgggaggcgcaacccacaggaactctacgatgtgtattgctttgcccgggagctggggggcgaggtcttctacgtgggcccggcccgccgcctgacactggccggcgcgcgtgcacagtgccgccgccagggtgccgcgctggcctcggtgggacagctgcacctggcctggcatgagggcctggaccagtgcgacccgggctggctggccgacggcagcgtgcgctacccgatccagacgccgcgccggcgctgcgggggcccagccccgggcgtgcgcaccgtctaccgcttcgctaaccggaccggcttcccctcacccgccgagcgcttcgacgcctactgcttccgagctcatcaccccacgtcacaacatggagacctagagaccccatcctctggggatgagggggagattctgtcagcagaggggcccccagttagagaactggagcccaccctggaggaggaagaggtggtcacccctgacttccaggagcctctggtgtccagtggggaagaagaaaccctgattttggaggagaagcaggagtctcaacagaccctcagccctacccctggggaccccatgctggcctcatggcccactggggaagtgtggctaagcacggtggcccccagccctagcgacatgggggcaggcactgcagcaagttcacacacggaggtggccccaactgaccctatgcctaggagaagggggcgcttcaaagggttgaatgggcgctacttccagcagcaggaaccggagccggggctgcaaggggggatggaggccagcgcccagccccccacctcagaggctgcagtgaaccaaatggagcctccgttggccatggcagtcacagagatgttgggcagtggccagagccggagcccctgggctgatctgaccaatgaggtggatatgcctggagctggttctgctggtggcaagagctccccagagccctggctgtggccccctaccatggtcccacccagcatctcaggccacagcagggcccctgtcctggagctagagaaagccgagggccccagtgccaggccagccaccccagacctgttttggtcccccttggaggccactgtctcagctcccagccctgccccctgggaggcattccctgtggccacctccccagatctccctatgatggccatgctgcgtggtcccaaagagtggatgctaccacaccccacccccatctccaccgaggccaatagagttgaggcacatggtgaggccaccgccacggctccaccctcccctgctgcagagaccaaggtgtattccctgcctctctctttgaccccaacaggacagggtggagaggccatgcccacaacacctgagtcccccagggcagacttcagagaaactggggagaccagccctgctcaggtcaacaaagctgagcactccagctccagcccatggccttctgtaaacaggaatgtggctgtaggttttgtccccactgagactgccactgagccaacgggcctcaggggtatcccggggtctgagtctggggtcttcgacacagcagaaagccccacttctggcttgcaggccactgtagatgaggtgcaggacccctggccctcagtgtacagcaaagggctggatgcaagttccccatctgcccccctggggagccctggagtcttcttggtacccaaagtcaccccaaatttggagccttgggttgctacagatgaaggacccactgtgaatcccatggattccacagtcacgccggcccccagtgatgctagtggaatttgggaacctggatcccaggtgtttgaagaagccgaaagcaccaccttgagccctcaggtggccctggatacaagcattgtgacgcccctcacgaccctggagcagggggacaaggttggagttccagccatgtctacactgggctcctcaagctcccaaccccacccagagccagaggatcaggtggagacccagggaacatcaggagcttcagtgcctccgcatcagagcagtcccctagggaaaccggctgttcctcctgggacaccgactgcagccagtgtgggcgagtctgcctcagtttcctcaggggagcctacggtaccgtgggacccctccagcaccctgctgcctgtcaccctgggcatagaggacttcgaactggaggtcctggcagggagcccgggtgtagagagcttctgggaggaggtggcaagtggagaggagccagccctgccagggacccctatgaatgcaggtgcggaggaggtgcactcagatccctgtgagaacaacccttgtcttcatggagggacatgtaatgccaatggcaccatgtatggctgtagctgtgatcagggcttcgccggggagaactgtgagattgacattgatgactgcctctgcagcccctgtgagaatggaggcacctgtattgatgaggtcaatggctttgtctgcctttgcctccccagctatgggggcagcttttgtgagaaagacaccgagggctgtgaccgcggctggcataagttccagggccactgttaccgctattttgcccaccggagggcatgggaagatgccgagaaggactgccgccgccgctccggccacctgaccagcgtccactcaccggaggaacacagcttcattaatagctttgggcatgaaaacacgtggatcggcctgaacgacaggatcgtggagagagatttccagtggacggacaacaccgggctgcaatttgagaactggcgagagaaccagccggacaatttcttcgcgggtggcgaggactgtgtggtgatggtggcgcatgaaagcgggcgctggaacgatgtcccctgcaactacaacctaccctatgtctgcaagaagggcacagtgctctgtggtccccctccggcagtggagaatgcctcactcatcggtgcccgcaaggccaagtacaatgtccatgccactgtaaggtaccagtgcaatgaaggatttgcccagcaccatgtggccaccattcgatgccggagcaatggcaagtgggacaggccccaaattgtctgcaccaaacccagacgttcacatcggatgcggcgacaccaccaccaccaccaacaccaccaccagcatcaccaccacaaatcccgcaaggagcgcagaaaacacaagaaacacccaacggaggactgggagaaggacgaagggaatttctgctgaagaaccagaaaaaagaaagcacaacacctttcccatgcctcctctggagccttcgcctggggagacagaacccagagagaaacaagagagtccagaagtccctgaaccccaaaccacatcccccacacacataatcctttgtacaaagctcctcttttccctttttttacatacacaagatcctcttggcaggtggagccaggtgtctgaaaagttcattctcgtctggctgaactctgggagtgtgtcccagctgagggaagcacaagtagcaaagctcattggtctggtctcttgtttgccaggctgattgaagcaggccttgatgagggtgcatgagtgtatgtttgcattcacatgaaggaattgcttttcacaccagaaattcagacttagtcaatgttggctgaattcctaaatccaggaagaagcctggacgtagggtcattagctttgggaatagaaggctacacagaagcacactgtttttgaacttgacaacagctctccctttaccctggacttcagcccaagttccgtctttggtcttggtggataaacacacagtgtggagatcccacgtactgcattttagggatgtttttaggacaacctccctccatgccttcagagttaggagtgagaatgatcaaagcaatatgtaggtgatggagggagagtgtattgctaacccttccaggtctagtccagcgctgagatttggtggttctgcatgtgtgatgaatctctttcacacaaatagacgagaggatatttagggctagatgagcccagatttcttccccctccatctctcagggagacaaagaacctccttcctggaccaaggaggtgctgccaagttttctagcccagtgcacatacccagtccttaagcagacattggtagtgcccctgccctgggtcccactcctgccccaccccacccttgtccctggccattgcctggtggtctagaaacacttaaaacttgaagtagtgacacctacctgcggtcatattgtagagagatgctcagtgttaaaactgaaacacacaaacacacacacacacacatttttctcttgtagattttaattttttaagtgggaaagaactcaccttgccttcctcccccaaatgtgcaacctgtaaaaggtctctccacaccaggggccaggatccagttccctcatctctggcaggaaagatccacagcttttcctccatgtctgttactcactttcagcagtccgggtaaaatctgtggatcagggttaaaaaagcaccgtggagaatggccctcttcaggaaagaaaaataagcaaatgaatggtccacctaggggttcagtaaagaaagaaatgtgttaactgagcctgaatcccttctgggaagtaataatgaccattgacaactaagaagtagacaccatgctaaagacttacatacaatctccttgaatcttctcaatagcccattgacttagaaactgttactttcccattttacacacagtgaaactgaggctcagatataaaggaaaggtactggcttgaagtcacaaccacgacaggagtaaggatttggaataaggatttggtcctgttttctggaccaaatccttactctggctctgcttacactttctctccatcaccaaatccttactccaaatccagaagtcagagccaactcccatcttggttctgacccaaatcctgctctggactctggagaggagattgaaatataattgcaccctcatacacatttaggaaatggttaagaagtgtaaactgaacccttatccttgtcttcaatcttcctccctgtagacatctatcttattatggttattattcagaaaacccagggatacaggtttgtcttcttactttgataactcttcttagtttaaaataataataataacacatctttggtcatctatgtcacacaaaaattttcctttgtttgcggggggctggggatgcagtgttttttggggggtcttggtttatgctccctgcccttgagcccctcagccgtttgccctgcccccacctcggctccatggtgggagggggctctggtcttttctaaagtgggcggtttgtcttttgatctttcccttttggatgtgcgtgtgtgtctgcgtgtgccatgtgcgtggcacgcatatgagtgtgtgtgcgtgtgaacggctttgggtcctgctggttttgctgtgagctgcagtgttctgtgggtctgtggtatctgacactgtggacattaatgtacttcttggacattttaataaattttttaacagttcaa

SEQ ID NO: 4 - RNA, 8103 nt, Homo sapiens
  source 1..8103: mol_type = mRNA
agaccgcggccgccgcagccggcgaaagaggcaaagtcccgcacgccggaggacatgcgcctcggctagcggccccgggccccaccaccgtgcggctttctccagattattcctctctcgctgtctctgactgtctctctctgtctctgtctctgtctctctctctctcacacacacacacacacacacaaacacacatacgcacgcacgatctcacttcgatctatacactggaggattaaaacaaacaaacaaaaaaaacatttccttcgctccccctccctctccactctgagaagcagaggagccgcacggcgaggggccgcagaccgtctggaaatgcgaatcctaaagcgtttcctcgcttgcattcagctcctctgtgtttgccgcctggattgggctaatggatactacagacaacagagaaaacttgttgaagagattggctggtcctatacaggagcactgaatcaaaaaaattggggaaagaaatatccaacatgtaatagcccaaaacaatctcctatcaatattgatgaagatcttacacaagtaaatgtgaatcttaagaaacttaaatttcagggttgggataaaacatcattggaaaacacattcattcataacactgggaaaacagtggaaattaatctcactaatgactaccgtgtcagcggaggagtttcagaaatggtgtttaaagcaagcaagataacttttcactggggaaaatgcaatatgtcatctgatggatcagagcatagtttagaaggacaaaaatttccacttgagatgcaaatctactgctttgatgcggaccgattttcaagttttgaggaagcagtcaaaggaaaagggaagttaagagctttatccattttgtttgaggttgggacagaagaaaatttggatttcaaagcgattattgatggagtcgaaagtgttagtcgttttgggaagcaggctgctttagatccattcatactgttgaaccttctgccaaactcaactgacaagtattacatttacaatggctcattgacatctcctccctgcacagacacagttgactggattgtttttaaagatacagttagcatctctgaaagccagttggctgttttttgtgaagttcttacaatgcaacaatctggttatgtcatgctgatggactacttacaaaacaattttcgagagcaacagtacaagttctctagacaggtgttttcctcatacactggaaaggaagagattcatgaagcagtttgtagttcagaaccagaaaatgttcaggctgacccagagaattataccagccttcttgttacatgggaaagacctcgagtcgtttatgataccatgattgagaagtttgcagttttgtaccagcagttggatggagaggaccaaaccaagcatgaatttttgacagatggctatcaagacttgggtgctattctcaataatttgctacccaatatgagttatgttcttcagatagtagccatatgcactaatggcttatatggaaaatacagcgaccaactgattgtcgacatgcctactgataatcctgaacttgatcttttccctgaattaattggaactgaagaaataatcaaggaggaggaagagggaaaagacattgaagaaggcgctattgtgaatcctggtagagacagtgctacaaaccaaatcaggaaaaaggaaccccagatttctaccacaacacactacaatcgcatagggacgaaatacaatgaagccaagactaaccgatccccaacaagaggaagtgaattctctggaaagggtgatgttcccaatacatctttaaattccacttcccaaccagtcactaaattagccacagaaaaagatatttccttgacttctcagactgtgactgaactgccacctcacactgtggaaggtacttcagcctctttaaatgatggctctaaaactgttcttagatctccacatatgaacttgtcggggactgcagaatccttaaatacagtttctataacagaatatgaggaggagagtttattgaccagtttcaagcttgatactggagctgaagattcttcaggctccagtcccgcaacttctgctatcccattcatctctgagaacatatcccaagggtatatattttcctccgaaaacccagagacaataacatatgatgtccttataccagaatctgctagaaatgcttccgaagattcaacttcatcaggttcagaagaatcactaaaggatccttctatggagggaaatgtgtggtttcctagctctacagacataacagcacagcccgatgttggatcaggcagagagagctttctccagactaattacactgagatacgtgttgatgaatctgagaagacaaccaagtccttttctgcaggcccagtgatgtcacagggtccctcagttacagatctggaaatgccacattattctacctttgcctacttcccaactgaggtaacacctcatgcttttaccccatcctccagacaacaggatttggtctccacggtcaacgtggtatactcgcagacaacccaaccggtatacaatggtgagacacctcttcaaccttcctacagtagtgaagtctttcctctagtcacccctttgttgcttgacaatcagatcctcaacactacccctgctgcttcaagtagtgattcggccttgcatgctacgcctgtatttcccagtgtcgatgtgtcatttgaatccatcctgtcttcctatgatggtgcacctttgcttccattttcctctgcttccttcagtagtgaattgtttcgccatctgcatacagtttctcaaatccttccacaagttacttcagctaccgagagtgataaggtgcccttgcatgcttctctgccagtggctgggggtgatttgctattagagcccagccttgctcagtattctgatgtgctgtccactactcatgctgcttcagagacgctggaatttggtagtgaatctggtgttctttataaaacgcttatgttttctcaagttgaaccacccagcagtgatgccatgatgcatgcacgttcttcagggcctgaaccttcttatgccttgtctgataatgagggctcccaacacatcttcactgtttcttacagttctgcaatacctgtgcatgattctgtgggtgtaacttatcagggttccttatttagcggccctagccatataccaatacctaagtcttcgttaataaccccaactgcatcattactgcagcctactcatgccctctctggtgatggggaatggtctggagcctcttctgatagtgaatttcttttacctgacacagatgggctgacagcccttaacatttcttcacctgtttctgtagctgaatttacatatacaacatctgtgtttggtgatgataataaggcgctttctaaaagtgaaataatatatggaaatgagactgaactgcaaattccttctttcaatgagatggtttacccttctgaaagcacagtcatgcccaacatgtatgataatgtaaataagttgaatgcgtctttacaagaaacctctgtttccatttctagcaccaagggcatgtttccagggtcccttgctcataccaccactaaggtttttgatcatgagattagtcaagttccagaaaataacttttcagttcaacctacacatactgtctctcaagcatctggtgacacttcgcttaaacctgtgcttagtgcaaactcagagccagcatcctctgaccctgcttctagtgaaatgttatctccttcaactcagctcttattttatgagacctcagcttcttttagtactgaagtattgctacaaccttcctttcaggcttctgatgttgacaccttgcttaaaactgttcttccagctgtgcccagtgatccaatattggttgaaacccccaaagttgataaaattagttctacaatgttgcatctcattgtatcaaattctgcttcaagtgaaaacatgctgcactctacatctgtaccagtttttgatgtgtcgcctacttctcatatgcactctgcttcacttcaaggtttgaccatttcctatgcaagtgagaaatatgaaccagttttgttaaaaagtgaaagttcccaccaagtggtaccttctttgtacagtaatgatgagttgttccaaacggccaatttggagattaaccaggcccatcccccaaaaggaaggcatgtatttgctacacctgttttatcaattgatgaaccattaaatacactaataaataagcttatacattccgatgaaattttaacctccaccaaaagttctgttactggtaaggtatttgctggtattccaacagttgcttctgatacatttgtatctactgatcattctgttcctataggaaatgggcatgttgccattacagctgtttctccccacagagatggttctgtaacctcaacaaagttgctgtttccttctaaggcaacttctgagctgagtcatagtgccaaatctgatgccggtttagtgggtggtggtgaagatggtgacactgatgatgatggtgatgatgatgatgatgacagaggtagtgatggcttatccattcataagtgtatgtcatgctcatcctatagagaatcacaggaaaaggtaatgaatgattcagacacccacgaaaacagtcttatggatcagaataatccaatctcatactcactatctgagaattctgaagaagataatagagtcacaagtgtatcctcagacagtcaaactggtatggacagaagtcctggtaaatcaccatcagcaaatgggctatcccaaaagcacaatgatggaaaagaggaaaatgacattcagactggtagtgctctgcttcctctcagccctgaatctaaagcatgggcagttctgacaagtgatgaagaaagtggatcagggcaaggtacctcagatagccttaatgagaatgagacttccacagatttcagttttgcagacactaatgaaaaagatgctgatgggatcctggcagcaggtgactcagaaataactcctggattcccacagtccccaacatcatctgttactagcgagaactcagaagtgttccacgtttcagaggcagaggccagtaatagtagccatgagtctcgtattggtctagctgaggggttggaatccgagaagaaggcagttataccccttgtgatcgtgtcagccctgacttttatctgtctagtggttcttgtgggtattctcatctactggaggaaatgcttccagactgcacacttttacttagaggacagtacatcccctagagttatatccacacctccaacacctatctttccaatttcagatgatgtcggagcaattccaataaagcactttccaaagcatgttgcagatttacatgcaagtagtgggtttactgaagaatttgagacactgaaagagttttaccaggaagtgcagagctgtactgttgacttaggtattacagcagacagctccaaccacccagacaacaagcacaagaatcgatacataaatatcgttgcctatgatcatagcagggttaagctagcacagcttgctgaaaaggatggcaaactgactgattatatcaatgccaattatgttgatggctacaacagaccaaaagcttatattgctgcccaaggcccactgaaatccacagctgaagatttctggagaatgatatgggaacataatgtggaagttattgtcatgataacaaacctcgtggagaaaggaaggagaaaatgtgatcagtactggcctgccgatgggagtgaggagtacgggaactttctggtcactcagaagagtgtgcaagtgcttgcctattatactgtgaggaattttactctaagaaacacaaaaataaaaaagggctcccagaaaggaagacccagtggacgtgtggtcacacagtatcactacacgcagtggcctgacatgggagtaccagagtactccctgccagtgctgacctttgtgagaaaggcagcctatgccaagcgccatgcagtggggcctgttgtcgtccactgcagtgctggagttggaagaacaggcacatatattgtgctagacagtatgttgcagcagattcaacacgaaggaactgtcaacatatttggcttcttaaaacacatccgttcacaaagaaattatttggtacaaactgaggagcaatatgtcttcattcatgatacactggttgaggccatacttagtaaagaaactgaggtgctggacagtcatattcatgcctatgttaatgcactcctcattcctggaccagcaggcaaaacaaagctagagaaacaattccagctcctgagccagtcaaatatacagcagagtgactattctgcagccctaaagcaatgcaacagggaaaagaatcgaacttcttctatcatccctgtggaaagatcaagggttggcatttcatccctgagtggagaaggcacagactacatcaatgcctcctatatcatgggctattaccagagcaatgaattcatcattacccagcaccctctccttcataccatcaaggatttctggaggatgatatgggaccataatgcccaactggtggttatgattcctgatggccaaaacatggcagaagatgaatttgtttactggccaaataaagatgagcctataaattgtgagagctttaaggtcactcttatggctgaagaacacaaatgtctatctaatgaggaaaaacttataattcaggactttatcttagaagctacacaggatgattatgtacttgaagtgaggcactttcagtgtcctaaatggccaaatccagatagccccattagtaaaacttttgaacttataagtgttataaaagaagaagctgccaatagggatgggcctatgattgttcatgatgagcatggaggagtgacggcaggaactttctgtgctctgacaacccttatgcaccaactagaaaaagaaaattccgtggatgtttaccaggtagccaagatgatcaatctgatgaggccaggagtctttgctgacattgagcagtatcagtttctctacaaagtgatcctcagccttgtgagcacaaggcaggaagagaatccatccacctctctggacagtaatggtgcagcattgcctgatggaaatatagctgagagcttagagtctttagtttaacacagaaaggggtgggggaactcacatctgagcattgttttcctcttcctaaaattaggcaggaaaatcagtctagttctgttatctgttgatttcccatcacctgacagtaactttcatgacataggattctgccgccaaatttatatcattaacaatgtgtgcctttttgcaagacttgtaatttacttattatgtttgaactaaaatgattgaattttacagtatttctaagaatggaattgtggtatttttttctgtattgattttaacagaaaatttcaatttatagaggttaggaattccaaactacagaaaatgtttgtttttagtgtcaaatttttagctgtatttgtagcaattatcaggtttgctagaaatataacttttaatacagtagcctgtaaataaaacactcttccatatgatattcaacattttacaactgcagtattcacctaaagtagaaataatctgttacttattgtaaatactgccctagtgtctccatggaccaaatttatatttataattgtagatttttatattttactactgagtcaagttttctagttctgtgtaattgtttagtttaatgacgtagttcattagctggtcttactctaccagttttctgacattgtattgtgttacctaagtcattaactttgtttcagcatgtaattttaacttttgtggaaaatagaaataccttcattttgaaagaagtttttatgagaataacaccttaccaaacattgttcaaatggtttttatccaaggaattgcaaaaataaatataaatattgccattaa

SEQ ID NO: 5 - RNA, 5523 nt, Homo sapiens
  source 1..5523: mol_type = mRNA
agaccgcggccgccgcagccggcgaaagaggcaaagtcccgcacgccggaggacatgcgcctcggctagcggccccgggccccaccaccgtgcggctttctccagattattcctctctcgctgtctctgactgtctctctctgtctctgtctctgtctctctctctctcacacacacacacacacacacaaacacacatacgcacgcacgatctcacttcgatctatacactggaggattaaaacaaacaaacaaaaaaaacatttccttcgctccccctccctctccactctgagaagcagaggagccgcacggcgaggggccgcagaccgtctggaaatgcgaatcctaaagcgtttcctcgcttgcattcagctcctctgtgtttgccgcctggattgggctaatggatactacagacaacagagaaaacttgttgaagagattggctggtcctatacaggagcactgaatcaaaaaaattggggaaagaaatatccaacatgtaatagcccaaaacaatctcctatcaatattgatgaagatcttacacaagtaaatgtgaatcttaagaaacttaaatttcagggttgggataaaacatcattggaaaacacattcattcataacactgggaaaacagtggaaattaatctcactaatgactaccgtgtcagcggaggagtttcagaaatggtgtttaaagcaagcaagataacttttcactggggaaaatgcaatatgtcatctgatggatcagagcatagtttagaaggacaaaaatttccacttgagatgcaaatctactgctttgatgcggaccgattttcaagttttgaggaagcagtcaaaggaaaagggaagttaagagctttatccattttgtttgaggttgggacagaagaaaatttggatttcaaagcgattattgatggagtcgaaagtgttagtcgttttgggaagcaggctgctttagatccattcatactgttgaaccttctgccaaactcaactgacaagtattacatttacaatggctcattgacatctcctccctgcacagacacagttgactggattgtttttaaagatacagttagcatctctgaaagccagttggctgttttttgtgaagttcttacaatgcaacaatctggttatgtcatgctgatggactacttacaaaacaattttcgagagcaacagtacaagttctctagacaggtgttttcctcatacactggaaaggaagagattcatgaagcagtttgtagttcagaaccagaaaatgttcaggctgacccagagaattataccagccttcttgttacatgggaaagacctcgagtcgtttatgataccatgattgagaagtttgcagttttgtaccagcagttggatggagaggaccaaaccaagcatgaatttttgacagatggctatcaagacttgggtgctattctcaataatttgctacccaatatgagttatgttcttcagatagtagccatatgcactaatggcttatatggaaaatacagcgaccaactgattgtcgacatgcctactgataatcctgaacttgatcttttccctgaattaattggaactgaagaaataatcaaggaggaggaagagggaaaagacattgaagaaggcgctattgtgaatcctggtagagacagtgctacaaaccaaatcaggaaaaaggaaccccagatttctaccacaacacactacaatcgcatagggacgaaatacaatgaagccaagactaaccgatccccaacaagaggaagtgaattctctggaaagggtgatgttcccaatacatctttaaattccacttcccaaccagtcactaaattagccacagaaaaagatatttccttgacttctcagactgtgactgaactgccacctcacactgtggaaggtacttcagcctctttaaatgatggctctaaaactgttcttagatctccacatatgaacttgtcggggactgcagaatccttaaatacagtttctataacagaatatgaggaggagagtttattgaccagtttcaagcttgatactggagctgaagattcttcaggctccagtcccgcaacttctgctatcccattcatctctgagaacatatcccaagggtatatattttcctccgaaaacccagagacaataacatatgatgtccttataccagaatctgctagaaatgcttccgaagattcaacttcatcaggttcagaagaatcactaaaggatccttctatggagggaaatgtgtggtttcctagctctacagacataacagcacagcccgatgttggatcaggcagagagagctttctccagactaattacactgagatacgtgttgatgaatctgagaagacaaccaagtccttttctgcaggcccagtgatgtcacagggtccctcagttacagatctggaaatgccacattattctacctttgcctacttcccaactgaggtaacacctcatgcttttaccccatcctccagacaacaggatttggtctccacggtcaacgtggtatactcgcagacaacccaaccggtatacaatgaggccagtaatagtagccatgagtctcgtattggtctagctgaggggttggaatccgagaagaaggcagttataccccttgtgatcgtgtcagccctgacttttatctgtctagtggttcttgtgggtattctcatctactggaggaaatgcttccagactgcacacttttacttagaggacagtacatcccctagagttatatccacacctccaacacctatctttccaatttcagatgatgtcggagcaattccaataaagcactttccaaagcatgttgcagatttacatgcaagtagtgggtttactgaagaatttgagacactgaaagagttttaccaggaagtgcagagctgtactgttgacttaggtattacagcagacagctccaaccacccagacaacaagcacaagaatcgatacataaatatcgttgcctatgatcatagcagggttaagctagcacagcttgctgaaaaggatggcaaactgactgattatatcaatgccaattatgttgatggctacaacagaccaaaagcttatattgctgcccaaggcccactgaaatccacagctgaagatttctggagaatgatatgggaacataatgtggaagttattgtcatgataacaaacctcgtggagaaaggaaggagaaaatgtgatcagtactggcctgccgatgggagtgaggagtacgggaactttctggtcactcagaagagtgtgcaagtgcttgcctattatactgtgaggaattttactctaagaaacacaaaaataaaaaagggctcccagaaaggaagacccagtggacgtgtggtcacacagtatcactacacgcagtggcctgacatgggagtaccagagtactccctgccagtgctgacctttgtgagaaaggcagcctatgccaagcgccatgcagtggggcctgttgtcgtccactgcagtgctggagttggaagaacaggcacatatattgtgctagacagtatgttgcagcagattcaacacgaaggaactgtcaacatatttggcttcttaaaacacatccgttcacaaagaaattatttggtacaaactgaggagcaatatgtcttcattcatgatacactggttgaggccatacttagtaaagaaactgaggtgctggacagtcatattcatgcctatgttaatgcactcctcattcctggaccagcaggcaaaacaaagctagagaaacaattccagctcctgagccagtcaaatatacagcagagtgactattctgcagccctaaagcaatgcaacagggaaaagaatcgaacttcttctatcatccctgtggaaagatcaagggttggcatttcatccctgagtggagaaggcacagactacatcaatgcctcctatatcatgggctattaccagagcaatgaattcatcattacccagcaccctctccttcataccatcaaggatttctggaggatgatatgggaccataatgcccaactggtggttatgattcctgatggccaaaacatggcagaagatgaatttgtttactggccaaataaagatgagcctataaattgtgagagctttaaggtcactcttatggctgaagaacacaaatgtctatctaatgaggaaaaacttataattcaggactttatcttagaagctacacaggatgattatgtacttgaagtgaggcactttcagtgtcctaaatggccaaatccagatagccccattagtaaaacttttgaacttataagtgttataaaagaagaagctgccaatagggatgggcctatgattgttcatgatgagcatggaggagtgacggcaggaactttctgtgctctgacaacccttatgcaccaactagaaaaagaaaattccgtggatgtttaccaggtagccaagatgatcaatctgatgaggccaggagtctttgctgacattgagcagtatcagtttctctacaaagtgatcctcagccttgtgagcacaaggcaggaagagaatccatccacctctctggacagtaatggtgcagcattgcctgatggaaatatagctgagagcttagagtctttagtttaacacagaaaggggtgggggaactcacatctgagcattgttttcctcttcctaaaattaggcaggaaaatcagtctagttctgttatctgttgatttcccatcacctgacagtaactttcatgacataggattctgccgccaaatttatatcattaacaatgtgtgcctttttgcaagacttgtaatttacttattatgtttgaactaaaatgattgaattttacagtatttctaagaatggaattgtggtatttttttctgtattgattttaacagaaaatttcaatttatagaggttaggaattccaaactacagaaaatgtttgtttttagtgtcaaatttttagctgtatttgtagcaattatcaggtttgctagaaatataacttttaatacagtagcctgtaaataaaacactcttccatatgatattcaacattttacaactgcagtattcacctaaagtagaaataatctgttacttattgtaaatactgccctagtgtctccatggaccaaatttatatttataattgtagatttttatattttactactgagtcaagttttctagttctgtgtaattgtttagtttaatgacgtagttcattagctggtcttactctaccagttttctgacattgtattgtgttacctaagtcattaactttgtttcagcatgtaattttaacttttgtggaaaatagaaataccttcattttgaaagaagtttttatgagaataacaccttaccaaacattgttcaaatggtttttatccaaggaattgcaaaaataaatataaatattgccattaa

SEQ ID NO: 6 - RNA, 5502 nt, Homo sapiens
  source 1..5502: mol_type = mRNA
agaccgcggccgccgcagccggcgaaagaggcaaagtcccgcacgccggaggacatgcgcctcggctagcggccccgggccccaccaccgtgcggctttctccagattattcctctctcgctgtctctgactgtctctctctgtctctgtctctgtctctctctctctcacacacacacacacacacacaaacacacatacgcacgcacgatctcacttcgatctatacactggaggattaaaacaaacaaacaaaaaaaacatttccttcgctccccctccctctccactctgagaagcagaggagccgcacggcgaggggccgcagaccgtctggaaatgcgaatcctaaagcgtttcctcgcttgcattcagctcctctgtgtttgccgcctggattgggctaatggatactacagacaacagagaaaacttgttgaagagattggctggtcctatacaggagcactgaatcaaaaaaattggggaaagaaatatccaacatgtaatagcccaaaacaatctcctatcaatattgatgaagatcttacacaagtaaatgtgaatcttaagaaacttaaatttcagggttgggataaaacatcattggaaaacacattcattcataacactgggaaaacagtggaaattaatctcactaatgactaccgtgtcagcggaggagtttcagaaatggtgtttaaagcaagcaagataacttttcactggggaaaatgcaatatgtcatctgatggatcagagcatagtttagaaggacaaaaatttccacttgagatgcaaatctactgctttgatgcggaccgattttcaagttttgaggaagcagtcaaaggaaaagggaagttaagagctttatccattttgtttgaggttgggacagaagaaaatttggatttcaaagcgattattgatggagtcgaaagtgttagtcgttttgggaagcaggctgctttagatccattcatactgttgaaccttctgccaaactcaactgacaagtattacatttacaatggctcattgacatctcctccctgcacagacacagttgactggattgtttttaaagatacagttagcatctctgaaagccagttggctgttttttgtgaagttcttacaatgcaacaatctggttatgtcatgctgatggactacttacaaaacaattttcgagagcaacagtacaagttctctagacaggtgttttcctcatacactggaaaggaagagattcatgaagcagtttgtagttcagaaccagaaaatgttcaggctgacccagagaattataccagccttcttgttacatgggaaagacctcgagtcgtttatgataccatgattgagaagtttgcagttttgtaccagcagttggatggagaggaccaaaccaagcatgaatttttgacagatggctatcaagacttgggtgctattctcaataatttgctacccaatatgagttatgttcttcagatagtagccatatgcactaatggcttatatggaaaatacagcgaccaactgattgtcgacatgcctactgataatcctgaacttgatcttttccctgaattaattggaactgaagaaataatcaaggaggaggaagagggaaaagacattgaagaaggcgctattgtgaatcctggtagagacagtgctacaaaccaaatcaggaaaaaggaaccccagatttctaccacaacacactacaatcgcatagggacgaaatacaatgaagccaagactaaccgatccccaacaagaggaagtgaattctctggaaagggtgatgttcccaatacatctttaaattccacttcccaaccagtcactaaattagccacagaaaaagatatttccttgacttctcagactgtgactgaactgccacctcacactgtggaaggtacttcagcctctttaaatgatggctctaaaactgttcttagatctccacatatgaacttgtcggggactgcagaatccttaaatacagtttctataacagaatatgaggaggagagtttattgaccagtttcaagcttgatactggagctgaagattcttcaggctccagtcccgcaacttctgctatcccattcatctctgagaacatatcccaagggtatatattttcctccgaaaacccagagacaataacatatgatgtccttataccagaatctgctagaaatgcttccgaagattcaacttcatcaggttcagaagaatcactaaaggatccttctatggagggaaatgtgtggtttcctagctctacagacataacagcacagcccgatgttggatcaggcagagagagctttctccagactaattacactgagatacgtgttgatgaatctgagaagacaaccaagtccttttctgcaggcccagtgatgtcacagggtccctcagttacagatctggaaatgccacattattctacctttgcctacttcccaactgaggtaacacctcatgcttttaccccatcctccagacaacaggatttggtctccacggtcaacgtggtatactcgcagacaacccaaccggtatacaatgaggccagtaatagtagccatgagtctcgtattggtctagctgaggggttggaatccgagaagaaggcagttataccccttgtgatcgtgtcagccctgacttttatctgtctagtggttcttgtgggtattctcatctactggaggaaatgcttccagactgcacacttttacttagaggacagtacatcccctagagttatatccacacctccaacacctatctttccaatttcagatgatgtcggagcaattccaataaagcactttccaaagcatgttgcagatttacatgcaagtagtgggtttactgaagaatttgaggaagtgcagagctgtactgttgacttaggtattacagcagacagctccaaccacccagacaacaagcacaagaatcgatacataaatatcgttgcctatgatcatagcagggttaagctagcacagcttgctgaaaaggatggcaaactgactgattatatcaatgccaattatgttgatggctacaacagaccaaaagcttatattgctgcccaaggcccactgaaatccacagctgaagatttctggagaatgatatgggaacataatgtggaagttattgtcatgataacaaacctcgtggagaaaggaaggagaaaatgtgatcagtactggcctgccgatgggagtgaggagtacgggaactttctggtcactcagaagagtgtgcaagtgcttgcctattatactgtgaggaattttactctaagaaacacaaaaataaaaaagggctcccagaaaggaagacccagtggacgtgtggtcacacagtatcactacacgcagtggcctgacatgggagtaccagagtactccctgccagtgctgacctttgtgagaaaggcagcctatgccaagcgccatgcagtggggcctgttgtcgtccactgcagtgctggagttggaagaacaggcacatatattgtgctagacagtatgttgcagcagattcaacacgaaggaactgtcaacatatttggcttcttaaaacacatccgttcacaaagaaattatttggtacaaactgaggagcaatatgtcttcattcatgatacactggttgaggccatacttagtaaagaaactgaggtgctggacagtcatattcatgcctatgttaatgcactcctcattcctggaccagcaggcaaaacaaagctagagaaacaattccagctcctgagccagtcaaatatacagcagagtgactattctgcagccctaaagcaatgcaacagggaaaagaatcgaacttcttctatcatccctgtggaaagatcaagggttggcatttcatccctgagtggagaaggcacagactacatcaatgcctcctatatcatgggctattaccagagcaatgaattcatcattacccagcaccctctccttcataccatcaaggatttctggaggatgatatgggaccataatgcccaactggtggttatgattcctgatggccaaaacatggcagaagatgaatttgtttactggccaaataaagatgagcctataaattgtgagagctttaaggtcactcttatggctgaagaacacaaatgtctatctaatgaggaaaaacttataattcaggactttatcttagaagctacacaggatgattatgtacttgaagtgaggcactttcagtgtcctaaatggccaaatccagatagccccattagtaaaacttttgaacttataagtgttataaaagaagaagctgccaatagggatgggcctatgattgttcatgatgagcatggaggagtgacggcaggaactttctgtgctctgacaacccttatgcaccaactagaaaaagaaaattccgtggatgtttaccaggtagccaagatgatcaatctgatgaggccaggagtctttgctgacattgagcagtatcagtttctctacaaagtgatcctcagccttgtgagcacaaggcaggaagagaatccatccacctctctggacagtaatggtgcagcattgcctgatggaaatatagctgagagcttagagtctttagtttaacacagaaaggggtgggggaactcacatctgagcattgttttcctcttcctaaaattaggcaggaaaatcagtctagttctgttatctgttgatttcccatcacctgacagtaactttcatgacataggattctgccgccaaatttatatcattaacaatgtgtgcctttttgcaagacttgtaatttacttattatgtttgaactaaaatgattgaattttacagtatttctaagaatggaattgtggtatttttttctgtattgattttaacagaaaatttcaatttatagaggttaggaattccaaactacagaaaatgtttgtttttagtgtcaaatttttagctgtatttgtagcaattatcaggtttgctagaaatataacttttaatacagtagcctgtaaataaaacactcttccatatgatattcaacattttacaactgcagtattcacctaaagtagaaataatctgttacttattgtaaatactgccctagtgtctccatggaccaaatttatatttataattgtagatttttatattttactactgagtcaagttttctagttctgtgtaattgtttagtttaatgacgtagttcattagctggtcttactctaccagttttctgacattgtattgtgttacctaagtcattaactttgtttcagcatgtaattttaacttttgtggaaaatagaaataccttcattttgaaagaagtttttatgagaataacaccttaccaaacattgttcaaatggtttttatccaaggaattgcaaaaataaatataaatattgccattaa

SEQ ID NO: 7 - RNA, 8082 nt, Homo sapiens
  source 1..8082: mol_type = mRNA
agaccgcggccgccgcagccggcgaaagaggcaaagtcccgcacgccggaggacatgcgcctcggctagcggccccgggccccaccaccgtgcggctttctccagattattcctctctcgctgtctctgactgtctctctctgtctctgtctctgtctctctctctctcacacacacacacacacacacaaacacacatacgcacgcacgatctcacttcgatctatacactggaggattaaaacaaacaaacaaaaaaaacatttccttcgctccccctccctctccactctgagaagcagaggagccgcacggcgaggggccgcagaccgtctggaaatgcgaatcctaaagcgtttcctcgcttgcattcagctcctctgtgtttgccgcctggattgggctaatggatactacagacaacagagaaaacttgttgaagagattggctggtcctatacaggagcactgaatcaaaaaaattggggaaagaaatatccaacatgtaatagcccaaaacaatctcctatcaatattgatgaagatcttacacaagtaaatgtgaatcttaagaaacttaaatttcagggttgggataaaacatcattggaaaacacattcattcataacactgggaaaacagtggaaattaatctcactaatgactaccgtgtcagcggaggagtttcagaaatggtgtttaaagcaagcaagataacttttcactggggaaaatgcaatatgtcatctgatggatcagagcatagtttagaaggacaaaaatttccacttgagatgcaaatctactgctttgatgcggaccgattttcaagttttgaggaagcagtcaaaggaaaagggaagttaagagctttatccattttgtttgaggttgggacagaagaaaatttggatttcaaagcgattattgatggagtcgaaagtgttagtcgttttgggaagcaggctgctttagatccattcatactgttgaaccttctgccaaactcaactgacaagtattacatttacaatggctcattgacatctcctccctgcacagacacagttgactggattgtttttaaagatacagttagcatctctgaaagccagttggctgttttttgtgaagttcttacaatgcaacaatctggttatgtcatgctgatggactacttacaaaacaattttcgagagcaacagtacaagttctctagacaggtgttttcctcatacactggaaaggaagagattcatgaagcagtttgtagttcagaaccagaaaatgttcaggctgacccagagaattataccagccttcttgttacatgggaaagacctcgagtcgtttatgataccatgattgagaagtttgcagttttgtaccagcagttggatggagaggaccaaaccaagcatgaatttttgacagatggctatcaagacttgggtgctattctcaataatttgctacccaatatgagttatgttcttcagatagtagccatatgcactaatggcttatatggaaaatacagcgaccaactgattgtcgacatgcctactgataatcctgaacttgatcttttccctgaattaattggaactgaagaaataatcaaggaggaggaagagggaaaagacattgaagaaggcgctattgtgaatcctggtagagacagtgctacaaaccaaatcaggaaaaaggaaccccagatttctaccacaacacactacaatcgcatagggacgaaatacaatgaagccaagactaaccgatccccaacaagaggaagtgaattctctggaaagggtgatgttcccaatacatctttaaattccacttcccaaccagtcactaaattagccacagaaaaagatatttccttgacttctcagactgtgactgaactgccacctcacactgtggaaggtacttcagcctctttaaatgatggctctaaaactgttcttagatctccacatatgaacttgtcggggactgcagaatccttaaatacagtttctataacagaatatgaggaggagagtttattgaccagtttcaagcttgatactggagctgaagattcttcaggctccagtcccgcaacttctgctatcccattcatctctgagaacatatcccaagggtatatattttcctccgaaaacccagagacaataacatatgatgtccttataccagaatctgctagaaatgcttccgaagattcaacttcatcaggttcagaagaatcactaaaggatccttctatggagggaaatgtgtggtttcctagctctacagacataacagcacagcccgatgttggatcaggcagagagagctttctccagactaattacactgagatacgtgttgatgaatctgagaagacaaccaagtccttttctgcaggcccagtgatgtcacagggtccctcagttacagatctggaaatgccacattattctacctttgcctacttcccaactgaggtaacacctcatgcttttaccccatcctccagacaacaggatttggtctccacggtcaacgtggtatactcgcagacaacccaaccggtatacaatggtgagacacctcttcaaccttcctacagtagtgaagtctttcctctagtcacccctttgttgcttgacaatcagatcctcaacactacccctgctgcttcaagtagtgattcggccttgcatgctacgcctgtatttcccagtgtcgatgtgtcatttgaatccatcctgtcttcctatgatggtgcacctttgcttccattttcctctgcttccttcagtagtgaattgtttcgccatctgcatacagtttctcaaatccttccacaagttacttcagctaccgagagtgataaggtgcccttgcatgcttctctgccagtggctgggggtgatttgctattagagcccagccttgctcagtattctgatgtgctgtccactactcatgctgcttcagagacgctggaatttggtagtgaatctggtgttctttataaaacgcttatgttttctcaagttgaaccacccagcagtgatgccatgatgcatgcacgttcttcagggcctgaaccttcttatgccttgtctgataatgagggctcccaacacatcttcactgtttcttacagttctgcaatacctgtgcatgattctgtgggtgtaacttatcagggttccttatttagcggccctagccatataccaatacctaagtcttcgttaataaccccaactgcatcattactgcagcctactcatgccctctctggtgatggggaatggtctggagcctcttctgatagtgaatttcttttacctgacacagatgggctgacagcccttaacatttcttcacctgtttctgtagctgaatttacatatacaacatctgtgtttggtgatgataataaggcgctttctaaaagtgaaataatatatggaaatgagactgaactgcaaattccttctttcaatgagatggtttacccttctgaaagcacagtcatgcccaacatgtatgataatgtaaataagttgaatgcgtctttacaagaaacctctgtttccatttctagcaccaagggcatgtttccagggtcccttgctcataccaccactaaggtttttgatcatgagattagtcaagttccagaaaataacttttcagttcaacctacacatactgtctctcaagcatctggtgacacttcgcttaaacctgtgcttagtgcaaactcagagccagcatcctctgaccctgcttctagtgaaatgttatctccttcaactcagctcttattttatgagacctcagcttcttttagtactgaagtattgctacaaccttcctttcaggcttctgatgttgacaccttgcttaaaactgttcttccagctgtgcccagtgatccaatattggttgaaacccccaaagttgataaaattagttctacaatgttgcatctcattgtatcaaattctgcttcaagtgaaaacatgctgcactctacatctgtaccagtttttgatgtgtcgcctacttctcatatgcactctgcttcacttcaaggtttgaccatttcctatgcaagtgagaaatatgaaccagttttgttaaaaagtgaaagttcccaccaagtggtaccttctttgtacagtaatgatgagttgttccaaacggccaatttggagattaaccaggcccatcccccaaaaggaaggcatgtatttgctacacctgttttatcaattgatgaaccattaaatacactaataaataagcttatacattccgatgaaattttaacctccaccaaaagttctgttactggtaaggtatttgctggtattccaacagttgcttctgatacatttgtatctactgatcattctgttcctataggaaatgggcatgttgccattacagctgtttctccccacagagatggttctgtaacctcaacaaagttgctgtttccttctaaggcaacttctgagctgagtcatagtgccaaatctgatgccggtttagtgggtggtggtgaagatggtgacactgatgatgatggtgatgatgatgatgatgacagaggtagtgatggcttatccattcataagtgtatgtcatgctcatcctatagagaatcacaggaaaaggtaatgaatgattcagacacccacgaaaacagtcttatggatcagaataatccaatctcatactcactatctgagaattctgaagaagataatagagtcacaagtgtatcctcagacagtcaaactggtatggacagaagtcctggtaaatcaccatcagcaaatgggctatcccaaaagcacaatgatggaaaagaggaaaatgacattcagactggtagtgctctgcttcctctcagccctgaatctaaagcatgggcagttctgacaagtgatgaagaaagtggatcagggcaaggtacctcagatagccttaatgagaatgagacttccacagatttcagttttgcagacactaatgaaaaagatgctgatgggatcctggcagcaggtgactcagaaataactcctggattcccacagtccccaacatcatctgttactagcgagaactcagaagtgttccacgtttcagaggcagaggccagtaatagtagccatgagtctcgtattggtctagctgaggggttggaatccgagaagaaggcagttataccccttgtgatcgtgtcagccctgacttttatctgtctagtggttcttgtgggtattctcatctactggaggaaatgcttccagactgcacacttttacttagaggacagtacatcccctagagttatatccacacctccaacacctatctttccaatttcagatgatgtcggagcaattccaataaagcactttccaaagcatgttgcagatttacatgcaagtagtgggtttactgaagaatttgaggaagtgcagagctgtactgttgacttaggtattacagcagacagctccaaccacccagacaacaagcacaagaatcgatacataaatatcgttgcctatgatcatagcagggttaagctagcacagcttgctgaaaaggatggcaaactgactgattatatcaatgccaattatgttgatggctacaacagaccaaaagcttatattgctgcccaaggcccactgaaatccacagctgaagatttctggagaatgatatgggaacataatgtggaagttattgtcatgataacaaacctcgtggagaaaggaaggagaaaatgtgatcagtactggcctgccgatgggagtgaggagtacgggaactttctggtcactcagaagagtgtgcaagtgcttgcctattatactgtgaggaattttactctaagaaacacaaaaataaaaaagggctcccagaaaggaagacccagtggacgtgtggtcacacagtatcactacacgcagtggcctgacatgggagtaccagagtactccctgccagtgctgacctttgtgagaaaggcagcctatgccaagcgccatgcagtggggcctgttgtcgtccactgcagtgctggagttggaagaacaggcacatatattgtgctagacagtatgttgcagcagattcaacacgaaggaactgtcaacatatttggcttcttaaaacacatccgttcacaaagaaattatttggtacaaactgaggagcaatatgtcttcattcatgatacactggttgaggccatacttagtaaagaaactgaggtgctggacagtcatattcatgcctatgttaatgcactcctcattcctggaccagcaggcaaaacaaagctagagaaacaattccagctcctgagccagtcaaatatacagcagagtgactattctgcagccctaaagcaatgcaacagggaaaagaatcgaacttcttctatcatccctgtggaaagatcaagggttggcatttcatccctgagtggagaaggcacagactacatcaatgcctcctatatcatgggctattaccagagcaatgaattcatcattacccagcaccctctccttcataccatcaaggatttctggaggatgatatgggaccataatgcccaactggtggttatgattcctgatggccaaaacatggcagaagatgaatttgtttactggccaaataaagatgagcctataaattgtgagagctttaaggtcactcttatggctgaagaacacaaatgtctatctaatgaggaaaaacttataattcaggactttatcttagaagctacacaggatgattatgtacttgaagtgaggcactttcagtgtcctaaatggccaaatccagatagccccattagtaaaacttttgaacttataagtgttataaaagaagaagctgccaatagggatgggcctatgattgttcatgatgagcatggaggagtgacggcaggaactttctgtgctctgacaacccttatgcaccaactagaaaaagaaaattccgtggatgtttaccaggtagccaagatgatcaatctgatgaggccaggagtctttgctgacattgagcagtatcagtttctctacaaagtgatcctcagccttgtgagcacaaggcaggaagagaatccatccacctctctggacagtaatggtgcagcattgcctgatggaaatatagctgagagcttagagtctttagtttaacacagaaaggggtgggggaactcacatctgagcattgttttcctcttcctaaaattaggcaggaaaatcagtctagttctgttatctgttgatttcccatcacctgacagtaactttcatgacataggattctgccgccaaatttatatcattaacaatgtgtgcctttttgcaagacttgtaatttacttattatgtttgaactaaaatgattgaattttacagtatttctaagaatggaattgtggtatttttttctgtattgattttaacagaaaatttcaatttatagaggttaggaattccaaactacagaaaatgtttgtttttagtgtcaaatttttagctgtatttgtagcaattatcaggtttgctagaaatataacttttaatacagtagcctgtaaataaaacactcttccatatgatattcaacattttacaactgcagtattcacctaaagtagaaataatctgttacttattgtaaatactgccctagtgtctccatggaccaaatttatatttataattgtagatttttatattttactactgagtcaagttttctagttctgtgtaattgtttagtttaatgacgtagttcattagctggtcttactctaccagttttctgacattgtattgtgttacctaagtcattaactttgtttcagcatgtaattttaacttttgtggaaaatagaaataccttcattttgaaagaagtttttatgagaataacaccttaccaaacattgttcaaatggtttttatccaaggaattgcaaaaataaatataaatattgccattaa

SEQ ID NO: 8 - RNA, 8206 nt, Homo sapiens
  source 1..8206: mol_type = mRNA
agaccgcggccgccgcagccggcgaaagaggcaaagtcccgcacgccggaggacatgcgcctcggctagcggccccgggccccaccaccgtgcggctttctccagattattcctctctcgctgtctctgactgtctctctctgtctctgtctctgtctctctctctctcacacacacacacacacacacaaacacacatacgcacgcacgatctcacttcgatctatacactggaggattaaaacaaacaaacaaaaaaaacatttccttcgctccccctccctctccactctgagaagcagaggagccgcacggcgaggggccgcagaccgtctggaaatgcgaatcctaaagcgtttcctcgcttgcattcagctcctctgtgtttgccgcctggatacaatgcagaaattgcactcttcagtttgccagaacttagccgcaagtccacaaagaagactggaaaatttagcatttattctggatggccaggtgctcagattgggctaatggatactacagacaacagagaaaacttgttgaagagattggctggtcctatacaggagcactgaatcaaaaaaattggggaaagaaatatccaacatgtaatagcccaaaacaatctcctatcaatattgatgaagatcttacacaagtaaatgtgaatcttaagaaacttaaatttcagggttgggataaaacatcattggaaaacacattcattcataacactgggaaaacagtggaaattaatctcactaatgactaccgtgtcagcggaggagtttcagaaatggtgtttaaagcaagcaagataacttttcactggggaaaatgcaatatgtcatctgatggatcagagcatagtttagaaggacaaaaatttccacttgagatgcaaatctactgctttgatgcggaccgattttcaagttttgaggaagcagtcaaaggaaaagggaagttaagagctttatccattttgtttgaggttgggacagaagaaaatttggatttcaaagcgattattgatggagtcgaaagtgttagtcgttttgggaagcaggctgctttagatccattcatactgttgaaccttctgccaaactcaactgacaagtattacatttacaatggctcattgacatctcctccctgcacagacacagttgactggattgtttttaaagatacagttagcatctctgaaagccagttggctgttttttgtgaagttcttacaatgcaacaatctggttatgtcatgctgatggactacttacaaaacaattttcgagagcaacagtacaagttctctagacaggtgttttcctcatacactggaaaggaagagattcatgaagcagtttgtagttcagaaccagaaaatgttcaggctgacccagagaattataccagccttcttgttacatgggaaagacctcgagtcgtttatgataccatgattgagaagtttgcagttttgtaccagcagttggatggagaggaccaaaccaagcatgaatttttgacagatggctatcaagacttgggtgctattctcaataatttgctacccaatatgagttatgttcttcagatagtagccatatgcactaatggcttatatggaaaatacagcgaccaactgattgtcgacatgcctactgataatcctgaacttgatcttttccctgaattaattggaactgaagaaataatcaaggaggaggaagagggaaaagacattgaagaaggcgctattgtgaatcctggtagagacagtgctacaaaccaaatcaggaaaaaggaaccccagatttctaccacaacacactacaatcgcatagggacgaaatacaatgaagccaagactaaccgatccccaacaagaggaagtgaattctctggaaagggtgatgttcccaatacatctttaaattccacttcccaaccagtcactaaattagccacagaaaaagatatttccttgacttctcagactgtgactgaactgccacctcacactgtggaaggtacttcagcctctttaaatgatggctctaaaactgttcttagatctccacatatgaacttgtcggggactgcagaatccttaaatacagtttctataacagaatatgaggaggagagtttattgaccagtttcaagcttgatactggagctgaagattcttcaggctccagtcccgcaacttctgctatcccattcatctctgagaacatatcccaagggtatatattttcctccgaaaacccagagacaataacatatgatgtccttataccagaatctgctagaaatgcttccgaagattcaacttcatcaggttcagaagaatcactaaaggatccttctatggagggaaatgtgtggtttcctagctctacagacataacagcacagcccgatgttggatcaggcagagagagctttctccagactaattacactgagatacgtgttgatgaatctgagaagacaaccaagtccttttctgcaggcccagtgatgtcacagggtccctcagttacagatctggaaatgccacattattctacctttgcctacttcccaactgaggtaacacctcatgcttttaccccatcctccagacaacaggatttggtctccacggtcaacgtggtatactcgcagacaacccaaccggtatacaatggtgagacacctcttcaaccttcctacagtagtgaagtctttcctctagtcacccctttgttgcttgacaatcagatcctcaacactacccctgctgcttcaagtagtgattcggccttgcatgctacgcctgtatttcccagtgtcgatgtgtcatttgaatccatcctgtcttcctatgatggtgcacctttgcttccattttcctctgcttccttcagtagtgaattgtttcgccatctgcatacagtttctcaaatccttccacaagttacttcagctaccgagagtgataaggtgcccttgcatgcttctctgccagtggctgggggtgatttgctattagagcccagccttgctcagtattctgatgtgctgtccactactcatgctgcttcagagacgctggaatttggtagtgaatctggtgttctttataaaacgcttatgttttctcaagttgaaccacccagcagtgatgccatgatgcatgcacgttcttcagggcctgaaccttcttatgccttgtctgataatgagggctcccaacacatcttcactgtttcttacagttctgcaatacctgtgcatgattctgtgggtgtaacttatcagggttccttatttagcggccctagccatataccaatacctaagtcttcgttaataaccccaactgcatcattactgcagcctactcatgccctctctggtgatggggaatggtctggagcctcttctgatagtgaatttcttttacctgacacagatgggctgacagcccttaacatttcttcacctgtttctgtagctgaatttacatatacaacatctgtgtttggtgatgataataaggcgctttctaaaagtgaaataatatatggaaatgagactgaactgcaaattccttctttcaatgagatggtttacccttctgaaagcacagtcatgcccaacatgtatgataatgtaaataagttgaatgcgtctttacaagaaacctctgtttccatttctagcaccaagggcatgtttccagggtcccttgctcataccaccactaaggtttttgatcatgagattagtcaagttccagaaaataacttttcagttcaacctacacatactgtctctcaagcatctggtgacacttcgcttaaacctgtgcttagtgcaaactcagagccagcatcctctgaccctgcttctagtgaaatgttatctccttcaactcagctcttattttatgagacctcagcttcttttagtactgaagtattgctacaaccttcctttcaggcttctgatgttgacaccttgcttaaaactgttcttccagctgtgcccagtgatccaatattggttgaaacccccaaagttgataaaattagttctacaatgttgcatctcattgtatcaaattctgcttcaagtgaaaacatgctgcactctacatctgtaccagtttttgatgtgtcgcctacttctcatatgcactctgcttcacttcaaggtttgaccatttcctatgcaagtgagaaatatgaaccagttttgttaaaaagtgaaagttcccaccaagtggtaccttctttgtacagtaatgatgagttgttccaaacggccaatttggagattaaccaggcccatcccccaaaaggaaggcatgtatttgctacacctgttttatcaattgatgaaccattaaatacactaataaataagcttatacattccgatgaaattttaacctccaccaaaagttctgttactggtaaggtatttgctggtattccaacagttgcttctgatacatttgtatctactgatcattctgttcctataggaaatgggcatgttgccattacagctgtttctccccacagagatggttctgtaacctcaacaaagttgctgtttccttctaaggcaacttctgagctgagtcatagtgccaaatctgatgccggtttagtgggtggtggtgaagatggtgacactgatgatgatggtgatgatgatgatgatgacagaggtagtgatggcttatccattcataagtgtatgtcatgctcatcctatagagaatcacaggaaaaggtaatgaatgattcagacacccacgaaaacagtcttatggatcagaataatccaatctcatactcactatctgagaattctgaagaagataatagagtcacaagtgtatcctcagacagtcaaactggtatggacagaagtcctggtaaatcaccatcagcaaatgggctatcccaaaagcacaatgatggaaaagaggaaaatgacattcagactggtagtgctctgcttcctctcagccctgaatctaaagcatgggcagttctgacaagtgatgaagaaagtggatcagggcaaggtacctcagatagccttaatgagaatgagacttccacagatttcagttttgcagacactaatgaaaaagatgctgatgggatcctggcagcaggtgactcagaaataactcctggattcccacagtccccaacatcatctgttactagcgagaactcagaagtgttccacgtttcagaggcagaggccagtaatagtagccatgagtctcgtattggtctagctgaggggttggaatccgagaagaaggcagttataccccttgtgatcgtgtcagccctgacttttatctgtctagtggttcttgtgggtattctcatctactggaggaaatgcttccagactgcacacttttacttagaggacagtacatcccctagagttatatccacacctccaacacctatctttccaatttcagatgatgtcggagcaattccaataaagcactttccaaagcatgttgcagatttacatgcaagtagtgggtttactgaagaatttgagacactgaaagagttttaccaggaagtgcagagctgtactgttgacttaggtattacagcagacagctccaaccacccagacaacaagcacaagaatcgatacataaatatcgttgcctatgatcatagcagggttaagctagcacagcttgctgaaaaggatggcaaactgactgattatatcaatgccaattatgttgatggctacaacagaccaaaagcttatattgctgcccaaggcccactgaaatccacagctgaagatttctggagaatgatatgggaacataatgtggaagttattgtcatgataacaaacctcgtggagaaaggaaggagaaaatgtgatcagtactggcctgccgatgggagtgaggagtacgggaactttctggtcactcagaagagtgtgcaagtgcttgcctattatactgtgaggaattttactctaagaaacacaaaaataaaaaagggctcccagaaaggaagacccagtggacgtgtggtcacacagtatcactacacgcagtggcctgacatgggagtaccagagtactccctgccagtgctgacctttgtgagaaaggcagcctatgccaagcgccatgcagtggggcctgttgtcgtccactgcagtgctggagttggaagaacaggcacatatattgtgctagacagtatgttgcagcagattcaacacgaaggaactgtcaacatatttggcttcttaaaacacatccgttcacaaagaaattatttggtacaaactgaggagcaatatgtcttcattcatgatacactggttgaggccatacttagtaaagaaactgaggtgctggacagtcatattcatgcctatgttaatgcactcctcattcctggaccagcaggcaaaacaaagctagagaaacaattccagctcctgagccagtcaaatatacagcagagtgactattctgcagccctaaagcaatgcaacagggaaaagaatcgaacttcttctatcatccctgtggaaagatcaagggttggcatttcatccctgagtggagaaggcacagactacatcaatgcctcctatatcatgggctattaccagagcaatgaattcatcattacccagcaccctctccttcataccatcaaggatttctggaggatgatatgggaccataatgcccaactggtggttatgattcctgatggccaaaacatggcagaagatgaatttgtttactggccaaataaagatgagcctataaattgtgagagctttaaggtcactcttatggctgaagaacacaaatgtctatctaatgaggaaaaacttataattcaggactttatcttagaagctacacaggatgattatgtacttgaagtgaggcactttcagtgtcctaaatggccaaatccagatagccccattagtaaaacttttgaacttataagtgttataaaagaagaagctgccaatagggatgggcctatgattgttcatgatgagcatggaggagtgacggcaggaactttctgtgctctgacaacccttatgcaccaactagaaaaagaaaattccgtggatgtttaccaggtagccaagatgatcaatctgatgaggccaggagtctttgctgacattgagcagtatcagtttctctacaaagtgatcctcagccttgtgagcacaaggcaggaagagaatccatccacctctctggacagtaatggtgcagcattgcctgatggaaatatagctgagagcttagagtctttagtttaacacagaaaggggtgggggaactcacatctgagcattgttttcctcttcctaaaattaggcaggaaaatcagtctagttctgttatctgttgatttcccatcacctgacagtaactttcatgacataggattctgccgccaaatttatatcattaacaatgtgtgcctttttgcaagacttgtaatttacttattatgtttgaactaaaatgattgaattttacagtatttctaagaatggaattgtggtatttttttctgtattgattttaacagaaaatttcaatttatagaggttaggaattccaaactacagaaaatgtttgtttttagtgtcaaatttttagctgtatttgtagcaattatcaggtttgctagaaatataacttttaatacagtagcctgtaaataaaacactcttccatatgatattcaacattttacaactgcagtattcacctaaagtagaaataatctgttacttattgtaaatactgccctagtgtctccatggaccaaatttatatttataattgtagatttttatattttactactgagtcaagttttctagttctgtgtaattgtttagtttaatgacgtagttcattagctggtcttactctaccagttttctgacattgtattgtgttacctaagtcattaactttgtttcagcatgtaattttaacttttgtggaaaatagaaataccttcattttgaaagaagtttttatgagaataacaccttaccaaacattgttcaaatggtttttatccaaggaattgcaaaaataaatataaatattgccattaa

SEQ ID NO: 9 - protein, 1321 aa, Homo sapiens
MGAPFVWALGLLMLQMLLFVAGEQGTQDITDASERGLHMQKLGSGSVQAALAELVALPCLFTLQPRPSAARDAPRIKWTKVRTASGQRQDLPILVAKDNVVRVAKSWQGRVSLPSYPRRRANATLLLGPLRASDSGLYRCQVVRGIEDEQDLVPLEVTGVVFHYRSARDRYALTFAEAQEACRLSSAIIAAPRHLQAAFEDGFDNCDAGWLSDRTVRYPITQSRPGCYGDRSSLPGVRSYGRRNPQELYDVYCFARELGGEVFYVGPARRLTLAGARAQCRRQGAALASVGQLHLAWHEGLDQCDPGWLADGSVRYPIQTPRRRCGGPAPGVRTVYRFANRTGFPSPAERFDAYCFRAHHPTSQHGDLETPSSGDEGEILSAEGPPVRELEPTLEEEEVVTPDFQEPLVSSGEEETLILEEKQESQQTLSPTPGDPMLASWPTGEVWLSTVAPSPSDMGAGTAASSHTEVAPTDPMPRRRGRFKGLNGRYFQQQEPEPGLQGGMEASAQPPTSEAAVNQMEPPLAMAVTEMLGSGQSRSPWADLTNEVDMPGAGSAGGKSSPEPWLWPPTMVPPSISGHSRAPVLELEKAEGPSARPATPDLFWSPLEATVSAPSPAPWEAFPVATSPDLPMMAMLRGPKEWMLPHPTPISTEANRVEAHGEATATAPPSPAAETKVYSLPLSLTPTGQGGEAMPTTPESPRADFRETGETSPAQVNKAEHSSSSPWPSVNRNVAVGFVPTETATEPTGLRGIPGSESGVFDTAESPTSGLQATVDEVQDPWPSVYSKGLDASSPSAPLGSPGVFLVPKVTPNLEPWVATDEGPTVNPMDSTVTPAPSDASGIWEPGSQVFEEAESTTLSPQVALDTSIVTPLTTLEQGDKVGVPAMSTLGSSSSQPHPEPEDQVETQGTSGASVPPHQSSPLGKPAVPPGTPTAASVGESASVSSGEPTVPWDPSSTLLPVTLGIEDFELEVLAGSPGVESFWEEVASGEEPALPGTPMNAGAEEVHSDPCENNPCLHGGTCNANGTMYGCSCDQGFAGENCEIDIDDCLCSPCENGGTCIDEVNGFVCLCLPSYGGSFCEKDTEGCDRGWHKFQGHCYRYFAHRRAWEDAEKDCRRRSGHLTSVHSPEEHSFINSFGHENTWIGLNDRIVERDFQWTDNTGLQFENWRENQPDNFFAGGEDCVVMVAHESGRWNDVPCNYNLPYVCKKGTVLCGPPPAVENASLIGARKAKYNVHATVRYQCNEGFAQHHVATIRCRSNGKWDRPQIVCTKPRRSHRMRRHHHHHQHHHQHHHHKSRKERRKHKKHPTEDWEKDEGNFC

SEQ ID NO: 10 - protein, 2315 aa, Homo sapiens
MRILKRFLACIQLLCVCRLDWANGYYRQQRKLVEEIGWSYTGALNQKNWGKKYPTCNSPKQSPINIDEDLTQVNVNLKKLKFQGWDKTSLENTFIHNTGKTVEINLTNDYRVSGGVSEMVFKASKITFHWGKCNMSSDGSEHSLEGQKFPLEMQIYCFDADRFSSFEEAVKGKGKLRALSILFEVGTEENLDFKAIIDGVESVSRFGKQAALDPFILLNLLPNSTDKYYIYNGSLTSPPCTDTVDWIVFKDTVSISESQLAVFCEVLTMQQSGYVMLMDYLQNNFREQQYKFSRQVFSSYTGKEEIHEAVCSSEPENVQADPENYTSLLVTWERPRVVYDTMIEKFAVLYQQLDGEDQTKHEFLTDGYQDLGAILNNLLPNMSYVLQIVAICTNGLYGKYSDQLIVDMPTDNPELDLFPELIGTEEIIKEEEEGKDIEEGAIVNPGRDSATNQIRKKEPQISTTTHYNRIGTKYNEAKTNRSPTRGSEFSGKGDVPNTSLNSTSQPVTKLATEKDISLTSQTVTELPPHTVEGTSASLNDGSKTVLRSPHMNLSGTAESLNTVSITEYEEESLLTSFKLDTGAEDSSGSSPATSAIPFISENISQGYIFSSENPETITYDVLIPESARNASEDSTSSGSEESLKDPSMEGNVWFPSSTDITAQPDVGSGRESFLQTNYTEIRVDESEKTTKSFSAGPVMSQGPSVTDLEMPHYSTFAYFPTEVTPHAFTPSSRQQDLVSTVNVVYSQTTQPVYNGETPLQPSYSSEVFPLVTPLLLDNQILNTTPAASSSDSALHATPVFPSVDVSFESILSSYDGAPLLPFSSASFSSELFRHLHTVSQILPQVTSATESDKVPLHASLPVAGGDLLLEPSLAQYSDVLSTTHAASETLEFGSESGVLYKTLMFSQVEPPSSDAMMHARSSGPEPSYALSDNEGSQHIFTVSYSSAIPVHDSVGVTYQGSLFSGPSHIPIPKSSLITPTASLLQPTHALSGDGEWSGASSDSEFLLPDTDGLTALNISSPVSVAEFTYTTSVFGDDNKALSKSEIIYGNETELQIPSFNEMVYPSESTVMPNMYDNVNKLNASLQETSVSISSTKGMFPGSLAHTTTKVFDHEISQVPENNFSVQPTHTVSQASGDTSLKPVLSANSEPASSDPASSEMLSPSTQLLFYETSASFSTEVLLQPSFQASDVDTLLKTVLPAVPSDPILVETPKVDKISSTMLHLIVSNSASSENMLHSTSVPVFDVSPTSHMHSASLQGLTISYASEKYEPVLLKSESSHQVVPSLYSNDELFQTANLEINQAHPPKGRHVFATPVLSIDEPLNTLINKLIHSDEILTSTKSSVTGKVFAGIPTVASDTFVSTDHSVPIGNGHVAITAVSPHRDGSVTSTKLLFPSKATSELSHSAKSDAGLVGGGEDGDTDDDGDDDDDDRGSDGLSIHKCMSCSSYRESQEKVMNDSDTHENSLMDQNNPISYSLSENSEEDNRVTSVSSDSQTGMDRSPGKSPSANGLSQKHNDGKEENDIQTGSALLPLSPESKAWAVLTSDEESGSGQGTSDSLNENETSTDFSFADTNEKDADGILAAGDSEITPGFPQSPTSSVTSENSEVFHVSEAEASNSSHESRIGLAEGLESEKKAVIPLVIVSALTFICLVVLVGILIYWRKCFQTAHFYLEDSTSPRVISTPPTPIFPISDDVGAIPIKHFPKHVADLHASSGFTEEFETLKEFYQEVQSCTVDLGITADSSNHPDNKHKNRYINIVAYDHSRVKLAQLAEKDGKLTDYINANYVDGYNRPKAYIAAQGPLKSTAEDFWRMIWEHNVEVIVMITNLVEKGRRKCDQYWPADGSEEYGNFLVTQKSVQVLAYYTVRNFTLRNTKIKKGSQKGRPSGRVVTQYHYTQWPDMGVPEYSLPVLTFVRKAAYAKRHAVGPVVVHCSAGVGRTGTYIVLDSMLQQIQHEGTVNIFGFLKHIRSQRNYLVQTEEQYVFIHDTLVEAILSKETEVLDSHIHAYVNALLIPGPAGKTKLEKQFQLLSQSNIQQSDYSAALKQCNREKNRTSSIIPVERSRVGISSLSGEGTDYINASYIMGYYQSNEFIITQHPLLHTIKDFWRMIWDHNAQLVVMIPDGQNMAEDEFVYWPNKDEPINCESFKVTLMAEEHKCLSNEEKLIIQDFILEATQDDYVLEVRHFQCPKWPNPDSPISKTFELISVIKEEAANRDGPMIVHDEHGGVTAGTFCALTTLMHQLEKENSVDVYQVAKMINLMRPGVFADIEQYQFLYKVILSLVSTRQEENPSTSLDSNGAALPDGNIAESLESLV

SEQ ID NO: 11 - protein, 1455 aa, Homo sapiens
MRILKRFLACIQLLCVCRLDWANGYYRQQRKLVEEIGWSYTGALNQKNWGKKYPTCNSPKQSPINIDEDLTQVNVNLKKLKFQGWDKTSLENTFIHNTGKTVEINLTNDYRVSGGVSEMVFKASKITFHWGKCNMSSDGSEHSLEGQKFPLEMQIYCFDADRFSSFEEAVKGKGKLRALSILFEVGTEENLDFKAIIDGVESVSRFGKQAALDPFILLNLLPNSTDKYYIYNGSLTSPPCTDTVDWIVFKDTVSISESQLAVFCEVLTMQQSGYVMLMDYLQNNFREQQYKFSRQVFSSYTGKEEIHEAVCSSEPENVQADPENYTSLLVTWERPRVVYDTMIEKFAVLYQQLDGEDQTKHEFLTDGYQDLGAILNNLLPNMSYVLQIVAICTNGLYGKYSDQLIVDMPTDNPELDLFPELIGTEEIIKEEEEGKDIEEGAIVNPGRDSATNQIRKKEPQISTTTHYNRIGTKYNEAKTNRSPTRGSEFSGKGDVPNTSLNSTSQPVTKLATEKDISLTSQTVTELPPHTVEGTSASLNDGSKTVLRSPHMNLSGTAESLNTVSITEYEEESLLTSFKLDTGAEDSSGSSPATSAIPFISENISQGYIFSSENPETITYDVLIPESARNASEDSTSSGSEESLKDPSMEGNVWFPSSTDITAQPDVGSGRESFLQTNYTEIRVDESEKTTKSFSAGPVMSQGPSVTDLEMPHYSTFAYFPTEVTPHAFTPSSRQQDLVSTVNVVYSQTTQPVYNEASNSSHESRIGLAEGLESEKKAVIPLVIVSALTFICLVVLVGILIYWRKCFQTAHFYLEDSTSPRVISTPPTPIFPISDDVGAIPIKHFPKHVADLHASSGFTEEFETLKEFYQEVQSCTVDLGITADSSNHPDNKHKNRYINIVAYDHSRVKLAQLAEKDGKLTDYINANYVDGYNRPKAYIAAQGPLKSTAEDFWRMIWEHNVEVIVMITNLVEKGRRKCDQYWPADGSEEYGNFLVTQKSVQVLAYYTVRNFTLRNTKIKKGSQKGRPSGRVVTQYHYTQWPDMGVPEYSLPVLTFVRKAAYAKRHAVGPVVVHCSAGVGRTGTYIVLDSMLQQIQHEGTVNIFGFLKHIRSQRNYLVQTEEQYVFIHDTLVEAILSKETEVLDSHIHAYVNALLIPGPAGKTKLEKQFQLLSQSNIQQSDYSAALKQCNREKNRTSSIIPVERSRVGISSLSGEGTDYINASYIMGYYQSNEFIITQHPLLHTIKDFWRMIWDHNAQLVVMIPDGQNMAEDEFVYWPNKDEPINCESFKVTLMAEEHKCLSNEEKLIIQDFILEATQDDYVLEVRHFQCPKWPNPDSPISKTFELISVIKEEAANRDGPMIVHDEHGGVTAGTFCALTTLMHQLEKENSVDVYQVAKMINLMRPGVFADIEQYQFLYKVILSLVSTRQEENPSTSLDSNGAALPDGNIAESLESLV

SEQ ID NO: 12 - protein, 1448 aa, Homo sapiens
MRILKRFLACIQLLCVCRLDWANGYYRQQRKLVEEIGWSYTGALNQKNWGKKYPTCNSPKQSPINIDEDLTQVNVNLKKLKFQGWDKTSLENTFIHNTGKTVEINLTNDYRVSGGVSEMVFKASKITFHWGKCNMSSDGSEHSLEGQKFPLEMQIYCFDADRFSSFEEAVKGKGKLRALSILFEVGTEENLDFKAIIDGVESVSRFGKQAALDPFILLNLLPNSTDKYYIYNGSLTSPPCTDTVDWIVFKDTVSISESQLAVFCEVLTMQQSGYVMLMDYLQNNFREQQYKFSRQVFSSYTGKEEIHEAVCSSEPENVQADPENYTSLLVTWERPRVVYDTMIEKFAVLYQQLDGEDQTKHEFLTDGYQDLGAILNNLLPNMSYVLQIVAICTNGLYGKYSDQLIVDMPTDNPELDLFPELIGTEEIIKEEEEGKDIEEGAIVNPGRDSATNQIRKKEPQISTTTHYNRIGTKYNEAKTNRSPTRGSEFSGKGDVPNTSLNSTSQPVTKLATEKDISLTSQTVTELPPHTVEGTSASLNDGSKTVLRSPHMNLSGTAESLNTVSITEYEEESLLTSFKLDTGAEDSSGSSPATSAIPFISENISQGYIFSSENPETITYDVLIPESARNASEDSTSSGSEESLKDPSMEGNVWFPSSTDITAQPDVGSGRESFLQTNYTEIRVDESEKTTKSFSAGPVMSQGPSVTDLEMPHYSTFAYFPTEVTPHAFTPSSRQQDLVSTVNVVYSQTTQPVYNEASNSSHESRIGLAEGLESEKKAVIPLVIVSALTFICLVVLVGILIYWRKCFQTAHFYLEDSTSPRVISTPPTPIFPISDDVGAIPIKHFPKHVADLHASSGFTEEFEEVQSCTVDLGITADSSNHPDNKHKNRYINIVAYDHSRVKLAQLAEKDGKLTDYINANYVDGYNRPKAYIAAQGPLKSTAEDFWRMIWEHNVEVIVMITNLVEKGRRKCDQYWPADGSEEYGNFLVTQKSVQVLAYYTVRNFTLRNTKIKKGSQKGRPSGRVVTQYHYTQWPDMGVPEYSLPVLTFVRKAAYAKRHAVGPVVVHCSAGVGRTGTYIVLDSMLQQIQHEGTVNIFGFLKHIRSQRNYLVQTEEQYVFIHDTLVEAILSKETEVLDSHIHAYVNALLIPGPAGKTKLEKQFQLLSQSNIQQSDYSAALKQCNREKNRTSSIIPVERSRVGISSLSGEGTDYINASYIMGYYQSNEFIITQHPLLHTIKDFWRMIWDHNAQLVVMIPDGQNMAEDEFVYWPNKDEPINCESFKVTLMAEEHKCLSNEEKLIIQDFILEATQDDYVLEVRHFQCPKWPNPDSPISKTFELISVIKEEAANRDGPMIVHDEHGGVTAGTFCALTTLMHQLEKENSVDVYQVAKMINLMRPGVFADIEQYQFLYKVILSLVSTRQEENPSTSLDSNGAALPDGNIAESLESLV

SEQ ID NO: 13 - protein, 2308 aa, Homo sapiens
MRILKRFLACIQLLCVCRLDWANGYYRQQRKLVEEIGWSYTGALNQKNWGKKYPTCNSPKQSPINIDEDLTQVNVNLKKLKFQGWDKTSLENTFIHNTGKTVEINLTNDYRVSGGVSEMVFKASKITFHWGKCNMSSDGSEHSLEGQKFPLEMQIYCFDADRFSSFEEAVKGKGKLRALSILFEVGTEENLDFKAIIDGVESVSRFGKQAALDPFILLNLLPNSTDKYYIYNGSLTSPPCTDTVDWIVFKDTVSISESQLAVFCEVLTMQQSGYVMLMDYLQNNFREQQYKFSRQVFSSYTGKEEIHEAVCSSEPENVQADPENYTSLLVTWERPRVVYDTMIEKFAVLYQQLDGEDQTKHEFLTDGYQDLGAILNNLLPNMSYVLQIVAICTNGLYGKYSDQLIVDMPTDNPELDLFPELIGTEEIIKEEEEGKDIEEGAIVNPGRDSATNQIRKKEPQISTTTHYNRIGTKYNEAKTNRSPTRGSEFSGKGDVPNTSLNSTSQPVTKLATEKDISLTSQTVTELPPHTVEGTSASLNDGSKTVLRSPHMNLSGTAESLNTVSITEYEEESLLTSFKLDTGAEDSSGSSPATSAIPFISENISQGYIFSSENPETITYDVLIPESARNASEDSTSSGSEESLKDPSMEGNVWFPSSTDITAQPDVGSGRESFLQTNYTEIRVDESEKTTKSFSAGPVMSQGPSVTDLEMPHYSTFAYFPTEVTPHAFTPSSRQQDLVSTVNVVYSQTTQPVYNGETPLQPSYSSEVFPLVTPLLLDNQILNTTPAASSSDSALHATPVFPSVDVSFESILSSYDGAPLLPFSSASFSSELFRHLHTVSQILPQVTSATESDKVPLHASLPVAGGDLLLEPSLAQYSDVLSTTHAASETLEFGSESGVLYKTLMFSQVEPPSSDAMMHARSSGPEPSYALSDNEGSQHIFTVSYSSAIPVHDSVGVTYQGSLFSGPSHIPIPKSSLITPTASLLQPTHALSGDGEWSGASSDSEFLLPDTDGLTALNISSPVSVAEFTYTTSVFGDDNKALSKSEIIYGNETELQIPSFNEMVYPSESTVMPNMYDNVNKLNASLQETSVSISSTKGMFPGSLAHTTTKVFDHEISQVPENNFSVQPTHTVSQASGDTSLKPVLSANSEPASSDPASSEMLSPSTQLLFYETSASFSTEVLLQPSFQASDVDTLLKTVLPAVPSDPILVETPKVDKISSTMLHLIVSNSASSENMLHSTSVPVFDVSPTSHMHSASLQGLTISYASEKYEPVLLKSESSHQVVPSLYSNDELFQTANLEINQAHPPKGRHVFATPVLSIDEPLNTLINKLIHSDEILTSTKSSVTGKVFAGIPTVASDTFVSTDHSVPIGNGHVAITAVSPHRDGSVTSTKLLFPSKATSELSHSAKSDAGLVGGGEDGDTDDDGDDDDDDRGSDGLSIHKCMSCSSYRESQEKVMNDSDTHENSLMDQNNPISYSLSENSEEDNRVTSVSSDSQTGMDRSPGKSPSANGLSQKHNDGKEENDIQTGSALLPLSPESKAWAVLTSDEESGSGQGTSDSLNENETSTDFSFADTNEKDADGILAAGDSEITPGFPQSPTSSVTSENSEVFHVSEAEASNSSHESRIGLAEGLESEKKAVIPLVIVSALTFICLVVLVGILIYWRKCFQTAHFYLEDSTSPRVISTPPTPIFPISDDVGAIPIKHFPKHVADLHASSGFTEEFEEVQSCTVDLGITADSSNHPDNKHKNRYINIVAYDHSRVKLAQLAEKDGKLTDYINANYVDGYNRPKAYIAAQGPLKSTAEDFWRMIWEHNVEVIVMITNLVEKGRRKCDQYWPADGSEEYGNFLVTQKSVQVLAYYTVRNFTLRNTKIKKGSQKGRPSGRVVTQYHYTQWPDMGVPEYSLPVLTFVRKAAYAKRHAVGPVVVHCSAGVGRTGTYIVLDSMLQQIQHEGTVNIFGFLKHIRSQRNYLVQTEEQYVFIHDTLVEAILSKETEVLDSHIHAYVNALLIPGPAGKTKLEKQFQLLSQSNIQQSDYSAALKQCNREKNRTSSIIPVERSRVGISSLSGEGTDYINASYIMGYYQSNEFIITQHPLLHTIKDFWRMIWDHNAQLVVMIPDGQNMAEDEFVYWPNKDEPINCESFKVTLMAEEHKCLSNEEKLIIQDFILEATQDDYVLEVRHFQCPKWPNPDSPISKTFELISVIKEEAANRDGPMIVHDEHGGVTAGTFCALTTLMHQLEKENSVDVYQVAKMINLMRPGVFADIEQYQFLYKVILSLVSTRQEENPSTSLDSNGAALPDGNIAESLESLV

SEQ ID NO: 14 - protein, 2301 aa, Homo sapiens
MARCSDWANGYYRQQRKLVEEIGWSYTGALNQKNWGKKYPTCNSPKQSPINIDEDLTQVNVNLKKLKFQGWDKTSLENTFIHNTGKTVEINLTNDYRVSGGVSEMVFKASKITFHWGKCNMSSDGSEHSLEGQKFPLEMQIYCFDADRFSSFEEAVKGKGKLRALSILFEVGTEENLDFKAIIDGVESVSRFGKQAALDPFILLNLLPNSTDKYYIYNGSLTSPPCTDTVDWIVFKDTVSISESQLAVFCEVLTMQQSGYVMLMDYLQNNFREQQYKFSRQVFSSYTGKEEIHEAVCSSEPENVQADPENYTSLLVTWERPRVVYDTMIEKFAVLYQQLDGEDQTKHEFLTDGYQDLGAILNNLLPNMSYVLQIVAICTNGLYGKYSDQLIVDMPTDNPELDLFPELIGTEEIIKEEEEGKDIEEGAIVNPGRDSATNQIRKKEPQISTTTHYNRIGTKYNEAKTNRSPTRGSEFSGKGDVPNTSLNSTSQPVTKLATEKDISLTSQTVTELPPHTVEGTSASLNDGSKTVLRSPHMNLSGTAESLNTVSITEYEEESLLTSFKLDTGAEDSSGSSPATSAIPFISENISQGYIFSSENPETITYDVLIPESARNASEDSTSSGSEESLKDPSMEGNVWFPSSTDITAQPDVGSGRESFLQTNYTEIRVDESEKTTKSFSAGPVMSQGPSVTDLEMPHYSTFAYFPTEVTPHAFTPSSRQQDLVSTVNVVYSQTTQPVYNGETPLQPSYSSEVFPLVTPLLLDNQILNTTPAASSSDSALHATPVFPSVDVSFESILSSYDGAPLLPFSSASFSSELFRHLHTVSQILPQVTSATESDKVPLHASLPVAGGDLLLEPSLAQYSDVLSTTHAASETLEFGSESGVLYKTLMFSQVEPPSSDAMMHARSSGPEPSYALSDNEGSQHIFTVSYSSAIPVHDSVGVTYQGSLFSGPSHIPIPKSSLITPTASLLQPTHALSGDGEWSGASSDSEFLLPDTDGLTALNISSPVSVAEFTYTTSVFGDDNKALSKSEIIYGNETELQIPSFNEMVYPSESTVMPNMYDNVNKLNASLQETSVSISSTKGMFPGSLAHTTTKVFDHEISQVPENNFSVQPTHTVSQASGDTSLKPVLSANSEPASSDPASSEMLSPSTQLLFYETSASFSTEVLLQPSFQASDVDTLLKTVLPAVPSDPILVETPKVDKISSTMLHLIVSNSASSENMLHSTSVPVFDVSPTSHMHSASLQGLTISYASEKYEPVLLKSESSHQVVPSLYSNDELFQTANLEINQAHPPKGRHVFATPVLSIDEPLNTLINKLIHSDEILTSTKSSVTGKVFAGIPTVASDTFVSTDHSVPIGNGHVAITAVSPHRDGSVTSTKLLFPSKATSELSHSAKSDAGLVGGGEDGDTDDDGDDDDDDRGSDGLSIHKCMSCSSYRESQEKVMNDSDTHENSLMDQNNPISYSLSENSEEDNRVTSVSSDSQTGMDRSPGKSPSANGLSQKHNDGKEENDIQTGSALLPLSPESKAWAVLTSDEESGSGQGTSDSLNENETSTDFSFADTNEKDADGILAAGDSEITPGFPQSPTSSVTSENSEVFHVSEAEASNSSHESRIGLAEGLESEKKAVIPLVIVSALTFICLVVLVGILIYWRKCFQTAHFYLEDSTSPRVISTPPTPIFPISDDVGAIPIKHFPKHVADLHASSGFTEEFETLKEFYQEVQSCTVDLGITADSSNHPDNKHKNRYINIVAYDHSRVKLAQLAEKDGKLTDYINANYVDGYNRPKAYIAAQGPLKSTAEDFWRMIWEHNVEVIVMITNLVEKGRRKCDQYWPADGSEEYGNFLVTQKSVQVLAYYTVRNFTLRNTKIKKGSQKGRPSGRVVTQYHYTQWPDMGVPEYSLPVLTFVRKAAYAKRHAVGPVVVHCSAGVGRTGTYIVLDSMLQQIQHEGTVNIFGFLKHIRSQRNYLVQTEEQYVFIHDTLVEAILSKETEVLDSHIHAYVNALLIPGPAGKTKLEKQFQLLSQSNIQQSDYSAALKQCNREKNRTSSIIPVERSRVGISSLSGEGTDYINASYIMGYYQSNEFIITQHPLLHTIKDFWRMIWDHNAQLVVMIPDGQNMAEDEFVYWPNKDEPINCESFKVTLMAEEHKCLSNEEKLIIQDFILEATQDDYVLEVRHFQCPKWPNPDSPISKTFELISVIKEEAANRDGPMIVHDEHGGVTAGTFCALTTLMHQLEKENSVDVYQVAKMINLMRPGVFADIEQYQFLYKVILSLVSTRQEENPSTSLDSNGAALPDGNIAESLESLV